SEQUENCE LISTING (56 sequences)

SEQ ID NO: 1 - protein, 6 aa, Artificial Sequence
  consensus sequence
XXGFXX

SEQ ID NO: 2 - protein, 6 aa, Artificial Sequence
  preferred peptide
DEGXWE

SEQ ID NO: 3 - protein, 6 aa, Artificial Sequence
  preferred peptide
DEGXWE

SEQ ID NO: 4 - protein, 6 aa, Artificial Sequence
  preferred peptide
DEGXWD

SEQ ID NO: 5 - protein, 6 aa, Artificial Sequence
  preferred peptide
DDGXWD

SEQ ID NO: 6 - protein, 5 aa, Artificial Sequence
  preferred peptide
EGXWE

SEQ ID NO: 7 - protein, 6 aa, Artificial Sequence
  preferred peptide
DEGXXE

SEQ ID NO: 8 - protein, 5 aa, Artificial Sequence
  preferred peptide
EGXXE

SEQ ID NO: 9 - protein, 21 aa, phosphopeptide substrate
  misc_feature 12, 16: Xaa is phosphorylated serine
KKERLLDDRHDXGLDXMKDEE

SEQ ID NO: 10 - protein, 7 aa, Artificial Sequence
  optimisation starting sequence
LDXGIHS

SEQ ID NO: 11 - protein, 6 aa, vpu phosphodegeneron
  misc_feature 2: Xaa is phosphorylated serine
DXGIHS

SEQ ID NO: 12 - protein, 6 aa, Artificial Sequence
  binding peptide
DXGIFE

SEQ ID NO: 13 - protein, 6 aa, Artificial Sequence
  binding peptide
DEGIFE

SEQ ID NO: 14 - protein, 15 aa, Artificial Sequence
  binding peptide
ERAEDXGNEXEGEIS

SEQ ID NO: 15 - protein, 15 aa, Artificial Sequence
  binding peptide
ERAEDAGNEXEGEIS

SEQ ID NO: 16 - protein, 15 aa, Artificial Sequence
  binding peptide
ERAEDXGNEXEGEHS

SEQ ID NO: 17 - protein, 15 aa, Artificial Sequence
  binding peptide
ERADDXGNEXEGEIS

SEQ ID NO: 18 - protein, 6 aa, Artificial Sequence
  binding peptide
DEGIFE

SEQ ID NO: 19 - protein, 6 aa, Artificial Sequence
  binding peptide
DEGIFD

SEQ ID NO: 20 - protein, 6 aa, Artificial Sequence
  binding peptide
DNGIFR

SEQ ID NO: 21 - protein, 6 aa, Artificial Sequence
  binding peptide
DEGFFE

SEQ ID NO: 22 - protein, 6 aa, Artificial Sequence
  binding peptide
DNGFFR

SEQ ID NO: 23 - protein, 5 aa, Artificial Sequence
  binding peptide
EGIFE

SEQ ID NO: 24 - protein, 5 aa, Artificial Sequence
  binding peptide
EGFFE

SEQ ID NO: 25 - protein, 6 aa, Artificial Sequence
  binding peptide
DEGYFE

SEQ ID NO: 26 - protein, 6 aa, Artificial Sequence
  binding peptide
EXGIFE

SEQ ID NO: 27 - protein, 6 aa, Artificial Sequence
  binding peptide
DXGIFH

SEQ ID NO: 28 - protein, 6 aa, Artificial Sequence
  binding peptide
DXGNFE

SEQ ID NO: 29 - protein, 8 aa, Artificial Sequence
  binding peptide
DDXSGIHS

SEQ ID NO: 30 - protein, 8 aa, Artificial Sequence
  binding peptide
LDXSGIHS

SEQ ID NO: 31 - protein, 7 aa, Artificial Sequence
  binding peptide
GDXGIHS

SEQ ID NO: 32 - protein, 7 aa, Artificial Sequence
  binding peptide
ADXGIHS

SEQ ID NO: 33 - protein, 7 aa, Artificial Sequence
  binding peptide
VDXGIHS

SEQ ID NO: 34 - protein, 6 aa, Artificial Sequence
  control peptide
DAGIFE

SEQ ID NO: 35 - protein, 5 aa, Artificial Sequence
  control peptide
AGIFE

SEQ ID NO: 36 - protein, 5 aa, Artificial Sequence
  control peptide
AGFFE

SEQ ID NO: 37 - protein, 6 aa, Artificial Sequence
  control peptide
DAGIFR

SEQ ID NO: 38 - protein, 6 aa, Artificial Sequence
  control peptide
DAGIFD

SEQ ID NO: 39 - protein, 6 aa, Artificial Sequence
  control peptide
DAGIFE

SEQ ID NO: 40 - protein, 6 aa, Artificial Sequence
  control peptide
DAGFFE

SEQ ID NO: 41 - protein, 6 aa, Artificial Sequence
  control peptide
DAGYFE

SEQ ID NO: 42 - protein, 6 aa, Artificial Sequence
  control peptide
EAGIFE

SEQ ID NO: 43 - protein, 6 aa, Artificial Sequence
  control peptide
DAGIFH

SEQ ID NO: 44 - protein, 6 aa, Artificial Sequence
  control peptidesynthetic
DAGNFE

SEQ ID NO: 45 - protein, 6 aa, Artificial Sequence
  control peptide
DAGIHS

SEQ ID NO: 46 - protein, 8 aa, Artificial Sequence
  control peptide
DDASGIHS

SEQ ID NO: 47 - protein, 8 aa, Artificial Sequence
  control peptide
LDASGIHS

SEQ ID NO: 48 - protein, 7 aa, Artificial Sequence
  control peptide
GDAGIHS

SEQ ID NO: 49 - protein, 7 aa, Artificial Sequence
  control peptide
ADAGIHS

SEQ ID NO: 50 - protein, 7 aa, Artificial Sequence
  control peptide
VDAGIHS

SEQ ID NO: 51 - protein, 5 aa, Artificial Sequence
  binding peptide
EGXHE

SEQ ID NO: 52 - protein, 6 aa, Artificial Sequence
  binding peptide
DEGXXE

SEQ ID NO: 53 - protein, 5 aa, Artificial Sequence
  binding peptide
EGIXE

SEQ ID NO: 54 - protein, 5 aa, Artificial Sequence
  binding peptide
EGXXQ

SEQ ID NO: 55 - protein, 5 aa, Artificial Sequence
  binding peptide
EGXXE

SEQ ID NO: 56 - protein, 6 aa, Artificial Sequence
  binding peptide
DEGXWD

(19) United States
(12) Patent Application Publication
(10) Pub. No.: US 2014/0315784 A1
(43) Pub. Date: Oct. 23, 2014

(54) Binding Inhibitors of the Beta. Transducin Repeat-Containing Protein

(71) Applicants: Mark Bradley, Edinburgh (GB); Jeffrey George Andrew Walton, Edinburgh (GB); Sunay Vijaykumar Chankeshwara, Edinburgh (GB); Mazen Sleiman, Edinburgh (GB); George S. Baillie, Glasgow (GB); Lucien Gibson, Glasgow (GB)

(72) Inventors: Mark Bradley, Edinburgh (GB); Jeffrey George Andrew Walton, Edinburgh (GB); Sunay Vijaykumar Chankeshwara, Edinburgh (GB); Mazen Sleiman, Edinburgh (GB); George S. Baillie, Glasgow (GB); Lucien Gibson, Glasgow (GB)

(21) Appl. No.: 14/127,430

(22) PCT Filed: Jun. 27, 2012
(86) PCT No.: PCT/GB2012/051507

§ 371 Date: Jun. 30, 2014

(30) Foreign Application Priority Data
Jun. 27, 2011 (GB) 1110938.6

(51) Int. Cl. C07K 14/00; C07K 7/08; C07K 7/64; C07K 7/06

(52) U.S. Cl. CPC C07K 14/00; C07K 7/06; C07K 7/08; C07K 7/64
USPC 514/1.3; 530/329; 514/21.8; 530/326; 514/21.4; 514/21.5; 530/328; 514/21.7; 530/317

(57) ABSTRACT

The present invention relates to compounds which bind to Beta Trans-ducin repeat-containing protein (PTrCP), and modulate the activity of 13TrCP. In particular, the invention relates to compounds which demonstrate optimised binding to PTrCP. The invention also relates to pharmaceutical compositions comprising such compounds and the use of such compounds as medicaments, specifically for the treatment of disorders associated with aberrant protein degradation, such as cancer. The preferred binding inhibitors are peptides derived from the motive DSGXXS, e.g. DEGFWE, DDGFWD and Succinyl-EGFWE.

### FIELD OF THE INVENTION

The present invention relates to compounds which bind to Beta Transducin repeat-containing protein (βTrCP), and modulate the activity of βTrCP. In particular, the invention relates to compounds which demonstrate optimised binding to βTrCP. The invention also relates to pharmaceutical compositions comprising such compounds and the use of such compounds as medicaments, specifically for the treatment of disorders associated with aberrant protein degradation, such as cancer.

### BACKGROUND OF THE INVENTION

In order to maintain the delicate homeostatic balance of a cell, unneeded or damaged proteins must be degraded. Protein degradation is performed by the proteasome, which dismantles unwanted proteins into small peptides of about eight amino acids in length. These peptides are then further degraded by proteases in the cell, and the resulting amino acids are used to synthesise new proteins.

To ensure that only unwanted proteins are degraded, and that healthy functioning proteins remain intact, target proteins are tagged for degradation by the ubiquitin proteasome system (UPS). The aim of the UPS is to attach a chain of approximately four ubiquitin monomers to any unwanted proteins in order to direct entry of the target protein into the proteasome.

The major components of the UPS are ubiauitin-activating enzymes (E1), ubiquitin-coniugating enzymes (E2) and ubiuitin ligaes (E3). There are several members of each of these groups of enzymes, which generally recognise different groups of target proteins.

The first step of the UPS is the hydrolysation of ATP by an ubiquitin-activatina enzyme in order to facilitate the adenylation of an ubiquitin molecule. Following this, the ubiquitin molecule is transferred to a cysteine residue in the active site of the ubiquitin-activating enzyme at the same time as a second ubiquitin molecule is adenylated. The second adenylated ubiquitin molecule is subsequently transferred to a cysteine residue in the active site of an ubiquitin-coniugating enzyme. The final step requires the recognition of the target protein by an ubiquitin ligase, which catalyses the transfer of the ubiquitin molecule from the ubiquitin-coniugating enzyme to the target protein. Following the addition of four ubiquitin molecules, the target protein is recognised by the proteasome, and sent for degradation.

βTrCP is an E3 ubiquitin ligase forming part of the UPS. It recognises a variety of target proteins, including inhibitor of nuclear factor κB (IκB), β-catenin, REST (repressor-element-1-silencing transcription factor), CDC25A/B, ATF4 (Activating Transcription Factor 4), and pro-caspase 3 and is known to function by binding to a phosphodegeneron motif DSGXXS in which the two serines are phosphorylated.

βTrCP is involved in apoptotic regulation through the targeted degradation of pro-apoptotic factors. βTrCP has been shown to be over-expressed in a variety of cancers including colorectal cancer, chemoresistant pancreatic cancer, hepatoblastomas and breast cancer. Human hepatocellular carcinomas (HCCs), pancreatic tumours and melanomas have also been shown to display an aberrant loss of IB, which is thought to be caused by βTrCP over-expression. This over-expression increases the degradation of pro-apoptotic factors, leading to a reduction in apoptotic cell death and subsequent aberrant cell growth.

Inhibition of βTrCP prevents the degradation of pro-apoptotic factors such as IB and programmed cell death 4 (PDCD4). This has been shown to induce apoptosis in human malignant melanoma, breast cancer and prostate cancer cells, augmenting the cytotoxic effects of anticancer drugs and ionizing radiation.

### SUMMARY OF THE INVENTION

The inventors hypothesised that compounds that bond PTrCP may be able to prevent βTrCP binding to its substrates, thus preventing the ubiquitination of target proteins. The prolonged presence in a cell of pro-apoptotic factors will increase cellular apoptosis, providing a useful tool for the treatment of disorders associated with aberrant protein degradation such as hyperproliferative disorders including cancer.

The inventors have therefore designed a series of compounds which bind PTrCP and which will be therapeutically useful.

### Compounds and Modifled Peptides

The present invention relates to compounds which bind βTrCP.

Accordingly, in the first aspect, the invention provides a compound of Formula Ia:

X¹—X²—X³—X⁴—X⁵—X⁶—X⁷ Formula Ia

wherein,
X¹ is a group A¹-B—Z¹—;

X² is a group —N(R^{a})—Y¹(-L¹-A²)-Z²—;

X³ is a group —N(R^{b})—Y²—Z³—;

X⁴ is a group —N(R^{c})—Y³(-L²-A³)-Z⁴—; or X⁴ is a group —N(R^{c})—Y³(-L²)-Z⁴

X⁵ is a group —N(R^{d})—Y⁴(-L³-A⁴)-Z⁵—;

X⁶ is a group —N(R)—Y⁵(-L⁴-A⁵)-Z⁶—;

X⁷ is a group —N(R^{N1})(R^{N2});

wherein,

B is C₁-C₁₀ alkyl, C₂-C₁₀ alkenyl, C₂-C₁₀ alkynyl or aryl;

wherein, B may be substituted with one or more R^{E}, wherein R^{E} is selected from the group consisting of C₁-C₄ alkyl, —NH₂, —NH(R^{N2}) and —N(R^{N2})₂;

R^{a}, R^{b}, R^{c}, R^{d}, and R^{e} are each independently selected from the group consisting of —H, C₁-C₁₀ alkyl, aryl and heteroaryl;

L¹, L², L³ and L⁴ are each independently C₀-C₅ alkyl, C₂-C₅ alkenyl or C₂-C₅ alkynyl;

wherein,

L¹ may be substituted with one or more R^{L3}, wherein R^{L3} is C₁-C₄ alkyl;

L² may be substituted with one or more R^{L2}, wherein R^{L2} is C₁-C₄ alkyl or C₂-C₄ alkenyl;

L³ may be substituted with one or more R^{L3}, wherein R^{L3} is C₁-C₄ alkyl;

L⁴ may be substituted with one or more R^{L4}, wherein R^{L4} is C₁-C₄ alkyl;

Y¹, Y³, Y⁴ and Y⁵ are each independently CH or N;

Y² is CF₂, CH₂, N(R^{Y2}) or O; wherein, R^{Y2} is —H or C₁-C₄ alkyl;

Z¹ is a bond, C═O, C═S, CH₂, S═O, S(O)₂, C═N(C₁-C₄ alkyl) or C═NH;

Z², Z³, Z⁴, Z⁵, and Z⁶ are independently selected from the group consisting of C═O, C═S, CH₂, S═O, S(O)₂, C═N(C₁-C₄ alkyl) and C═NH;

A¹ and A⁵ are each independently carboxylic acid (—CO₂H) or a bioisostere thereof and A² is a carboxylic acid (—CO₂H) or a bioisostere thereof or —C(O)N(R^{N1})₂;

wherein,

A¹ may be substituted with one or more R^{A1}, wherein R^{A1} is selected from the group consisting of —H, C₁-C₄ alkyl, C₂-C₄ alkenyl and aryl;

A² may be substituted with one or more R^{A2}, wherein R^{A2} is selected from the group consisting of —H, C₁-C₄ alkyl, C₂-C₄ alkenyl and aryl;

A⁵ may be substituted with one or more R^{A}, wherein RAS is selected from the group consisting of —H, C₁-C₄ alkyl, C₂-C₄ alkenyl and aryl;

A³ and A⁴ are each independently aryl or heteroaryl; wherein, A³ may be substituted with one or more R^{A3}, wherein, R^{A3} is selected from the group consisting of —H, —F, —Cl, —Br, —I, —OH, —O(C₁-C₁₀ alkyl), —CN, —NO₂, —CF₃, —OCF₃, —CO₂H, —C₁-C₁₀ alkyl, —NH₂, —NH(C₁-C₂ alkyl) and —N(C₁-C₂ alkyl)₂;

A⁴ may be substituted with one or more R^{A4}, wherein R^{A4} is selected from the group consisting of —H, —F, —Cl, —Br, —I, —OH, —O(C₁-C₁₀ alkyl), —CN, —NO₂, —CF₃, —OCF₃, —CO₂H, —C₁-C₁₀ alkyl, —NH₂, —NH(C₁-C₂ alkyl), —N(C₁-C₂ alkyl)₂;

R^{N1} is selected from the group consisting of —H, C₁-C₁₀ alkyl and aryl;

R^{N2} is selected from the group consisting of R^{N1}, —(CH₂)₀₋₁₀—(Z⁷)₀₋₁-A^{a}, —(CH₂O)₀₋₁₀—CH₂—(Z⁷)₀₋₁-A^{a}, —(CH₂CH₂O)₁₋₁₀—CH₂CH₃, —(CH₂CH₂O)₁₋₁₀—(CH₂)₁₋₃—(Z⁷)₀₋₁-A^{a};

wherein,

Z⁷ is (C═O);

A^{a} is —OH, —NH₂, —C(O)NH₂, a cholesteryl derivative, a chain of one or more non-naturally occurring amino acids, or a chain of one or more naturally occurring amino acids or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids;

wherein,
when the compound of Formula Ia is substituted with an amino/amine group, said amino/amine group may be optionally capped, by replacement of a H atom, with a capping group.

Formula Ia may also be represented by Formula Ib:

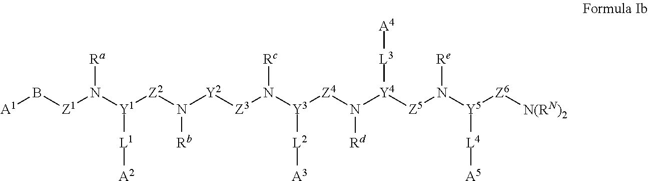

In a second aspect, the invention provides a modified peptide comprising a sequence of amino acids:

X¹-E/D/pS-G-X⁴-X⁵-E/D/pS-NHR^{N2} Formula Ic

wherein,
each of the amino acids are selected from L-amino acids, D-amino acids, aza-amino acids and substituted amino acids; and
wherein,

X¹ is a group A¹-B—Z¹—;

X⁴ is a group —N(R^{c})—Y³(-L²-A³)-Z⁴—;

X⁵ is a group —N(Rd)—Y⁴(-L³-A⁴)-Z⁵—;

wherein,

B, R^{c}, R^{d}, L², L³, Y³, Y⁴, Z¹, Z⁴, Z⁵, A¹, A³, A⁴, and R^{N2} are as previously defined.

In a third aspect, the invention provides a prodrug comprising a methyl, ethyl, propyl, butyl, pentyl, cyclopentyl, hexyl, benzyl, aryl or heteroaryl ester of a compound of Formula Ia or a modified peptide of Formula Ic.

In a fourth aspect, the invention provides a prodrug comprising a —CO₂(CH₂CH₂O)₁₋₁₀CH₂CH₃ ester of a compound of Formula Ia or a modified peptide of Formula Ic.

In a fifth aspect, the invention provides a pharmaceutical composition comprising a compound of Formula Ia or a modified peptide of Formula Ic; or a prodrug of a compound of Formula Ia or a modified peptide of Formula Ic.

In a sixth aspect, the invention provides a compound of Formula Ia, a modified peptide of Formula Ic, a prodrug of a compound of Formula Ia, a prodrug of a modified peptide of Formula Ic, or a pharmaceutical composition comprising a compound of Formula Ia or a modified peptide of Formula Ic, for use in medicine.

In a seventh aspect, the invention provides a compound of Formula Ia, a modified peptide of Formula Ic, a prodrug of a compound of Formula Ia, a prodrug of a modified peptide of Formula Ic, or a pharmaceutical composition comprising a compound of Formula Ia or a modified peptide of Formula Ic, for use in the treatment of a disease associated with aberrant protein degradation.

In an eighth aspect, the invention provides a method of treating a disease associated with aberrant protein degradation comprising administering a compound of Formula Ia, a modified peptide of Formula Ic, a prodrug of a compound of Formula Ia, a prodrug of a modified peptide of Formula Ic, or a pharmaceutical composition comprising a compound of Formula Ia or a modified peptide of Formula Ic, in a pharmaceutically effective amount.

In a ninth aspect, the invention provides a diagnostic kit comprising a compound of Formula Ia, a modified peptide of Formula Ic, a prodrug of a compound of Formula Ia, or a prodrug of a modified peptide of Formula Ic.

### SUMMARY OF THE INVENTION

### Embodiments of Compounds of Formula Ia

Various embodiments of the invention are described herein. It will be recognised that features specified in each embodiment may be combined with other specified features to provide further embodiments.

In the first aspect, the invention provides a compound of Formula Ia:

X¹-X²-X³-X⁴-X⁵-X⁶-X⁷ Formula Ia

wherein, X¹, X², X³, X⁴, X⁵, X⁶ and X⁷ are as hereinbefore defined.

### Carboxylic Acid Isosteres

Groups A¹, A² and A⁵ are each independently carboxylic acid (—CO₂H) groups or bioisosteres thereof (and A² can also be (—C(O)N(R^{N1})₂) “Bioisostere” is a term with which the skilled person will be familiar. In particular, bioisosteres (also known as non-classical isosteres) are functional groups or molecules which have chemical and physical similarities producing broadly similar biological properties to those of the replaced moiety (Stocks et al. On Medicinal Chemistry, 2007).

Carboxylic acids are weak organic acids with pKas in the range of 0-5, although this can be affected by the electronegative or electropositive nature of any substituents. For example, acetic acid (CH₃CO₂H) has a pKa of 4.8. Bioisosteres of carboxylic acids may have comparable pKa values to those of carboxylic acids, i.e. they may be deprotonated at physiological pH (pH 7.3-7.5, Werle et al. British Journal of Cancer, 1997).

Common bioisosteric replacements for carboxylic acids include functional groups such as sulfonamides (pKa˜4-9), sulfamides (pKa˜6-10), acylsulfonamides (pKa˜5), sulfonyl ureas (pKa˜3-5), hydroxaminc acids (pKa˜9), acylcyanamides (pKa 8), sulfonic acids (pKa˜2), sulfonates (pka-1-2), phosphates (pKa˜2), phosphonic acids/phosphonates (pKa 6.5) and phosphinic acids (pKa 4). Heterocycles with intrinsic acidity may also be used as bioisosteres for carboxylic acids. Common heterocyclic bioisosteric replacements for carboxylic acids include tetrazoles (pKa˜4-8), triazoles (pKa˜9), isoxazolones (pKa˜5), 1,2,4-oxadiazolones (pKa˜6), and 1,2-dihydro-pyrazolones (pKa˜8). Examples of carboxylic acid bioisosteric functional groups include:

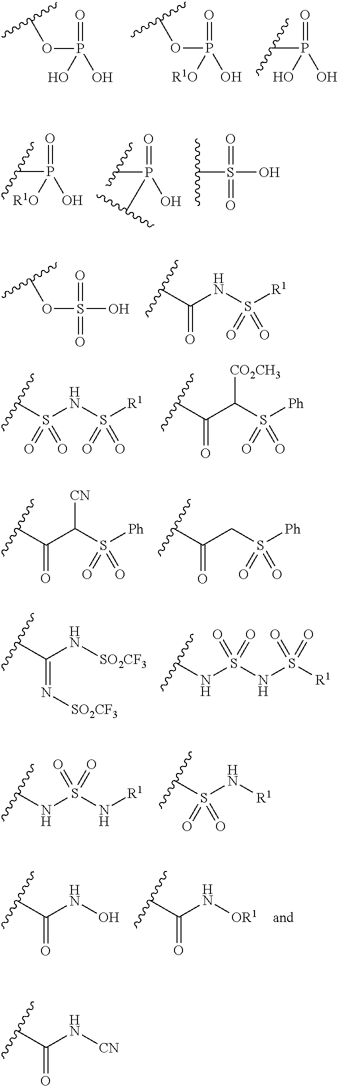

wherein, R¹ is R^{A1}, R^{A2} or RAS respectively, wherein R^{A1}, R^{A2} and R^{A5} are as previously defined.

Examples of heterocyclic carboxylic acid bioisosteres include:

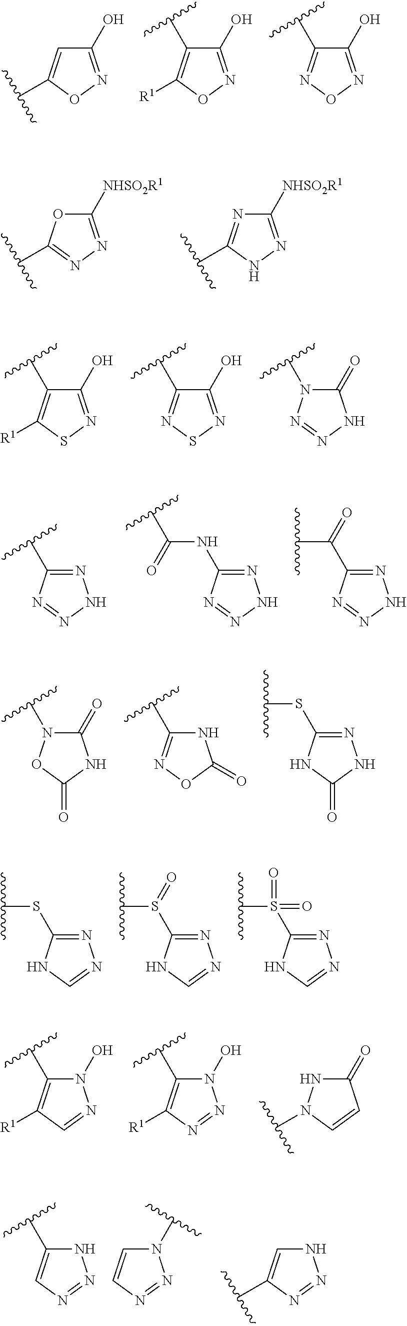

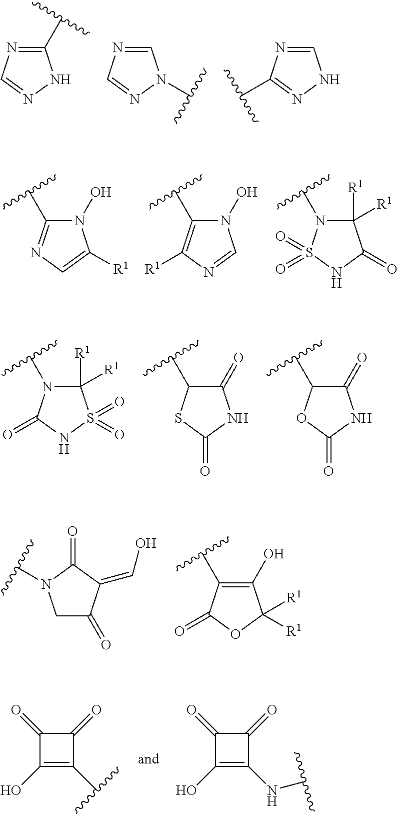

wherein, R¹ is R^{A1}, R^{A2} or RAS respectively, wherein R^{A1}, R^{A2} and R^{A5} are as previously defined.

### Group X¹

It is believed that the side chain of group X¹ interacts with the βTrCP binding domain to form an ionic bridge. Accordingly, X¹ is a functional group which is ionisable at physiological pH, in particular a carboxylic acid group or bioisostere thereof, in order to sustain such a binding interaction with the βTrCP binding domain.

### X¹ is a Group A¹-B—Z¹—;

wherein,

A¹ is carboxylic acid (—CO₂H) or a bioisostere thereof;

wherein, A¹ may be substituted with one or more R^{A1}, wherein R^{A1} is selected from the group consisting of —H, C₁-C₄ alkyl, C₂-C₄ alkenyl and aryl;

B is C₁-C₁₀ alkyl, C₂-C₁₀ alkenyl or C₂-C₁₀ alkynyl or aryl;

wherein, B may be substituted with one or more R^{E}, wherein R^{E} is selected from the group consisting of C₁-C₄ alkyl, —NH₂, —NH(RN²) and —N(R^{N2})₂; and

Z¹ is a bond, C═O, C═S, CH₂, S═O, S(O)₂, C═N(C₁-C₄ alkyl) or C═NH.

The term bioisostere is as hereinbefore described. In one embodiment, A¹ is carboxylic acid. In one embodiment, A¹ is selected from the group consisting of

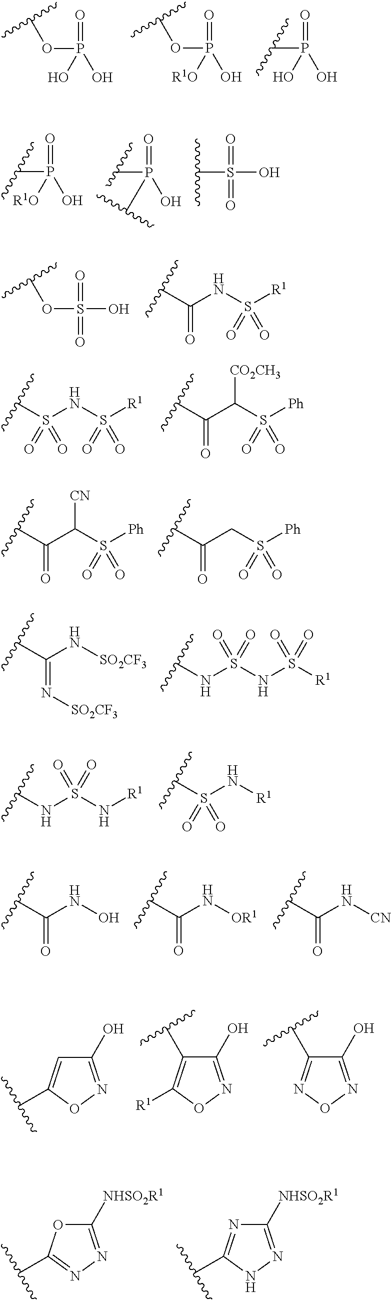

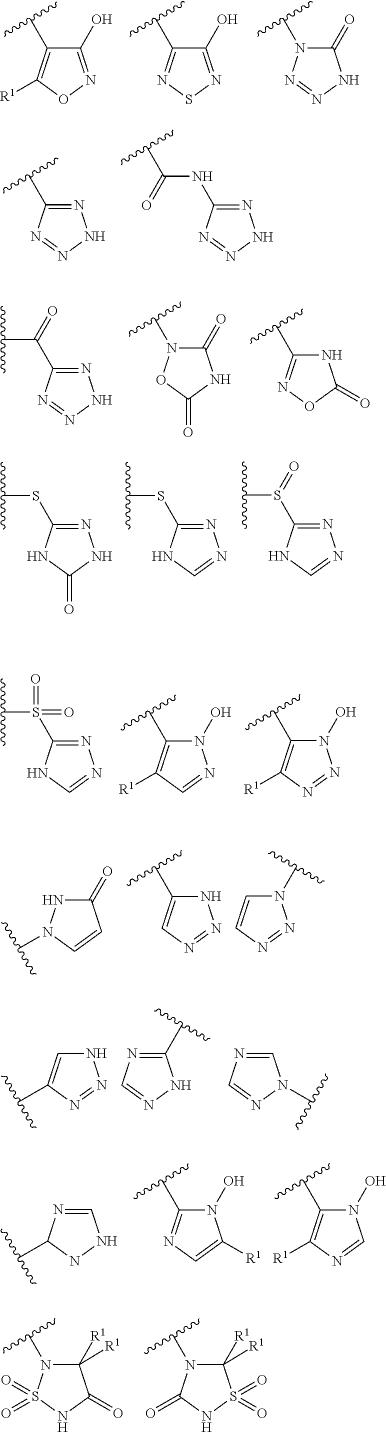

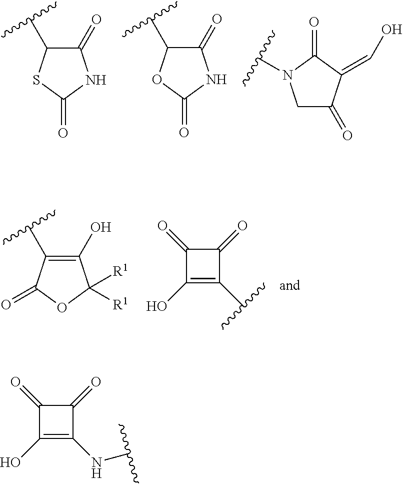

wherein, R¹ is A¹.

In one embodiment, A¹ is selected from the group consisting of

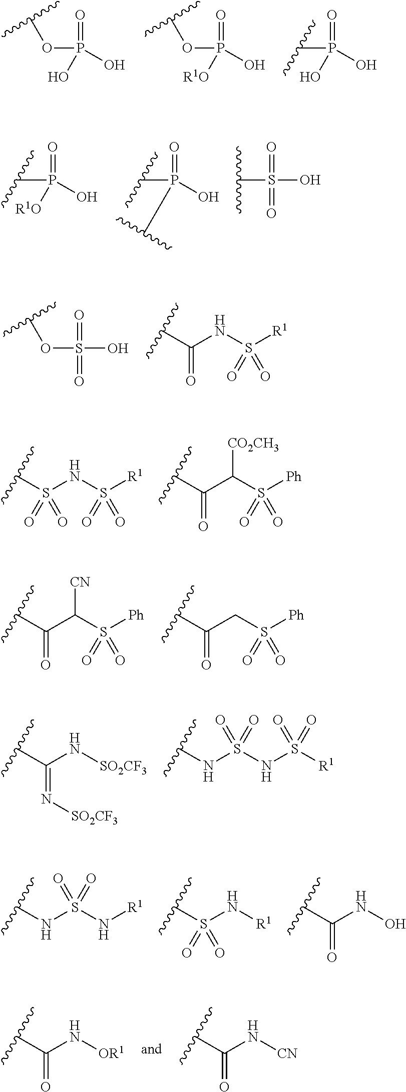

wherein, R¹ is R^{A1}.

In one embodiment, A¹ is selected from the group consisting of

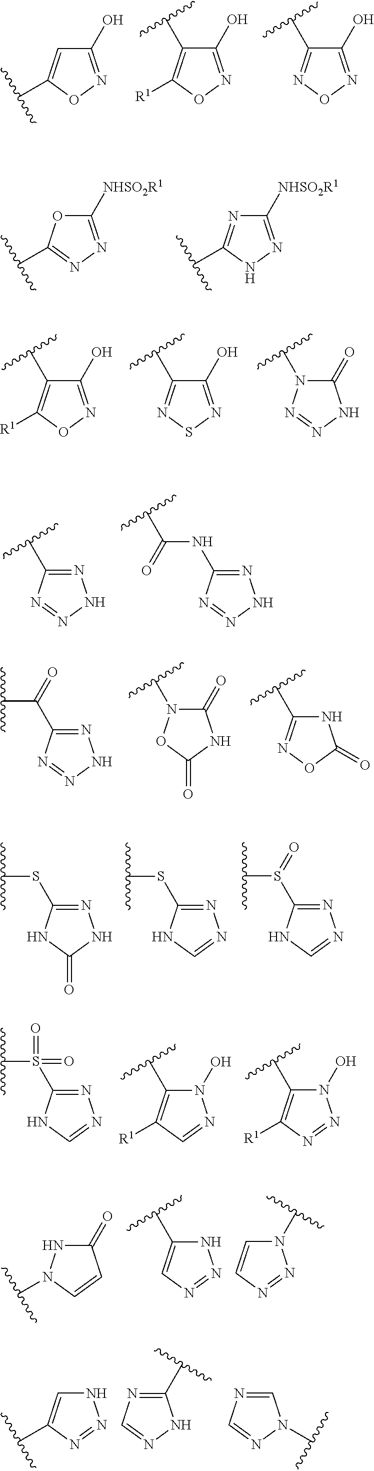

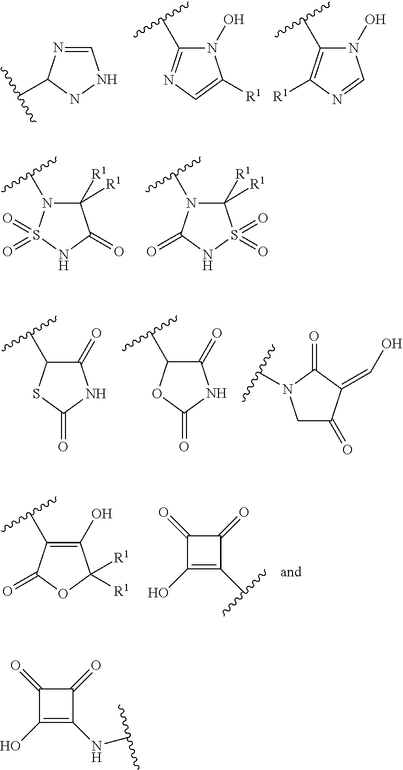

wherein, R¹ is R^{A1}.

In one embodiment, A¹ is selected from the group consisting of carboxylic acid (—CO₂H), phosphate, phosphonate, phosphonic acid, tetrazole and sulphate.

In one embodiment, A¹ is selected from the group consisting of carboxylic acid and phosphate. In one embodiment, preferably A¹ is carboxylic acid.

In one embodiment, A¹ is substituted by R^{A1}, wherein R^{A1} is selected from the group consisting of —H, C₁-C₄ alkyl, C₂-C₄ alkenyl and aryl.

In one embodiment, B is C₁-C₄ alkyl, C₂-C₄ alkenyl or C₂-C₄ alkynyl. In one embodiment, B is C₁-C₂ alkyl or C₂ alkenyl. In one embodiment B is aryl, particularly B is phenyl.

In one embodiment, B is substituted with one or more R^{E}, wherein R^{E} is selected from the group consisting of C₁-C₄ alkyl, —NH₂, —NH(R^{N2}) and —N(R^{N2})₂. In one embodiment, B is substituted with —NH₂. In one embodiment, B is substituted with —NH(R^{N2}), wherein R^{N2} is a chain of one or more naturally or non-naturally occurring amino acids.

In one embodiment, B is substituted with —N(R^{N2})₂. In one embodiment, B is substituted with —N(R^{N2})₂, wherein both R^{N2} are R^{N1}, wherein one R^{N1} is —H and the other R^{N1} is C₁-C₁₀ alkyl, aryl or heteroaryl.

In one embodiment, Z¹ is C═O, C═S, or CH₂. In one embodiment, Z¹ is C═O.

In one embodiment, X¹ is of Formula HO₂C—B—Z¹—; wherein Z¹ is a bond, C═O, C═S, CH₂, S═O or S(O)₂; and B is C₁-C₁₀ alkyl, C₂-C₁₀ alkenyl or C₂-C₁₀ alkynyl. In one embodiment, X¹ is of Formula HO₂C—B—Z¹—, wherein Z¹ is C═O and B is C₁-C₄ alkyl, C₂-C₄ alkenyl or C₂-C₄ alkynyl. In one embodiment, X¹ is of Formula HO₂C—B—Z¹—, wherein Z¹ is C═O and B is C₁ alkyl. In one embodiment, X¹ is of Formula HO₂C—B—Z¹—, wherein Z¹ is C═O and B is C₂ alkyl, C₂ alkenyl or C₂ alkynyl. In one embodiment, X¹ is of Formula HO₂C—B—Z¹—, wherein Z¹ is C═O and B is C₃ alkyl, C₃ alkenyl or C₃ alkynyl. In one embodiment, X¹ is of Formula HO₂C—B—Z¹—, wherein Z¹ is C—O and B is C₄ alkyl, C₄ alkenyl or C₄ alkynyl. In one embodiment, X¹ is of Formula HO₂C—B—Z¹—, wherein Z¹ is C═O and B is C₅ alkyl, C₅ alkenyl or C₅ alkynyl.

In one embodiment, X¹ is of Formula HO₂C—B—Z¹—, wherein Z¹ is C═O and B is C₆ alkyl, C₆ alkenyl or C₆ alkynyl. In one embodiment, X¹ is of Formula HO₂C-E-Z¹—, wherein Z¹ is C═O and B is C₂ alkyl. In one embodiment, X¹ is of Formula HO₂C—B—Z¹—, wherein Z¹ is C═O and B is C₂ alkenyl. In one embodiment, X¹ is of Formula HO₂C—B—Z¹—, wherein Z¹ is C═O and B is C₂ alkyl; wherein C₂ alkyl is substituted with one or more R^{E}. In one embodiment, X¹ is of Formula HO₂C—B—Z¹—, wherein Z¹ is C═O and B is C₂ alkyl; wherein C₂ alkyl is substituted with one or more —N(R^{N2})₂. In one embodiment, X¹ is of Formula HO₂C—B—Z¹—, wherein Z¹ is C═O and B is C₂ alkenyl; wherein C₂ alkenyl is substituted with one or more —N(R^{N2})₂. In one embodiment, X¹ is of Formula HO₂C—B—Z¹—, wherein Z¹ is C≡O and B is C₃ alkyl; wherein C₃ alkyl is substituted with one or more —N(R^{N2})₂. In one embodiment, X¹ is of Formula HO₂C—B—Z¹—, wherein Z¹ is C═O and B is C₃ alkyl; wherein C₃ alkyl is substituted with one or more —N(R^{N1})(R^{N2}). In one embodiment, X¹ is of Formula HO₂C—B—Z¹—, wherein Z¹ is C═O and B is C₃ alkenyl; wherein C₃ alkenyl is substituted with one or more —N(RNIXR^{N2}). In one embodiment, X¹ is of Formula HO₂C—B—Z¹—, wherein Z¹ is C═O and B is C₃ alkyl; wherein C₃ alkyl is substituted with —NH₂. In one embodiment, X¹ is of Formula HO₂C—B—Z¹—, wherein Z¹ is C═O and B is C₂ alkyl; wherein C₂ alkyl is substituted with —NH₂ at the carbon atom adjacent to Z¹. In one embodiment, X¹ is of Formula HO₂C—B—Z¹—, wherein Z¹ is C═O and B is C₂ or C₃ alkyl, wherein said C₂ or C₃ alkyl is substituted with one or more —N(R^{N1})(RN²) wherein R^{N2} is a chain of one or more amino acids. In one embodiment, X¹ is of Formula HO₂C—B—Z¹—, wherein Z¹ is C═O and B is C₂ or C₃ alkyl, wherein said C₂ or C₃ alkyl is substituted with one or more —N(R^{N1})(R^{N2}) wherein R^{N1} is —H and R^{N2} is a chain of one or more naturally or non-naturally occurring amino acids.

In another embodiment, X¹ may be selected from the group consisting of:

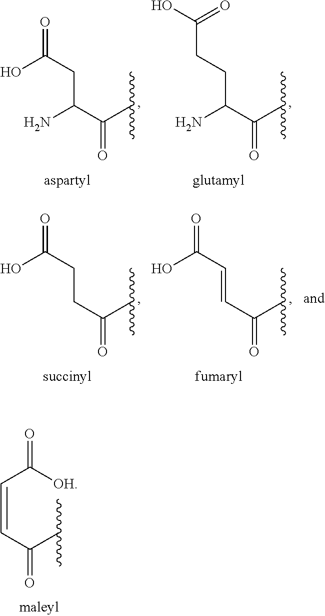 [SMILES: *C(=O)C(N)CC(=O)O ; *C(=O)C(N)CCC(=O)O ; *C(=O)CCC(=O)O ; *C(=O)/C=C/C(=O)O ; *C(=O)/C=C\C(=O)O]

In one embodiment, X¹ is aspartyl, succinyl or maleyl. In one embodiment, preferably X¹ is aspartyl. In one embodiment, X¹ is aspartyl or glutamyl, and is substituted at the N-terminus with a chain of one or more naturally or non-naturally occurring amino acids.

When X¹ is aspartyl, it is preferably L-aspartyl (D) or D-aspartyl (d). When X¹ is glutamyl it is preferably L-glutamyl (E) or D-glutamyl (e).

### Group X²

It is believed that the side chain of group X² interacts with the βTrCP binding domain to form an ionic bridge. Accordingly, X² is a functional group which is ionisable at physiological pH, in particular carboxylic acid groups or bioisosteres thereof, in order to sustain such a binding interaction with the βTrCP binding domain.

X² is a group —N(R^{a})—Y¹(-L¹-A²)-Z²—;
wherein;

R^{a} is selected from the group consisting of —H, C₁-C₁₀ alkyl, aryl and heteroaryl;

L¹ is C₀-C₅ alkyl, C₂-C₈ alkenyl or C₂-C₈ alkynyl; wherein, L¹ may be substituted with one or more R^{L1}, wherein R^{L1} is C₁-C₄ alkyl;

Y¹ is CH or N;

Z² is selected from the group consisting of C═O, C═S, CH₂, S═O, S(O)₂, C═N(C₁-C₄ alkyl) and C═NH; and

A² is carboxylic acid (—CO₂H) or a bioisostere thereof or —C(O)N(R^{N})₂; wherein, A² may be substituted with one or more R^{A2}, wherein R^{A2} is selected from the group consisting of —H, C₁-C₄ alkyl, C₂-C₄ alkenyl and aryl.

In one embodiment, R^{a} is —H. In one embodiment, R¹ is C₁-C₁₀ alkyl.

In one embodiment, Y¹ is CH. In one embodiment, Y¹ is N.

In one embodiment, A² is carboxylic acid. In one embodiment, A² is selected from the group consisting of

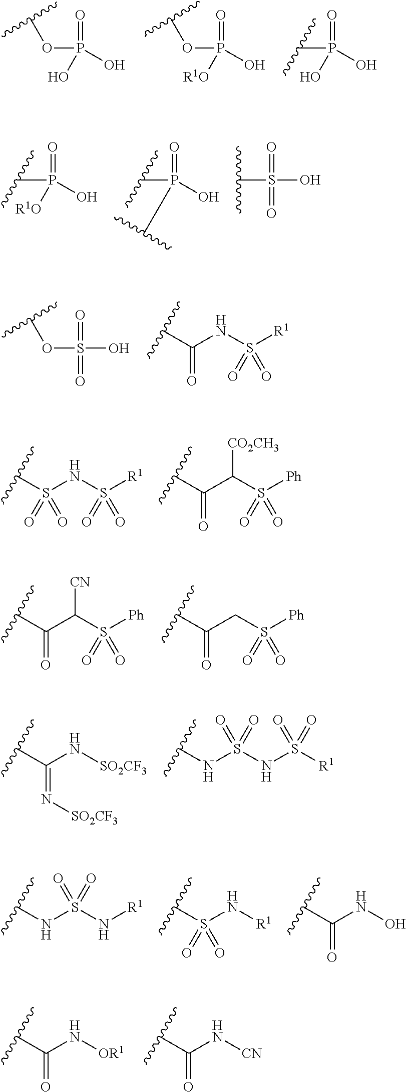

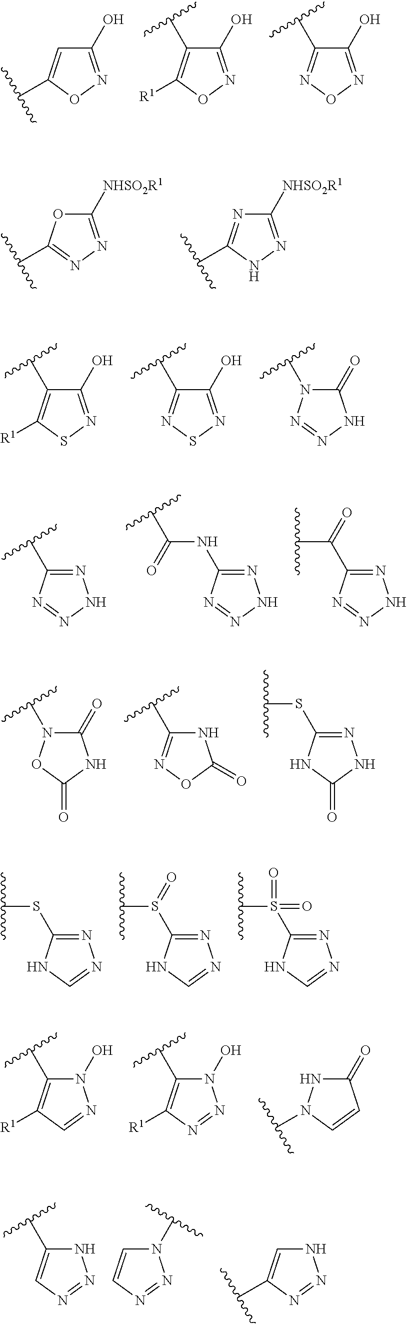

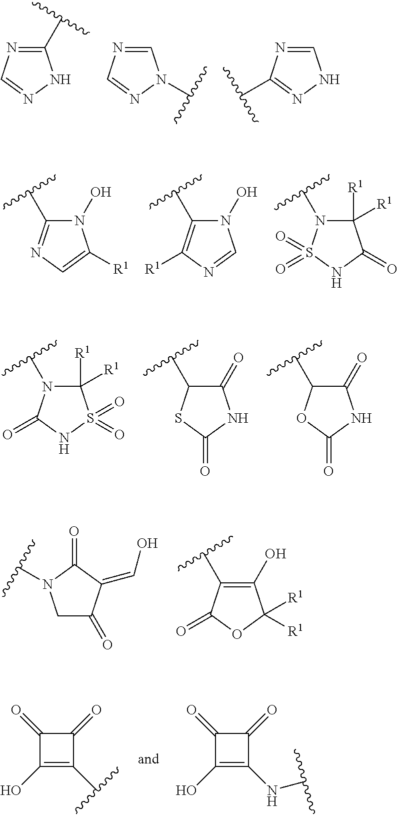

wherein, R¹ is R^{A2}.

In one embodiment, A² is selected froom the group consisting of

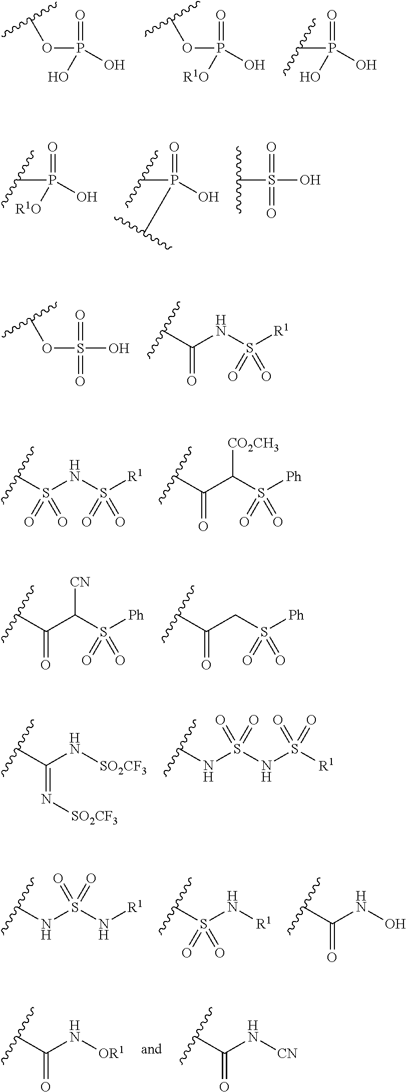

wherein, R¹ is R¹ is R^{A2}.

In one embodiment, A² is selected from the group consisting of

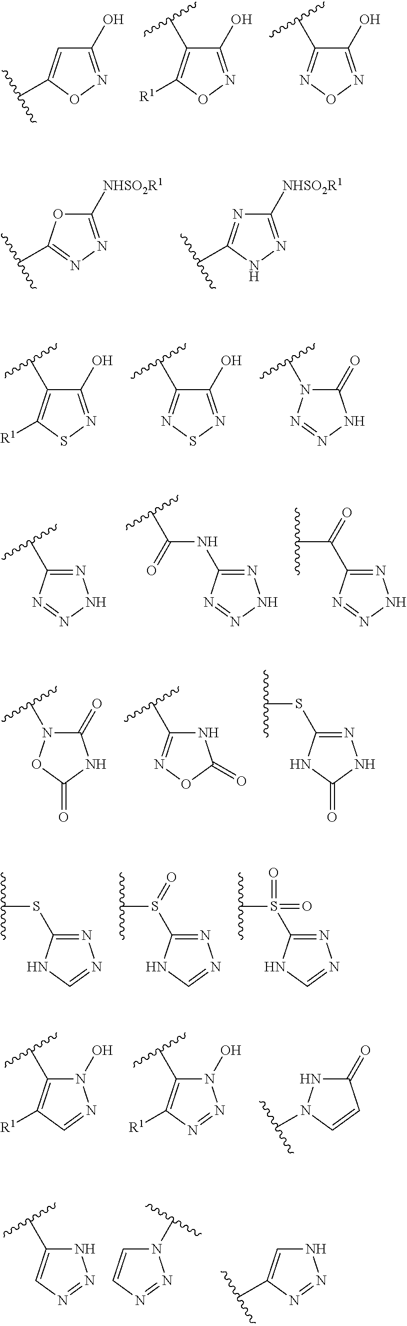

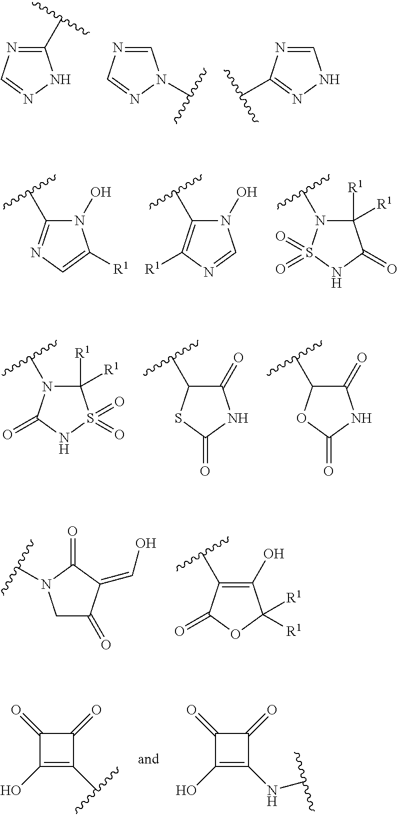

wherein, R¹ is R^{A2}.

In one embodiment, A² is selected from the group consisting of carboxylic acid (—CO₂H), phosphate, phosphonate, phosphonic acid, tetrazole and sulphate.

In one embodiment, A² is selected from the group consisting of carboxylic acid and phosphate. In one embodiment, preferably A² is carboxylic acid.

In one embodiment, A² is substituted with one or more R^{A2}, wherein R^{A2} is selected from the group consisting of —H, C₁-C₄ alkyl, C₂-C₄ alkenyl and aryl. In one embodiment, A² is substituted with one or more R^{A2}, wherein RU is methyl or ethyl.

In one embodiment, A² is substituted with one or more R^{A2}, wherein R^{A2} is methyl.

In one embodiment A² is —C(O)N(R^{N1})₂ wherein each R^{N1} may be the same or different. Particularly A² is C(O)NH(R^{N1}), more particularly A² is C(O)NH₂ In one embodiment, L¹ is C₀-C₅ alkyl or C₂-C₅ alkenyl. In one embodiment, L¹ is C₀-C₅ alkyl. In one embodiment, L¹ is preferably C₁-C₂ alkyl.

In one embodiment, L¹ is substituted with one or more R^{L1}, wherein R^{L1} is C₁-C₄ alkyl. In one embodimenet, L¹ is substituted with one or more R^{L1}, wherein R^{L} is methyl.

In one embodiment, X² is of Formula —NH—Y¹(-L¹-A²)-Z²—; wherein L¹ is C₁-C₈ alkyl, Y¹ is CH, Z² is C═O and A² is carboxylic acid (—CO₂H) or phosphate. In one embodiment, X² is of Formula —NH—Y¹(-L¹-A²)-Z²—; wherein L¹ is C₁-C₂ alkyl, Y¹ is CH, Z² is C═O and A² is carboxylic acid (—CO₂H) or phosphate. In one embodiment, X² is of Formula —NH—Y¹(-L¹-A²)-Z²—; wherein L¹ is C₁ alkyl, Y¹ is CH, Z² is C═O and A² is carboxylic acid (—CO₂H) or phosphate. In one embodiment, X² is of Formula —NH—Y¹(-L¹-A²)-Z²—; wherein L¹ is C₂ alkyl, Y¹ is CH, Z² is C═O and A² is carboxylic acid (—CO₂H) or phosphate. In one embodiment, X² is of Formula —NH—Y¹(-L¹-A²)-Z²—; wherein L¹ is C₁ alkyl, Y¹ is CH, Z² is C—O and A² is carboxylic acid (—CO₂H). In one embodiment, X² is of Formula —NH—Y¹(-L¹-A²)-Z²—; wherein L¹ is C₁ alkyl, Y¹ is CH, Z² is C═O and A² is phosphate. In one embodiment, X² is of Formula —NH—Y¹(-L¹-A²)-Z²—; wherein L¹ is C₂ alkyl, Y¹ is CH, Z² is C—O and A² is carboxylic acid (—CO₂H). In one embodiment, X^{Z} is of Formula —NH—Y¹(-L-A²)-Z²—; wherein L¹ is C₂ alkyl, Y¹ is CH, Z² is C═O and A² is phosphate. In one embodiment, X² is of Formula —NH—Y¹(-L¹-A²)-Z²—; wherein L¹ is C₁ alkyl substituted with R^{L1}, wherein R^{L1} is methyl, Y¹ is CH, Z² is C═O and A² is carboxylic acid (—CO₂H). In one embodiment, X² is of Formula —NH—Y¹(-L¹-A²)-Z²—; wherein L¹ is C₁ alkyl substituted with R^{L1}, wherein R^{L1} is methyl, Y¹ is CH, Z² is C═O and A² is phosphate.

In one embodiment, group X² may be a glutamate, an aspartate, or a phosphorylated serine residue. In one embodiment, preferably, X² is glutamate or aspartate. In one embodiment, the glutamate, aspartate, or phorphorylated serine residue of group X² is an L-amino acid. In a further embodiment, X² is phosphorylated threonine.

In one embodiment, preferably, group X² is a glutamate or aspartate residue. This eliminates the requirement for phosphorylated serine residues, which are naturally present within the phosphodegeneron sequence, whilst retaining binding. The negatively charged phosphorylated serine residues are not synthetically desirable.

In one embodiment, the glutamate, aspartate or phosphorylated serine residue of X² may be substituted with methyl. In another embodiment, the glutamate, aspartate or phosphorylated serine residue of group X² may be substituted with ethyl.

### Group X³

It is believed that group X³ associates with an area in the βTrCP binding domain which may accommodate a compound/modified peptide with a beta-turn.

Accordingly, X¹ is a functional group which is suitably configured to reside in this area of the βTrCP binding domain.

### X³ is a Group —N(R^{b})—Y²—Z³—;

wherein,

R^{b} is selected from the group consisting of —H, C₁-C₁₀ alkyl, aryl and heteroaryl;

Y² is CF₂, CH₂, N(R^{Y2}) or O; wherein, R^{Y2} is —H or C₁-C₄ alkyl; and

Z³ is selected from the group consisting of C═O, C═S, CH₂, S═O, S(O)₂, C═N(C₁-C₄ alkyl) and C═NH.

In one embodiment, Rb is —H or C₁-C₁₀ alkyl. In one embodiment, preferably Rb is —H.

In one embodiment, Y² is CH₂ or N(R²). In one embodiment, Y² is preferably CH₂.

In one embodiment, Y² is N(R^{Y2}). In one embodiment, Y² is N(R^{Y2}), wherein R^{Y2} is methyl.

In one embodiment, X³ is of Formula —NH—Y²—Z³—, wherein Y² is CH₂, N(R¹²) or O and Z³ is selected from the group consisting of C═O, C═S, CH₂, S═O and S(O)₂. In one embodiment, preferably, X³ is of Formula —NH—Y²—Z³—, wherein Y² is CH₂ and Z³ is C═O. In one embodiment, X³ is of Formula —NH—Y²—Z³—, wherein Y² is NH and Z³ is C═O.

In one embodiment, preferably group X³ is a glycine residue.

In one embodiment the glycine residue of group X³ is an aza glycine residue, wherein an “aza amino acid” is an L-/D-amino acid in which the α-carbon atom has been replaced by a nitrogen atom.

In one embodiment the glycine residue of group X³ is an oxo glycine residue, wherein an “oxo amino acid” is an L-/D-amino acid in which the α-carbon atom has been replaced by an oxygen atom.

### Group X⁴

Without wishing to be bound by theory, it is believed that the side chain of group X⁴ sustains a Van der Waals interaction with the βTrCP binding domain.

X¹ is a Group —N(R^{c})—Y³(-L²-A³)-Z⁴—; or —N(R^{c})—Y³(-L²)-Z⁴—
wherein,

R^{c} is selected from the group consisting of —H, C₁-C₁₀ alkyl, aryl and heteroaryl;

L² is C₀-C₅ alkyl, C₂-C₅ alkenyl or C₂-C₈ alkynyl; wherein, L² may be substituted with one or more Ru, wherein Ru is C₁-C₄ alkyl or C₂-C₄ alkenyl;

Y³ is CH or N;

Z⁴ is selected from the group consisting of C═O, C═S, CH₂, S═O, S(O)₂, C═N(C₁-C₄ alkyl) and C═NH; and

A³ is aryl or heteroaryl; wherein, A³ may be substituted with one or more R^{A3}, wherein, R^{A3} is selected from the group consisting of —H, —F, —Cl, —Br, —I, —OH, —O(C₁-C₁₀ alkyl), —CN, —NO₂, —CF₃, —OCF₃, —CO₂H, —C₁-C₁₀ alkyl, —NH₂, —NH(C₁-C₂ alkyl) and —N(C₁-C₂ alkyl)₂;

In one embodiment, X⁴ is a group —N(R^{c})—Y³(-L²-A³)-Z⁴—.

In one embodiment, X⁴ is a group —N(R^{c})—Y³(-L²)-Z⁴—.

In one embodiment, R^{c} is —H or C₁-C₁₀ alkyl. In one embodiment, preferably R^{c} is —H.

In one embodiment, Y³ is CH. In one embodiment, Y³ is N.

In one embodiment, L² is C₀-C₅ alkyl or C₂-C₈ alkenyl. In one embodiment, L² is C₀-C₅ alkyl. In one embodiment, L² is C₁-C₂ alkyl. In one embodiment, preferably L² is C₁ alkyl.

In one embodiment, L² is substituted with one or more Ru, wherein Ru is C₁-C₄ alkyl or C₂-C₄ alkenyl. In one embodiment, L² is substituted with Ru, wherein R^{U} is methyl.

In one embodiment, A³ is aryl. In one embodiment, A³ is phenyl. In one embodiment, A³ is heteroaryl. In one embodiment, A³ is aryl or heteroaryl substituted with one or more R^{A3}, wherein, R^{A3} is selected from the group consisting of —H, —F, —Cl, —Br, —I, —OH, —O(C₁-C₁₀ alkyl), —CN, —NO₂, —CF₃, —OCF₃, —CO₂H, —C₁-C₁₀ alkyl, —NH₂, —NH(C₁-C₂ alkyl) and —N(C₁-C₂ alkyl)₂.

In one embodiment, A³ is aryl or heteroaryl, wherein said aryl or heteroaryl is substituted with one or more R^{A3}, wherein R^{A3} is selected from the group consisting of —H, —F, —Cl, —Br, —I, —OH, —O(C₁-C₁₀ alkyl), —NO₂ and C₁-C₁₀ alkyl.

In one embodiment, A³ is aryl or heteroaryl, wherein said aryl or heteroaryl is substituted with one or more R^{A3}, wherein R^{A3} is selected from the group consisting of —F, —Cl, —OH and —NO₂.

In one embodiment, A³ is phenyl substituted at one or more of the 2-, 3- or 4-positions, with R^{A3}, wherein R^{A3} is a substituent selected from the group consisting of —H, —F, —Cl, —Br, —I, —OH, —O(C₁-C₁₀ alkyl), —CN, —NO₂, —CF₃, —OCF₃, —CO₂H and —C₁-C₁₀ alkyl.

In one embodiment, preferably A³ is phenyl substituted with one or more R^{A}, wherein R^{A3} is selected from the group consisting of —F, —Cl, —NO₂ and —OH.

In another embodiment, preferably A³ is phenyl substituted with R^{A3}, wherein R^{A3} is chlorine and/or fluorine.

In one embodiment, Z⁴ is selected from the group consisting of C—O, C═S and CH₂.

In one embodiment, Z⁴ is C═O. In one embodiment, X⁴ is of Formula —NH—Y³(-L²-A³)-Z⁴—; wherein Y³ is CH, Z⁴ is C═O, L² is C₁-C₅ alkyl and A³ is aryl or heteroaryl, wherein said aryl or heteroaryl is substituted with one or more R^{A3}.

In one embodiment, X⁴ is of Formula —NH—Y³(-L²-A³)-Z⁴—; wherein Y³ is CH, Z⁴ is C—O, L² is C₁-C₅ alkyl and A³ is aryl or heteroaryl, wherein said aryl or heteroaryl is substituted with one or more R^{A}, wherein R^{A3} is selected from the group consisting of —H, —F, —Cl, —Br, —I, —OH, —O(C₁-C₁₀ alkyl), —CN, —NO₂, —CF₃, —OCF₃, —CO₂H, —C₁-C₁₀ alkyl, —NH₂, —NH(C₁-C₂ alkyl) and —N(C₁-C₂ alkyl)₂.

In one embodiment, X⁴ is of Formula —NH—Y³(-L²-A³)-Z⁴—; wherein Y³ is CH, Z⁴ is C═O, L² is C₁-C₅ alkyl and A³ is aryl or heteroaryl, wherein said aryl or heteroaryl is substituted with one or more R^{A}, wherein R^{A3} is selected from the group consisting of —H, —F, —Cl, —Br, —I, —OH, —O(C₁-C₁₀ alkyl), C₁-C₁₀ alkyl and —NO₂.

In one embodiment, X⁴ is of Formula —NH—Y³(-L²-A³)-Z⁴—; wherein Y³ is CH, Z⁴ is C═O, L² is C₁ alkyl and A³ is phenyl, wherein said phenyl is substituted with one or more R^{A3}, wherein R^{A} is selected from the group consisting of —H, —F, —Cl, —Br, —I, —OH, —O(C₁-C₁₀ alkyl) and —NO₂.

In one embodiment, X⁴ is of Formula —NH—Y³(-L²-A)-Z⁴—; wherein Y³ is CH, Z⁴ is C═O, L² is C₁ alkyl and A³ is selected from the group consisting of indole and imidazole, wherein said indole or imidazole is substituted with one or more R^{A3}, wherein R^{A3} is selected from the group consisting of —H, —F, —Cl, —Br, —I, —OH, —O(C₁-C₁₀ alkyl), C₁-C₁₀ alkyl and —NO₂.

In one embodiment, X⁴ is of Formula —NH—Y³(-L²-A³)-Z⁴—; wherein Y³ is CH, Z⁴ is C═O, L² is C₁ alkyl and A³ is phenyl, wherein said phenyl is substituted at one or more of the 2-, 3- or 4-positions with R^{A3}, wherein R^{A3} is selected from the group consisting of —H, —F, —Cl, —Br, —I, —OH, —O(C₁-C₁₀ alkyl), C₁-C₁₀ alkyl and —NO₂.

In one embodiment, X⁴ is of Formula —NH—Y³(-L²-A³)-Z⁴—; wherein Y³ is CH, Z⁴ is C═O, L² is C alkyl and A³ is phenyl, wherein said phenyl is substituted at one or more of the 2-, 3- or 4-positions with R^{A3}, wherein R^{A3} is selected from the group consisting of —F, —Cl, —NO₂ and —OH.

In one embodiment, group X⁴ is an aromatic alanine derivative.

In one embodiment, preferably group X⁴ is selected from the group consisting of phenylalanine, tyrosine, tryptophan and histidine. In one embodiment, X⁴ is phenylalanine. In one embodiment, group X⁴ may be an L amino acid. In one embodiment, group X⁴ is selected from the group consisting of phenylalanine, tyrosine, tryptophan and histidine, wherein said phenylalanine, tyrosine, tryptophan and histidine is substituted with one or more R^{A3}.

In one embodiment, group X⁴ is selected from the group consisting of:

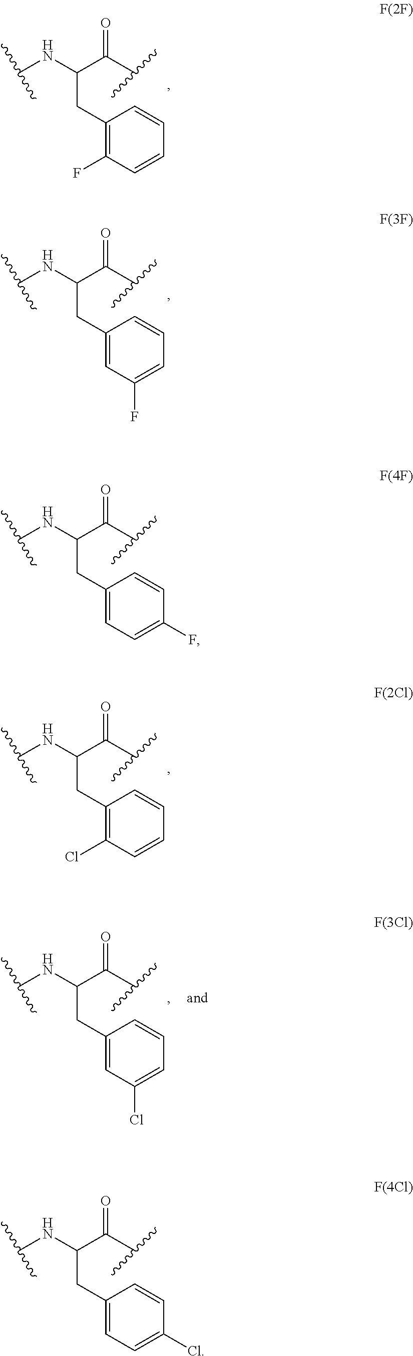 [SMILES: *NC(Cc1ccccc1F)C(*)=O ; *NC(Cc1cccc(F)c1)C(*)=O ; *NC(Cc1ccc(F)cc1)C(*)=O ; *NC(Cc1ccccc1Cl)C(*)=O ; *NC(Cc1cccc(Cl)c1)C(*)=O ; *NC(Cc1ccc(Cl)cc1)C(*)=O]

### Group X⁵

It is believed that the side chain of group X⁵ interacts with an alkyl portion within the βTrCP binding domain. Accordingly, X⁵ is a functional group which is capable of sustaining such an interaction within the βTrCP binding domain.

### X⁵ is a Group —N(R^{d})—Y⁴(-L³-A)-Z⁵—;

wherein;

R^{d} is selected from the group consisting of —H, C₁-C₁₀ alkyl, aryl and heteroaryl;

Y⁴ is CH or N;

L³ is C₀-C₅ alkyl, C₂-C₅ alkenyl or C₂-C₈ alkynyl; wherein, L³ may be substituted with one or more R^{L3}, wherein R^{L3} is C₁-C₄ alkyl;

A⁴ is aryl or heteroaryl; wherein, A⁴ may be substituted with one or more R^{A4},

wherein R^{A4} is selected from the group consisting of —H, —F, —Cl, —Br, —I, —OH, —O(C₁-C₁₀ alkyl), —CN, —NO₂, —CF₃, —OCF₃, —CO₂H, —C₁-C₁₀ alkyl, —NH₂, —NH(C₁-C₂ alkyl) and —N(C₁-C₂ alkyl)₂; and

Z⁵ is selected from the group consisting of C═O, C═S, CH₂, S═O, S(O)₂, C═N(C¹-C⁴ alkyl) and C═NH.

In one embodiment, R^{d} is —H or C₁-C₁₀ alkyl. In one embodiment, preferably Rd is —H.

In one embodiment, Y⁴ is CH. In one embodiment, Y¹ is N.

In one embodiment, L³ is C₀-C₅ alkyl or C₂-C₅ alkenyl. In one embodiment, L³ is C₀-C₅ alkyl. In one embodiment, L³ is C₁-C₂ alkyl. In one embodiment, preferably L³ is C₁ alkyl.

In one embodiment, L³ is substituted with R^{L3}, wherein R^{L3} is C₁-C₄ alkyl. In one embodiment, L¹ is substituted with R^{L3}, wherein R^{L3} is methyl.

A⁴ is aryl or heteroaryl. In one embodiment, A⁴ is aryl. In one embodiment, A⁴ is bi-aryl, monocyclic aryl or polycyclic fused ring aryl. In one embodiment, A⁴ is heteroaryl. In one embodiment, A⁴ is monocyclic heteroaryl. In one embodiment, A⁴ is polycyclic fused ring heteroaryl.

In one embodiment, A⁴ is selected from the group consisting of:

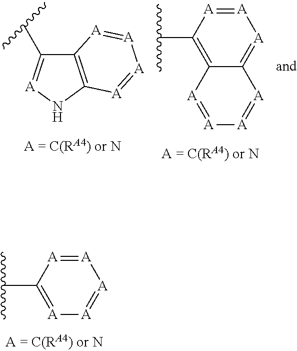

In one embodiment, A⁴ is selected from the group consisting of phenyl, biphenyl, naphthyl, indenyl, fluorenyl, anthracyl and phenanthryl. In one embodiment, A⁴ is selected from the group consisting of pyridyl, thienyl, furanyl, pyrrolyl, pyrazolyl, imidazoyl, oxazolyl, isoxazolyl, thiazolyl, isothiazolyl, triazoyl, oxadiazolyl, thiadiazolyl and tetrazolyl. In one embodiment, A⁴ is selected from the group consisting of indolyl, benzofuranyl, quinolyl, isoquinolyl, indazolyl, indolinyl, isoindolyl, indolizinyl, benzamidazolyl or quinolinyl. In one embodiment, A⁴ is selected from the group consisting of phenyl, naphthyl, indolyl and imidazoyl.

In one embodiment, A⁴ is aryl or heteroaryl, wherein said aryl or heteroaryl are substituted with one or more R^{A4}, wherein R^{A4} is selected from the group consisting of —H, —F, —Cl, —Br, —I, —OH, —O(C₁-C₁₀ alkyl), —CN, —NO₂, —CF₃, —OCF₃, —CO₂H, —C₁-C₁₀ alkyl, —NH₂, —NH(C₁-C₂ alkyl) and —N(C₁-C₂ alkyl)₂.

In one embodiment, X⁵ is of Formula —NH—Y⁴(-L³-A⁴)-Z⁵—; wherein Y⁴ is CH, Z⁵ is C═O, L³ is C₁-C₅ alkyl and A⁴ is aryl. In one embodiment, X⁵ is of Formula —NH—Y⁴(-L³-A⁴)-Z⁵—; wherein Y⁴ is CH, Z⁵ is C═O, L³ is C₁-C₅ alkyl and A⁴ is heteroaryl.

In one embodiment, preferably, X⁵ is of Formula —NH—Y⁴(-L³-A⁴)-Z⁵—; wherein Y⁴ is CH, Z⁵ is C═O, L³ is C₁ alkyl and A⁴ is selected from the group consisting of phenyl, naphthyl, indolyl and imidazoyl.

In one embodiment, preferably group X⁵ is selected from the group consisting of tryptophan, naphthyl-alanine, histidine and phenylalanine, wherein X⁵ may be substituted at one or more positions with R^{A4}. In one embodiment, group X⁵ is selected from the group consisting of tryptophan, 1 naphthyl-alanine, 2 napthyl-alanine, histidine and F(4NO₂). In one embodiment, group X⁵ is tryptophan. In one embodiment, group X⁵ is tryptophan, wherein the nitrogen of the indole group is substituted with methyl. In one embodiment, group X⁵ is naphthyl-alanine. In one embodiment, group X¹ is 2 naphthyl-alanine. In one embodiment, group Xs is 1 naphthyl-alanine. In one embodiment, group X⁵ is histidine. In one embodiment, group X⁵ is phenylalanine. In one embodiment, group Xs is phenylalanine, wherein in phenyl is substituted with one or more R^{A4}. In one embodiment, group X¹ is F(4NO₂) or F(3NO₂). In one embodiment, group X⁵ is an L amino acid. In one embodiment, the tryptophan, naphthyl-alanine, histidine or phenylalanine residue of group X⁵ may be substituted at one or more positions with R^{A4}.

### Group X⁶

It is believed that the side chain of group X⁶ interacts with the βTrCP binding domain to form an ionic bridge. Accordingly, X⁶ is a functional group which is ionisable at physiological pH, in particular a carboxylic acid group or bioisostere thereof, in order to sustain such a binding interaction with the βTrCP binding domain.

X⁶ is a group —N(R)—Y(-L⁴-A)-Z⁶—;
wherein;

R^{e} is selected from the group consisting of —H, C₁-C₁₀ alkyl, aryl and heteroaryl;

L⁴ is C₀-C₅ alkyl, C₂-C₅ alkenyl or C₂-C₅ alkynyl; wherein L⁴ may be substituted with one or more R^{L4}, wherein R^{L} is C₁-C₄ alkyl;

Y⁵ is CH or N;

Z⁶ is selected from the group consisting of C═O, C═S, CH₂, S═O, S(O)₂, C═N(C₁-C₄ alkyl) and C═NH; and

A⁵ is carboxylic acid (—CO₂H) or a bioisostere thereof; wherein, A⁵ may be substituted with one or more R^{A5}, wherein R^{A5} is selected from the group consisting of —H, C₁-C₄ alkyl, C₂-C₄ alkenyl and aryl.

In one embodiment, R^{e} is —H. In one embodiment, R^{e} is C₁-C₁₀ alkyl. In one embodiment, R^{e} is C₁-C₄ alkyl. In one embodiment, R^{e} is methyl or ethyl. In one embodiment, R^{e} is methyl. In one embodiment, R^{e} is aryl or heteroaryl. In one embodiment, R^{e} is phenyl.

In one embodiment, Y⁵ is CH. In one embodiment, Y⁵ is N.

In one embodiment, L⁴ is C₀-C₅ alkyl or C₂-C₅ alkenyl. In one embodiment, L⁴ is C₀-C₅ alkyl. In one embodiment, L⁴ is preferably C₁-C₂ alkyl.

In one embodiment, L⁴ is substituted with one or more R^{L4}, wherein R^{L4} is C₁-C₄ alkyl.

In one embodiment, A⁵ is carboxylic acid. In one embodiment, A⁵ is selected from the group consisting of

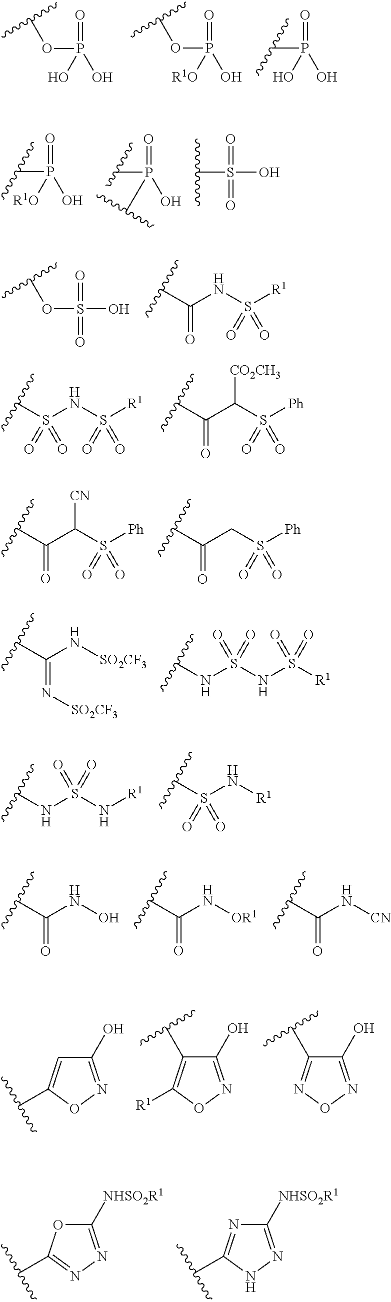

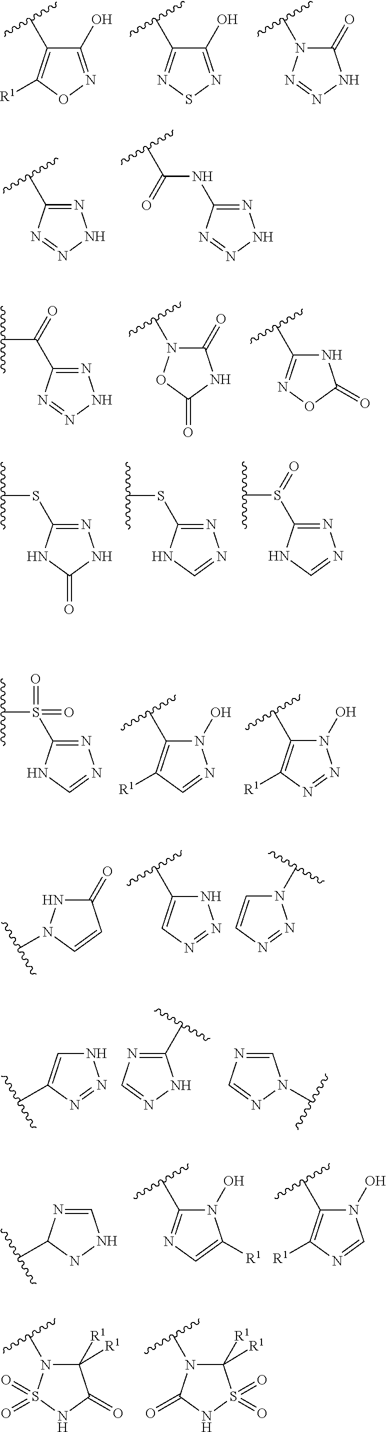

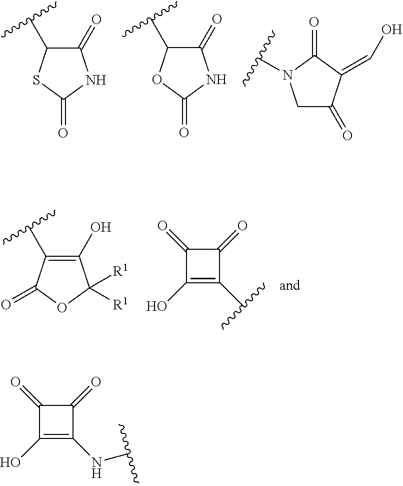

wherein, R¹ is R^{A5}.

In one embodiment, A⁵ is selected from the group consisting of

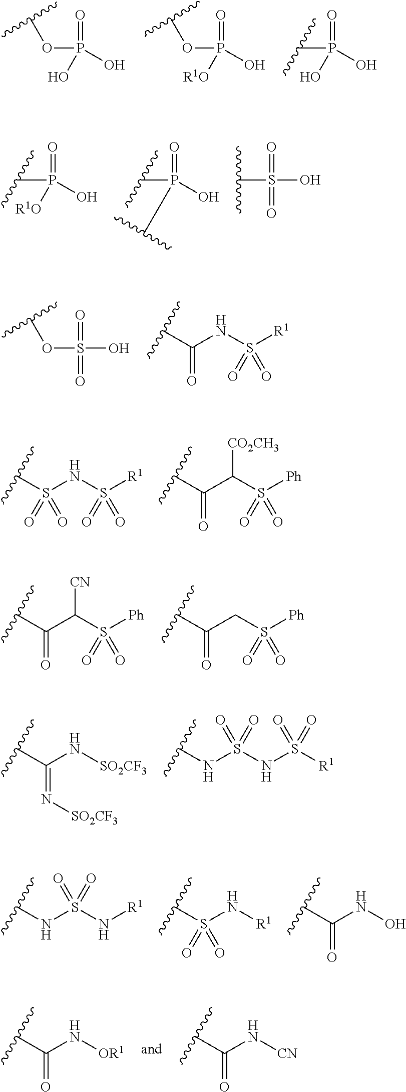

wherein, R¹ is R^{A5}.

In one embodiment, A⁵ is selected from the group consisting of

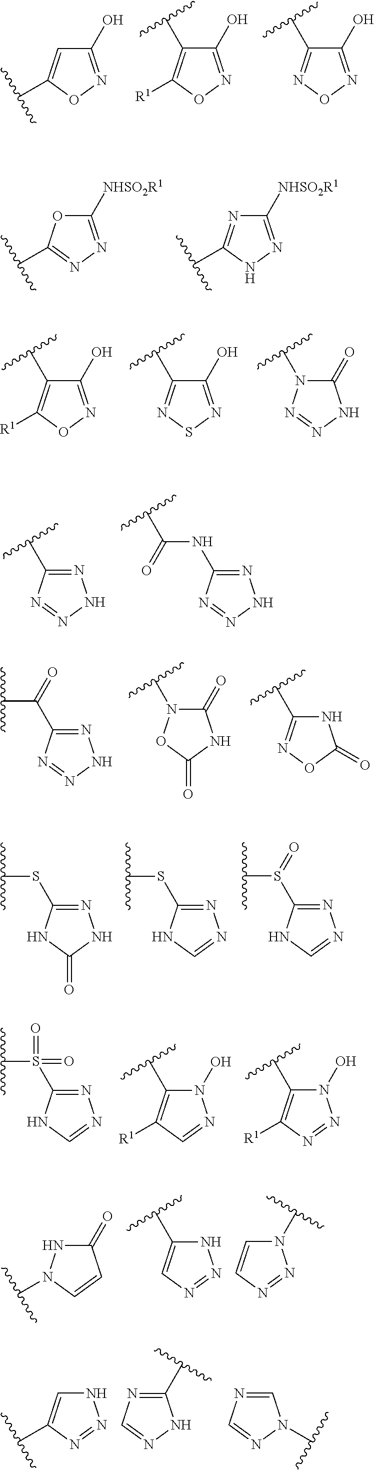

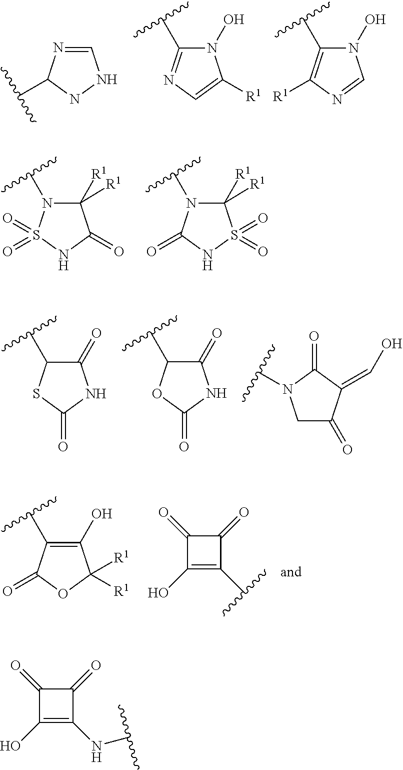

wherein, R¹ is R^{A5}.

In one embodiment, A⁵ is selected from the group consisting of carboxylic acid (—CO₂H), phosphate, phosphonate, phosphonic acid, tetrazole and sulphate.

In one embodiment, A⁵ is selected from the group consisting of carboxylic acid and phosphate. In one embodiment, preferably, A⁵ is carboxylic acid.

In one embodiment, A⁵ is substituted with one or more R^{A5}, wherein R^{A5} is selected from the group consisting of —H, C₁-C₄ alkyl, C₂-C₄ alkenyl and aryl.

In one embodiment, X⁶ is of Formula —N(R)—Y⁵(-L⁴-A^{S})-Z⁶—; wherein R^{e} is —H, C₁-C₁₀ alkyl, aryl or heteroaryl, L⁴ is C₁-C₅ alkyl, Y⁵ is CH, Z⁶ is C═O and A⁵ is carboxylic acid (—CO₂H) or phosphate. In one embodiment, X⁶ is of Formula —N(R^{e})—Y⁵(-L⁴-A⁵)-Z⁶—; wherein R^{e} is —H, C₁-C₁₀ alkyl, aryl or heteroaryl, L⁴ is C₁-C₂ alkyl, Y⁵ is CH, Z⁶ is C═O and A⁵ is carboxylic acid (—CO₂H) or phosphate. In one embodiment, X⁶ is of Formula —N(R^{e})—Y(-L⁴-A^{s})-Z⁶—; wherein R^{e} is —H, C₁-C₄ alkyl or aryl, L⁴ is C₁-C₂ alkyl, Y⁵ is CH, Z⁶ is C═O and A⁵ is carboxylic acid (—CO₂H) or phosphate. In one embodiment, X⁶ is of Formula —N(R^{e})—Y⁵(-L⁴-A^{S})-Z⁶—; wherein R^{e} is methyl, L⁴ is C₁-C₂ alkyl, Y⁵ is CH, Z⁶ is C═O and A⁵ is carboxylic acid (—CO₂H) or phosphate. In one embodiment, X⁶ is of Formula —NH—Y⁵(-L⁴-A^{s})-Z⁶—; wherein L⁴ is C₁ alkyl, Y⁵ is CH, Z⁶ is C═O and A⁵ is carboxylic acid (—CO₂H) or phosphate. In one embodiment, X⁶ is of Formula —NH—Y⁵(-L⁴-A^{s})-Z⁶—; wherein L⁴ is C₂ alkyl, Ys is CH, Z⁶ is C═O and A⁵ is carboxylic acid (—CO₂H) or phosphate. In one embodiment, X⁶ is of Formula —NH—Y⁵(-L⁴-A⁵)-Z⁶—; wherein L⁴ is C₁ alkyl, Y⁵ is CH, Z⁶ is C—O and A⁵ is carboxylic acid (—CO₂H). In one embodiment, X⁶ is of Formula —NH—Y⁵(-L⁴-A⁵)-Z⁶—; wherein L⁴ is C₁ alkyl, Y⁵ is CH, Z⁶ is C═O and A⁵ is phosphate. In one embodiment, X⁶ is of Formula —N(CH₃)—Y⁵(-L⁴-A⁵)-Z⁶—; wherein L⁴ is C₁ alkyl, Y⁵ is CH, Z⁶ is C═O and A⁵ is carboxylic acid (—CO₂H) or phosphate. In one embodiment, X⁶ is of Formula —N(CH₃)—Y⁵(-L⁴-A⁵)-Z⁶—; wherein L⁴ is C₂ alkyl, Y⁵ is CH, Z⁶ is C═O and A⁵ is carboxylic acid (—CO₂H) or phosphate. In one embodiment, X⁶ is of Formula —N(CH₃)—Y⁵(-L⁴-A⁵)-Z⁶—; wherein L⁴ is C₁ alkyl, Y⁵ is CH, Z⁶ is C═O and A⁵ is carboxylic acid (—CO₂H). In one embodiment, X⁶ is of Formula —N(CH₃)—Y⁵(-L⁴-A⁵)-Z⁶—; wherein L⁴ is C₁ alkyl, Y⁵ is CH, Z⁶ is C═O and A⁵ is phosphate. In one embodiment, X⁶ is of Formula —NH—Y⁵(-L⁴-A⁵)-Z⁶—; wherein L⁴ is C₂ alkyl, Y⁵ is CH, Z⁶ is C═O and A⁵ is carboxylic acid (—CO₂H). In one embodiment, X⁶ is of Formula —NH—Y⁵(-L⁴-A⁵)-Z⁶—; wherein L⁴ is C₂ alkyl, Y⁵ is CH, Z⁶ is C═O and A⁵ is phosphate. In one embodiment, X⁶ is of Formula —NH—Y⁵(-L⁴-A⁵)-Z⁶—; wherein L⁴ is C₁ alkyl substituted by methyl, Y⁵ is CH, Z⁶ is C═O and A⁵ is carboxylic acid (—CO₂H). In one embodiment, X⁶ is of Formula —NH—Y⁵(-L⁴-A⁵)-Z⁶—; wherein L⁴ is C₁ alkyl substituted by methyl, Y⁵ is CH, Z⁶ is C═O and A⁵ is phosphate.

In one embodiment, group X⁶ may be a glutamate, an aspartate or a phosphorylated serine residue. In one embodiment, the glutamate, aspartate or phorphorylated serine residue of group X⁶ is an L amino acid. In a further embodiment, X⁶ may be phosphorylated threonine.

In one embodiment, preferably group X⁶ is a glutamate or aspartate residue. This eliminates the requirement for phosphorylated serine residues, which are naturally present within the phosphodegeneron sequence, whilst retaining binding. The negatively charged phosphorylated serine residues are not synthetically desirable.

In one embodiment, the glutamate, aspartate or phosphorylated scrine residue of X⁶ may be substituted with methyl. In another embodiment, the glutamate, aspartate or phosphorylated serine residue of group X⁶ may be substituted with ethyl.

### Group X⁷

It is believed that group X⁷ forms a hydrogen bond with the βTrCP binding domain. Accordingly, X⁷ is a functional group which is capable of forming such a hydrogen bond with the βTrCP binding domain.

X⁷ is a group —N(R^{N1})(RN^{N2}); wherein R^{N1} and R^{N2} are as previously defined. In one embodiment, preferably X⁷ is —NH₂. In one embodiment, X⁷ is —N(R^{N1})₂, wherein one R^{N1} is —H and the other R^{N1} is C₁-C₁₀ alkyl or aryl. In one embodiment, X⁷ is —N(R^{N1})₂, wherein both R^{N1} are independently C₁-C₁₀ alkyl or aryl.

In one embodiment, X⁷ is —N(R^{N1})(R^{N2}), wherein R^{N2} is —(CH₂)₀₋₁₀—(Z⁷)₀₋₁-A^{a}, and wherein A^{a} is —OH. In one embodiment, X⁷ is —N(R^{N1})(R^{N2}), wherein R^{N2} is —(CH₂)₀₋₁₀—(Z⁷)₀₋₁-A^{a}, and wherein A^{a} is —NH₂. In one embodiment, X⁷ is —N(R^{N1})(R^{N2}), wherein R^{N2} is —(CH₂)₄₋₈—(Z⁷)₀₋₁-A^{a}, and wherein A^{a} is —NH₂. In one embodiment, X⁷ is —N(R^{N1})(R^{N2}), wherein R^{N2} is —(CH₂)₀₋₁₀—(Z⁷)₀₋₁₀-A^{a}, and wherein A^{a} is —C(O)NH₂. In one embodiment, X⁷ is —N(R^{N1})(R^{N2}), wherein R^{N2} is —(CH₂)₁₋₁₀—(Z⁷)₀₋₁-A^{a}, and wherein A^{a} is a chain of one or more naturally occurring amino acids. In one embodiment, X⁷ is —N(R^{N1})(R^{N2}), wherein R^{N2} is —(CH₂)₀₋₁₀—(Z⁷)₀₋₁-A^{a}, and wherein A^{a} is a chain of one or more non-naturally occurring amino acids. In one embodiment, X⁷ is —N(R^{N1})(R^{N2}), wherein R^{N2} is —(CH₂)₀₋₁₀—(Z⁷)₀₋₁-A^{a}, and wherein A^{a} is a chain of a mixture of one or more naturally occurring amino acids and one or more non-naturally occurring amino acids. In one embodiment, X⁷ is —N(R^{N1})(R^{N2}), wherein R^{N2} is —(CH₂)₀₋₁₀—(Z⁷)₀₋₁-A^{a}, and wherein A^{a} is a cholesteryl derivative.

In one embodiment, X⁷ is —N(R^{N1})(R^{N2}), wherein R_{N2} is —(CH₂CH₂O)₁₋₁₀ —CH₂CH₃. In one embodiment, X⁷ is —N(R^{N1})(R^{N2}), wherein R^{N2} is —(CH₂CH₂O)₁₋₁₀—(CH₂)₁₋₃—(Z⁷)₀₋₁-A^{a}, and wherein A^{a} is —NH₂ or —C(O)NH₂. In one embodiment, X⁷ is —N(R^{N1})(R^{N2}), wherein R^{N2} is —(CH₂CH₂O)₁₋₁₀—(CH₂)₁₋₃—(Z⁷)₀₋₁-A^{a}, and wherein A^{a} is a chain of one or more naturally occurring amino acids. In one embodiment, X⁷ is —N(R^{N1})(R^{N2}), wherein R^{N2}—(CH₂CH₂O)₄₋₈—(CH₂)₁₋₃—(Z⁷)₀₋₁-A^{a}, and wherein A^{a} is a chain of one or more naturally occurring amino acids. In one embodiment, X⁷ is —N(R^{N1})(R^{N2}), wherein R^{N2}—(CH₂CH₂O)₁₋₁₀—(CH₂)₁₋₃—(Z⁷)₀₋₁-A^{a}, and wherein A^{a} is a chain of one or more non-naturally occurring amino acids. In one embodiment, X⁷ is —N(R^{N1})(R^{N2}), wherein R^{N2}—(CH₂CH₂O)₄₋₈—(CH₂)₁₋₃—(Z⁷)₀₋₁-A^{a}, and wherein A^{a} is a chain of one or more non-naturally occurring amino acids. In one embodiment, X⁷ is —N(R^{N1})(R^{N2}), wherein R^{N2}—(CH₂CH₂O)₁₋₁₀—(CH₂)₁₋₃—(Z⁷)₀₋₁-A^{a}, and wherein A^{a} is a cholesteryl derivative. In one embodiment R^{N2} is —(CH₂)₀₋₁₀—(Z⁷)₀₋₁-A^{a} and A^{a} is a cholesteryl derivative, in particular the cholesteryl derivative is:

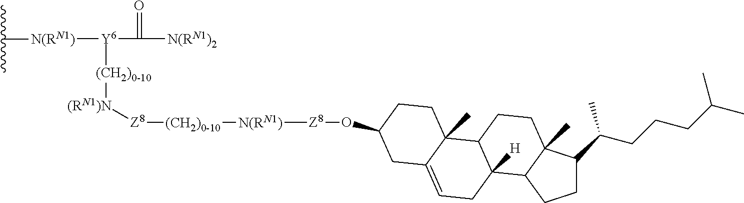

wherein R^{N1} is as previously defined, Y⁶ is CH or N and Z⁸ is selected from the group consisting of C═O, C═S, CH₂, S═O, S(O)₂, C═N(C₁-C₄) alkyl) and C═NH. In particular, the cholesteryl derivative is:

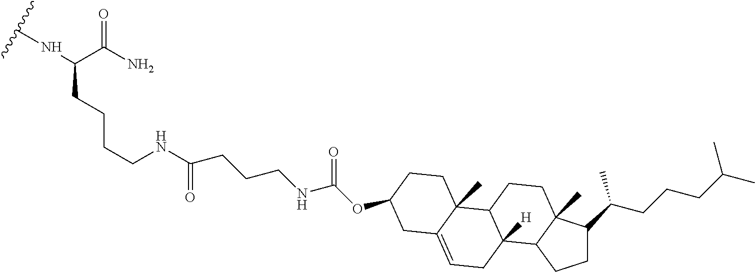

It is believed that the cholesteryl group enhances the cell penetratation of the compounds and modified peptides of the invention, without affecting the activity of the compounds and modified peptides of the invention against the targets of the invention.

### Additional Amino Acids/Chains of Substituents

Compounds of the invention may additionally contain one or two chains of 1, 2, 3, 4, 5, 6, 7, 8, 9, 10 or more amino acids. For example, in one embodiment, group B may be substituted with —NH(R^{N2}) or —N(R^{N2})₂, wherein one R^{N2} is a chain of 1, 2, 3, 4, 5, 6, 7, 8, 9, 10 or more naturally occurring or non-naturally occurring amino acids.

Alternatively, group X⁷ may be of Formula —N(R^{N1})(R^{N2}), wherein R^{N1} is preferably —H and R^{N2} is a chain of 1, 2, 3, 4, 5, 6, 7, 8, 9, 10 or more naturally or non-naturally occurring amino acids.

Alternatively, group E may be substituted with —NH(R^{N2}) or —N(R^{N2})₂, wherein one R^{N2} is a chain of 1, 2, 3, 4, 5, 6, 7, 8, 9, 10 or more naturally occurring or non-naturally occurring amino acids and X⁷ may be of Formula —N(R^{N1})(R^{N2}), wherein R^{N2} is a chain of 1, 2, 3, 4, 5, 6, 7, 8, 9, 10 or more amino acids, thus providing a compound containing two additional chains of amino acids. The one or more naturally occurring or non-naturally occurring amino acids are independently selected from the group consisting of L-amino acids, D-amino acids and aza-amino acids.

Chains of non-naturally occurring amino acids may include peptoids, which are peptidomimetics whose side chains are appended to the nitrogen atom of the peptide, rather than to the alpha-carbons.

### Modified Peptide Embodiment of Formula Ia

In one embodiment, the compound of Formula Ia may comprise a sequence of amino acids according to the following Formula Ic:

X¹-E/D/pS-G-X⁴-X⁵-E/D/pS-NHR^{N2} Formula Ic

X¹, X⁴, X⁵ and R^{N2} are as previously defined.

In one embodiment, the compound of Formula Ia may comprise a sequence of amino acids according to the following Formula Iv:

d-E-G-F(3F)-W-E-NHR^{N2} Formula Iv

Herein “comprise” is used in the open sense to indicate that additional amino acids may also be present in the sequence.

The amino acids of the Formula depicted above preferably form a contiguous sequence.

In order to arrive at Formula Ic, the inventors used the phosphodegeneron sequence as a starting point, and systematically substituted each of the amino acids to alternative natural and non-natural amino acids. At each stage the binding of the substituted peptides to βTrCP was assessed and further substituted peptides were designed in order to maximise binding.

Within the meaning of the present invention, the term “modified” indicates that the peptide is not naturally occurring. A modified peptide may contain one or more non-naturally occurring amino acids, and/or may include one or more moieties which are not classified as amino acids.

Within the present invention, the term “residue” will be used to refer to each of the component moieties of the modified peptide, whether these are amino acids or other chemical moieties. Within Formula Ic, the individual residues are shown separated by hyphens (“−”).

Where the residues of the modified peptide are amino acids, each of the amino acids may be independently selected from an L-amino acid, a D-amino acid and an aza-amino acid. One or more of the residues may additionally be independently substituted at one or more positions irrespective of which subtype of amino acid forms the basis for the residue.

An “L amino acid” is defined as an amino acid which can theoretically be synthesised from levorotatory glyceraldehyde. Amino acids found in naturally occurring proteins are usually L amino acids. According to generally accepted notation, L amino acids are depicted herein using the capital letter single letter amino acid code.

A “D amino acid” is the stereoisomer of an L amino acid and is defined as an amino acid which can theoretically be synthesised from dextrorotary glyceraldehyde. According to generally accepted notation, D amino acids are depicted herein using the lower case single letter amino acid code.

An “aza amino acid” is an L amino acid in which the α-carbon atom has been replaced by a nitrogen atom. The replacement of the αC—COOH bond found in naturally occurring amino acids with an αN—COOH bond can increase the stability of a peptide.

Herein, the suffix “p” is used to denote a phosphorylated residue, e.g. “pS” denotes phosphorylated serine.

The compounds of the present invention bind to βTrCP. In one embodiment the compounds are considered to “bind to βTrCP” if they bind with an affinity of less than about 10 μM. In some embodiments the compounds/peptides may bind to βTrCP with an affinity of less than about 900 nM, less than about 800 nM, less than about 700 nM, less than about 600 nM, less than about 500 nM, less than about 400 nM, less than about 300 nM, less than about 200 nM, less than about 150 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 9 nM, less than about 8 nM, less than about 7 nM, less than about 6 nM, less than about 5 nM, less than about 4 nM, less than about 3 nM, less than about 2 nM, less than about 1 nM or less.

### Capping Groups

The compounds or modified peptides of the present invention may comprise a capping group. The function of the capping group is to increase the stability of the compound towards enzymic degradation, thus improving cell penetration, and any groups which are known to perform this function may be used as capping groups. In embodiments where a capping group is present, the definitions given for compounds of Formula Ia, Ib and Ic above equally apply.

In one embodiment, any amino/amine group, in particular an —NH₂, —NH(R^{N1}), or —NH(R^{N2}) group which is present in a compound of the present invention may be capped, by replacement of a H atom with a capping group. Suitable capping groups include any groups which are known to prevent the compound from being degraded on entry into a cell.

In one embodiment, the capping group may be selected from the group consisting of

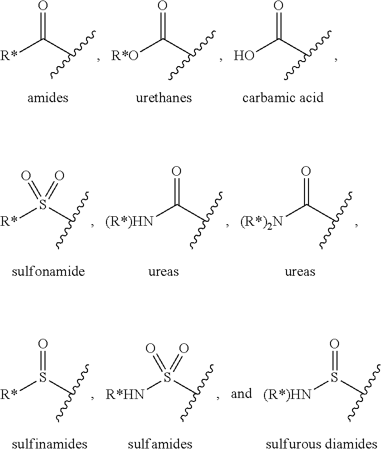

and the thiocarbonyl derivatives of any of these capping groups.

The person skilled in the art will recognise that R* is used to indicate a generic structure for the purposes of illustrating the various functional groups which may be suitable as amine/amino capping groups. Specific examples of capping groups are illustrated below.

In one embodiment, a compound of the present invention is substituted by an amino/amine group, in particular an —NH₂, —NH(R^{N1}), or —NH(R^{N2}) group as defined previously, wherein said amino/amine group, in particular the —NH₂, —NH(R^{N1}), or —NH(R^{N2}) group, is capped, by the replacement of a H atom with a capping group selected from the group consisting of:

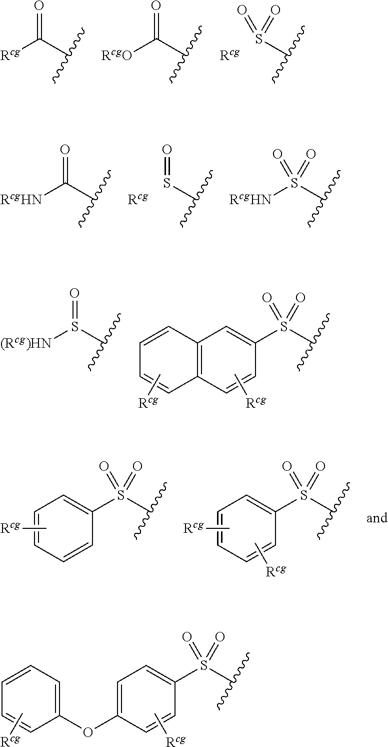

wherein,

R^{cg} is selected from the group consisting of —H, —F, —Cl, —Br, —I, —OH, —O(C₁-C₁₀ alkyl), —CN, —NO₂, —CF₃, —OCF₃, —CO₂H, —NH₂, —NH(C₁-C₂ alkyl), —N(C₁-C₂ alkyl)₂, —C₁-C₁₀ alkyl, aryl and heteroaryl.

In a further embodiment, the capping group may be selected from the group consisting of:

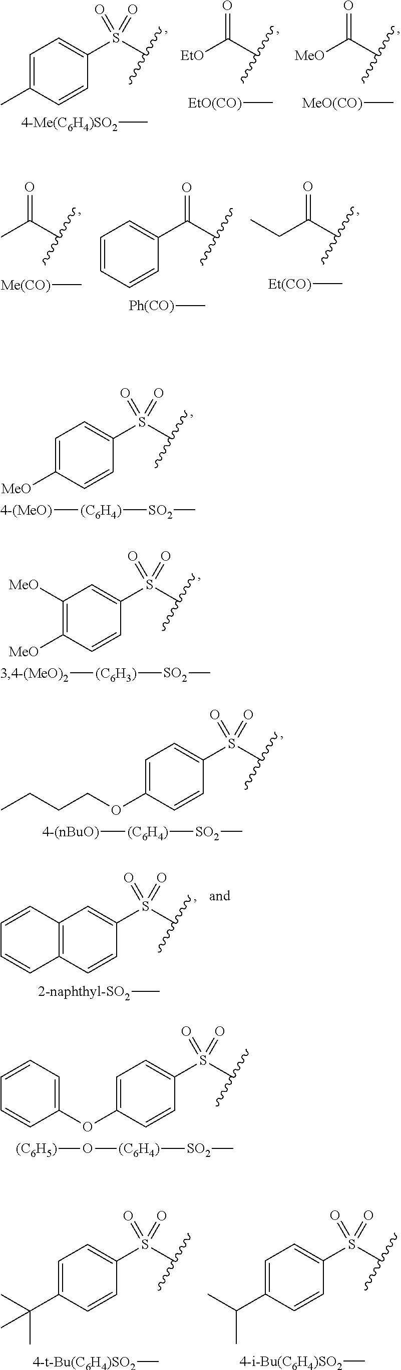

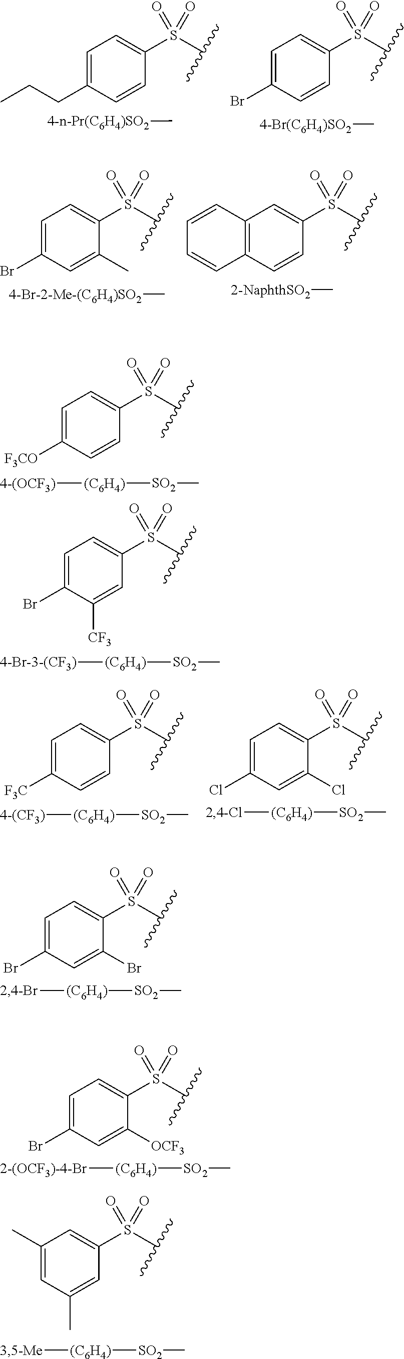

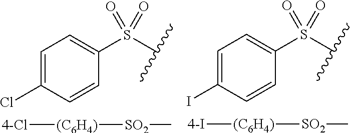

Further capping groups which may be used in the synthesis of compounds of the present invention are:

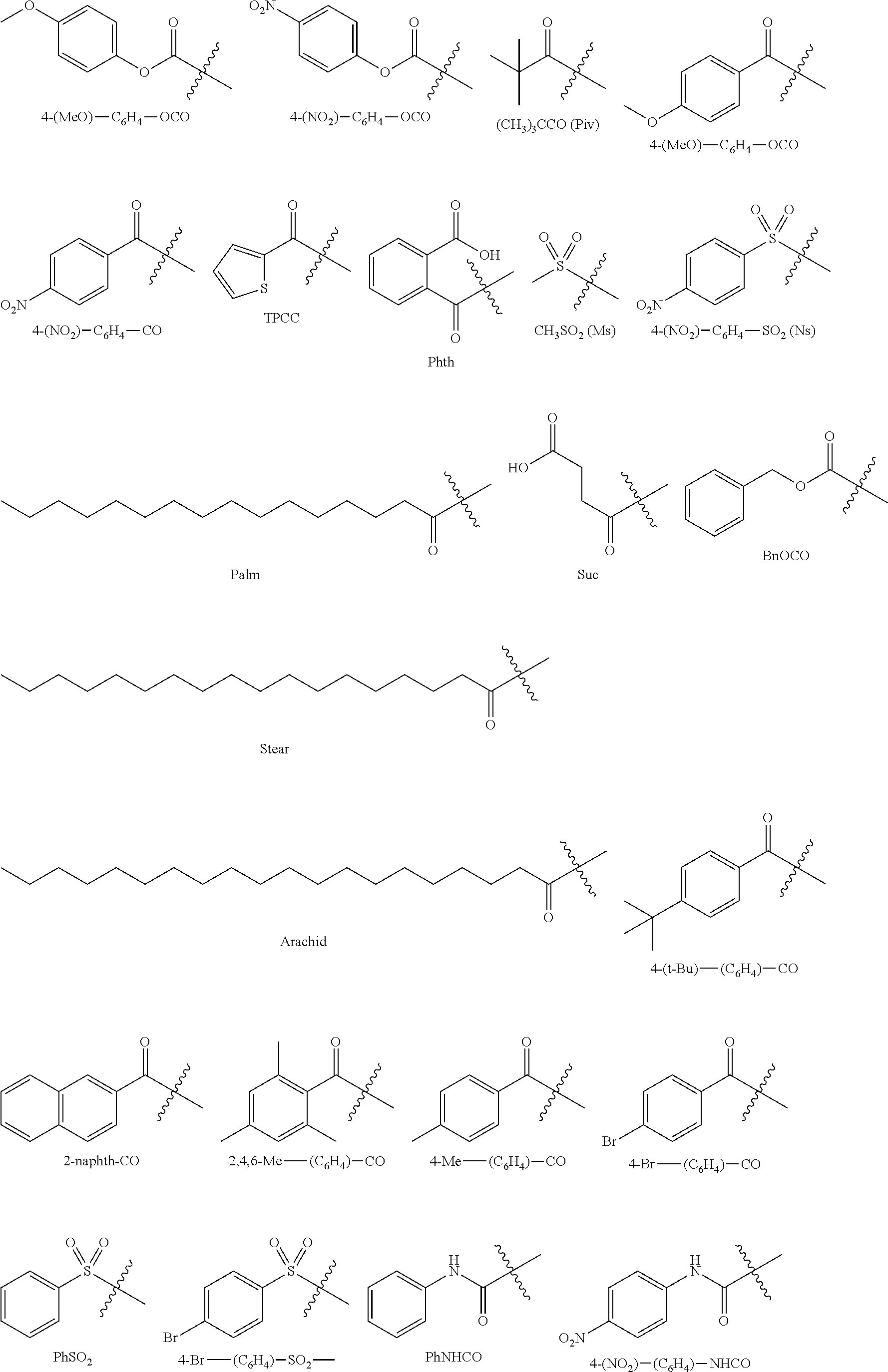

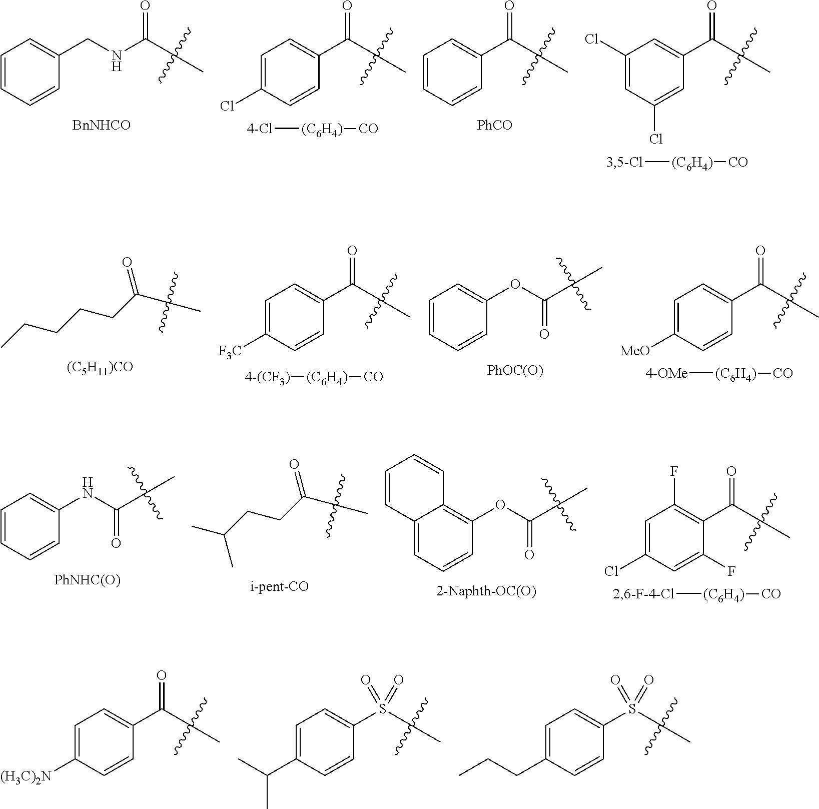

Acidic capping groups which may be used in the synthesis of compounds of the present invention are:

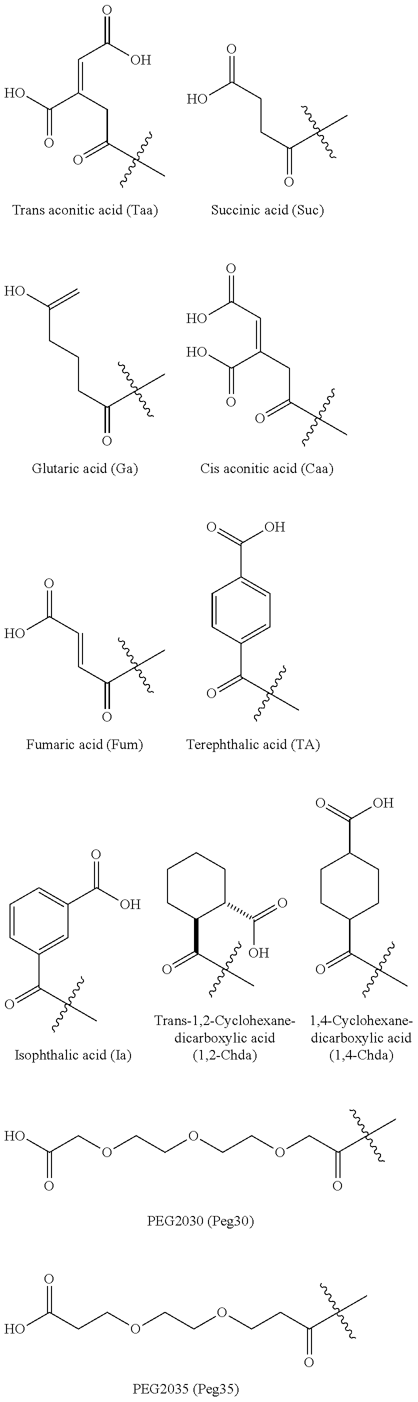

In one embodiment, group B is substituted by a substituent of Formula —NH₂, —NH(R^{N1}), or —NH(R^{N2}), wherein the —NH₂, —NH(R^{N1}) or —NH(RN²) substitucnt is capped, by the replacement of a H atom, with a capping group selected from the group consisting of:

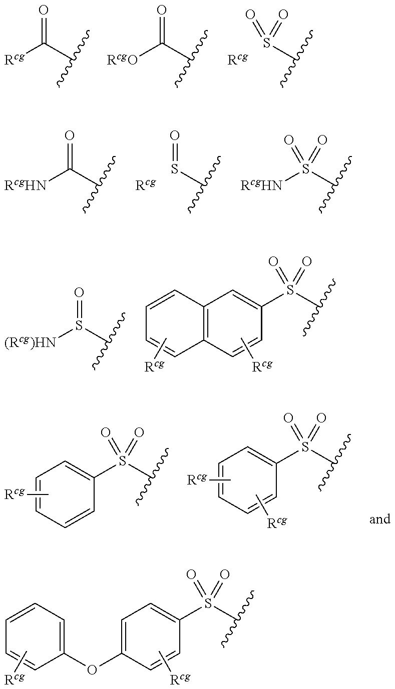

wherein, R^{cg} is as previously defined.

In a further embodiment, group B is substituted by a substituent of Formula —NH₂, —NH(R^{N1}), —NH(R^{N2}), wherein the —NH₂, —NH(R^{N}), —NH(R^{N2}) substituent is capped, by the replacement of a H atom, with a capping group selected from the group consisting of:

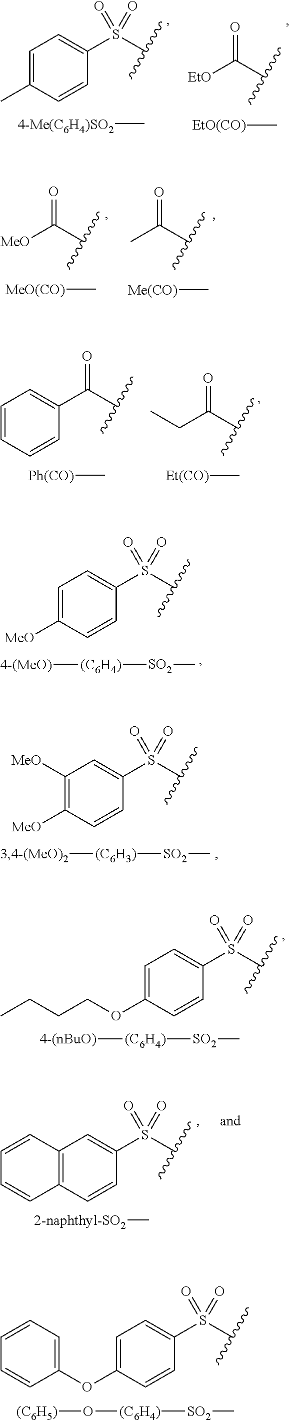

In one embodiment, X¹ is aspartyl or glutamyl and comprises a capping group. In one embodiment, X¹ is aspartyl and comprises a capping group on the N-terminus. In one embodiment, X¹ is aspartyl or glutamyl and comprises a capping group on the N-terminus, wherein the capping group is selected from the group consisting of:

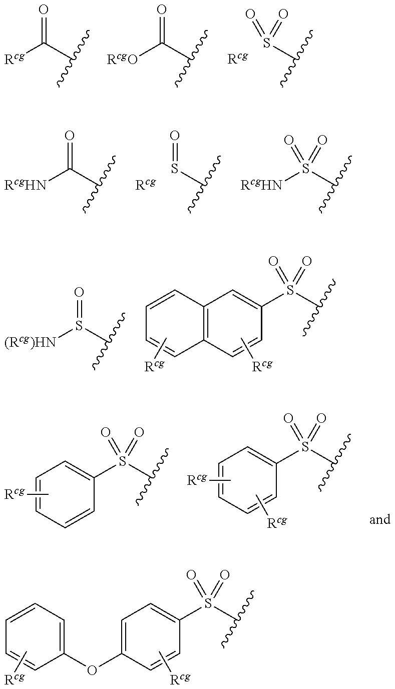

wherein, R^{cg} is as previously defined.

In a further embodiment, X¹ is aspartyl or glutamyl and comprises a capping group on the N-terminus, wherein the capping group is selected from the group consisting of:

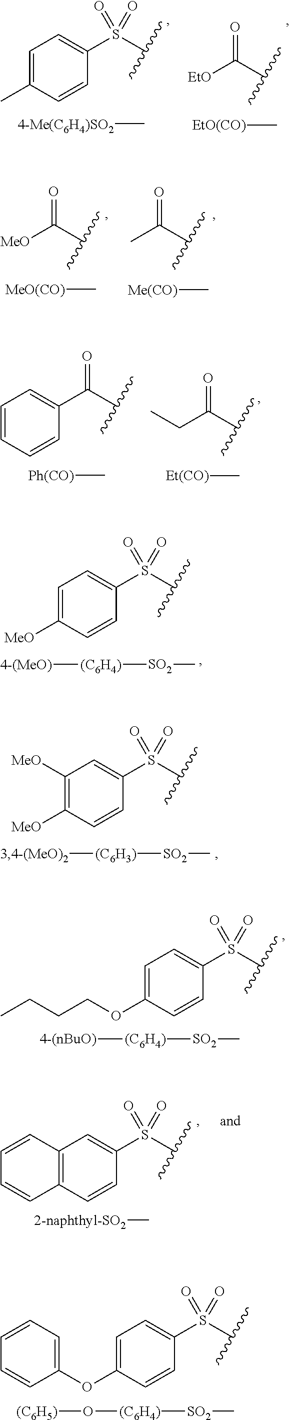

In one embodiment, X¹ is aspartyl and comprises a capping group on the N-terminus, wherein the capping group is selected from the group consisting of:

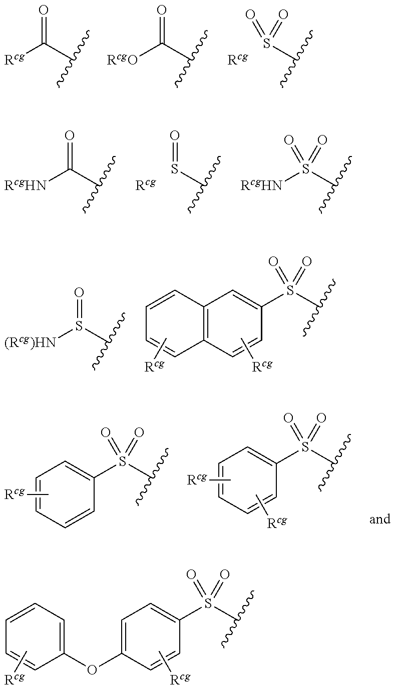

wherein, R^{cg} is as previously defined.

In one embodiment, X¹ is aspartyl and comprises a capping group on the N-terminus, wherein the capping group is selected from the group consisting of:

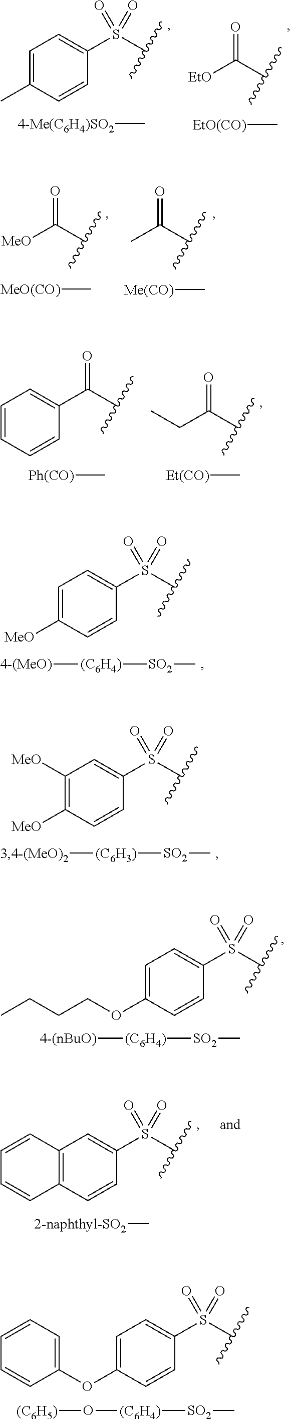

Preferred capping groups are those selected from List 1:

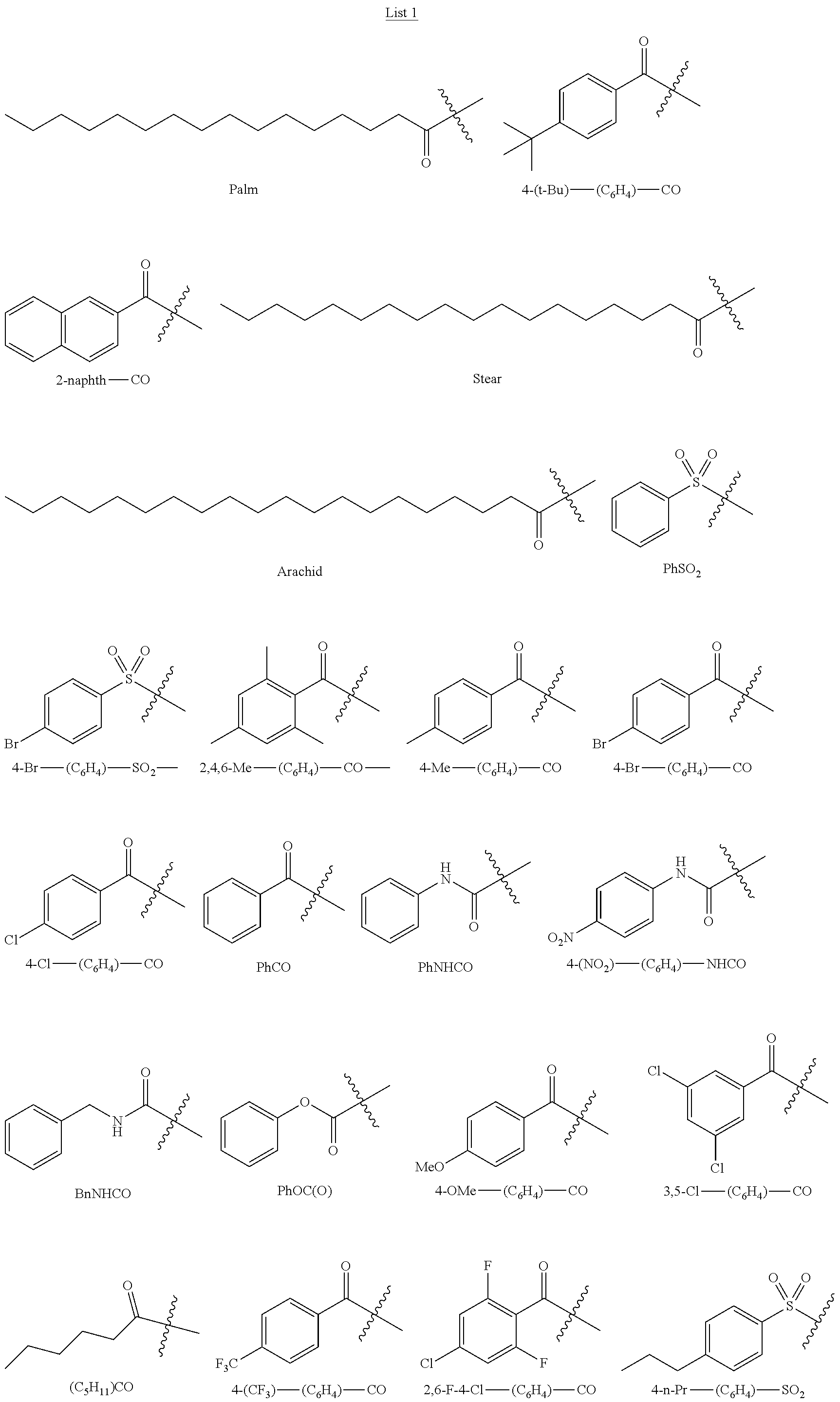

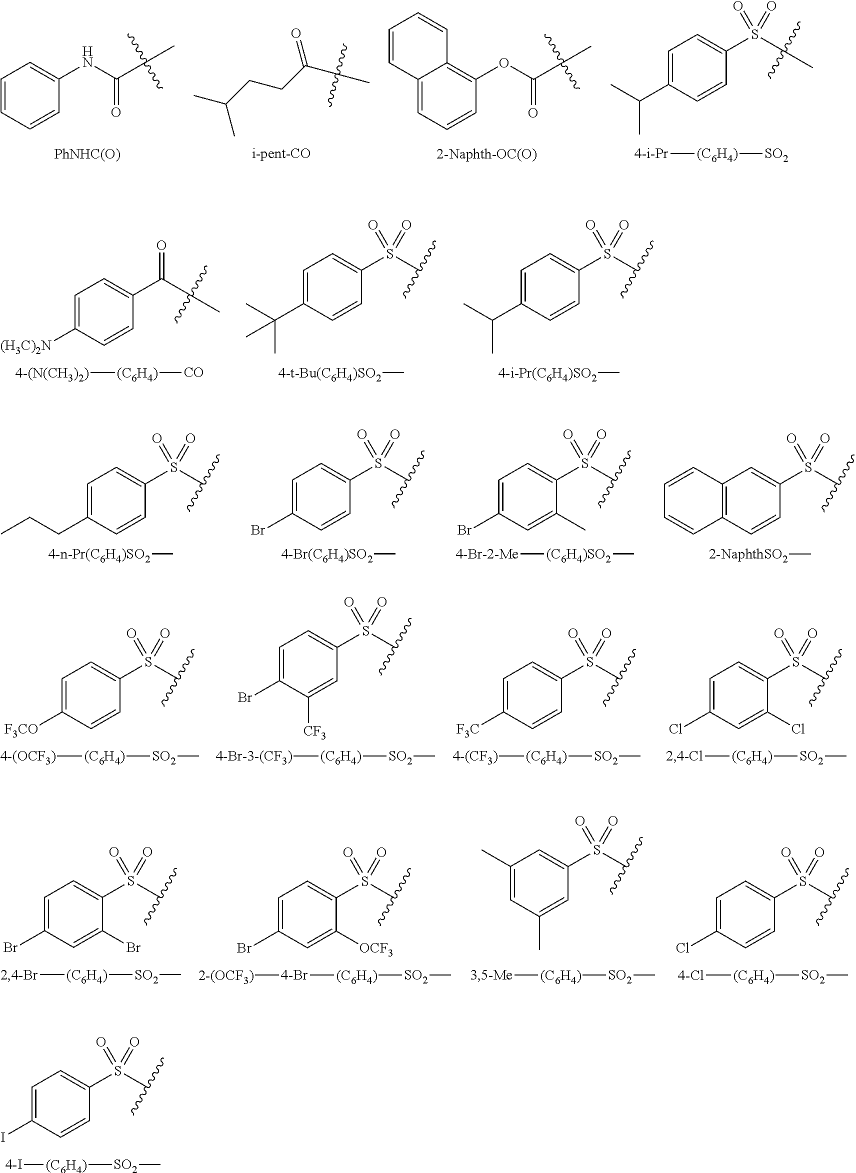

In a further embodiment, group B is substituted by a substituent of Formula —NH₂, —NH(R^{N1}), —NH(R^{N2}), wherein the —NH₂, —NH(R^{N1}), —NH(R^{N2}) substituent is capped, by the replacement of a H atom, with a capping group selected from the group consisting of those selected from List 2:

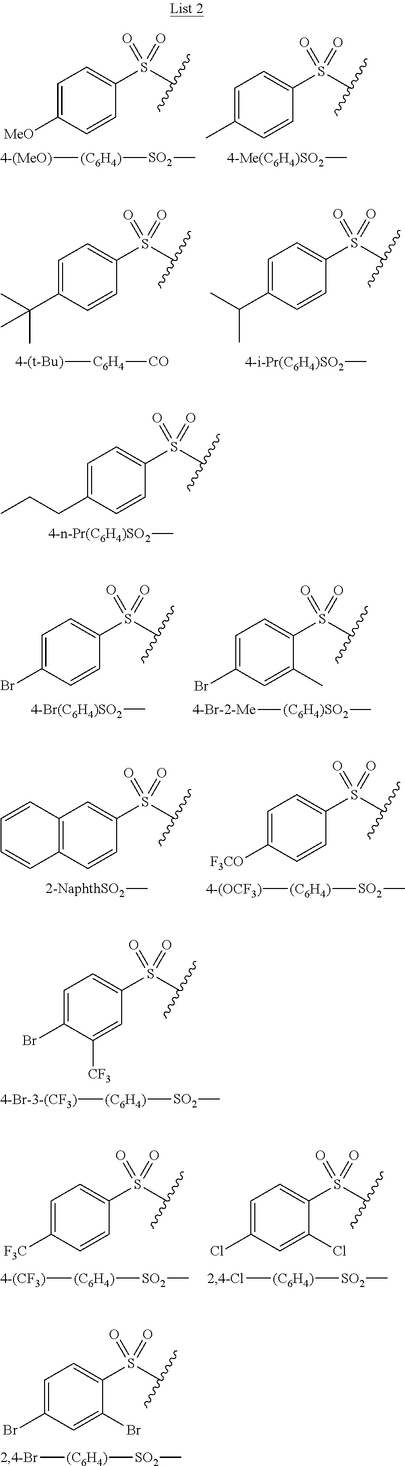

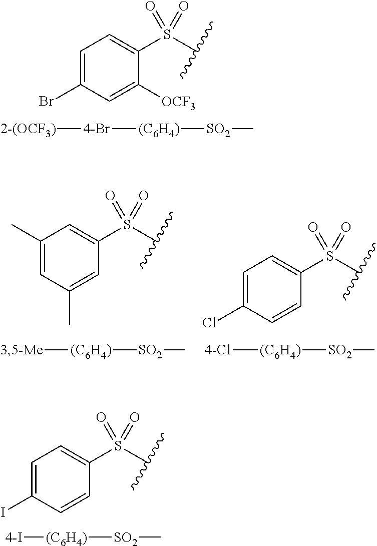

In such an embodiment, X¹ may be aspartyl or glutamyl, in particularly aspartyl, which comprises a capping group on the N-terminus, wherein the capping group is selected from the group consisting of List 2.

As described above, in some embodiments R^{N2} is —(CH₂)₀₋₁₀—(Z⁷)₀₋₁-A^{a}, wherein Z⁷ is C═O, and A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids and comprises a capping group, wherein the capping group is selected from the group consisting of List 3:

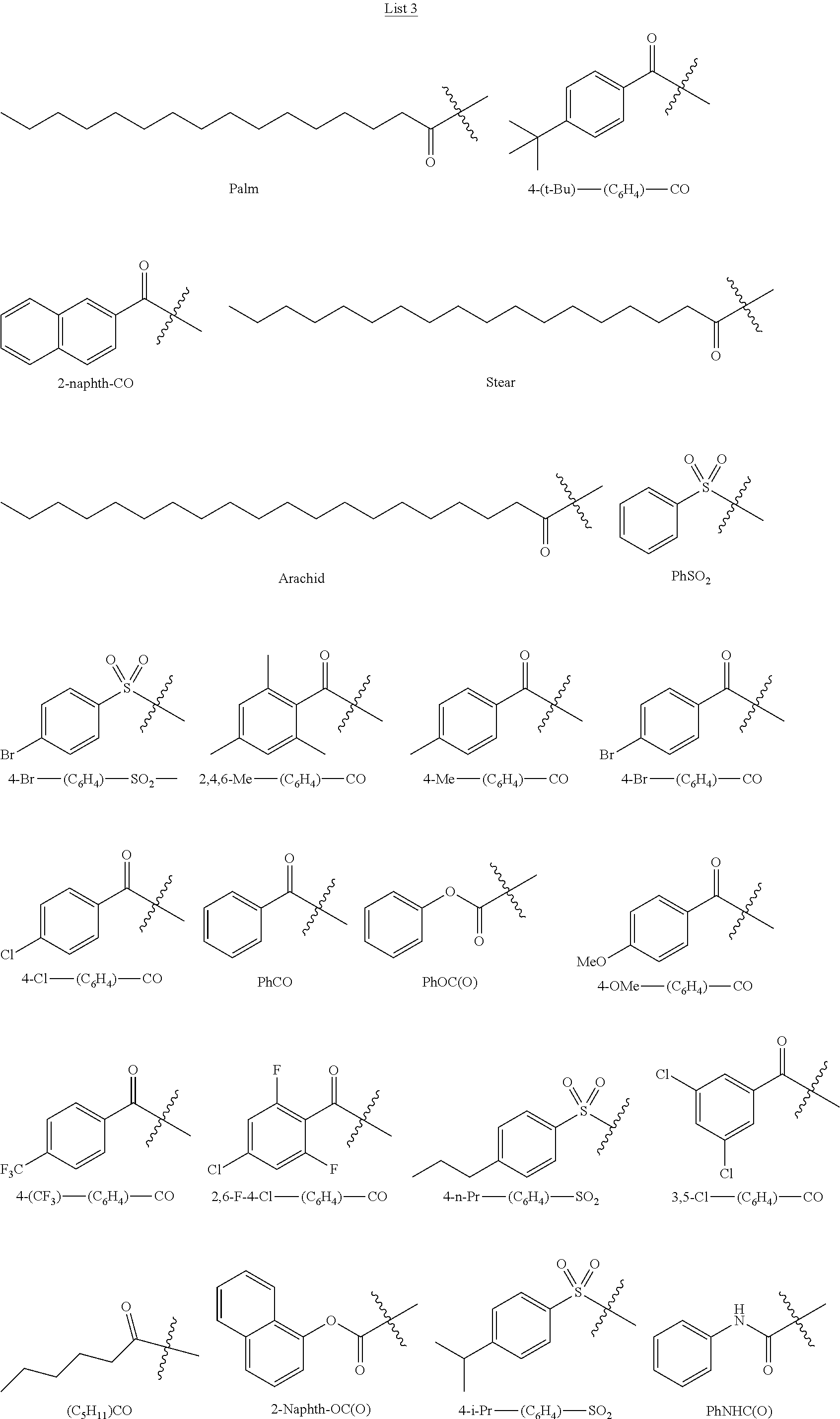

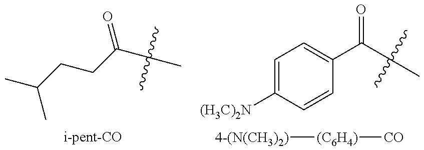

In particularly A^{a} may be lysyl, with a capping group on an N as illustrated below (Formula M), in particular where the capping group is a capping group selected from List 3.

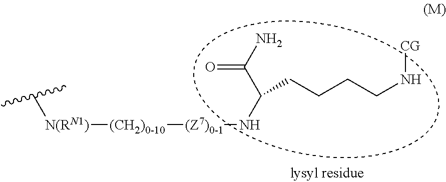

Advantageously, the capping group on A^{a} does not have a detrimental effect on the activity of the compounds or modified peptides of the invention.

Where the compounds or modified peptides of the present invention include more than one capping group, all combinations of the capping groups described herein are envisaged. In particular, when group B has a capping group selected from List 2, and R^{N2} is as defined above in association with List 3, and has a capping group selected from List 3, all combinations of capping groups from List 2 and List 3 are envisaged.

Particular combinations of capping groups may increase the ability of the compounds and modified peptides of the invention to penetrate cells.

Exemplary combinations of capping groups are as follows;

X¹ is aspartyl or glutamyl and comprises a capping group on the N-terminus, wherein the capping group is:

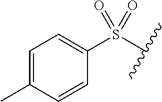 [SMILES: *S(=O)(=O)c1ccc(C)cc1]

4-Me-C₆H₄)SO₂—, and;

R^{N2} is, —(CH₂)₀₋₁₀—(Z⁷)₀₋₁-A^{a}, wherein Z⁷ is C═O, and A^{a} is lysyl and comprises a capping group as illustrated in Formula M, wherein the capping group is;

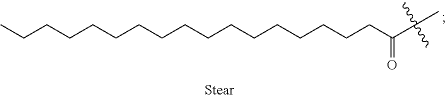 [SMILES: *C(=O)CCCCCCCCCCCCCCCCC]

X¹ is aspartyl or glutamyl and comprises a capping group on the N-terminus, wherein the capping group is:

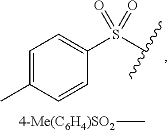

and;

R^{N2} is, —(CH₂)₀₋₁₀—(Z⁷)₀₋₁-A^{a}, wherein Z⁷ is C═O, and A^{a} is lysyl and comprises a capping group as illustrated in Formula M, wherein the capping group is;

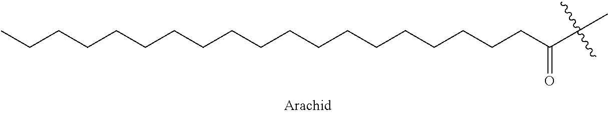 [SMILES: *C(=O)CCCCCCCCCCCCCCCCCCC]

X¹ is aspartyl or glutamyl and comprises a capping group on the N-terminus, wherein the capping group is:

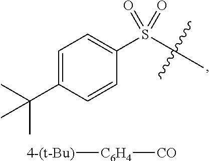

and;

R^{N2} is, —(CH₂)₀₋₁₀—(Z⁷)₀₋₁-A^{a}, wherein Z⁷ is C═O, and A^{a} is lysyl and comprises a capping group as illustrated in Formula M, wherein the capping group is;

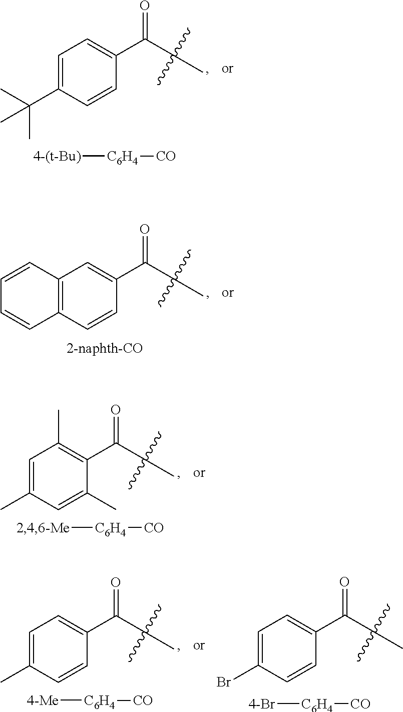

X¹ is aspartyl or glutamyl and comprises a capping group on the N-terminus, wherein the capping group is:

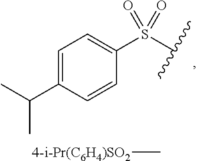

and;

R^{N2} is, —(CH₂)₀₋₁₀—(Z⁷)₀₋₁-A^{a}, wherein Z⁷ is C═O, and A^{a} is lysyl and comprises a capping group as illustrated in Formula M, wherein the capping group is;

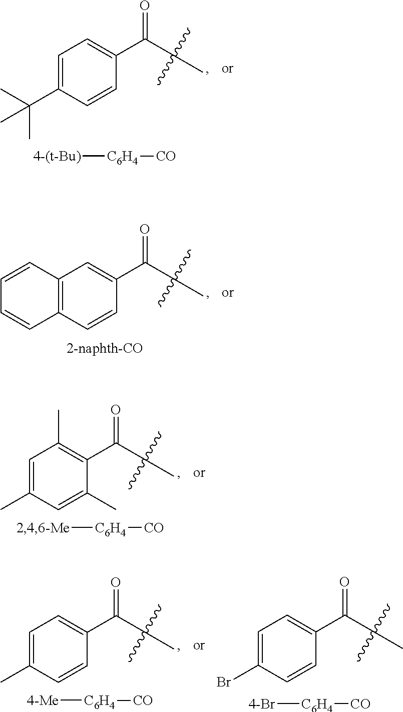

X¹ is aspartyl or glutamyl and comprises a capping group on the N-terminus, wherein the capping group is:

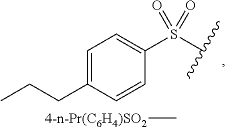

and;

R^{N2} is, —(CH₂)₀₋₁₀—(Z⁷)₀₋₁-A^{a}, wherein Z⁷ is C═O, and A^{a} is lysyl and comprises a capping group as illustrated in Formula M, wherein the capping group is;

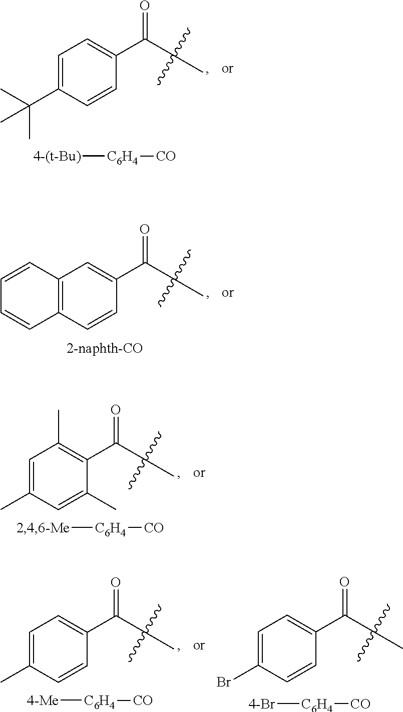

X¹ is aspartyl or glutamyl and comprises a capping group on the N-terminus, wherein the capping group is:

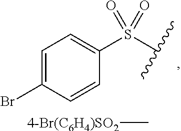

and;

R^{N2} is, —CH₂)₀₋₁₀—(Z⁷)₀₋₁-A^{a}, wherein Z⁷ is C═O, and A^{a} is lysyl and comprises a capping group as illustrated in Formula M, wherein the capping group is;

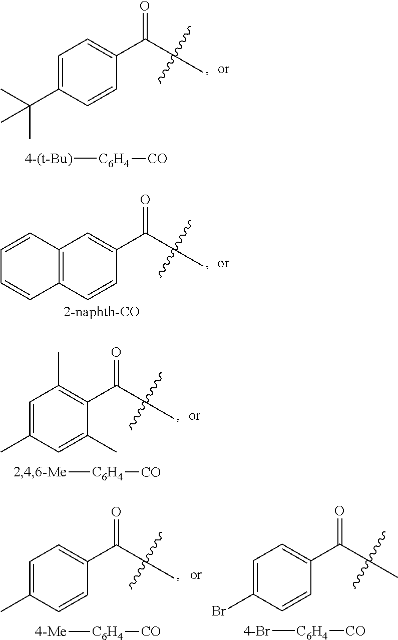

X¹ is aspartyl or glutamyl and comprises a capping group on the N-terminus, wherein the capping group is:

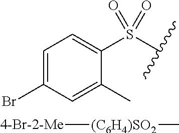

R^{N2} is, —(CH₂)₀₋₁₀—(Z⁷)₀₋₁-A^{a}, wherein Z⁷ is C═O, and A^{a} is lysyl and comprises a capping group as illustrated in Formula M, wherein the capping group is;

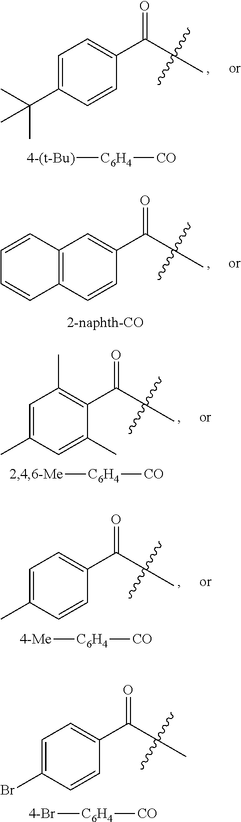

A capping group can be added to a compound according to any one of the above-described aspects of the invention. For example, the compound may be of Formula Id:

Capping group-X¹-X²-X³-X⁴-X⁵-X⁶-X⁷ Formula Id

In one embodiment, the compound may be of Formula Ie

X¹-X²-X³-X¹-X⁵-X⁶-X⁷-Capping group Formula Ie

In one embodiment, the compound may be of Formula If

Capping group-X¹-X²-X³-X⁴-X⁵-X⁶-X⁷-Capping group Formula If

In one embodiment, the compound may be a modified peptide of Formula Ig:

Capping group-X¹-E/D/pS-G-X⁴-X⁵-E/D/pS-NHR^{N2} Formula Ig

In particular, the compound may be a modified peptide of Formula Iw:

Capping group-d-E-G-F(3F)-W-E-NHR^{N2}

In particular, the compound may be a modified peptide of Formula Ix:

Capping group-d-E-G-F(3F)-W-E-NHR^{N2}-Capping group

### Cyclised Peptides

In one embodiment of the present invention, the compound may be a modified peptide, wherein said modified peptide may be cyclised.

Cyclisation of the modified peptide may require the addition of one or more additional residues to the peptide sequences described above. In particular, enough additional residues are required to enable a carboxy-terminal group at one end of the linear sequence to bind to the amino-terminal group at the other end of the sequence and form a cyclised peptide. 1, 2, 3, 4, 5, 6, 7, 8, 9 or 10 additional residues may be required for this purpose.

### Chemical Groups

### Halo

The term “halogen” (or “halo”) is used herein to refer to fluorine, chlorine, bromine and iodine. In one embodiment, “halogen” is fluorine. In another embodiment, “halogen” is chlorine.

### Carbonyl and Carboxy

Structure C═O represents a carbonyl group, which is a carbon atom connected with a double bond to an oxygen atom, and tautomeric forms thereof. A carbonyl group may also be denoted as —C(O)—. Examples of moieties that contain a carbonyl include but are not limited to aldehydes —C(O)H, ketones —C(O)—(C₁-C₁₀ alkyl)-, carboxylic acids —CO₂H, amides —C(O)NH₂, —C(O)—NH(C₁-C₁₀ alkyl), —C(O)—N(C₁-C₁₀ alkyl)₂, —NH—C(O)—(C₁-C₁₀ alkyl) and esters —C(O)—O(C₁-C₁₀ alkyl).

### Amine, Amino etc.

An amine group is denoted by —NH₂, in which a nitrogen atom is covalently bonded to two hydrogen atoms. An alkylamino group is denoted by —NH(C₁-C₁₀ alkyl), in which a nitrogen atom is covalently bonded to one hydrogen atom and one (C₁-C₁₀ alkyl) group. A dialkylamino group is denoted by —N(C₁-C₁₀ alkyl)₂, in which a nitrogen atom is bonded to at least two additional (C₁-C₁₀ alkyl) groups. Amines may be named in several ways. Typically, a compound is given the prefix “amino” or the suffix “amine”.

### Alkyl, Cycloalkyl, Heterocyclyl, Alkenyl, Alkynyl

The term “alkyl” is used herein to refer to monovalent, divalent or trivalent straight or branched, saturated, acyclic hydrocarbyl groups. In one embodiment, alkyl is C₁-C₁₀ alkyl, in another embodiment C₁-C₆ alkyl, in another embodiment C₁-C₄ alkyl, such as methyl, ethyl, n-propyl, i-propyl, n-butyl or t-butyl groups.

The term “cycloalkyl” is used herein to refer to monovalent, divalent or trivalent saturated, cyclic hydrocarbyl groups. In one embodiment cycloalkyl is C₃₋₁₀cycloalkyl, in another embodiment, C₃₋₆cycloalkyl, such as cyclopentyl and cyclohexyl.

The term “heterocyclyl” is used herein to refer to monovalent, divalent or trivalent cycloalkyl groups in which up to three carbon atoms, in one embodiment up to two carbon atoms, in another embodiment one carbon atom, are each replaced independently by O, S(O)₁₋₂ or N, provided at least one of the cycloalkyl carbon atoms remains.

Examples of heterocyclyl groups include oxiranyl, thiaranyl, aziridinyl, oxetanyl, thiatanyl, azetidinyl, tetrahydrofuranyl, tetrahydrothiophenyl, pyrrolidinyl, pyrazolidinyl, imidazolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thieazepanyl and 1,4-diazepanyl. Other examples include cyclic imides, cyclic anhydrides and thiazolidindiones. The heterocyclyl group may be C-linked or N-linked, i.e. it may be linked to the remainder of the molecule through a carbon atom or through a nitrogen atom.

The term “alkenyl” is used herein to refer to monovalent, divalent or trivalent straight or branched, unsaturated, acyclic hydrocarbyl groups having at least one carbon-carbon double bond and, in one embodiment, no carbon-carbon triple bonds. In one embodiment, alkenyl is C₂-C₁₀ alkenyl, in another embodiment, C₂-C₆ alkenyl, in another embodiment C₂-C₄ alkenyl.

The term “alkynyl” is used herein to refer to monovalent or divalent unsaturated, acyclic hydrocarbyl groups having at least one carbon-carbon triple bond. In one embodiment alkynyl is C₂-C₁₀ alkynyl, in another embodiment, C₂-C₆ alkynyl, in another embodiment C₂-C₄ alkynyl.

### Aryl

The term “aryl” is used herein to refer to monovalent, divalent or trivalent, aromatic, cyclic hydrocarbyl groups, such as phenyl or naphthyl (e.g. 1-naphthyl or 2-naphthyl).

In general, the aryl group may be a monocyclic or polycyclic fused ring aromatic group. Preferred aryl groups are C₆-C₁₄aryl. Aryl groups include phenyl, biphenyl, naphthyl, indenyl, fluorenyl, anthracyl and phenanthryl.

### Heteroaryl

The term “heteroaryl” is used herein to refer to monovalent, divalent or trivalent, heteroaromatic, cyclic hydrocarbyl groups additionally containing one or more heteroatoms independently selected from O, S, N and NR^{T}, wherein R^{T} is preferably H or C₁-C₁₀ alkyl. In general, the heteroaryl group may be a monocyclic or polycyclic fused ring heteroaromatic group. Examples of monocyclic heteroaromatic groups are pyridyl, thienyl, furanyl, pyrrolyl, pyrazolyl, imidazoyl, oxazolyl, isoxazolyl, thiazolyl, isothiazolyl, triazoyl, oxadiazolyl, thiadiazolyl and tetrazolyl. Examples of polycyclic heteroaromatic groups are indolyl, benzofuranyl, benzothienyl, quinolyl, isoquinolyl, indazolyl, indolinyl, isoindolyl, indolizinyl, benzimidazolyl, quinolinyl and isoquinolinyl. Further examples of heteroaromatic groups include:

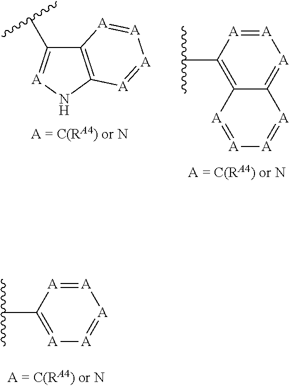

### Isomeric Forms

Compounds of the invention may exist in one or more geometrical, optical, enantiomeric, diastereomeric and tautomeric forms, including but not limited to cis- and trans-forms, E- and Z-forms, R-, S- and meso-forms, keto-, and enol-forms. All such isomeric forms are included within the invention. The isomeric forms may be in isomerically pure or enriched form, as well as in mixtures of isomers (e.g. racemic or diastereomeric mixtures).

### Exemplary Compounds

In one embodiment, the compounds of the invention comprise a sequence of amino acids according to the following Formula Ic:

X¹-E/D/pS-G-X⁴-X⁵-E/D/pS-NHR^{N2} Formula Ic

In a further embodiment, a compound of the invention may be a modified peptide of Formula Ig:

Capping group-X¹-E/D/pS-G-X⁴-X⁵-E/D/pS-NHR^{N2} Formula Ig

In one embodiment, the compound of the invention may be of Formula (IA)

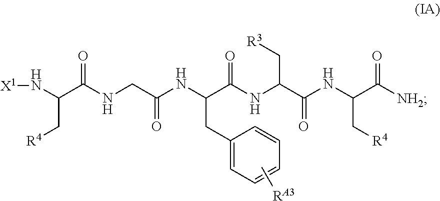

wherein each R⁴ is independently —CO₂H, —CH₂CO₂H—OP(O)(OH)₂, triazole, tetrazole, sulfonamide or sulphate;

R³ is

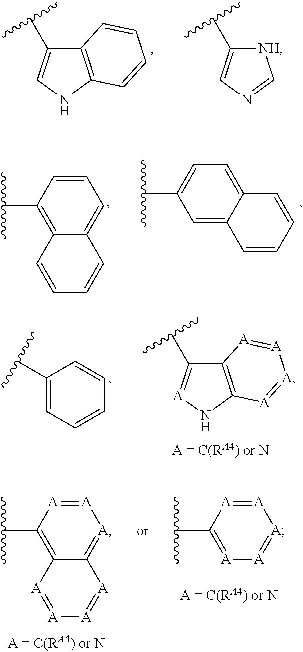

and R^{A3}, R^{A4} and X¹ are as previously defined. In one embodiment, R³ may be substituted at one or more positions with R^{A4}.

In one embodiment, the compound of the invention may be of Formula (IAA)

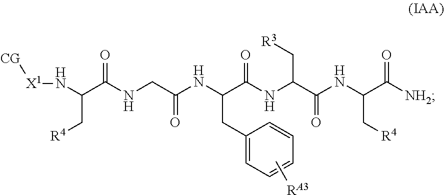

wherein each R⁴ is independently —CO₂H, —CH₂CO₂H—OP(O)(OH)₂, triazole, tetrazole, sulfonamide or sulphate;

R³ is

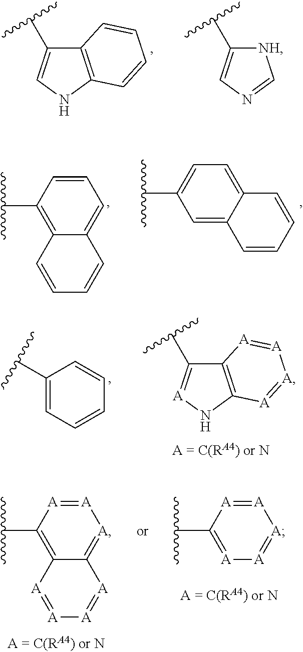

R^{A3}, R^{A4} and X¹ are as previously defined, and CG is a capping group. In one embodiment, R³ may be substituted at one or more positions with R^{A4}.

In one embodiment, the compound of the invention may be of formula (IAAA)

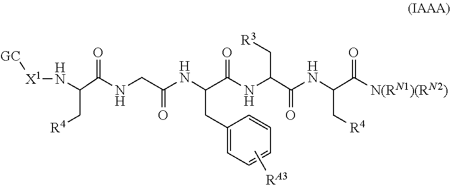

wherein each R⁴ is independently —CO₂H, —CH₂CO₂H—OP(O)(OH)₂, triazole, tetrazole, sulfonamide or sulphate;

R³ is

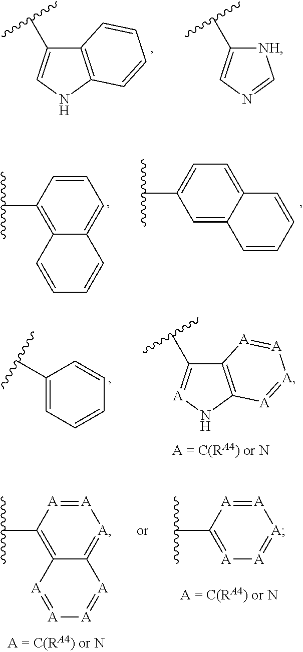

R^{A3}, R^{A4} and X¹ are as previously defined, R^{N1} and R^{N2} are as previously defined, and CG is a capping group. In one embodiment, R³ may be substituted at one or more positions with R^{A4}.

In one embodiment, the compound of the invention may be of formula (IAAAA)

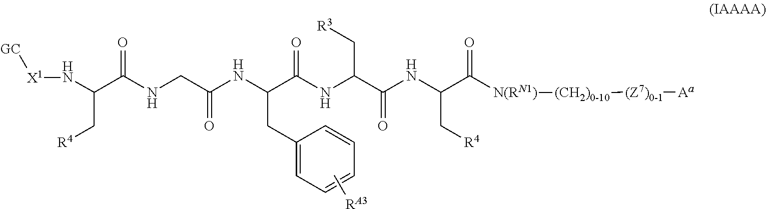

wherein each R⁴ is independently —CO₂H, —CH₂CO₂H—OP(O)(OH)₂, triazole, tetrazole, sulfonamide or sulphate;

R³ is

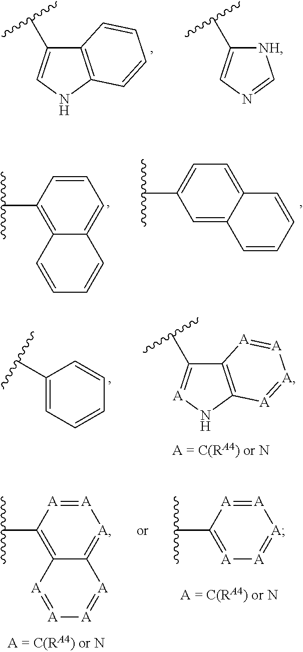

R^{A3}, R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of formula (IAAAAA)

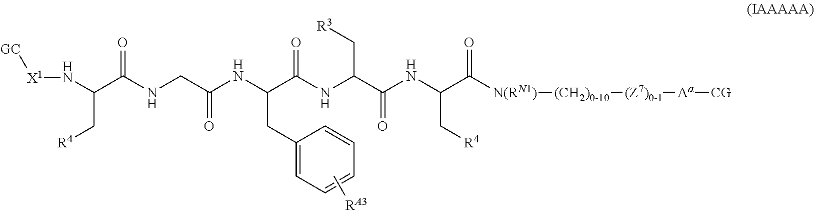

wherein each R⁴ is independently —CO₂H, —CH₂CO₂H—OP(O)(OH)₂, triazole, tetrazole, sulfonamide or sulphate;

R³ is

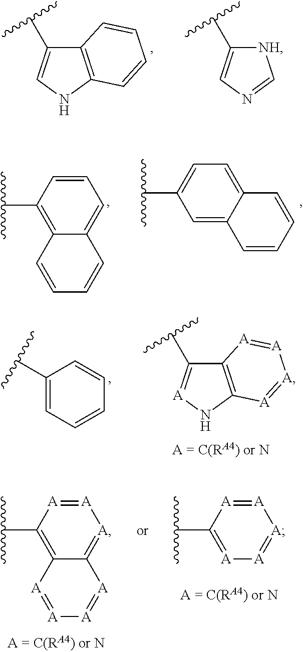

R^{A3}, R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of formula (IAAAAA), wherein each R⁴ is independently —CO₂H, —CH₂CO₂H—OP(O)(OH)₂, triazole, tetrazole, sulfonamide or sulphate;

R³ is

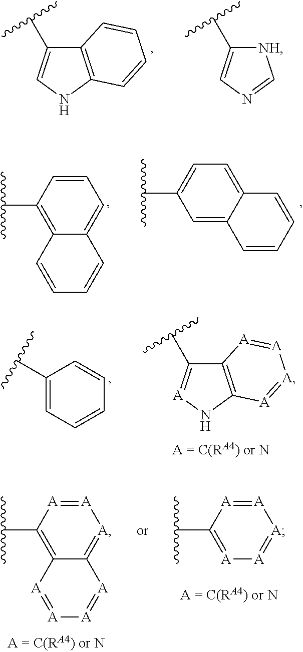

R^{A3}, R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group, wherein the capping group is selected from List 1. In one embodiment, the capping group on X¹ is selected from List 2 and/or the capping group on A^{a} is selected from List 3. In one embodiment, R³ may be substituted at one or more positions with R^{A4}.

In one embodiment, the compound of the invention may be of Formula (IB)

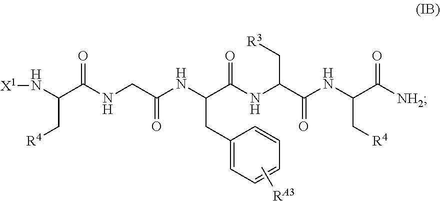

wherein each R⁴ is independently —CO₂H, —CH₂CO₂H, —OP(O)(OH)₂, triazole, tetrazole, sulfonamide or sulphate;

R³ is

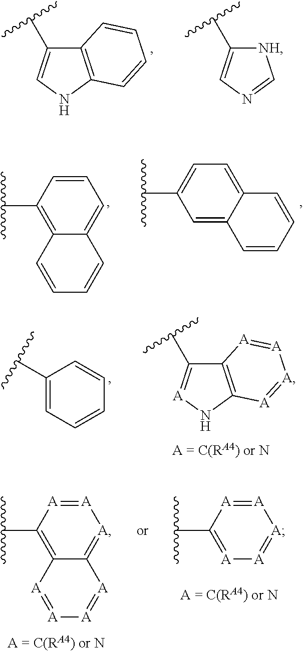

R^{A3} is selected from the group consisting of —H, —F, —Cl, —Br, —I, —OH, —O(C₁-C₁₀ alkyl), —NO₂ and C₁-C₁₀ alkyl; and R^{A4} and X¹ are as previously defined. In one embodiment, R³ may be substituted at one or more positions with R^{A4}.

In one embodiment, the compound of the invention may be of Formula (IBB)

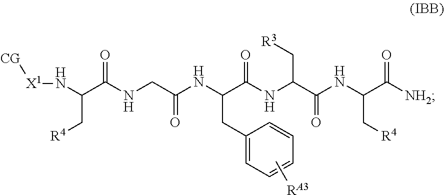

wherein each R⁴ is independently —CO₂H, —CH₂CO₂H, —OP(O)(OH)₂, triazole, tetrazole, sulfonamide or sulphate;

R³ is

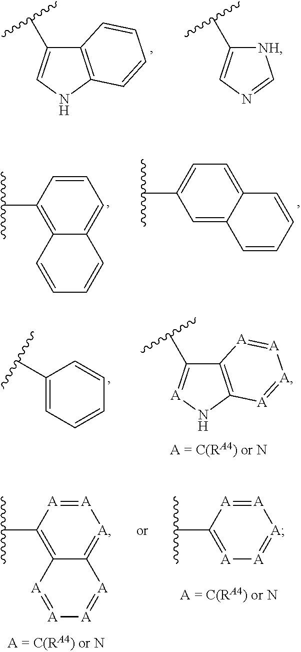

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, and CG is a capping group. In one embodiment, R³ may be substituted at one or more positions with R^{A4}.

In one embodiment, the compound of the invention may be of Formula (IBBB)

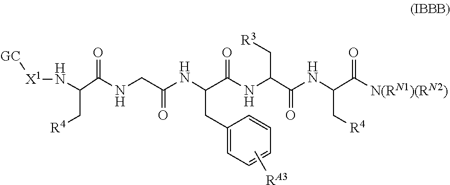

wherein each R⁴ is independently —CO₂H, —CH₂CO₂H, —OP(O)(OH)₂, triazole, tetrazole, sulfonamide or sulphate;

R³ is

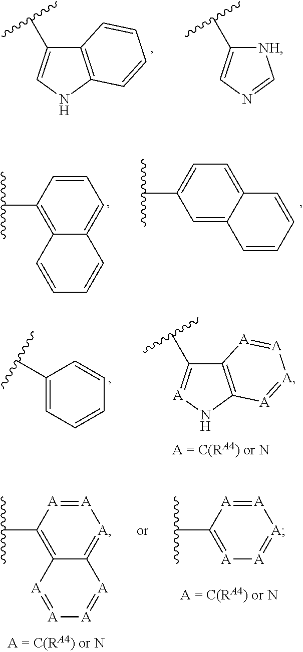

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} and R^{N2} are as previously defined, and CG is a capping group. In one embodiment, R³ may be substituted at one or more positions with R^{A4}.

In one embodiment, the compound of the invention may be of formula (IBBBB)

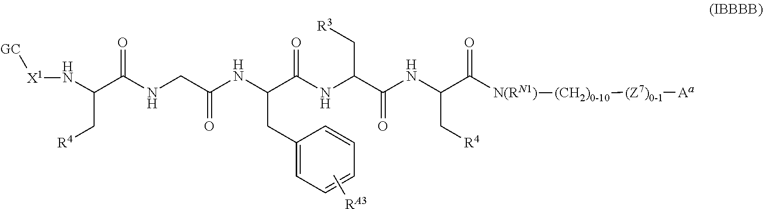

wherein each R⁴ is independently —CO₂H, —CH₂CO₂H, —OP(O)(OH)₂, triazole, tetrazole, sulfonamide or sulphate;

R³ is

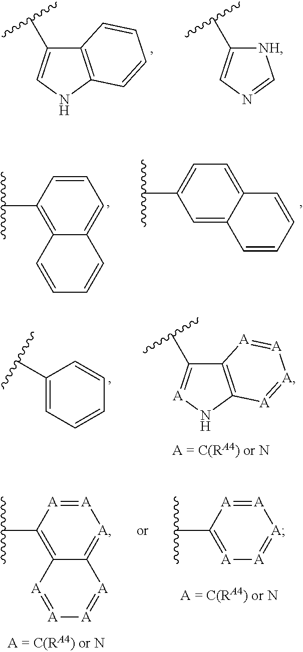

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of formula (IBBBBB)

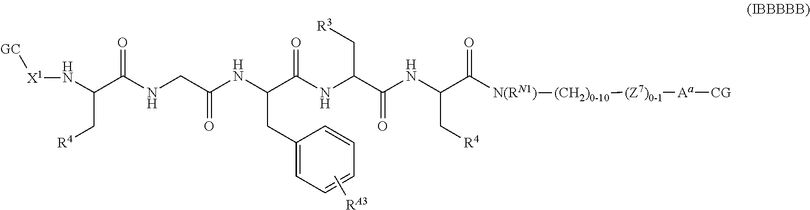

wherein each R⁴ is independently —CO₂H, —CH₂CO₂H, —OP(O)(OH)₂, triazole, tetrazole, sulfonamide or sulphate;

R³ is

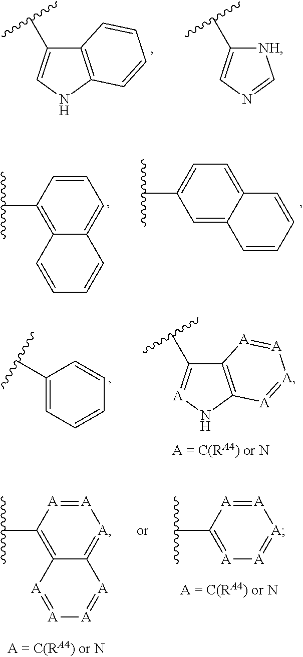

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of formula (IBBBBB), wherein each R⁴ is independently —CO₂H, —CH₂CO₂H, —OP(O)(OH)₂, triazole, tetrazole, sulfonamide or sulphate;

R³ is

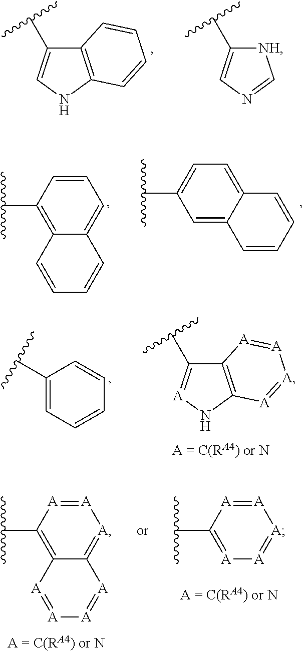

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group, wherein the capping group is selected from List 1. In one embodiment, the capping group on X¹ is selected from List 2 and/or the capping group on A^{a} is selected from List 3. In one embodiment, R³ may be substituted at one or more positions with R^{A4}.

In one embodiment, the compound of the invention may be of Formula (IC)

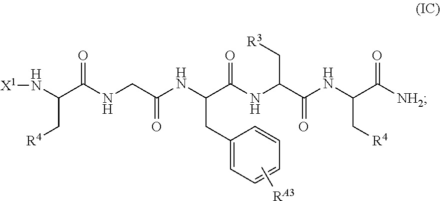

wherein each R⁴ is independently —CO₂H, —CH₂CO₂H or —OP(O)(OH)₂,

R³ is

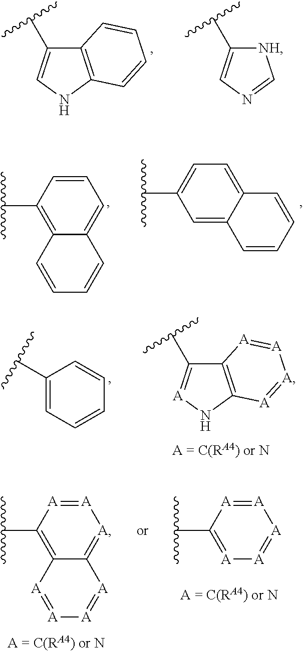

R^{A3} selected from the group consisting of —H, —F, —Cl, —Br, —I, —OH, —O(C₁-C₁₀ alkyl), —NO₂ and C₁-C₁₀ alkyl; and R^{A4} and X¹ are as previously defined. In one embodiment, R³ may be substituted at one or more positions with R^{A4}.

In one embodiment, the compound of the invention may be of Formula (ICC)

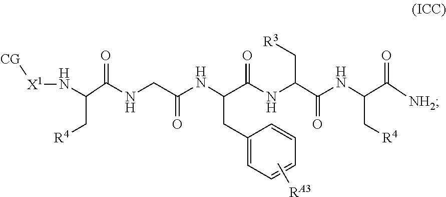

wherein each R⁴ is independently —CO₂H, —CH₂CO₂H or —OP(O)(OH)₂;

R³ is

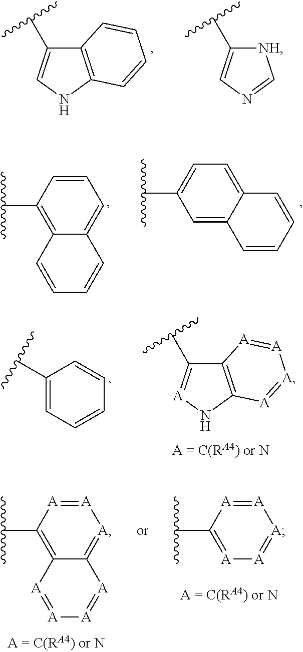

R^{A3} selected from the group consisting of —H, —F, —Cl, —Br, —I, —OH, —O(C₁-C₁₀ alkyl), —NO₂ and C₁-C₁₀ alkyl; R^{A4} and X¹ are as previously defined, and CG is a capping group. In one embodiment, R³ may be substituted at one or more positions with R^{A4}.

In one embodiment, the compound of the invention may be of Formula (ICCC)

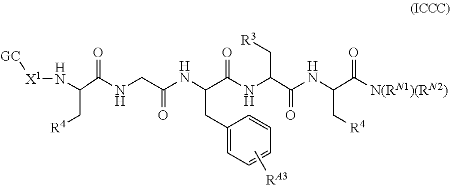

wherein each R is independently —CO₂H, —CH₂CO₂H or —OP(O)(OH)₂;

R³ is

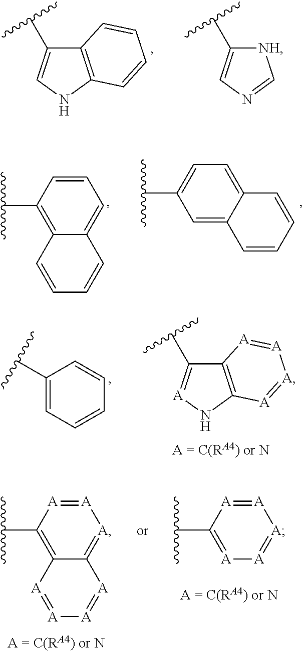

R^{A3} selected from the group consisting of —H, —F, —Cl, —Br, —I, —OH, —O(C₁-C₁₀ alkyl), —NO₂ and C₁-C₁₀ alkyl; R^{A} and X¹ are as previously defined, RN¹ and R^{N2} are as previously defined, and CG is a capping group. In one embodiment, R³ may be substituted at one or more positions with R^{A4}.

In one embodiment, the compound of the invention may be of Formula (ICCCC)

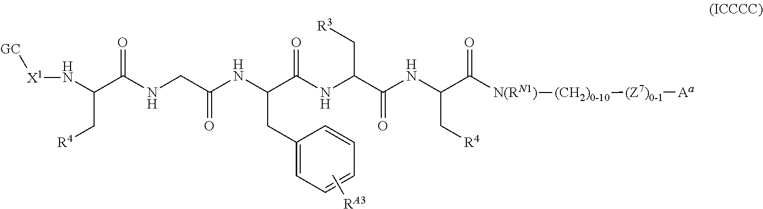

wherein each R⁴ is independently —CO₂H, —CH₂CO₂H or —OP(O)(OH)₂;

R³ is

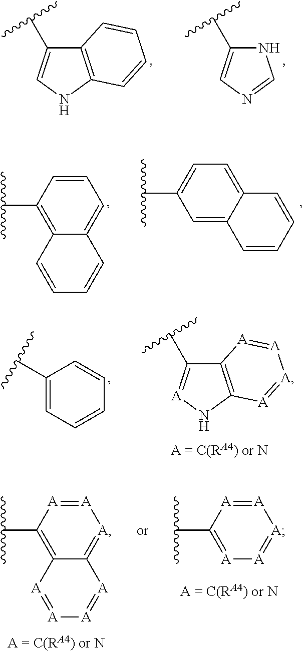

R^{A3} selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of Formula (ICCCCC)

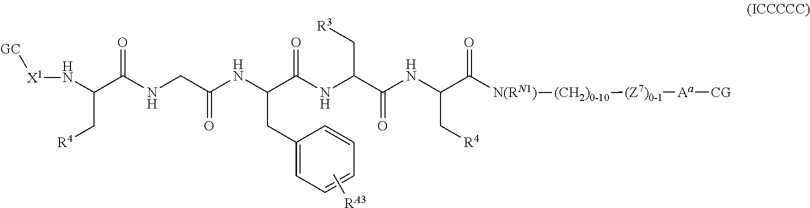

wherein each R⁴ is independently —CO₂H, —CH₂CO₂H or —OP(O)(OH)₂;

R³ is

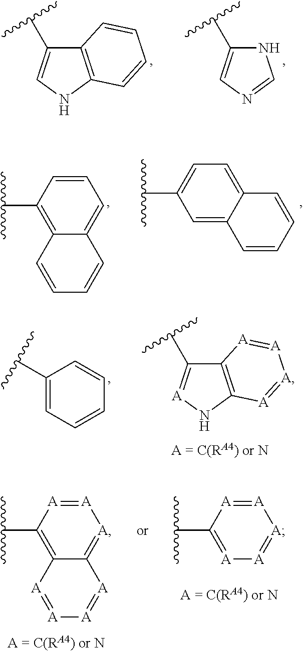

R^{A3} selected from the group consisting of —H, —F, —Cl, —Br, —I, —OH, —O(C₁-C₁₀ alkyl), —NO₂ and C₁-C₁₀ alkyl; R⁴ and X¹ are as previously defined, N^{R1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of Formula (ICCCCC) wherein each R⁴ is independently —CO₂H, —CH₂CO₂H or —OP(O)(OH)₂;

R³ is

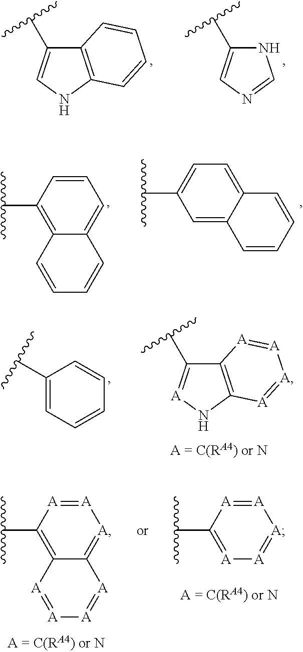

R^{A3} selected from the group consisting of —H, —F, —Cl, —Br, —I, —OH, —O(C₁-C₁₀ alkyl), —NO₂ and C₁-C₁₀ alkyl; R^{A4} and X^{I} are as previously defined, N^{R1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group, wherein the capping group is selected from List 1. In one embodiment, the capping group on X^{I} is selected from List 2, and the capping group on A^{a} is selected from List 3. In one embodiment, R³ may be substituted at one or more positions with R^{A4}.

In one embodiment, the compound of the invention may be of Formula (ID)

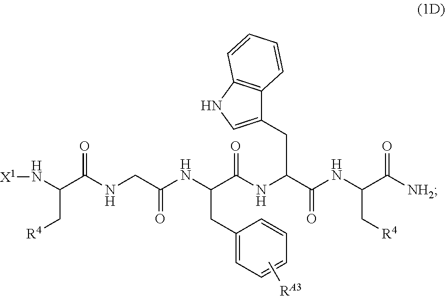

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ and C₁-C₁₀ alkyl; and X¹ is as previously defined.

In one embodiment, the compound of the invention may be of Formula (IDD)

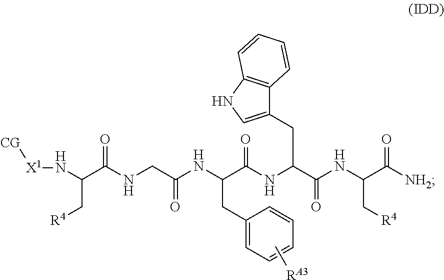

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ or C₁-C₁₀ alkyl; X¹ is as previously defined and CG is a capping group.

In one embodiment, the compound of the invention may be of Formula (IDDD)

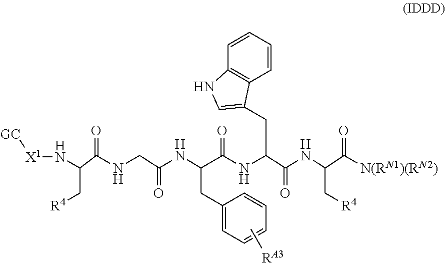

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ or C₁-C₁₀ alkyl; X¹ is as previously defined, R^{N1} and R^{N2} are as previously defined, and CG is a capping group;

In one embodiment, the compound of the invention may be of Formula (IDDDD)

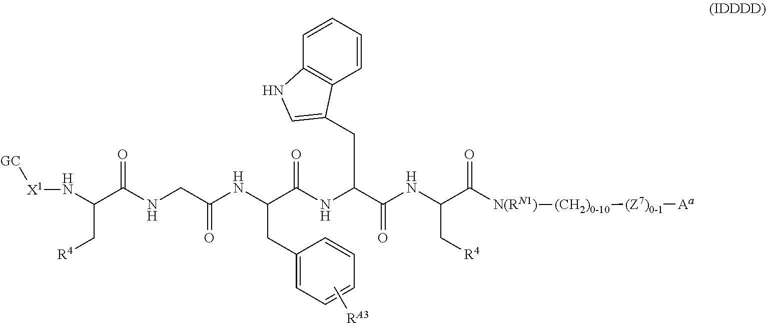

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ or C₁-C₁₀ alkyl; X¹ is as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group.

In one embodiment, the compound of the invention may be of Formula (IDDDDD)

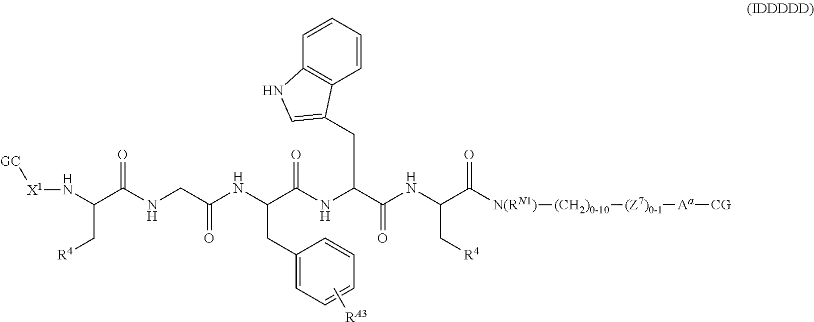

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ or C₁-C₁₀ alkyl; X¹ is as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group.

In one embodiment, the compound of the invention may be of Formula (IDDDDD), wherein each R¹ is independently —CO₂H, —CH₂CO₂H or —OP(O)(OH)₂;

R^{A} is selected from the group consisting of —H, —F, —Cl, —Br, —I, —OH, —O(C₁-C₁₀ alkyl), —NO₂ or C₁-C₁₀ alkyl, X¹ is as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group, wherein the capping group is selected from List 1. In one embodiment the capping group on X¹ is selected from List 2 and/or the capping group on A^{a} is selected from List 3.

In one embodiment, the compound of the invention may be of Formula (IE)

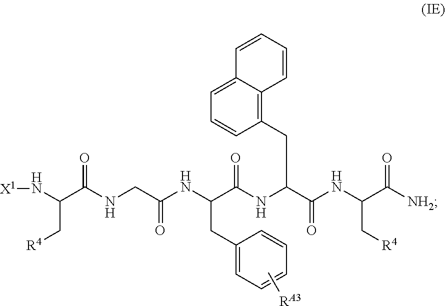

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ and C₁-C₁₀ alkyl; and X¹ is as previously defined.

In one embodiment, the compound of the invention may be of Formula (IEE)

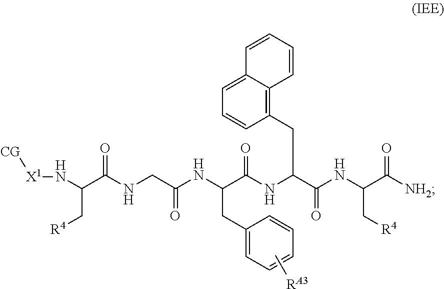

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ and C₁-C₁₀ alkyl; X¹ is as previously defined and CG is a capping group.

In one embodiment, the compound of the invention may be of Formula (IF)

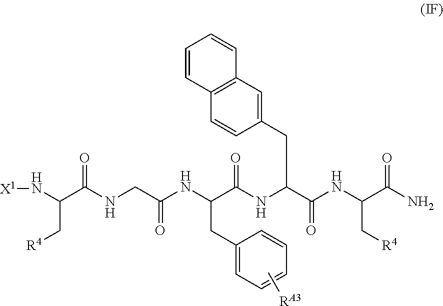

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ and C₁-C₁₀ alkyl; and X¹ is as previously defined.

In one embodiment, the compound of the invention may be of Formula (IFF)

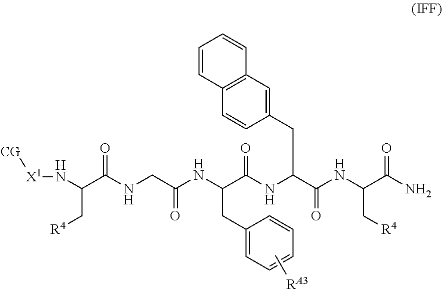

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ and C₁-C₁₀ alkyl; X¹ is as previously defined and CG is a capping group.

In one embodiment, the compound of the invention may be of Formula (IG)

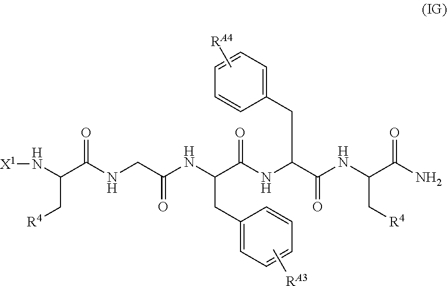

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ and C₁-C₁₀ alkyl; and R^{A4} and X¹ are as previously defined.

In one embodiment, the compound of the invention may be of Formula (IGG)

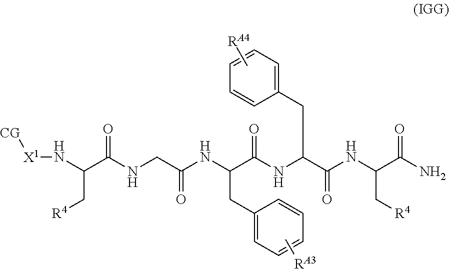

wherein each R⁴ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, and CG is a capping group.

In one embodiment, the compound of the invention may be of Formula (IH)

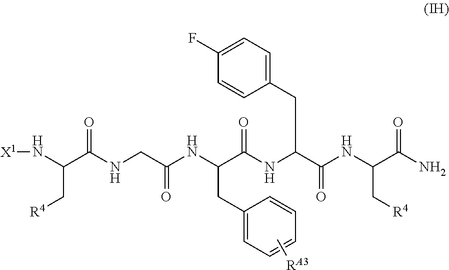

wherein each R⁴ is independently —CO₂H, —CH₂CO₂H or —OP(O)OH)₂,

R^{A3} is selected from the group consisting of —H, —F, —Cl, —Br, —I, —OH, —O(C₁-C₁₀ alkyl), —NO₂ and C₁-C₁₀ alkyl; and X¹ is as previously defined.

In one embodiment, the compound of the invention may be of Formula (IHH)

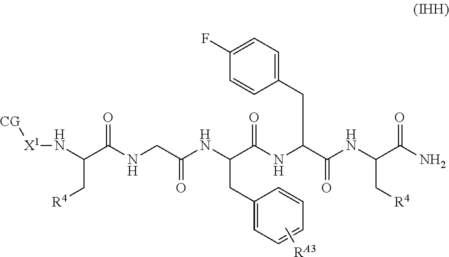

wherein each R¹ is independently —CO₂H, —CH₂CO₂H or —OP(O)(OH)₂;

R^{A3} is selected from the group consisting of —H, —F, —Cl, —Br, —I, —OH, —O(C₁-C₁₀ alkyl), —NO₂ and C₁-C₁₀ alkyl; X^{I} is as previously defined and CG is a capping group.

In one embodiment, the compound of the invention may be of Formula (II)

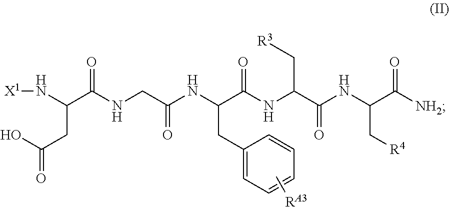

wherein R⁴ is —CO₂H, —CH₂CO₂H or —OP(O)(OH)₂;

R³ is

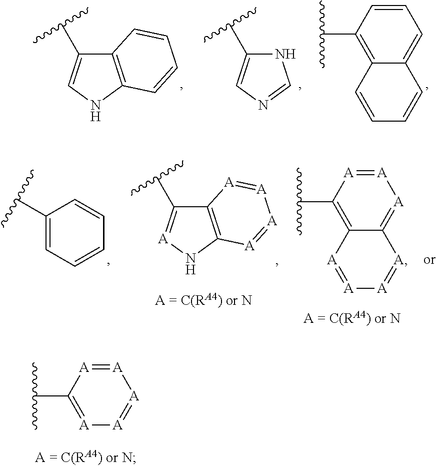

R^{A3} is selected from the group consisting of —H, —F, —Cl, —Br, —I, —OH, —O(C₁-C₁₀ alkyl), —NO₂ and C₁-C₁₀ alkyl; and R^{A4} and X¹ are as previously defined. In one embodiment, R³ may be substituted at one or more positions with R^{A4}.

In one embodiment, the compound of the invention may be of Formula (III)

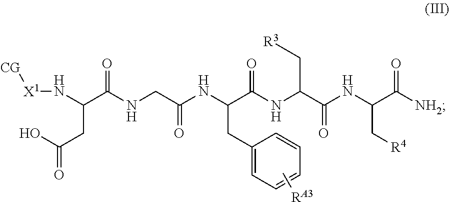

wherein R⁴ is —CO₂H, —CH₂CO₂H or —OP(O)(OH)₂;

R³ is

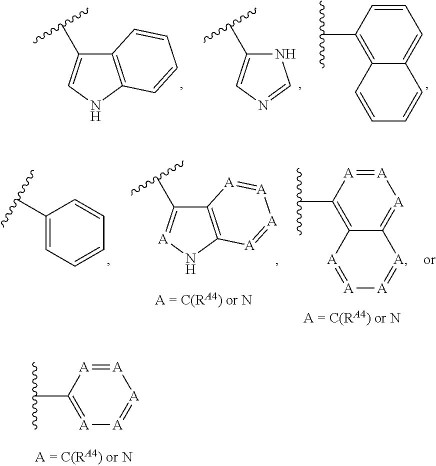

R^{A3} is selected from the group consisting of —H, —F, —Cl, —Br, —I, —OH, —O(C₁-C₁₀ alkyl), —NO₂ and C₁-C₁₀ alkyl; and R^{A4} and X¹ are as previously defined. In one embodiment, R³ may be substituted at one or more positions with R^{A4}.

In one embodiment, the compound of the invention may be of Formula (IJ)

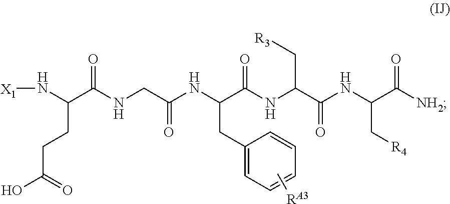

wherein R⁴ is —CO₂H, —CH₂CO₂H or —OP(O)(OH)₂;

R³ is

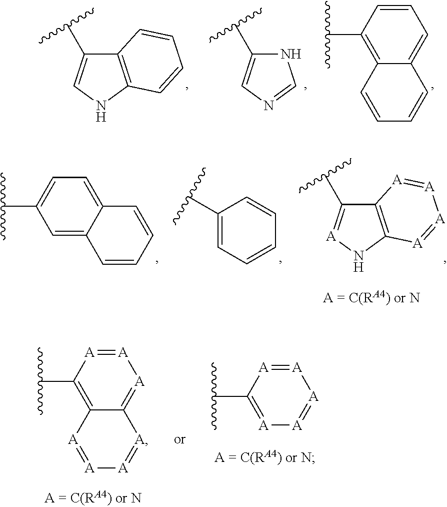

R^{A3} is selected from the group consisting of —H, —F, —Cl, —Br, —I, —OH, —O(C₁-C₁₀ alkyl), —NO₂ and C₁-C₁₀ alkyl; and R^{A4} and X¹ are as previously defined. In one embodiment, R³ may be substituted at one or more positions with R^{A4}.

In one embodiment, the compound of the invention may be of Formula (IJJ)

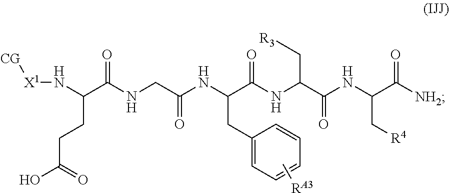

wherein R⁴ is —CO₂H, —CH₂CO₂H or —OP(O)(OH)₂;

R³ is

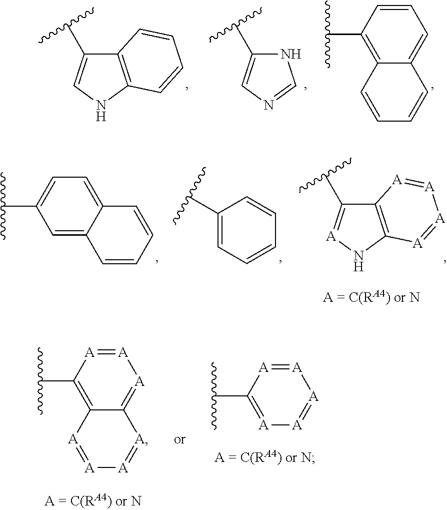

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, and CG is a capping group. In one embodiment, R³ may be substituted at one or more positions with R^{A4}.

In one embodiment, the compound of the invention may be of Formula (IJJJ)

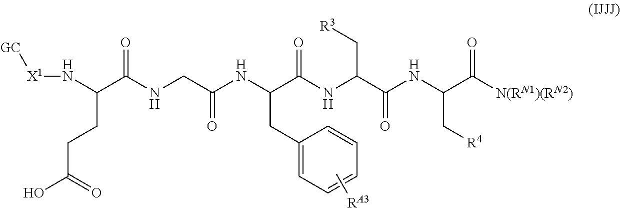

wherein R⁴ is —CO₂H, —CH₂CO₂H or —OP(O)(OH)₂;

R³ is

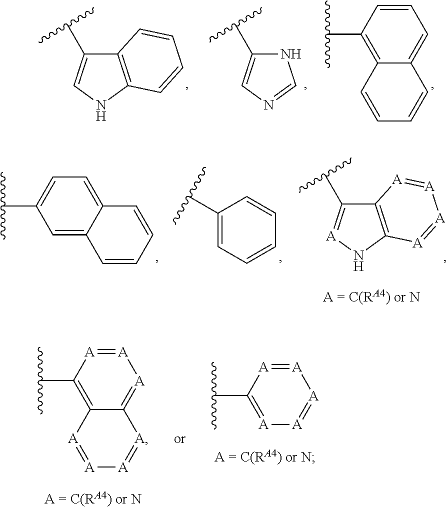

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} and R^{N2} are as previously defined, and CG is a capping group. In one embodiment, R³ may be substituted at one or more positions with R^{A4}.

In one embodiment, the compound of the invention may be of Formula (IJJJJ)

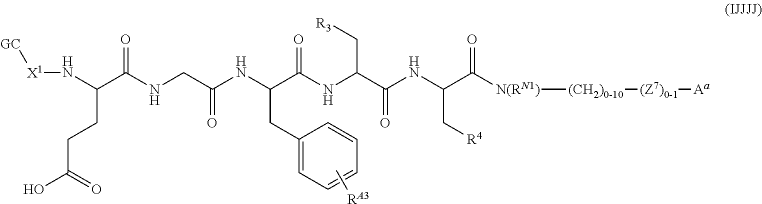

wherein R⁴ is —CO₂H, —CH₂CO₂H or —OP(O)(OH)₂;

R³ is

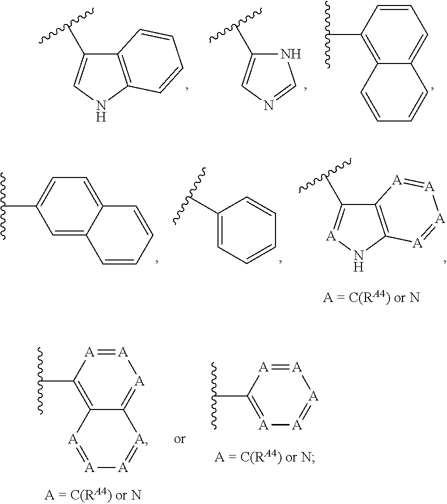

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of Formula (IJJJJJ)

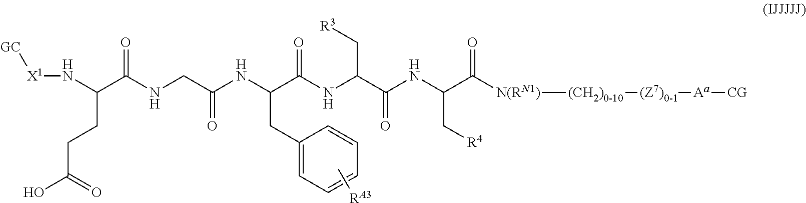

wherein R⁴ is —CO₂H, —CH₂CO₂H or —OP(O)(OH)₂;

R³ is

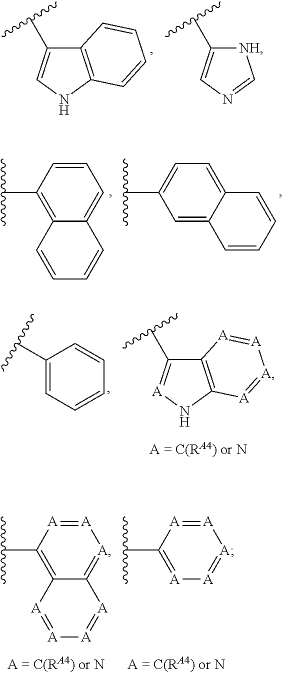

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of Formula (IJJJJJ), wherein R⁴ is —CO₂H, —CH₂CO₂H or —OP(O)(OH)₂;

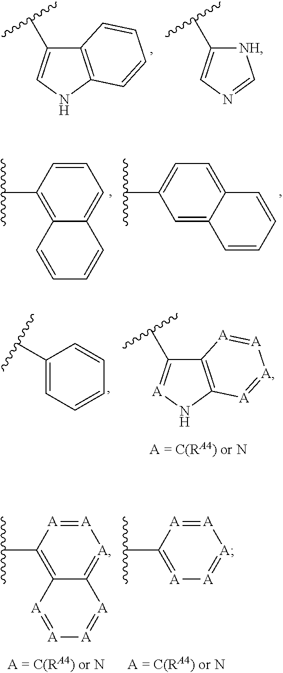

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group, wherein the capping group is selected from List 1. In one embodiment, the capping group on X¹ is selected from List 2 and/or the capping group on A^{a} is selected from List 3. In one embodiment, R³ may be substituted at one or more positions with R^{A4}.

In one embodiment, the compound of the invention may be of Formula (IK)

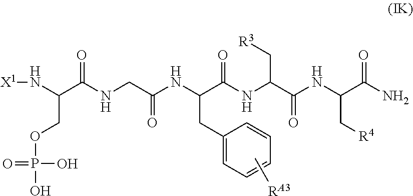

wherein R⁴ is —CO₂H, —CH₂CO₂H or —OP(O)(OH)₂;

R³ is

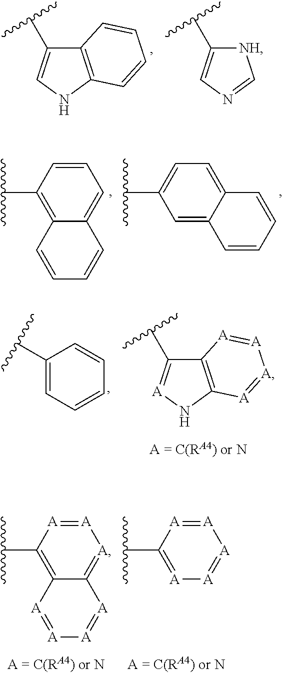

R^{A3} is selected from the group consisting of —H, —F, —Cl, —Br, —I, —OH, —O(C₁-C₁₀ alkyl), —NO₂ and C₁-C₁₀ alkyl; and R^{A4} and X¹ are as previously defined. In one embodiment, R³ may be substituted at one or more positions with R^{A4}.

In one embodiment, the compound of the invention may be of Formula (IKK)

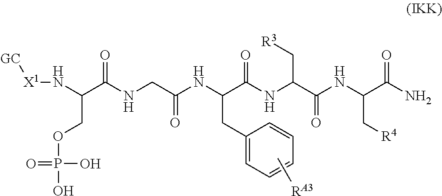

wherein R⁴ is —CO₂H, —CH₂CO₂H or —OP(O)(OH)₂;

R³ is

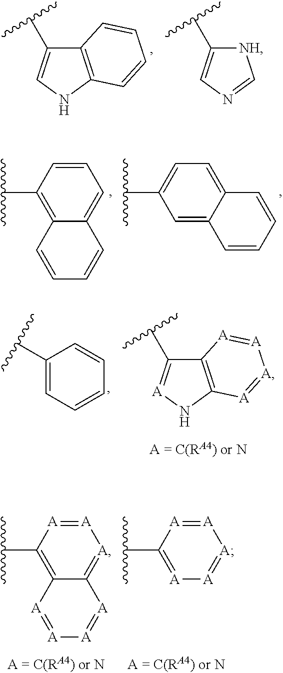

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined and CG is a capping group. In one embodiment, R³ may be substituted at one or more positions with R^{A4}.

In one embodiment, the compound of the invention may be of Formula (IL)

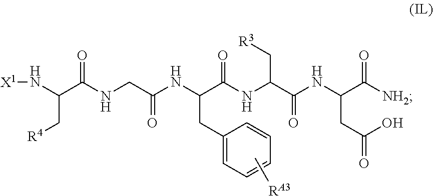

wherein R⁴ is —CO₂H, —CH₂CO₂H or —OP(O)(OH)₂,

R³ is

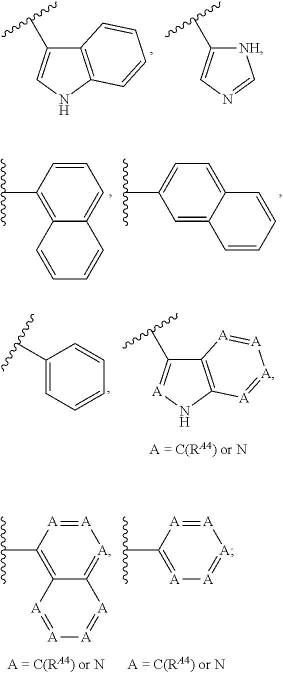

R^{A3} is selected from the group consisting of —H, —F, —Cl, —Br, —I, —OH, —O(C₁-C₁₀ alkyl), —NO₂ and C₁-C₁₀ alkyl; and R^{A4} and X¹ are as previously defined. In one embodiment, R³ may be substituted at one or more positions with R^{A4}.

In one embodiment, the compound of the invention may be of Formula (ILL)

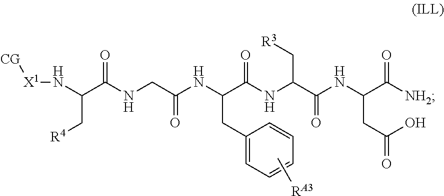

wherein R⁴ is —CO₂H, —CH₂CO₂H or —OP(O)(OH)₂; R³ is

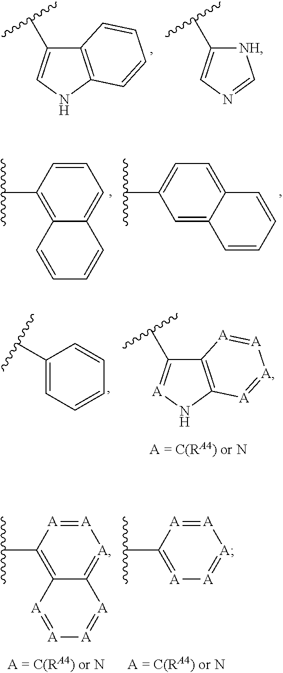

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, and CG is a capping group. In one embodiment, R³ may be substituted at one or more positions with R^{A4}.

In one embodiment, the compound of the invention may be of Formula (IM)

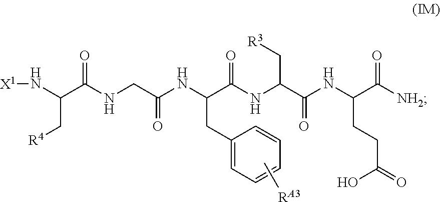

wherein R⁴ is —CO₂H, —CH₂CO₂H or —OP(O)(OH)₂;

R³ is

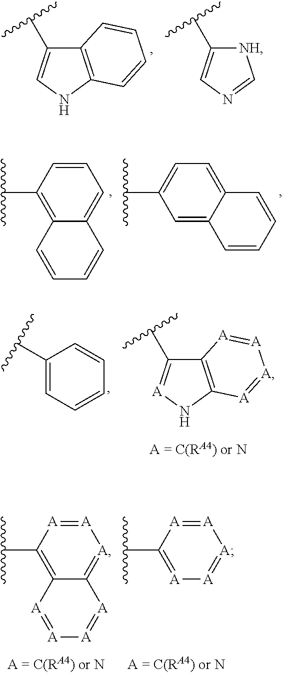

R^{A3} is selected from the group consisting of —H, —F, —Cl, —Br, —I, —OH, —O(C₁-C₁₀ alkyl), —NO₂ and C₁-C₁₀ alkyl; and R^{A4} and X¹ are as previously defined. In one embodiment, R³ may be substituted at one or more positions with R^{A4}.

In one embodiment, the compound of the invention may be of Formula (IMM)

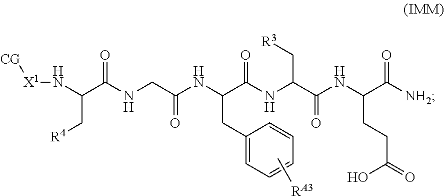

wherein R⁴ is —CO₂H, —CH₂CO₂H or —OP(O)(OH)₂;

R³ is

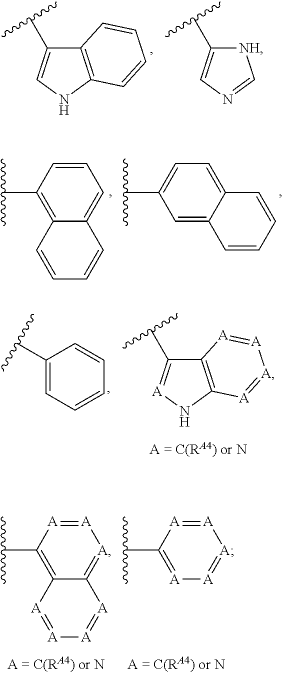

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined and CG is a capping group. In one embodiment, R³ may be substituted at one or more positions with R^{A4}.

In one embodiment, the compound of the invention may be of Formula (IMMM)

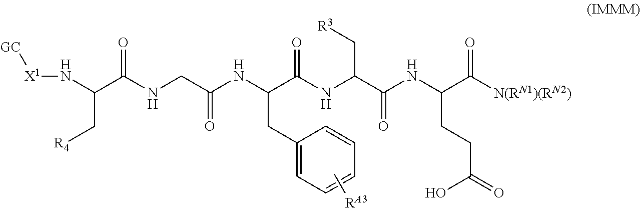

wherein R⁴ is —CO₂H, —CH₂CO₂H or —OP(O)(OH)₂;

R³ is

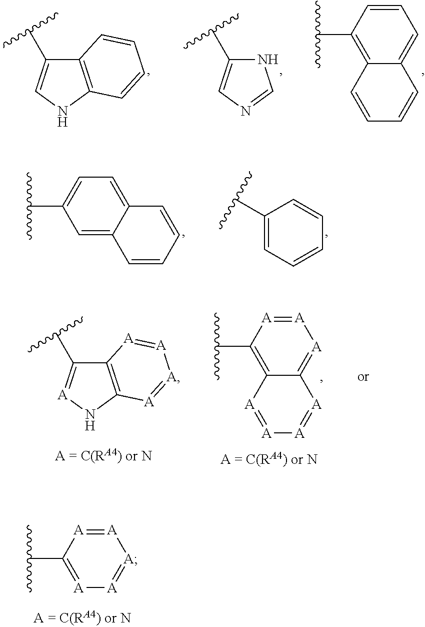

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} and R^{N2} are as previously defined, and CG is a capping group. In one embodiment, R³ may be substituted at one or more positions with R^{A4}.

In one embodiment, the compound of the invention may be of Formula (IMMMM)

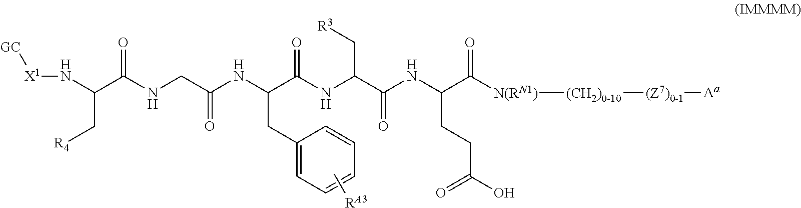

wherein R⁴ is —CO₂H, —CH₂CO₂H or —OP(O)(OH)₂;

R³ is

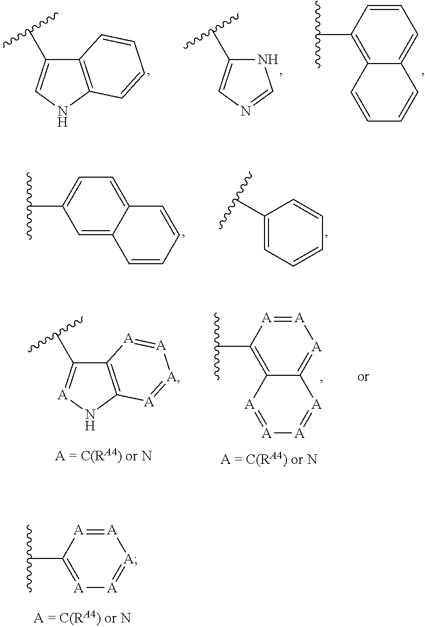

R^{A3} is selected from the group consisting of —H, —F, —Cl, —Br, —I, —OH, —O(C₁-C₁₀ alkyl), —NO₂ and C₁-C₁₀ alkyl, R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of Formula (IMMMMM)

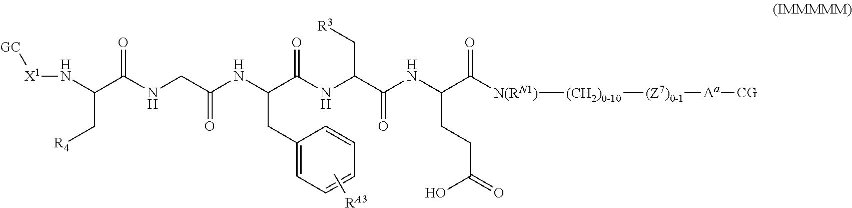

wherein R⁴ is —CO₂H, —CH₂CO₂H or —OP(O)(OH)₂;

R³ is

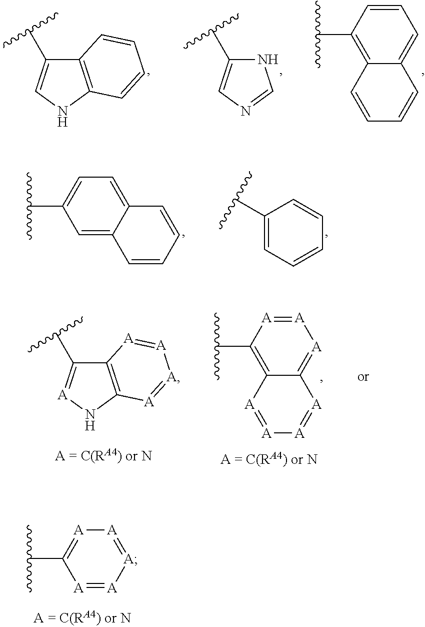

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of Formula (IMMMMM), wherein R⁴ is —CO₂H, —CH₂CO₂H or —OP(O)(OH)₂; R³ is

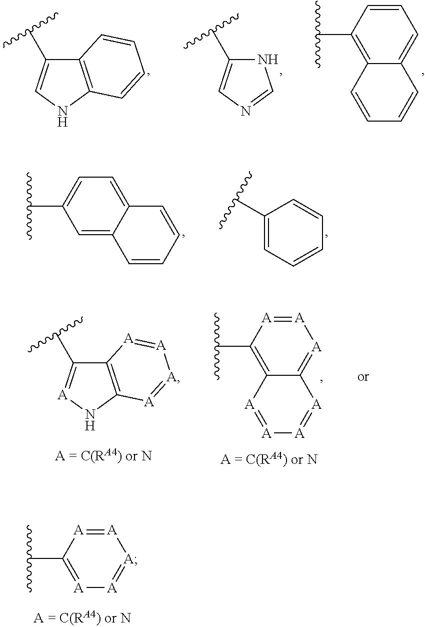

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined, R^{N1} is as previously defined, A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group, wherein the capping group is selected from List 1. In one embodiment, the capping group on X¹ is selected from List 2 and/or the capping group on A^{a} is selected from List 3. In one embodiment, R³ may be substituted at one or more positions with R^{A4}.

In one embodiment, the compound of the invention may be of Formula (IN)

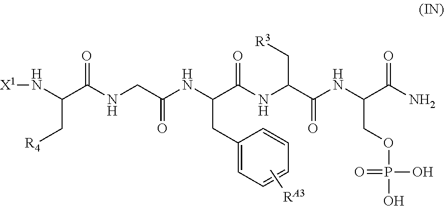

wherein R⁴ is —CO₂H, —CH₂CO₂H or —OP(O)(OH)₂;

R³ is

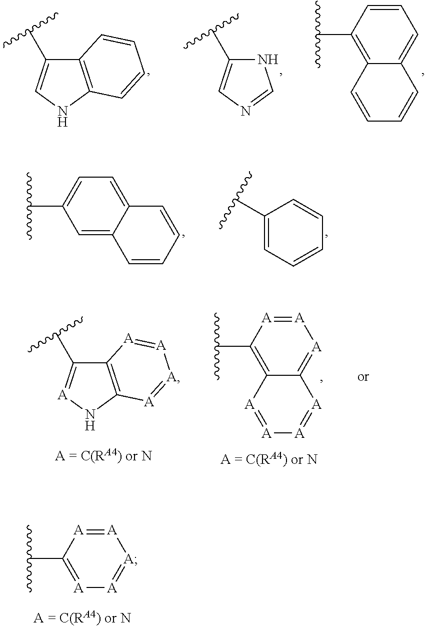

R^{A3} is selected from the group consisting of —H, —F, —Cl, —Br, —I, —OH, —O(C₁-C₁₀ alkyl), —NO₂ and C₁-C₁₀ alkyl; and R^{A4} and X¹ are as previously defined. In one embodiment, R³ may be substituted at one or more positions with R^{A4}.

In one embodiment, the compound of the invention may be of Formula (INN)

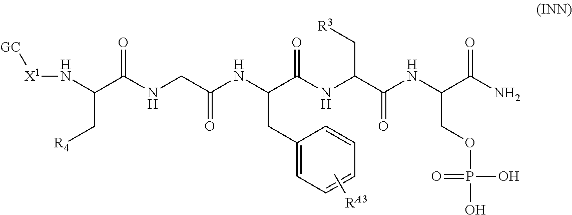

wherein R⁴ is —CO₂H, —CH₂CO₂H or —OP(O)(OH)₂;

R³ is

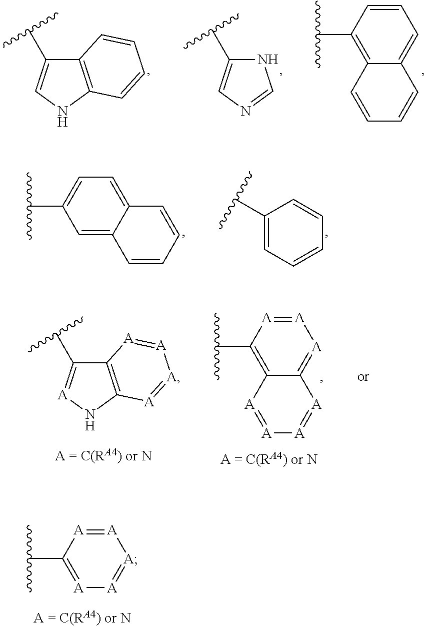

R^{A3} is selected from the group consisting of —H, —F, —Cl, —Br, —I, —OH, —O(C₁-C₁₀ alkyl), —NO₂ and C₁-C₁₀ alkyl; R^{A4} and X¹ are as previously defined; and CG is a capping group. In one embodiment, R³ may be substituted at one or more positions with R^{A4}.

In one embodiment, the compound of the invention may be of Formula (IO)

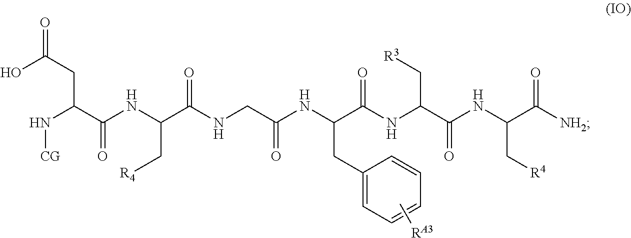

wherein each R⁴ is independently —CO₂H, —CH₂CO₂H or —OP(O)(OH)₂,

R³ is

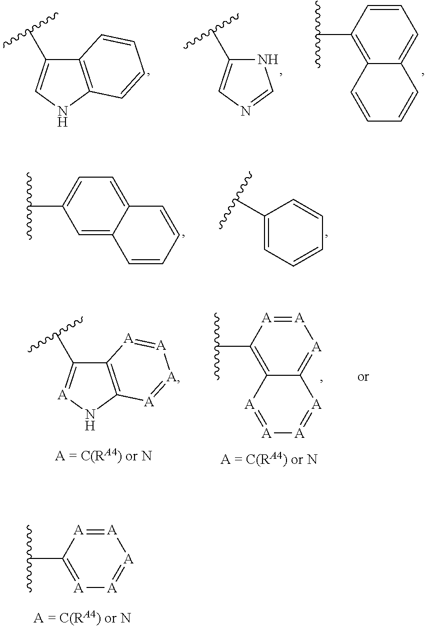

R^{A3} is selected from the group consisting of —H, —F, —Cl, —Br, —I, —OH, —O(C₁-C₁₀ alkyl), —NO₂ and C₁-C₁₀ alkyl; R^{A4} is as previously defined and CG is a capping group. In one embodiment, R³ may be substituted at one or more positions with R^{A4}. In one embodiment, the compound of the invention may be of Formula (IOO)

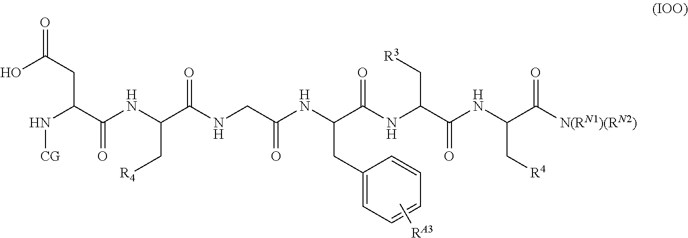

wherein each R⁴ is independently —CO₂H, —CH₂CO₂H or —OP(O)(OH)₂.

R³ is

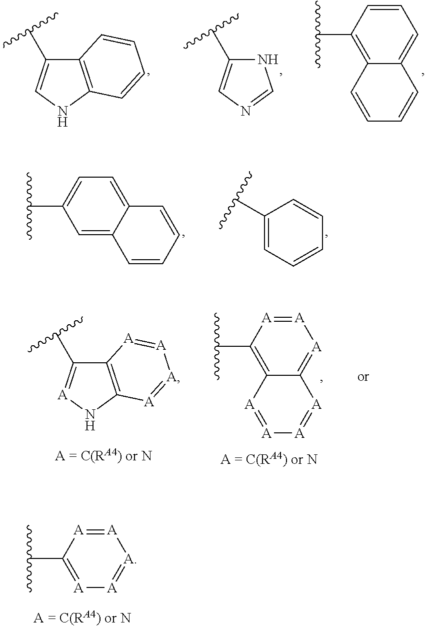

R^{A3} is selected from the group consisting of —H, —F, —Cl, —Br, —I, —OH, —O(C₁-C₁₀ alkyl), —NO₂ and C₁-C₁₀ alkyl; R^{A4} is as previously defined, R^{N1} and R^{N2} are as previously defined, and CG is a capping group. In one embodiment, R³ may be substituted at one or more positions with R^{A4}.

In one embodiment, the compound of the invention may be of Formula (IOOO)

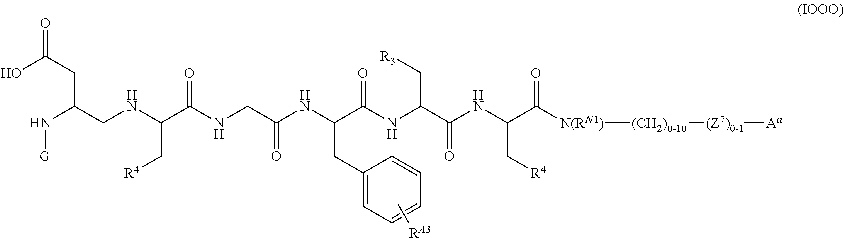

wherein each R¹ is independently —CO₂H, —CH₂CO₂H or —OP(O)(OH)₂,

R³ is

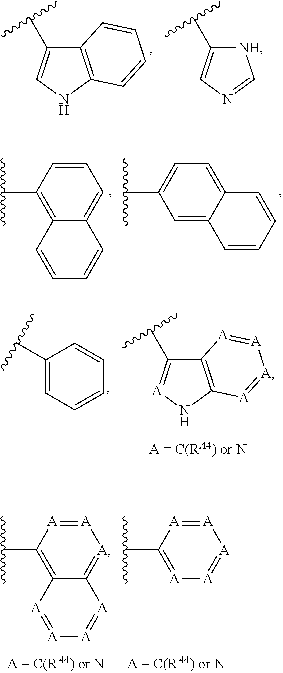

R^{A3} is selected from the group consisting of —H, —F, —Cl, —Br, —I, —OH, —O(C₁-C₁₀ alkyl), —NO₂ and C₁-C₁₀ alkyl; R^{A4} is as previously defined, R^{N1} is as previously defined; A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of Formula (IOOOO)

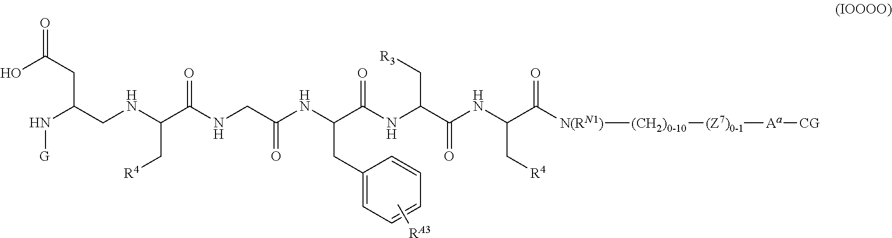

wherein each R⁴ is independently —CO₂H, —CH₂CO₂H or —OP(O)(OH)₂,

R³ is

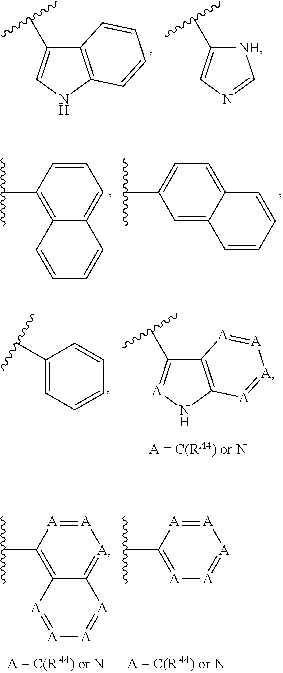

R^{A3} is selected from the group consisting of —H, —F, —Cl, —Br, —I, —OH, —O(C₁-C₁₀ alkyl), —NO₂ and C₁-C₁₀ alkyl; R^{A4} is as previously defined, R^{N1} is as previously defined; A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl, and CG is a capping group. In one embodiment, R³ may be substituted at one or more positions with R^{A4}.

In one embodiment, the compound of the invention may be of Formula (IOOOO), wherein each R⁴ is independently —CO₂H, —CH₂CO₂H or —OP(O)(OH)₂,

R³ is

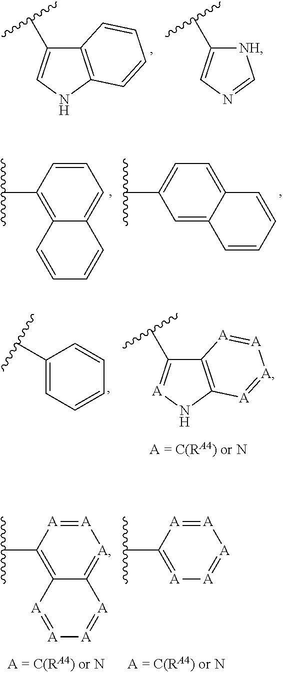

R^{A3} is selected from the group consisting of —H, —F, —Cl, —Br, —I, —OH, —O(C₁-C₁₀ alkyl), —NO₂ and C₁-C₁₀ alkyl; R^{A4} is as previously defined, R^{N1} is as previously defined; A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A⁵ is lysyl, and CG is a capping group, wherein the capping group is selected from List 1. In one embodiment, the capping group on X¹ is selected from List 2 and/or the capping group on A^{a} is selected from List 3. In one embodiment, R³ may be substituted at one or more positions with R^{A4}.

In one embodiment, the compound of the invention may be of Formula (IP)

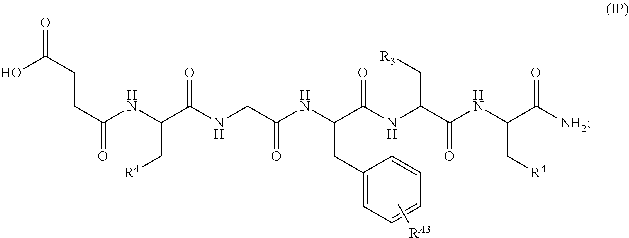

wherein each R⁴ is independently —CO₂H, —CH₂CO₂H or —OP(O)(OH)₂;

R³ is

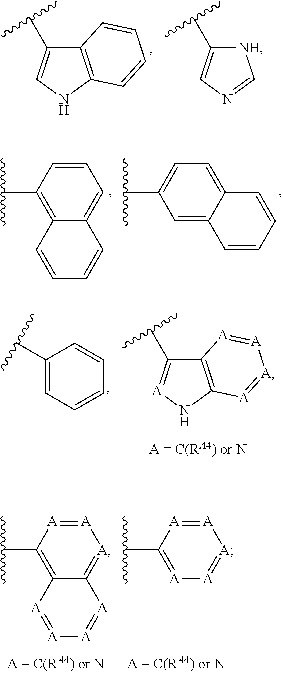

R^{A3} is selected from the group consisting of —H, —F, —Cl, —Br, —I, —OH, —O(C₁-C₁₀ alkyl), —NO₂ and C₁-C₁₀ alkyl; and R^{A4} is as previously defined. In one embodiment, R³ may be substituted at one or more positions with R^{A4}.

In one embodiment, the compound of the invention may be of Formula (IQ)

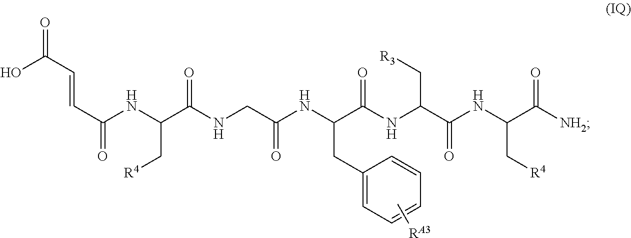

wherein each R⁴ is independently —CO₂H, —CH₂CO₂H or —OP(O)(OH)₂;

R³ is

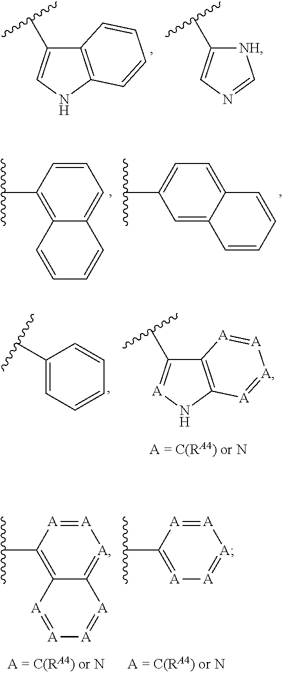

R^{A3} is —H, —F, —Cl, —Br, —I, —OH, —O(C₁-C₁₀ alkyl), —NO₂ or C₁-C₁₀ alkyl; and R^{A4} is as previously defined. In one embodiment, R³ may be substituted at one or more positions with R^{A4}.

In one embodiment, the compound of the invention may be of Formula (IS)

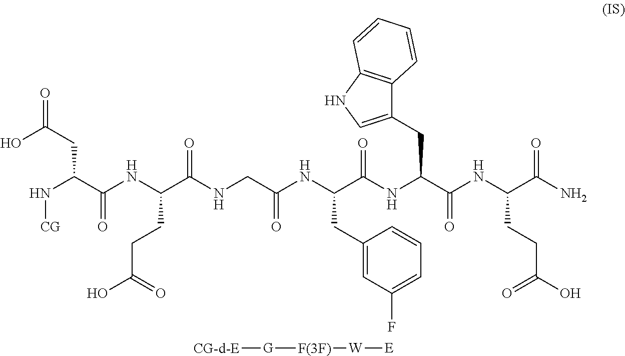

wherein CG is a capping group.

In one embodiment, the compound of the invention may be of Formula (ISS)

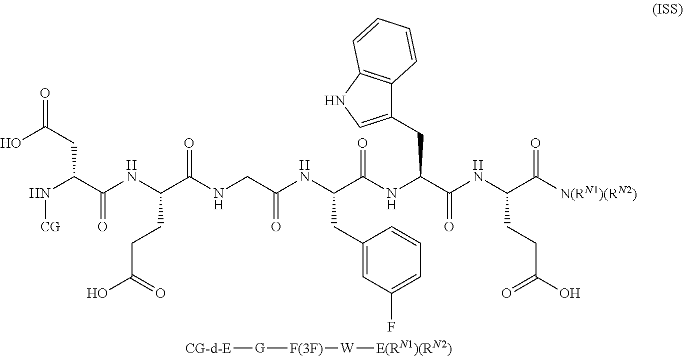

wherein R^{N1} and R^{N2} are as previously defined, and CG is a capping group.

In one embodiment, the compound of the invention may be of Formula (ISSS)

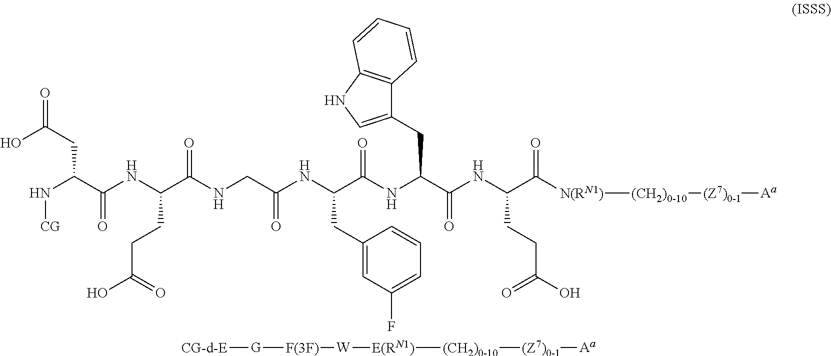

wherein R^{N1} is as previously defined and CG is a capping group;

A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl.

In one embodiment, the compound of the invention may be of Formula (ISSSS)

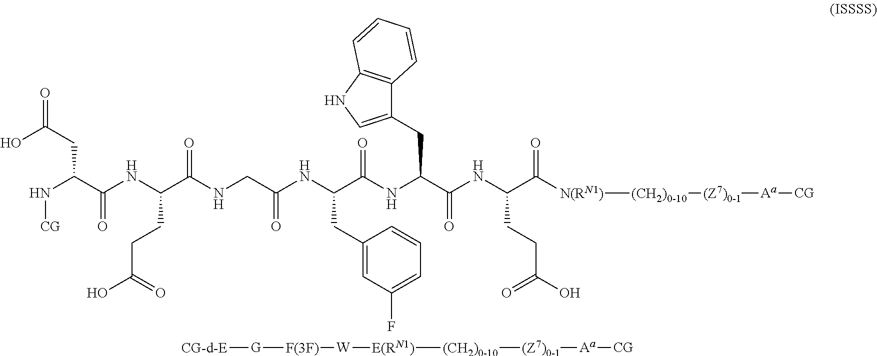

Wherein R^{N1} is as previously defined and CG is a capping group;

A^{a} is a chain of one or more non-naturally occurring amino acids, or a chain of one or more naturally occurring amino acids, or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids, in particular A^{a} is lysyl.

In one embodiment, the compound of the invention may be of Formula (ISSSSS)

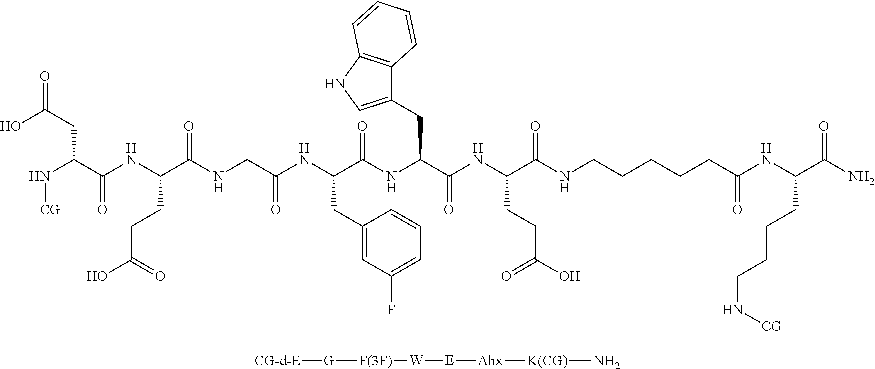

Wherein CG is a capping group.

In one embodiment, the compound of the invention may be of Formula (ISSSSS),

wherein CG is a capping group selected from List 1. In one embodiment, the capping group on the “d” terminus is selected from List 2 and/or the capping group on the “K” termunis is selected from List 3.

In one embodiment, the compound of the invention may comprise or consist of a sequence selected from the group consisting of:

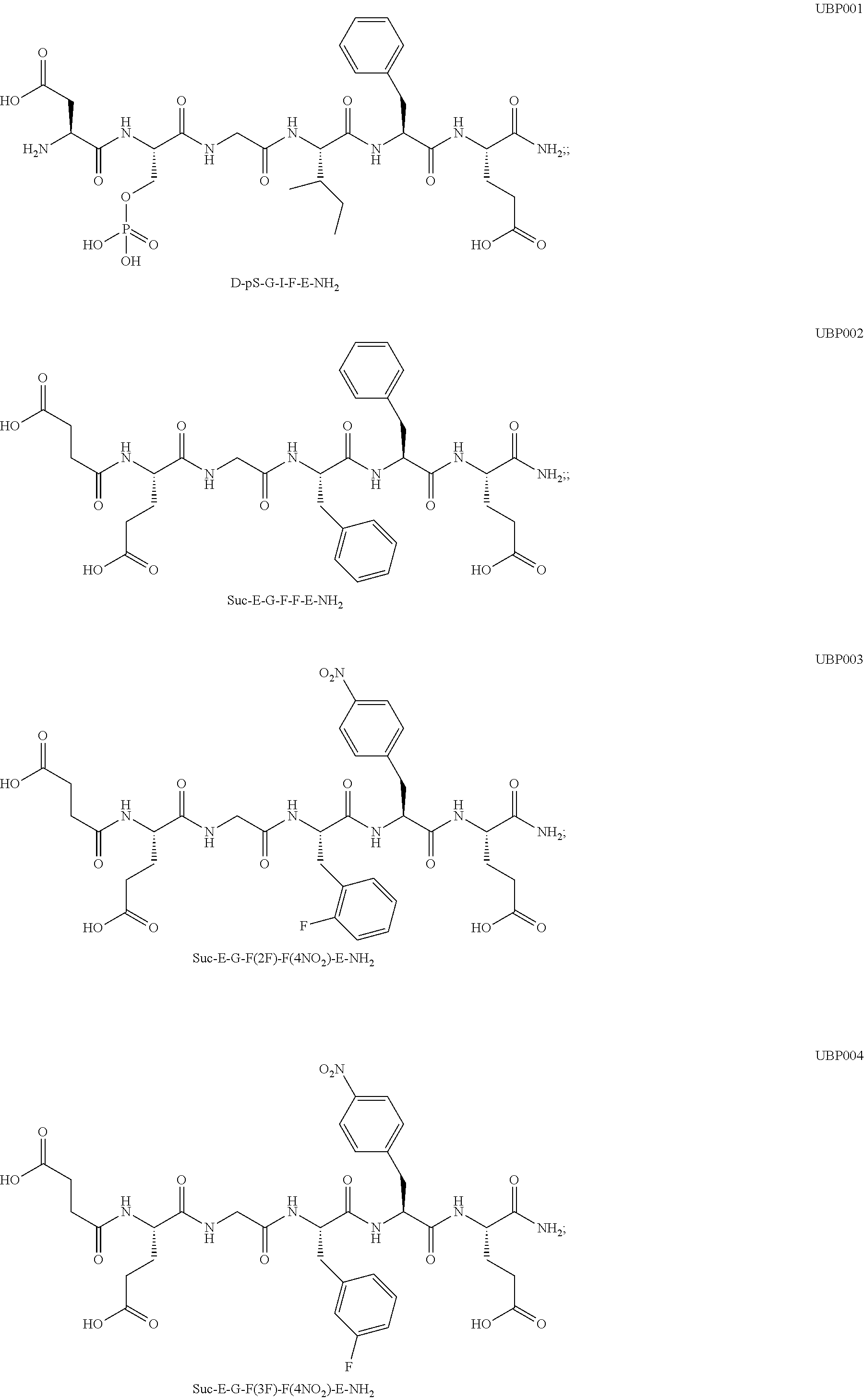 [SMILES: CCC(C)[C@H](NC(=O)CNC(=O)[C@H](COP(=O)(O)O)NC(=O)[C@@H](N)CC(=O)O)C(=O)N[C@@H](Cc1ccccc1)C(=O)N[C@@H](CCC(=O)O)C(N)=O ; NC(=O)[C@H](CCC(=O)O)NC(=O)[C@H](Cc1ccccc1)NC(=O)[C@H](Cc1ccccc1)NC(=O)CNC(=O)[C@H](CCC(=O)O)NC(=O)CCC(=O)O ; NC(=O)[C@H](CCC(=O)O)NC(=O)[C@H](Cc1ccc([N+](=O)[O-])cc1)NC(=O)[C@H](Cc1ccccc1F)NC(=O)CNC(=O)[C@H](CCC(=O)O)NC(=O)CCC(=O)O ; NC(=O)[C@H](CCC(=O)O)NC(=O)[C@H](Cc1ccc([N+](=O)[O-])cc1)NC(=O)[C@H](Cc1cccc(F)c1)NC(=O)CNC(=O)[C@H](CCC(=O)O)NC(=O)CCC(=O)O]

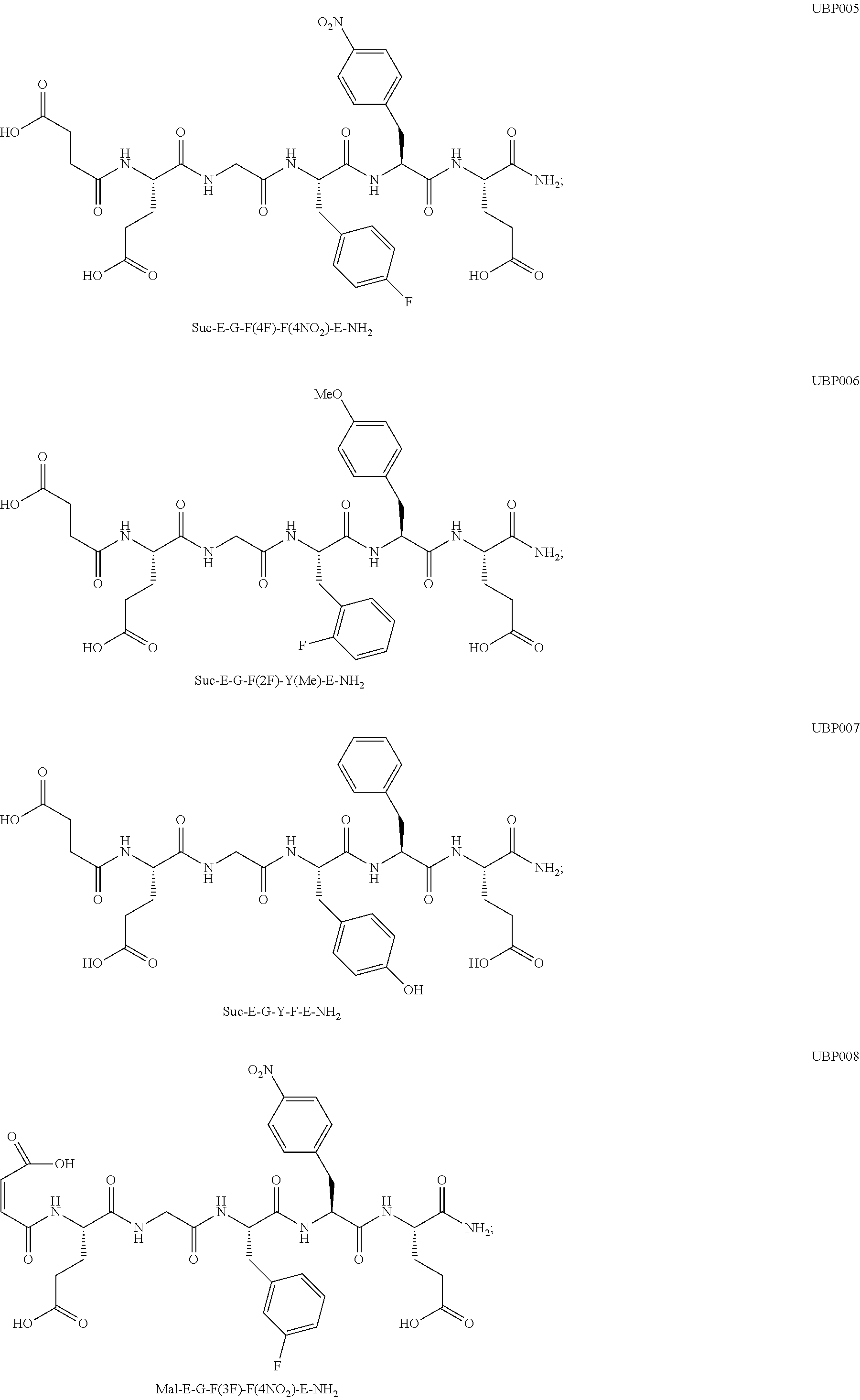 [SMILES: NC(=O)[C@H](CCC(=O)O)NC(=O)[C@H](Cc1ccc([N+](=O)[O-])cc1)NC(=O)[C@H](Cc1ccc(F)cc1)NC(=O)CNC(=O)[C@H](CCC(=O)O)NC(=O)CCC(=O)O ; COc1ccc(C[C@H](NC(=O)[C@H](Cc2ccccc2F)NC(=O)CNC(=O)[C@H](CCC(=O)O)NC(=O)CCC(=O)O)C(=O)N[C@@H](CCC(=O)O)C(N)=O)cc1 ; NC(=O)[C@H](CCC(=O)O)NC(=O)[C@H](Cc1ccccc1)NC(=O)[C@H](Cc1ccc(O)cc1)NC(=O)CNC(=O)[C@H](CCC(=O)O)NC(=O)CCC(=O)O ; NC(=O)[C@H](CCC(=O)O)NC(=O)[C@H](Cc1ccc([N+](=O)[O-])cc1)NC(=O)[C@H](Cc1cccc(F)c1)NC(=O)CNC(=O)[C@H](CCC(=O)O)NC(=O)/C=C\C(=O)O]

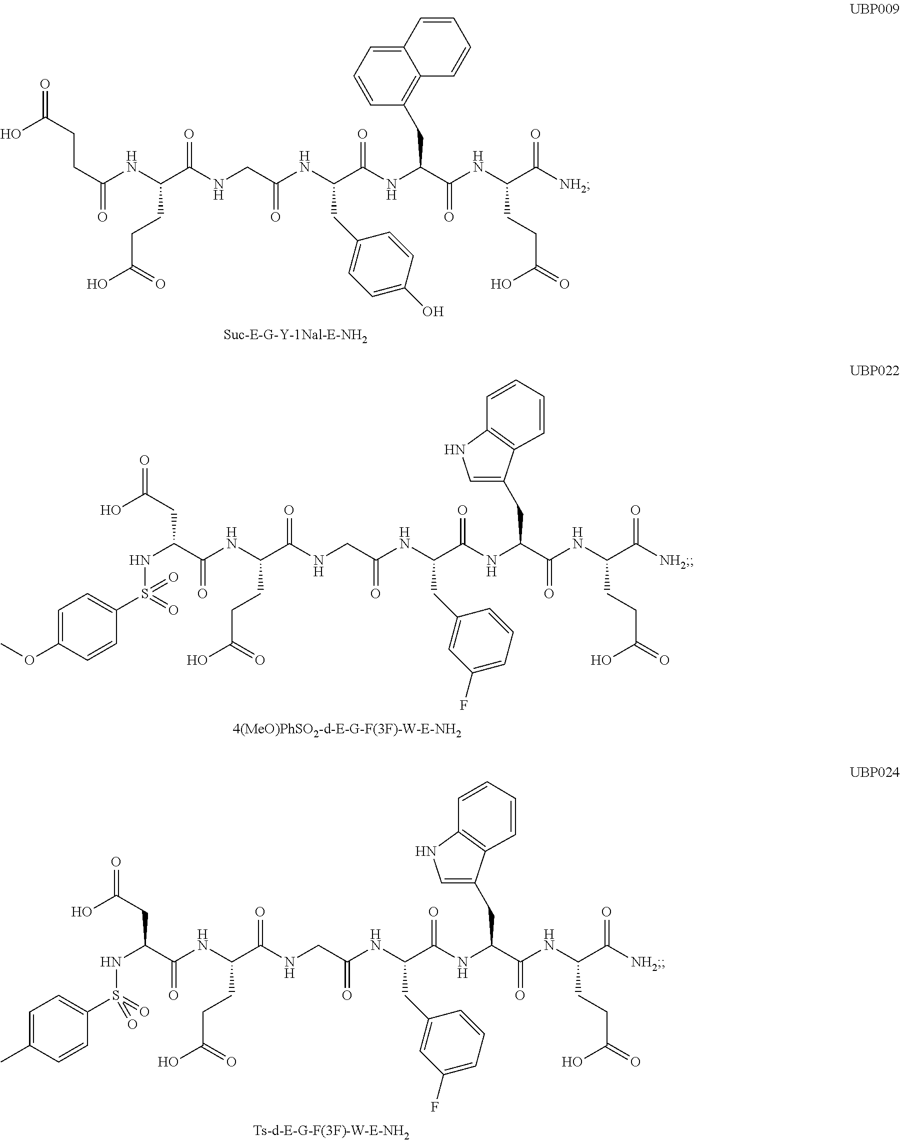 [SMILES: NC(=O)[C@H](CCC(=O)O)NC(=O)[C@H](Cc1cccc2ccccc12)NC(=O)[C@H](Cc1ccc(O)cc1)NC(=O)CNC(=O)[C@H](CCC(=O)O)NC(=O)CCC(=O)O ; COc1ccc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)cc1 ; Cc1ccc(S(=O)(=O)N[C@@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)cc1]

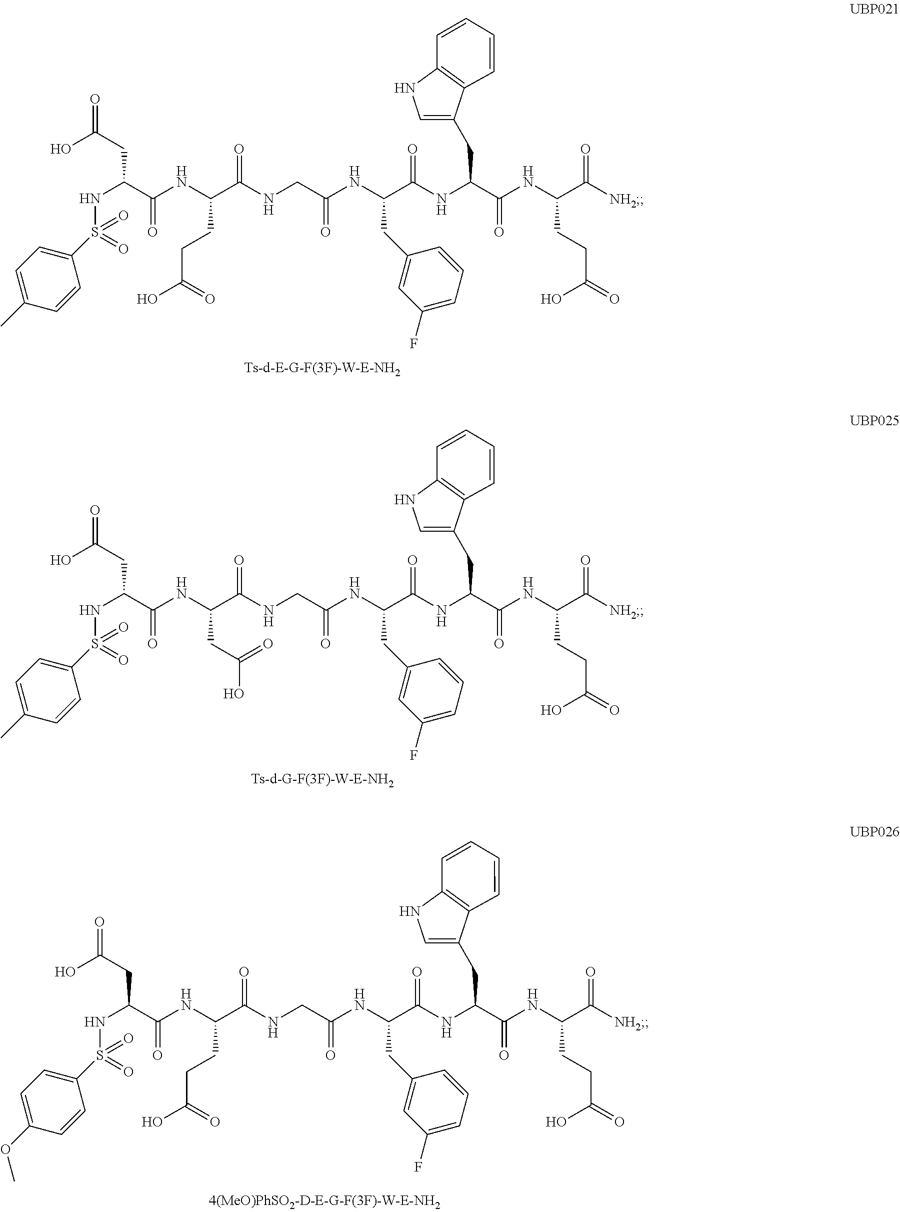 [SMILES: Cc1ccc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)cc1 ; Cc1ccc(S(=O)(=O)N[C@H](CC(=O)O)C(=O)N[C@@H](CC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)cc1 ; COc1ccc(S(=O)(=O)N[C@@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)cc1]

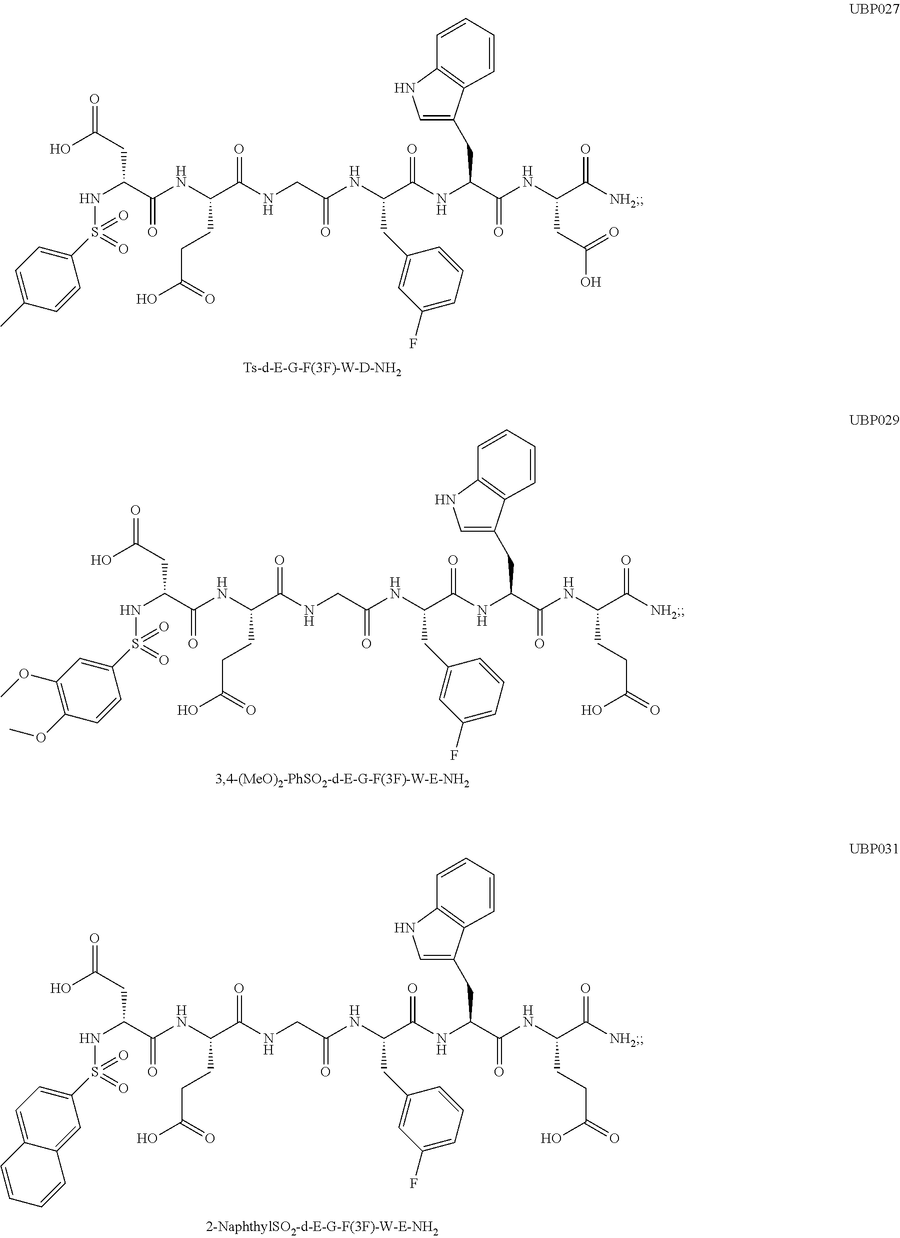 [SMILES: Cc1ccc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CC(=O)O)C(N)=O)cc1 ; COc1ccc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)cc1OC ; NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc2ccccc2c1]

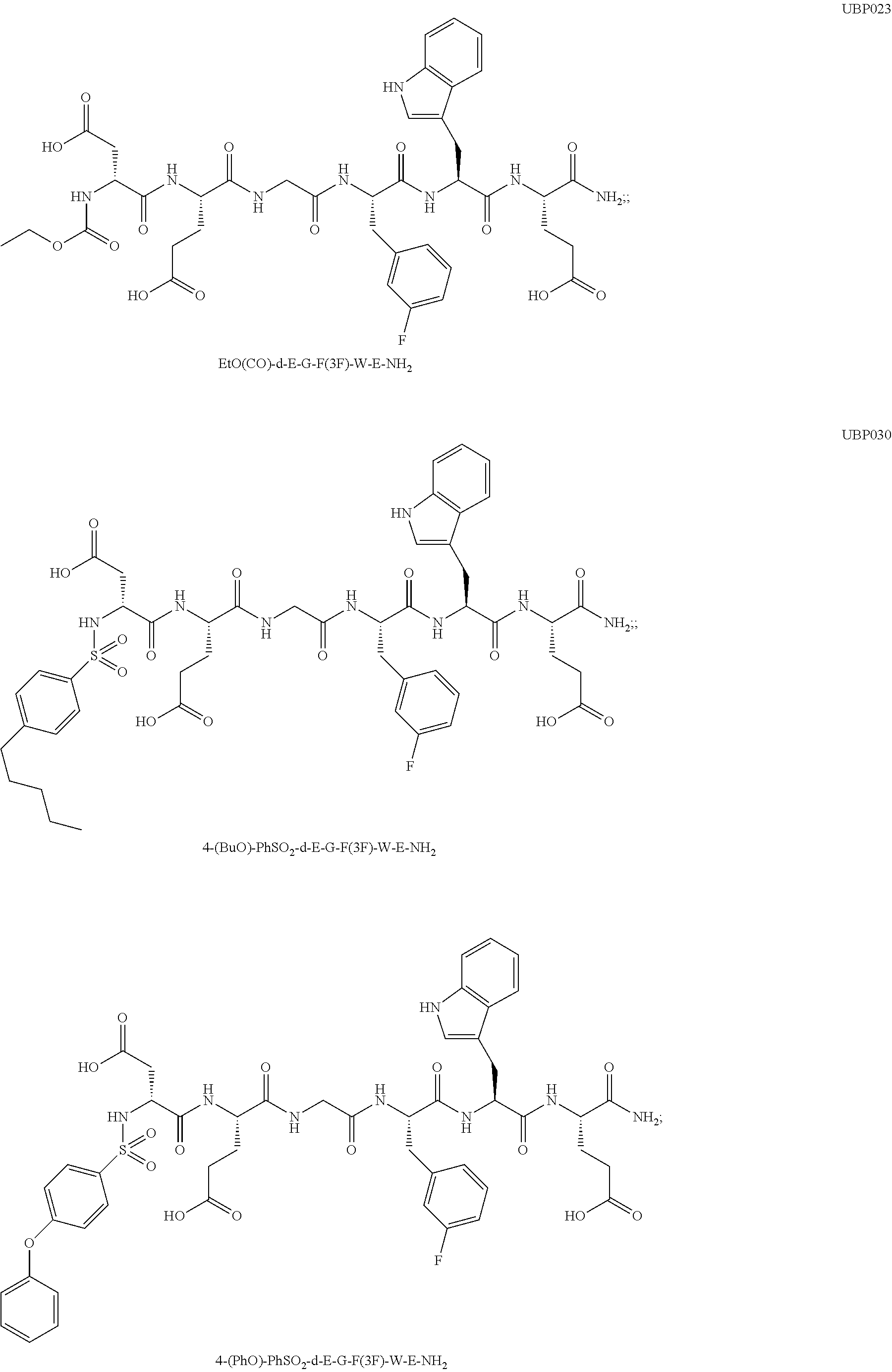 [SMILES: CCOC(=O)N[C@H](CC(=O)O)C(=O)N[C@@H](CCC(=O)O)C(=O)NCC(=O)N[C@@H](Cc1cccc(F)c1)C(=O)N[C@@H](Cc1c[nH]c2ccccc12)C(=O)N[C@@H](CCC(=O)O)C(N)=O ; CCCCCc1ccc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)cc1 ; NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(Oc2ccccc2)cc1]

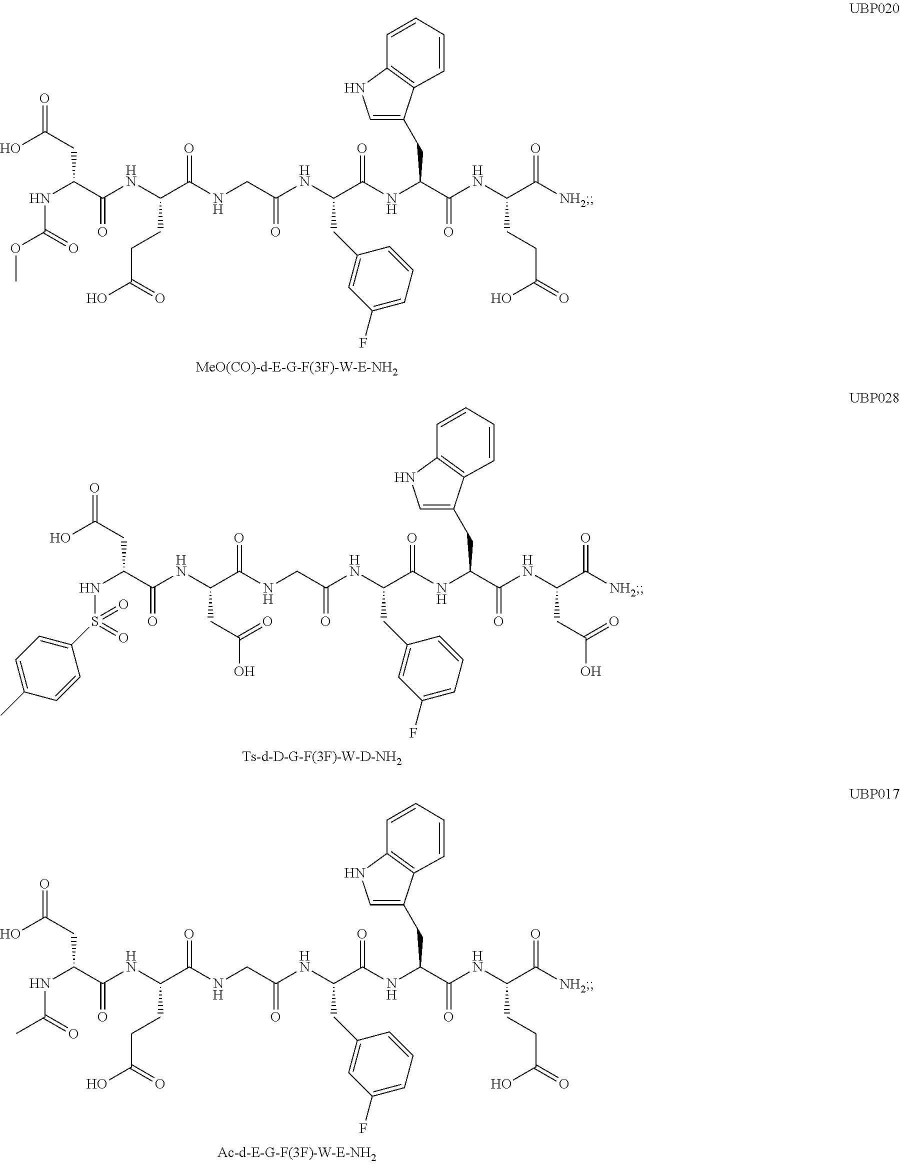 [SMILES: COC(=O)N[C@H](CC(=O)O)C(=O)N[C@@H](CCC(=O)O)C(=O)NCC(=O)N[C@@H](Cc1cccc(F)c1)C(=O)N[C@@H](Cc1c[nH]c2ccccc12)C(=O)N[C@@H](CCC(=O)O)C(N)=O ; Cc1ccc(S(=O)(=O)N[C@H](CC(=O)O)C(=O)N[C@@H](CC(=O)O)C(=O)NCC(=O)N[C@@H](Cc2cccc(F)c2)C(=O)N[C@@H](Cc2c[nH]c3ccccc23)C(=O)N[C@@H](CC(=O)O)C(N)=O)cc1 ; CC(=O)N[C@H](CC(=O)O)C(=O)N[C@@H](CCC(=O)O)C(=O)NCC(=O)N[C@@H](Cc1cccc(F)c1)C(=O)N[C@@H](Cc1c[nH]c2ccccc12)C(=O)N[C@@H](CCC(=O)O)C(N)=O]

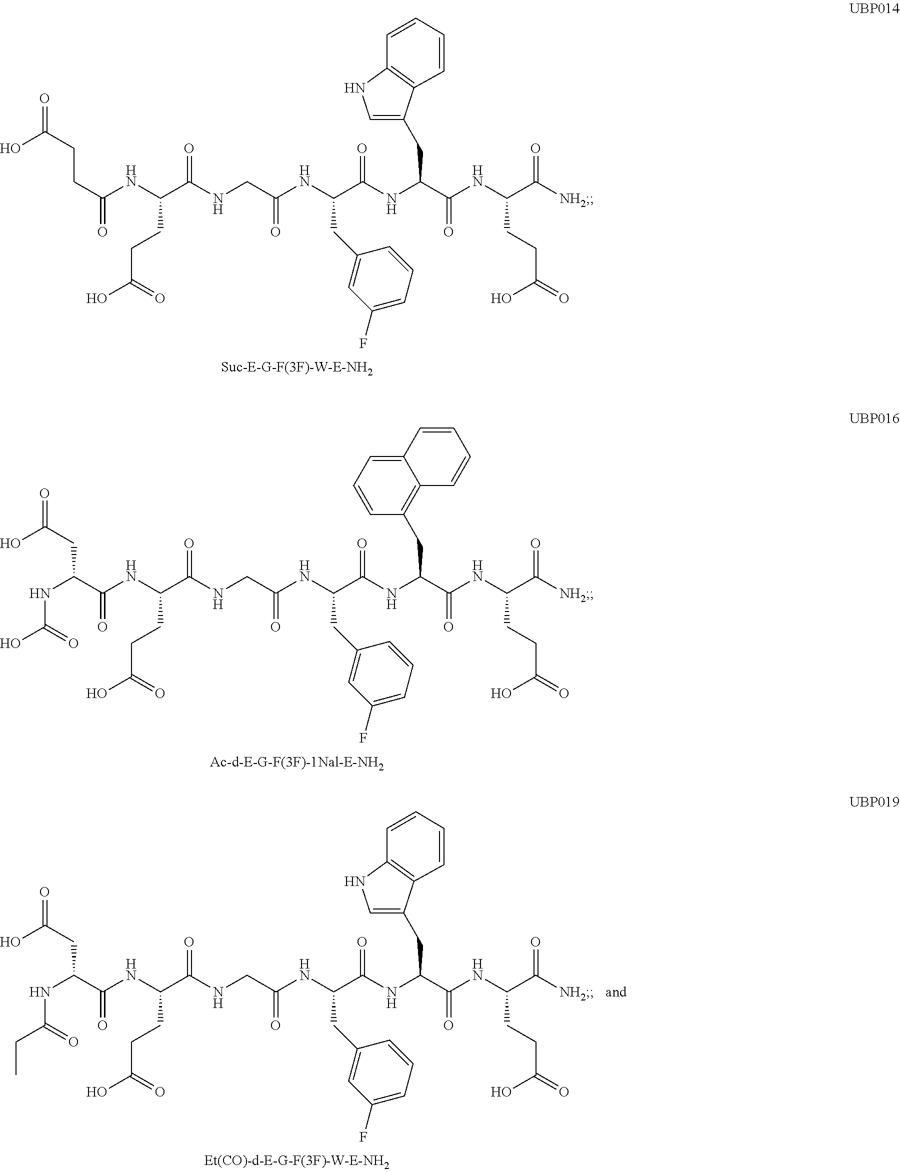 [SMILES: NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)CCC(=O)O ; NC(=O)[C@H](CCC(=O)O)NC(=O)[C@H](Cc1cccc2ccccc12)NC(=O)[C@H](Cc1cccc(F)c1)NC(=O)CNC(=O)[C@H](CCC(=O)O)NC(=O)[C@@H](CC(=O)O)NC(=O)O ; CCC(=O)N[C@H](CC(=O)O)C(=O)N[C@@H](CCC(=O)O)C(=O)NCC(=O)N[C@@H](Cc1cccc(F)c1)C(=O)N[C@@H](Cc1c[nH]c2ccccc12)C(=O)N[C@@H](CCC(=O)O)C(N)=O]

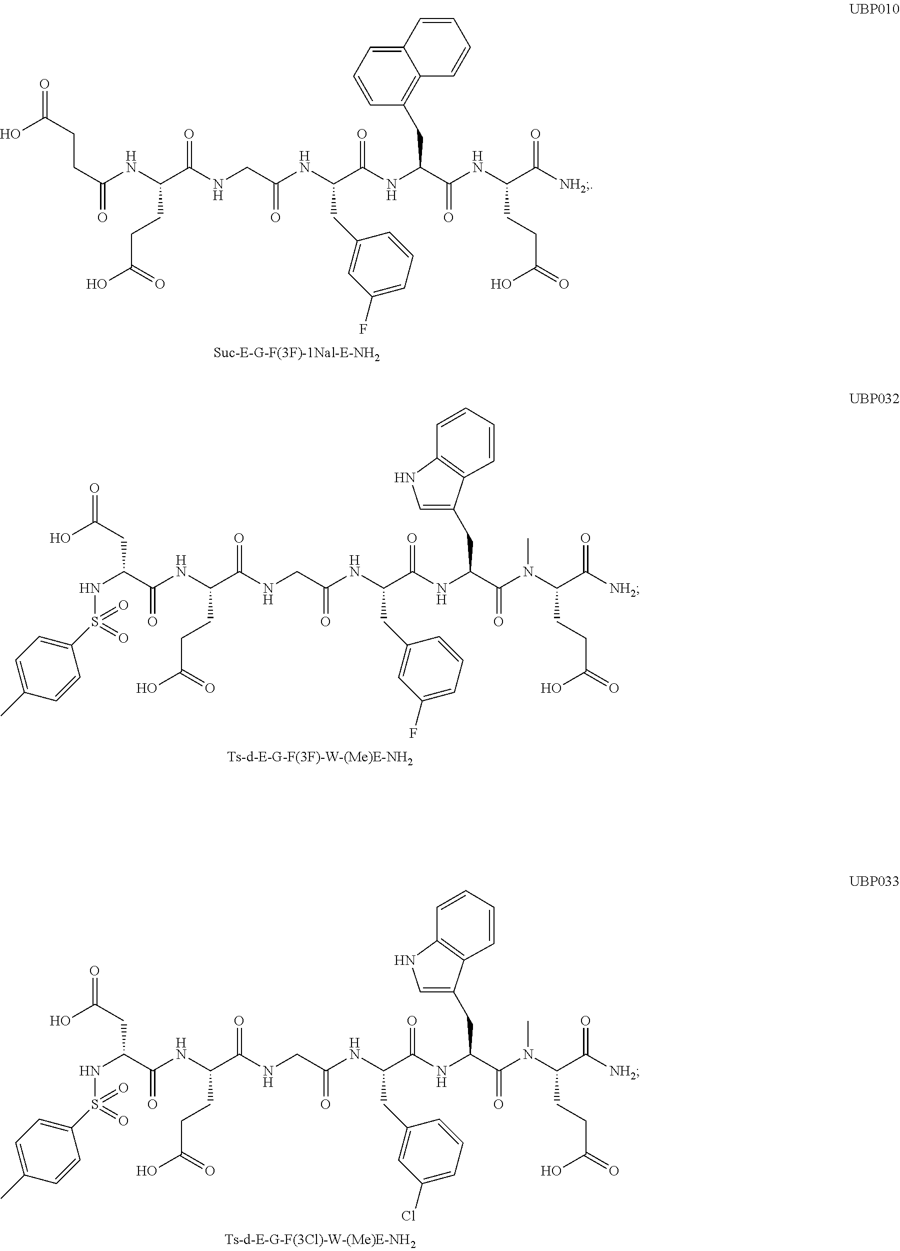 [SMILES: NC(=O)[C@H](CCC(=O)O)NC(=O)[C@H](Cc1cccc2ccccc12)NC(=O)[C@H](Cc1cccc(F)c1)NC(=O)CNC(=O)[C@H](CCC(=O)O)NC(=O)CCC(=O)O ; Cc1ccc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N(C)[C@@H](CCC(=O)O)C(N)=O)cc1 ; Cc1ccc(S(=O)(=O)N[C@H](CC(=O)O)C(=O)N[C@@H](CCC(=O)O)C(=O)NCC(=O)N[C@@H](Cc2cccc(Cl)c2)C(=O)N[C@@H](Cc2c[nH]c3ccccc23)C(=O)N(C)[C@@H](CCC(=O)O)C(N)=O)cc1]

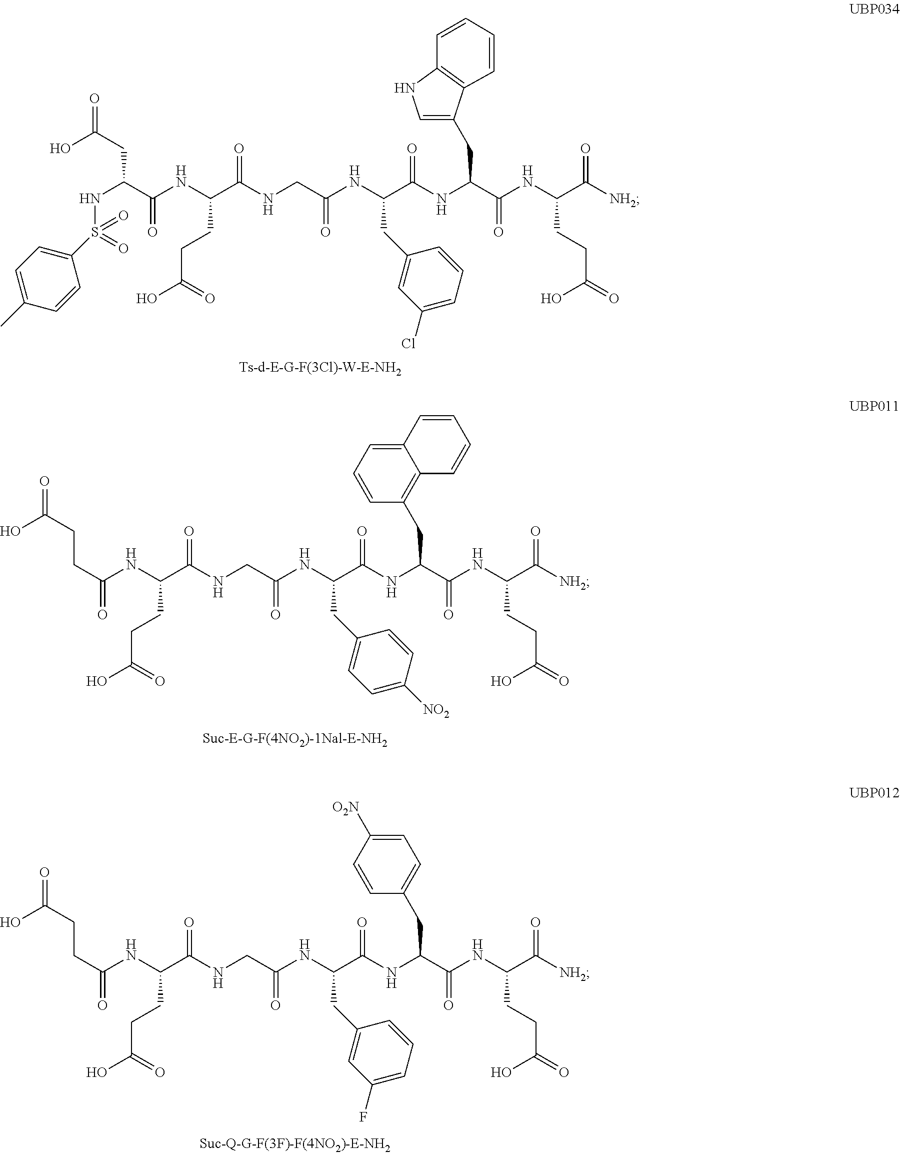 [SMILES: Cc1ccc(S(=O)(=O)N[C@H](CC(=O)O)C(=O)N[C@@H](CCC(=O)O)C(=O)NCC(=O)N[C@@H](Cc2cccc(Cl)c2)C(=O)N[C@@H](Cc2c[nH]c3ccccc23)C(=O)N[C@@H](CCC(=O)O)C(N)=O)cc1 ; NC(=O)[C@H](CCC(=O)O)NC(=O)[C@H](Cc1cccc2ccccc12)NC(=O)[C@H](Cc1ccc([N+](=O)[O-])cc1)NC(=O)CNC(=O)[C@H](CCC(=O)O)NC(=O)CCC(=O)O ; NC(=O)[C@H](CCC(=O)O)NC(=O)[C@H](Cc1ccc([N+](=O)[O-])cc1)NC(=O)[C@H](Cc1cccc(F)c1)NC(=O)CNC(=O)[C@H](CCC(=O)O)NC(=O)CCC(=O)O]

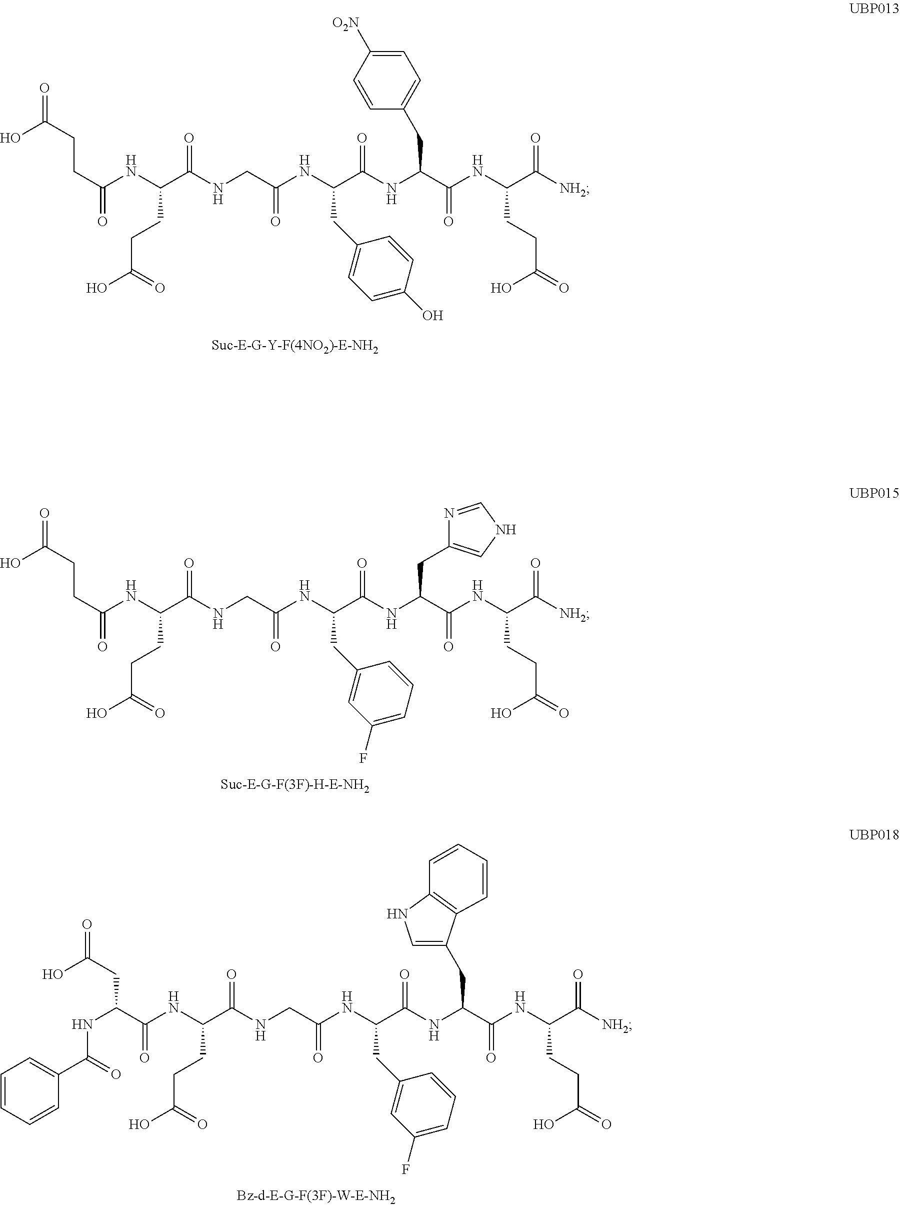 [SMILES: NC(=O)[C@H](CCC(=O)O)NC(=O)[C@H](Cc1ccc([N+](=O)[O-])cc1)NC(=O)[C@H](Cc1ccc(O)cc1)NC(=O)CNC(=O)[C@H](CCC(=O)O)NC(=O)CCC(=O)O ; NC(=O)[C@H](CCC(=O)O)NC(=O)[C@H](Cc1c[nH]cn1)NC(=O)[C@H](Cc1cccc(F)c1)NC(=O)CNC(=O)[C@H](CCC(=O)O)NC(=O)CCC(=O)O ; NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NC(=O)c1ccccc1]

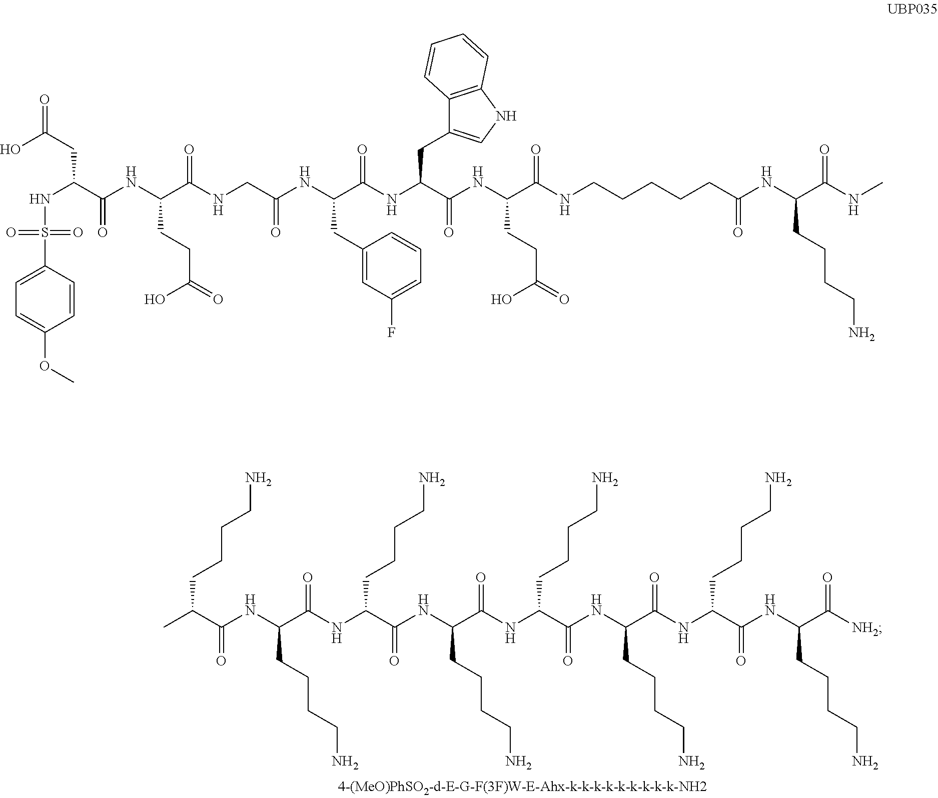

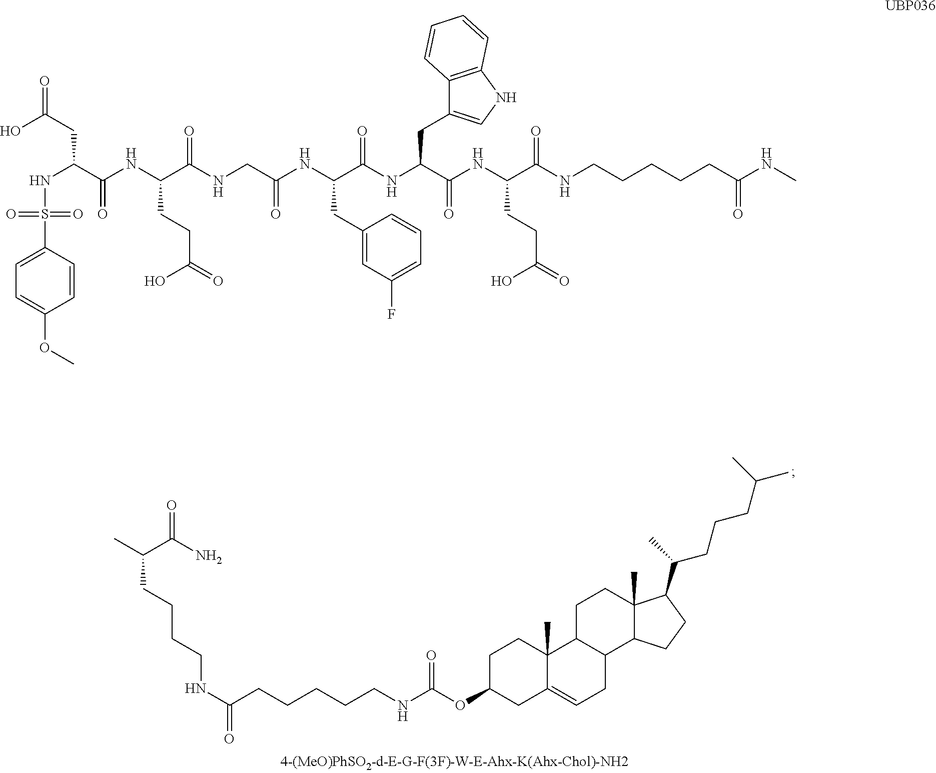

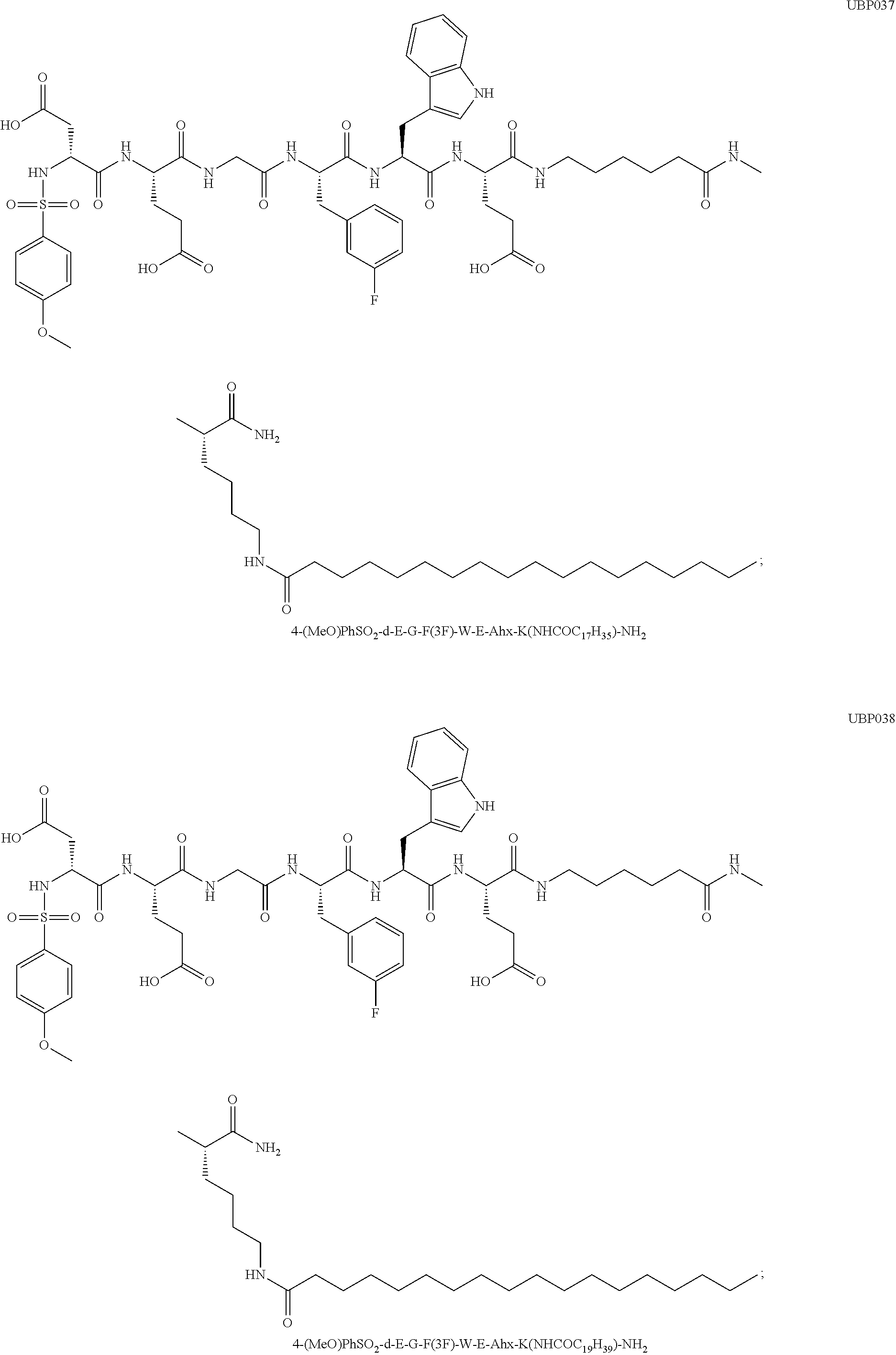

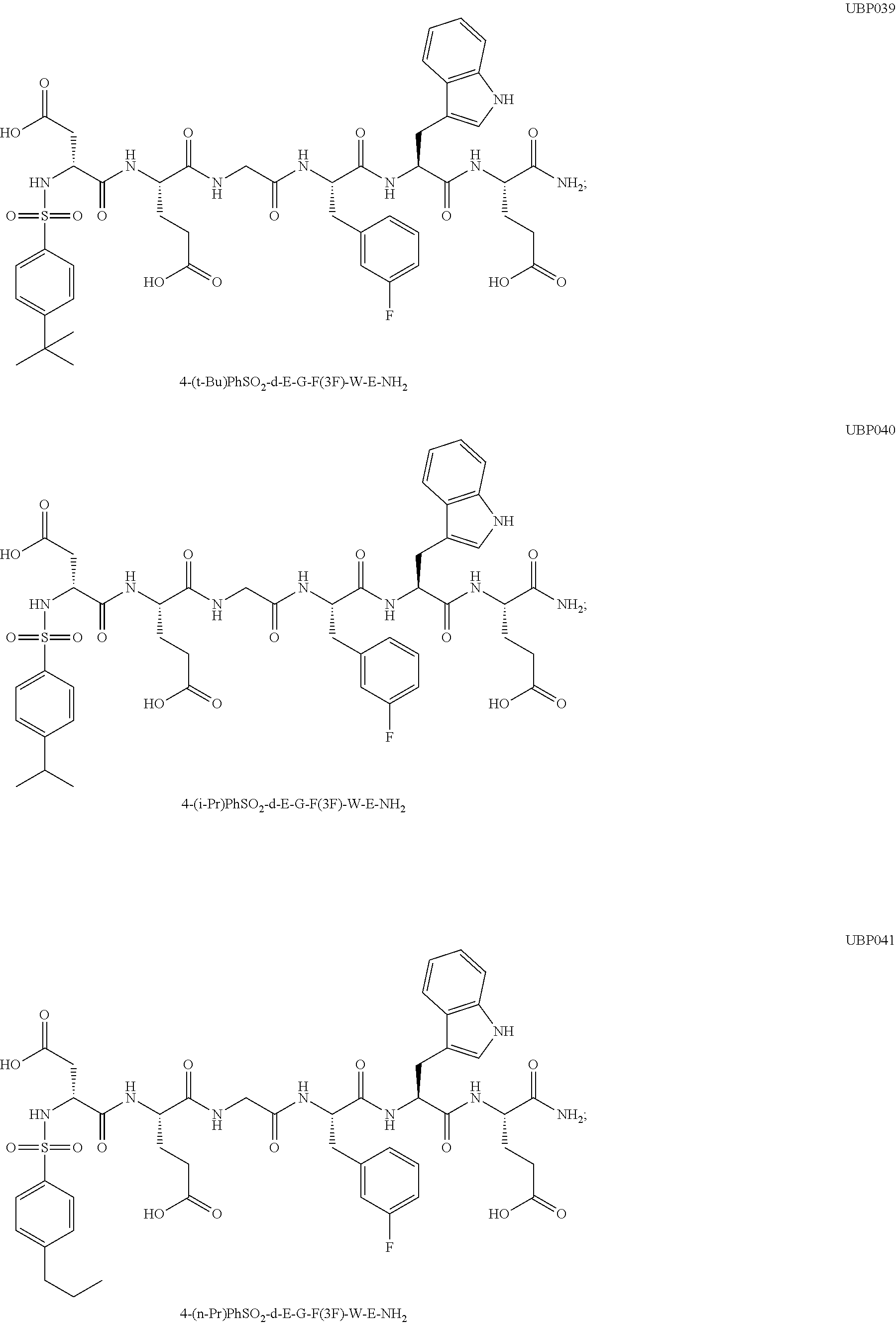 [SMILES: CC(C)(C)c1ccc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)cc1 ; CC(C)c1ccc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)cc1 ; CCCc1ccc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)cc1]

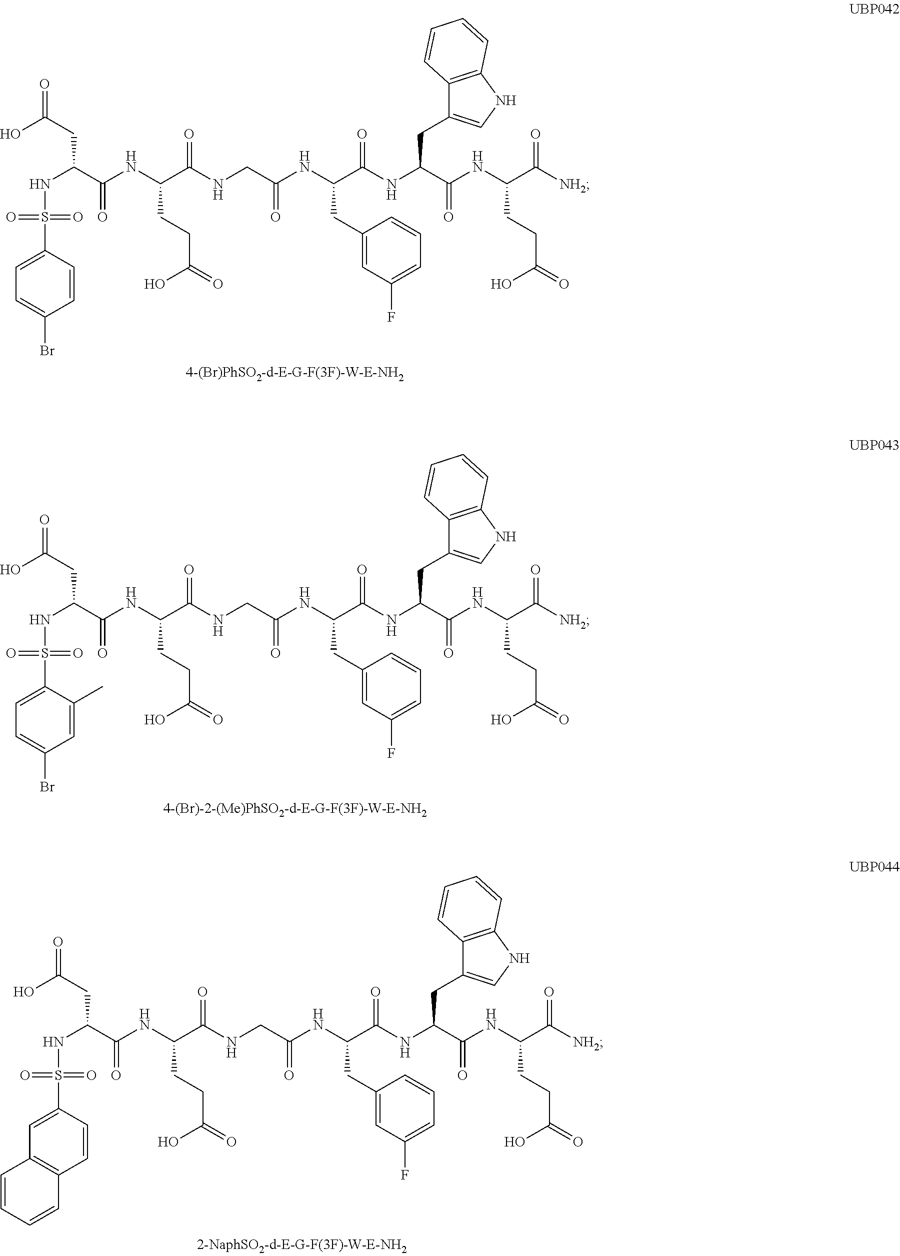 [SMILES: NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(Br)cc1 ; Cc1cc(Br)ccc1S(=O)(=O)N[C@H](CC(=O)O)C(=O)N[C@@H](CCC(=O)O)C(=O)NCC(=O)N[C@@H](Cc1cccc(F)c1)C(=O)N[C@@H](Cc1c[nH]c2ccccc12)C(=O)N[C@@H](CCC(=O)O)C(N)=O ; NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc2ccccc2c1]

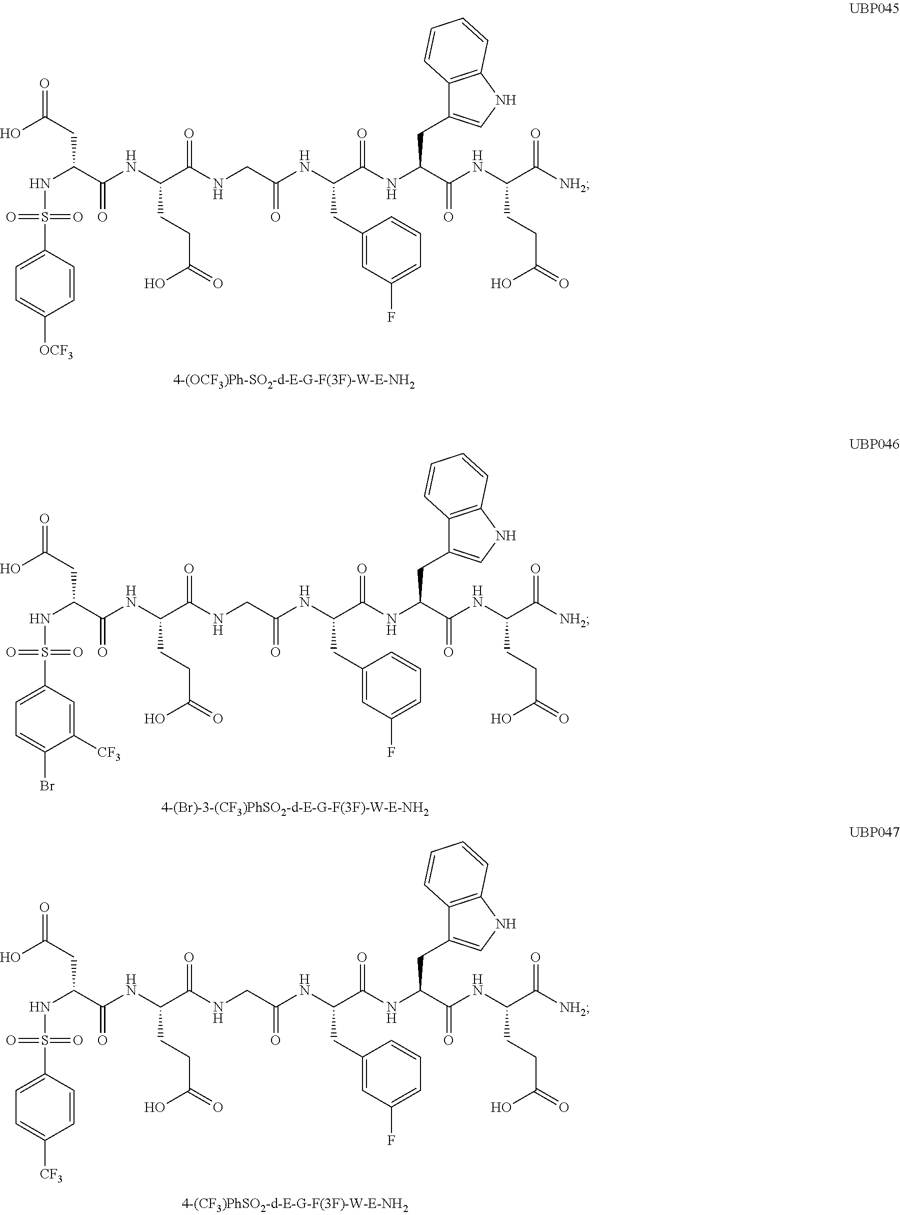 [SMILES: NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(OC(F)(F)F)cc1 ; NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(Br)c(C(F)(F)F)c1 ; NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(C(F)(F)F)cc1]

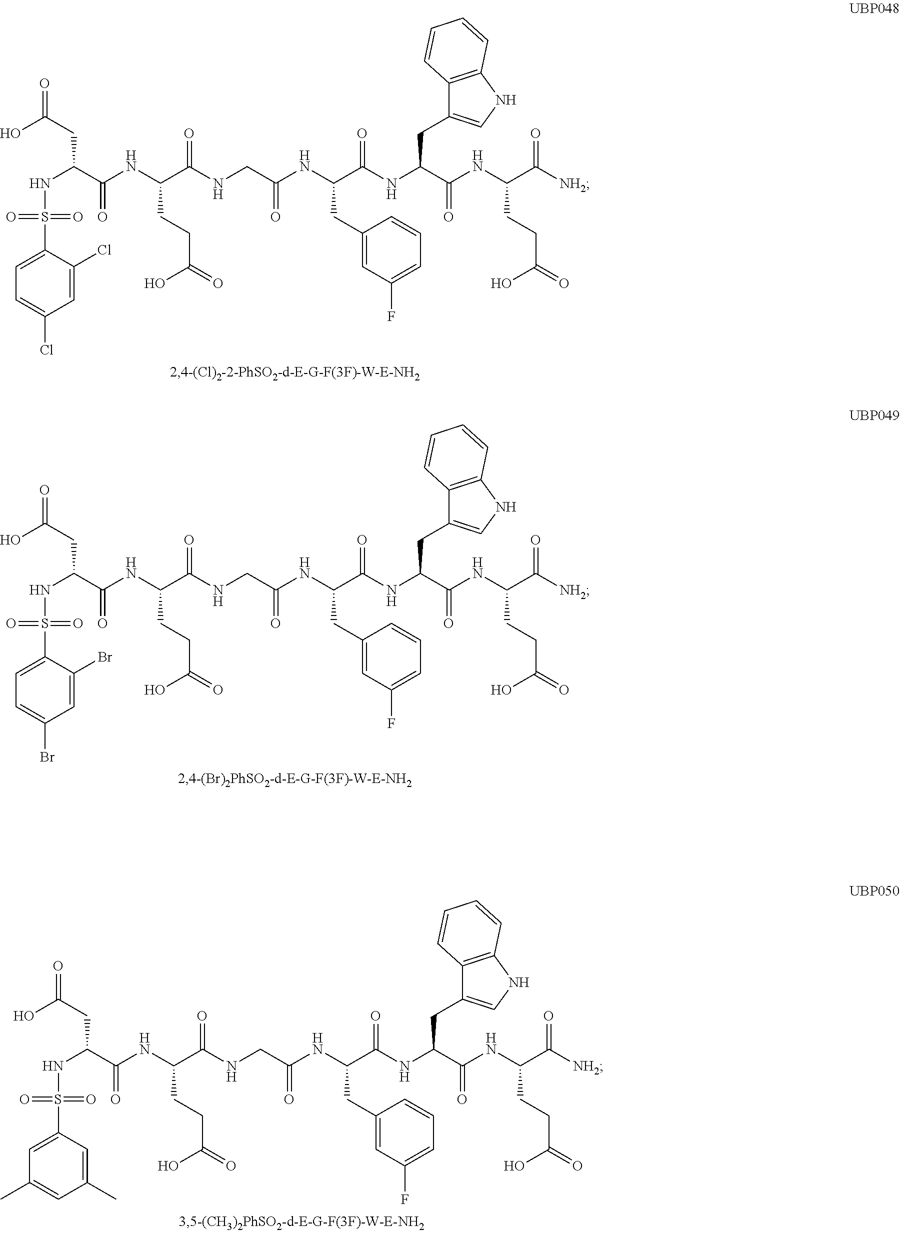 [SMILES: NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(Cl)cc1Cl ; NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(Br)cc1Br ; Cc1cc(C)cc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(N)=O)c1]

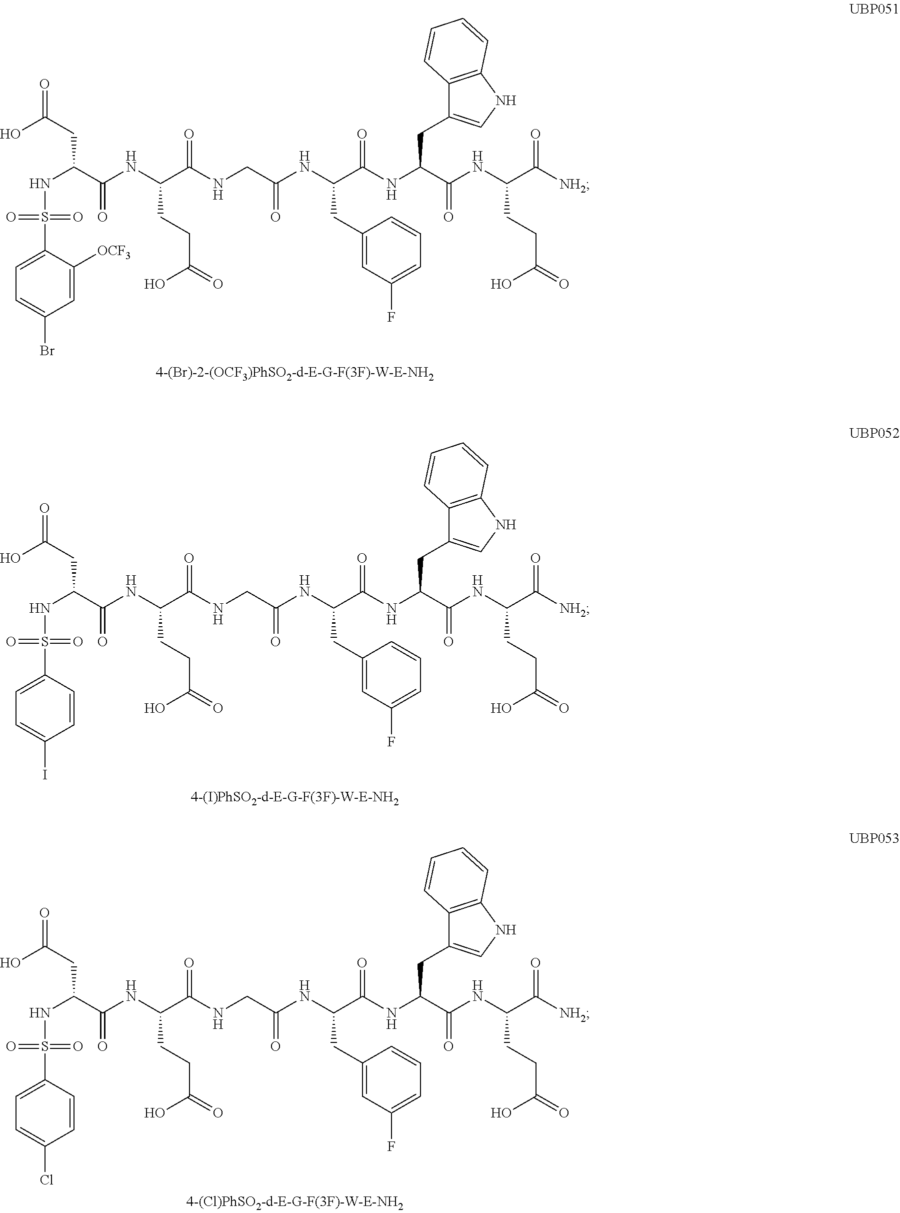 [SMILES: NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(Br)cc1OC(F)(F)F ; NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(I)cc1 ; NC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(Cl)cc1]

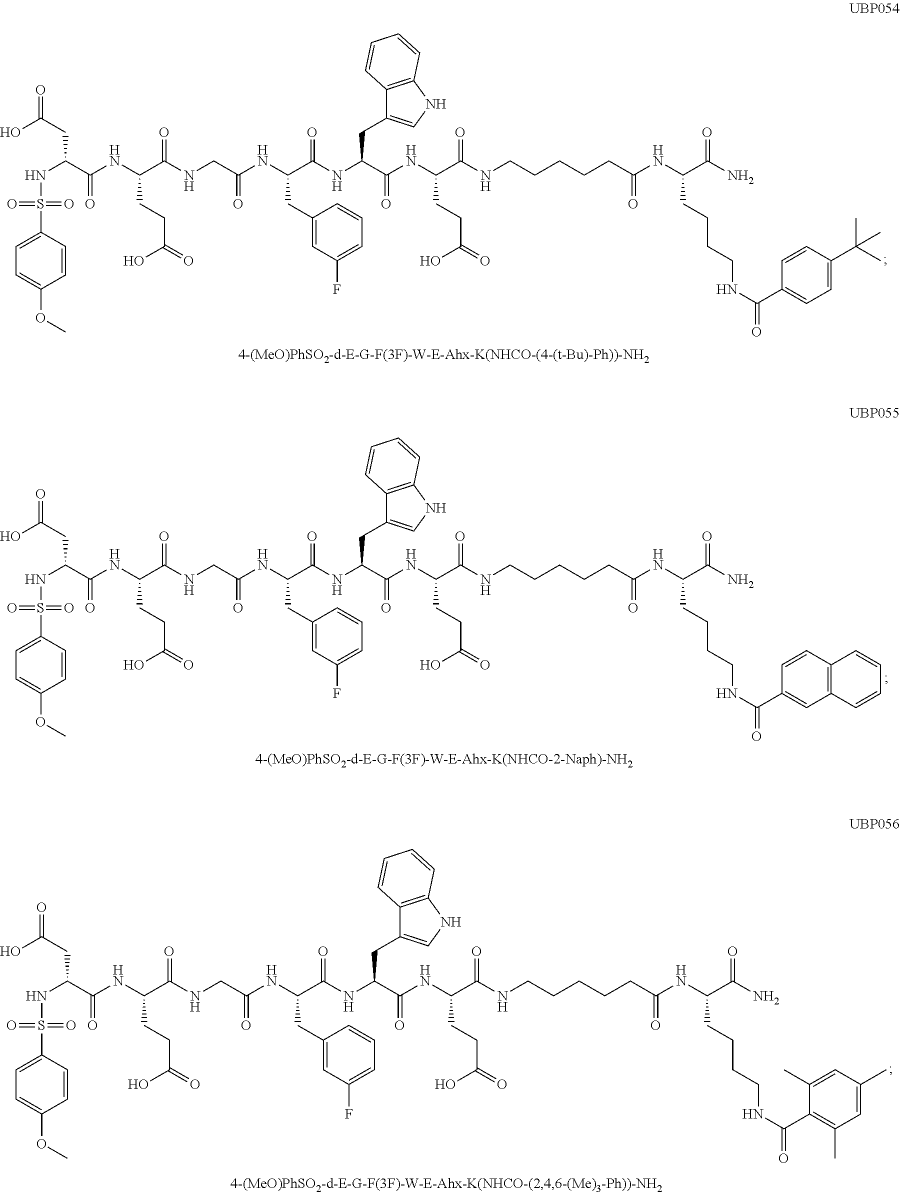

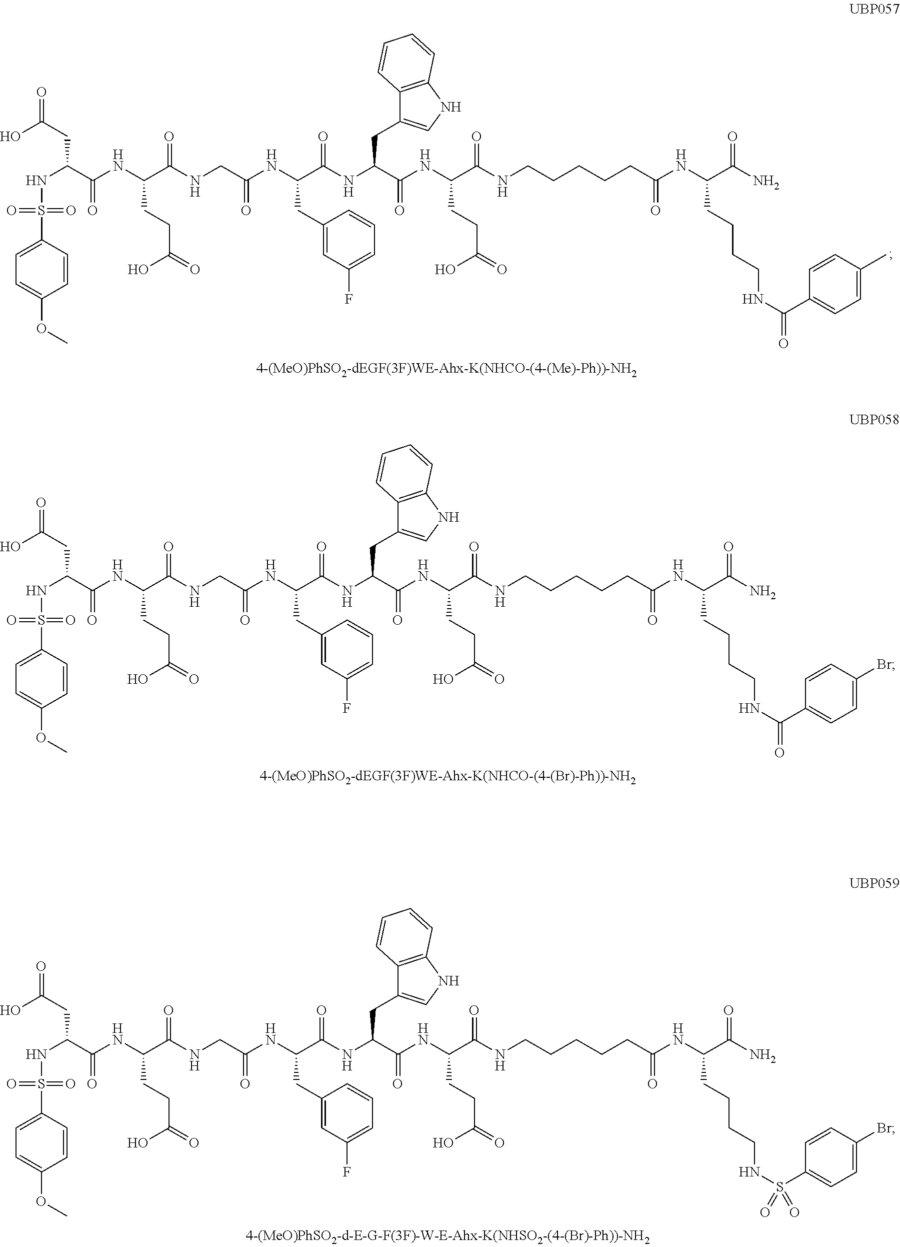

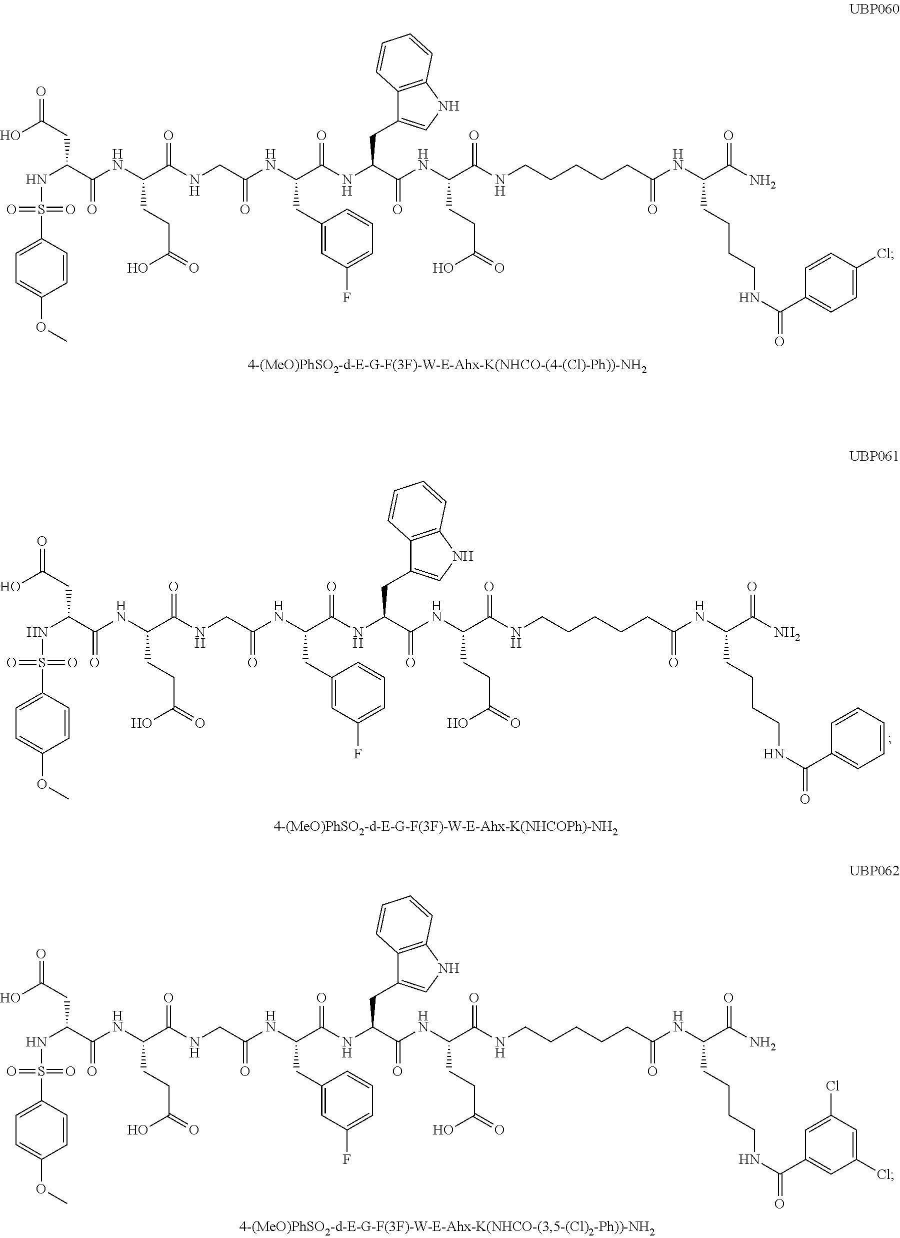

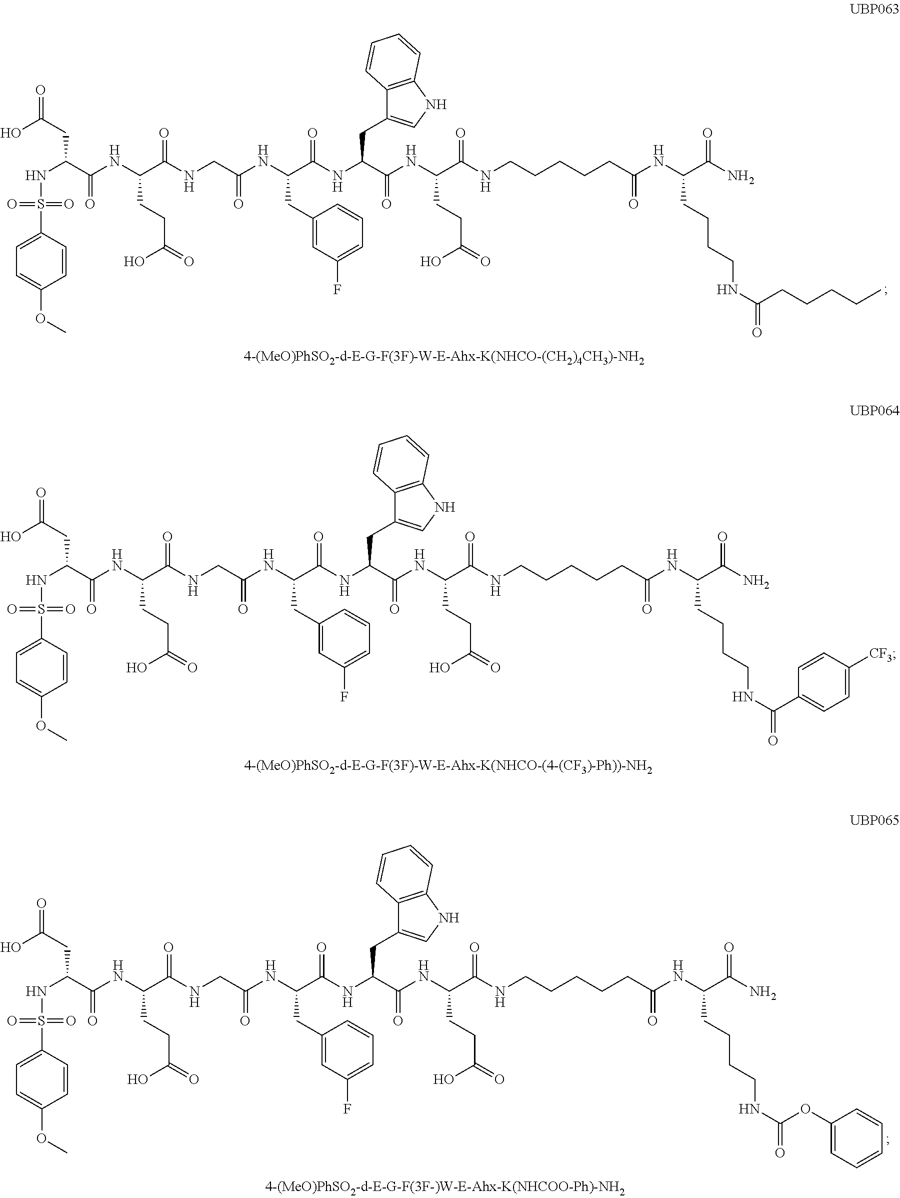

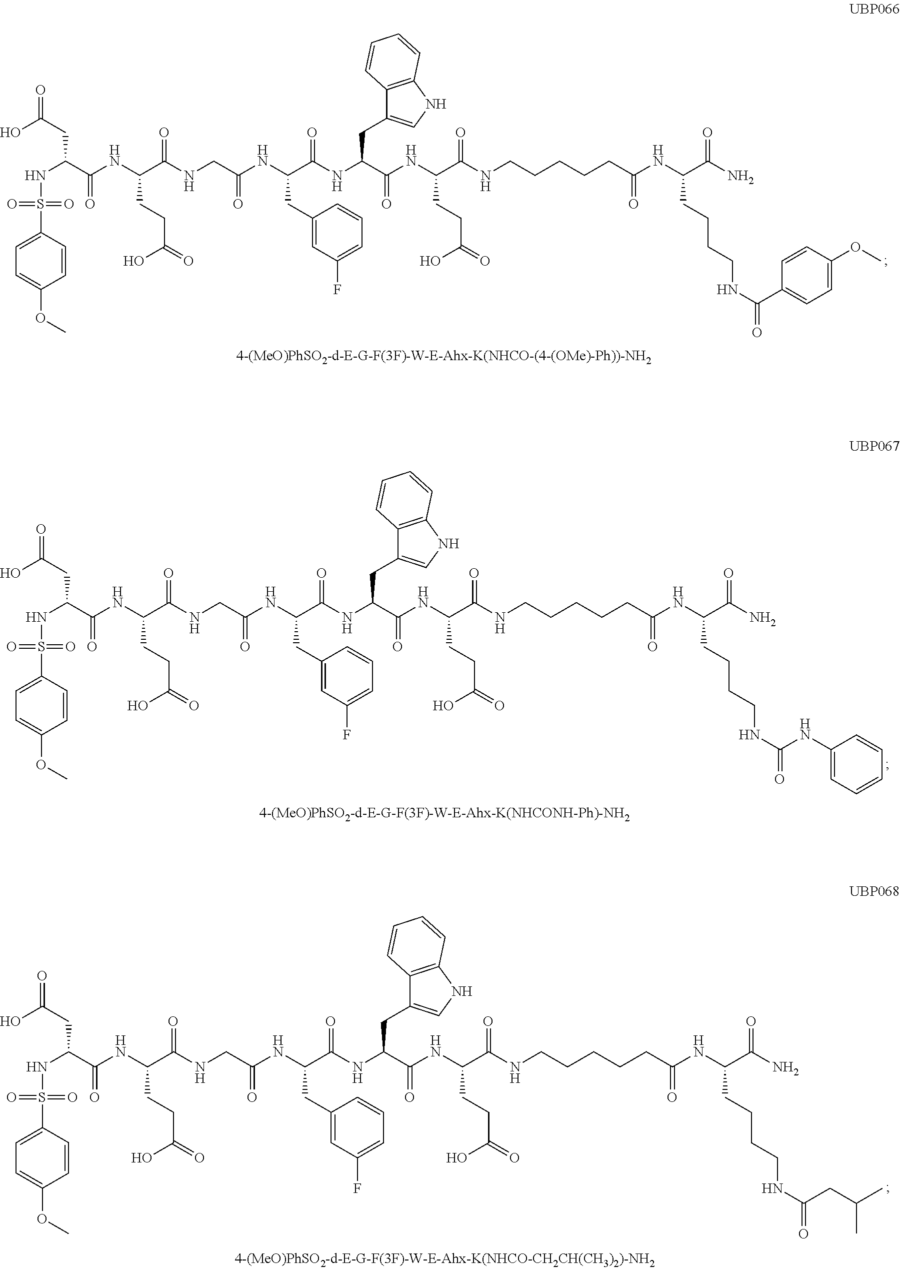

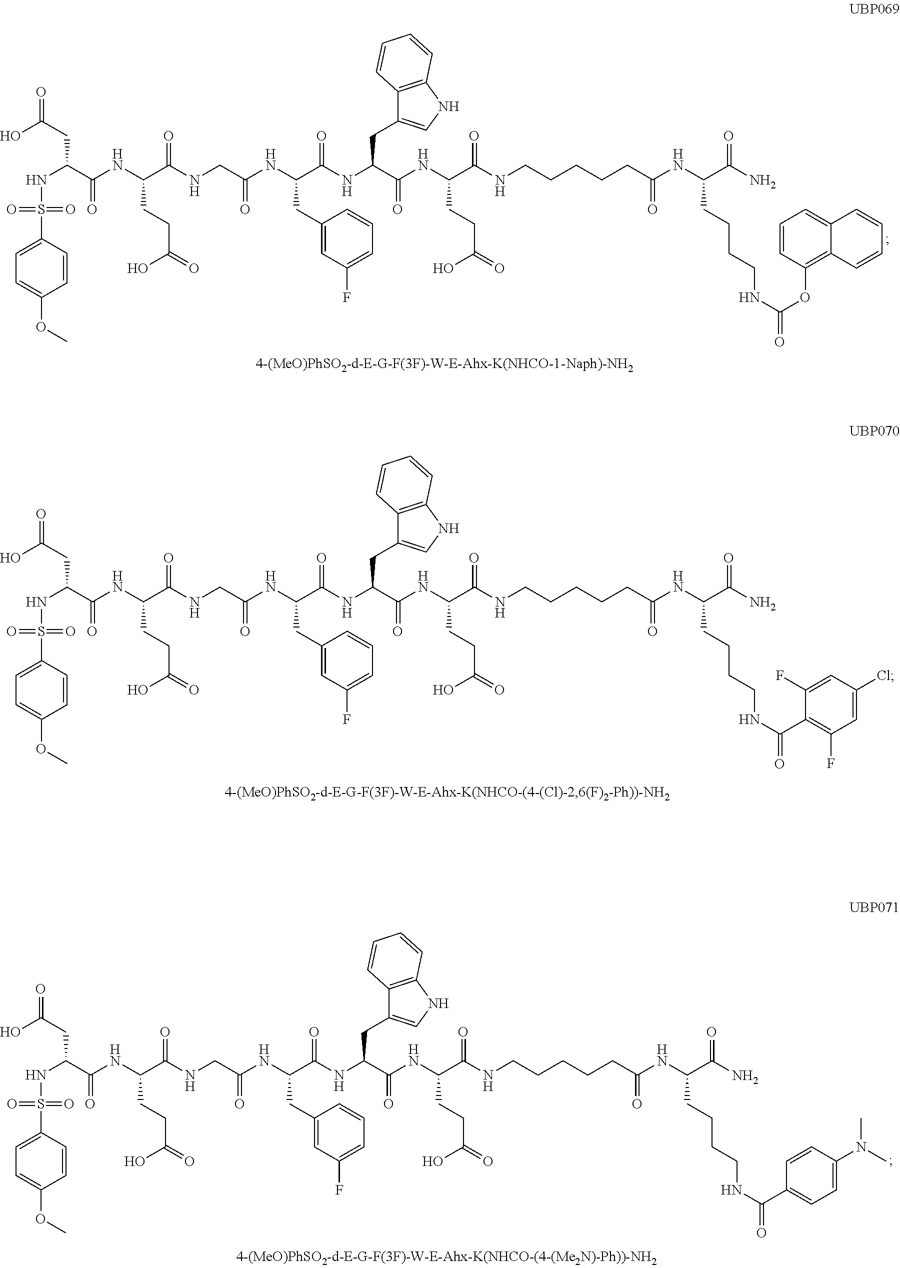

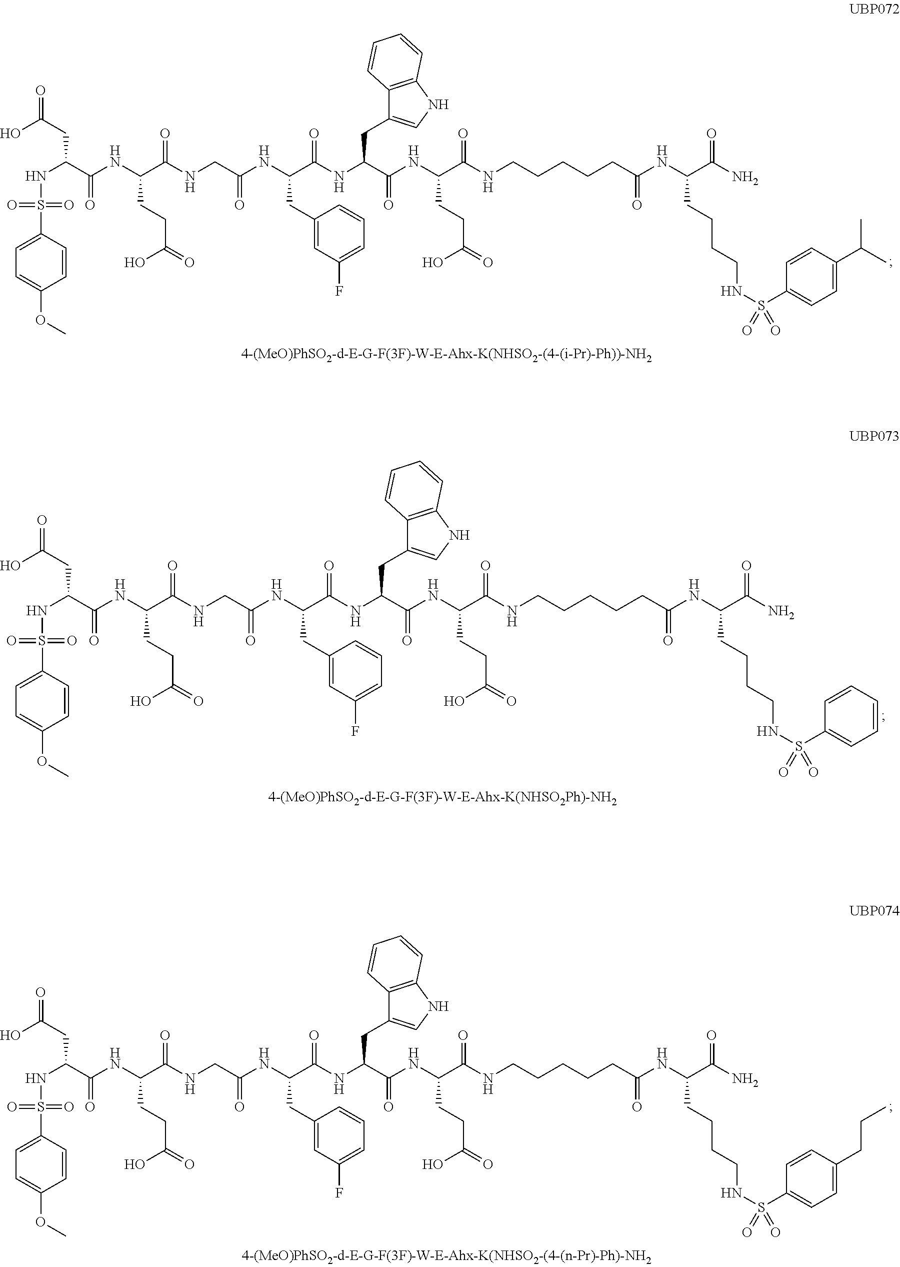

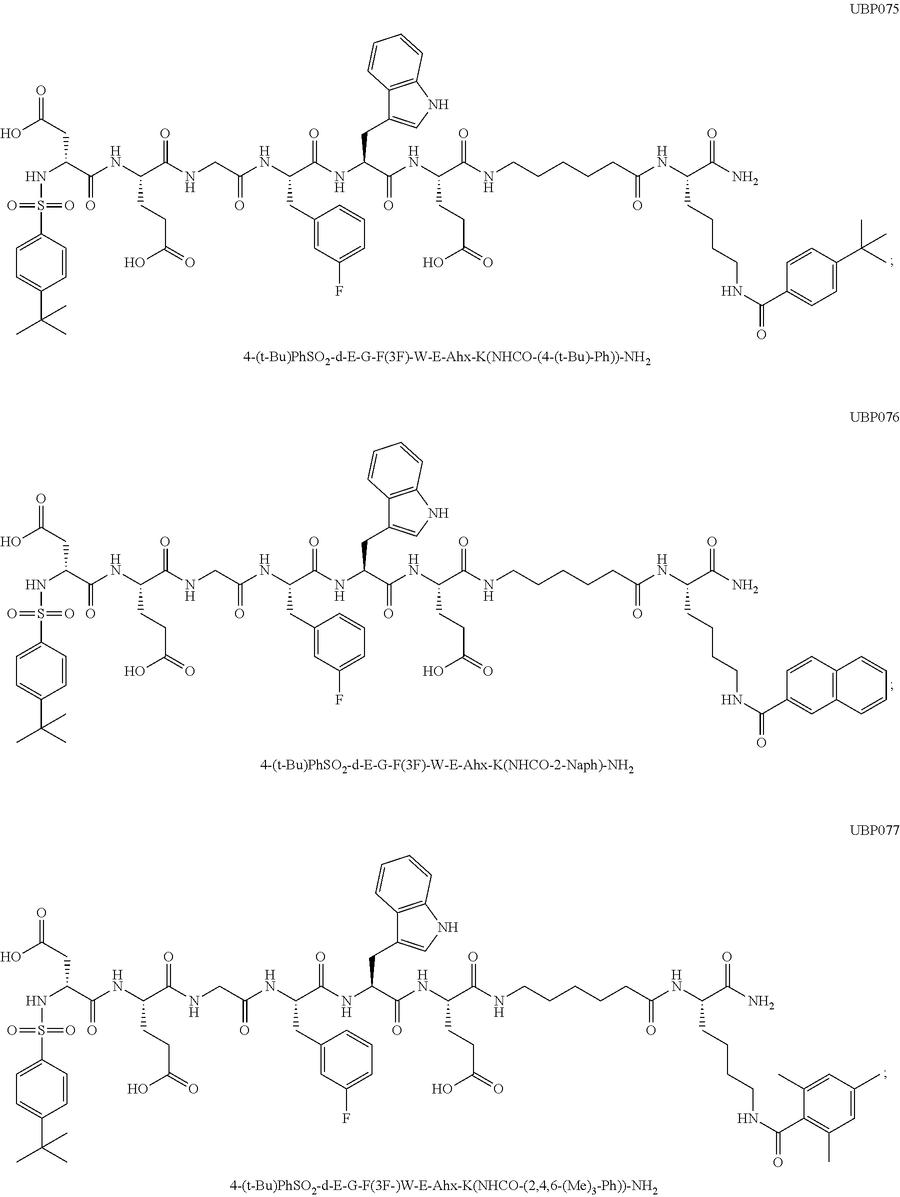

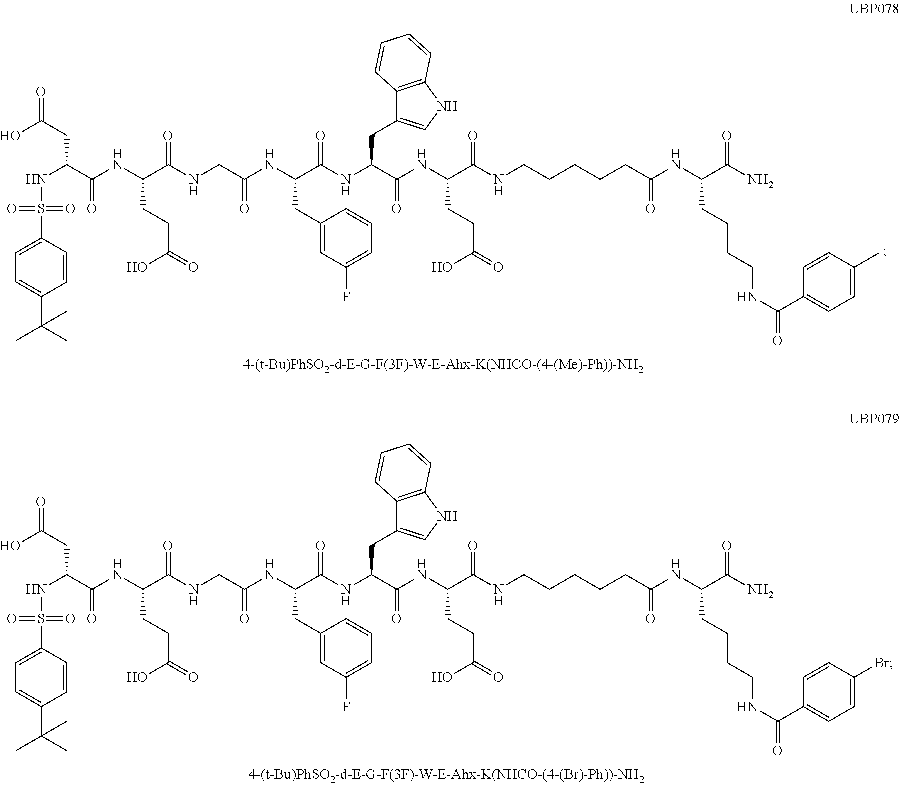 [SMILES: Cc1ccc(C(=O)NCCCC[C@H](NC(=O)CCCCCNC(=O)[C@H](CCC(=O)O)NC(=O)[C@H](Cc2c[nH]c3ccccc23)NC(=O)[C@H](Cc2cccc(F)c2)NC(=O)CNC(=O)[C@H](CCC(=O)O)NC(=O)[C@@H](CC(=O)O)NS(=O)(=O)c2ccc(C(C)(C)C)cc2)C(N)=O)cc1 ; CC(C)(C)c1ccc(S(=O)(=O)N[C@H](CC(=O)O)C(=O)N[C@@H](CCC(=O)O)C(=O)NCC(=O)N[C@@H](Cc2cccc(F)c2)C(=O)N[C@@H](Cc2c[nH]c3ccccc23)C(=O)N[C@@H](CCC(=O)O)C(=O)NCCCCCC(=O)N[C@@H](CCCCNC(=O)c2ccc(Br)cc2)C(N)=O)cc1]

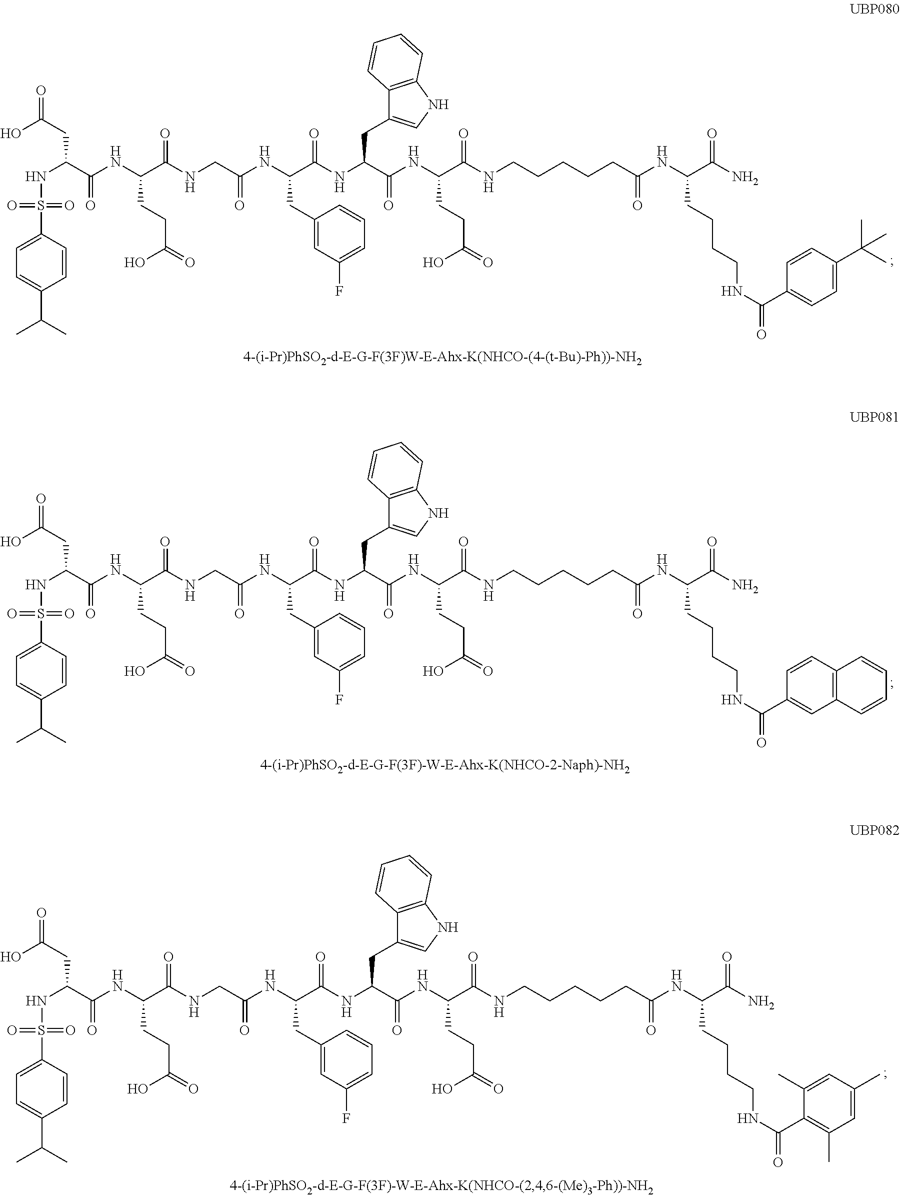

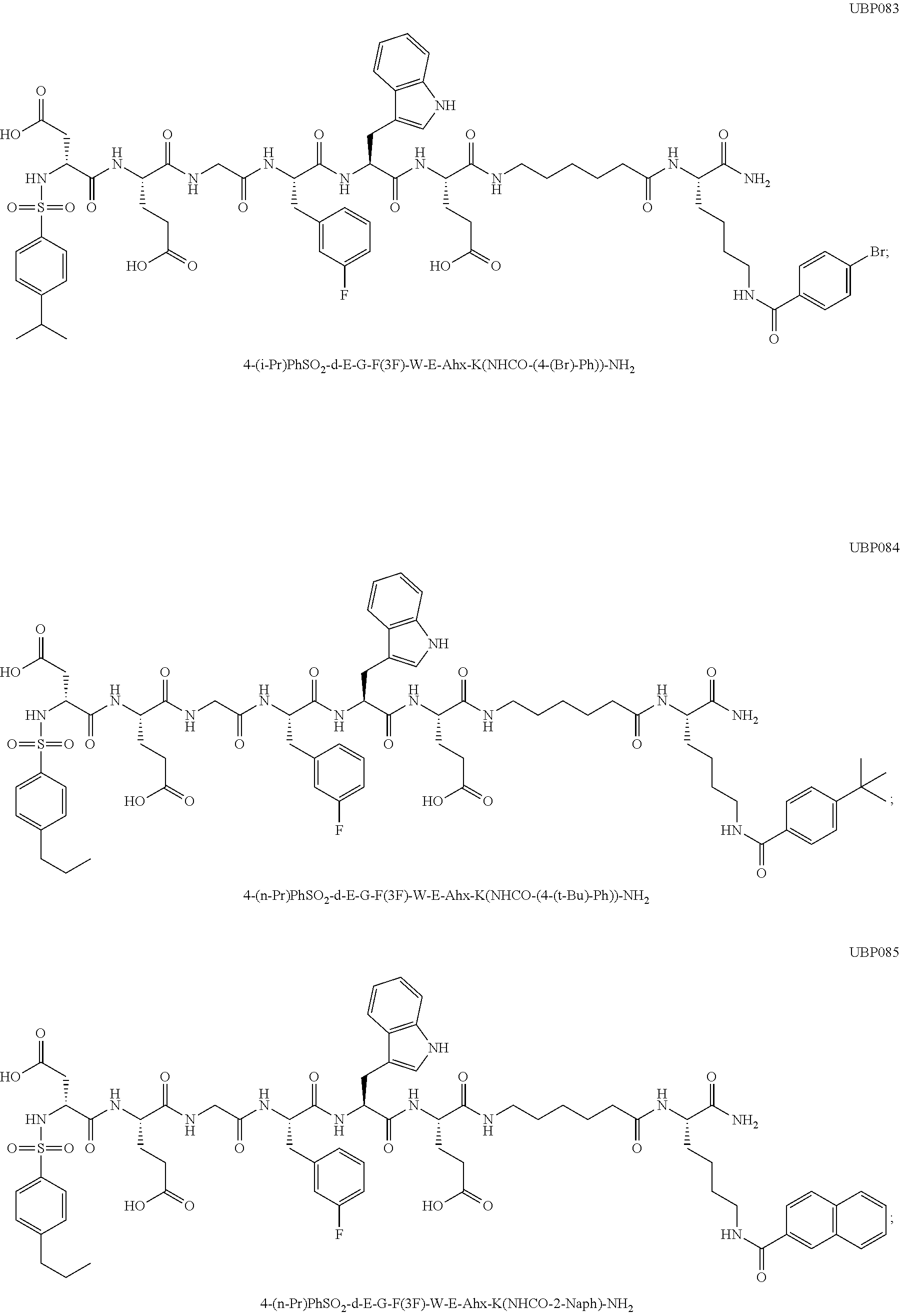

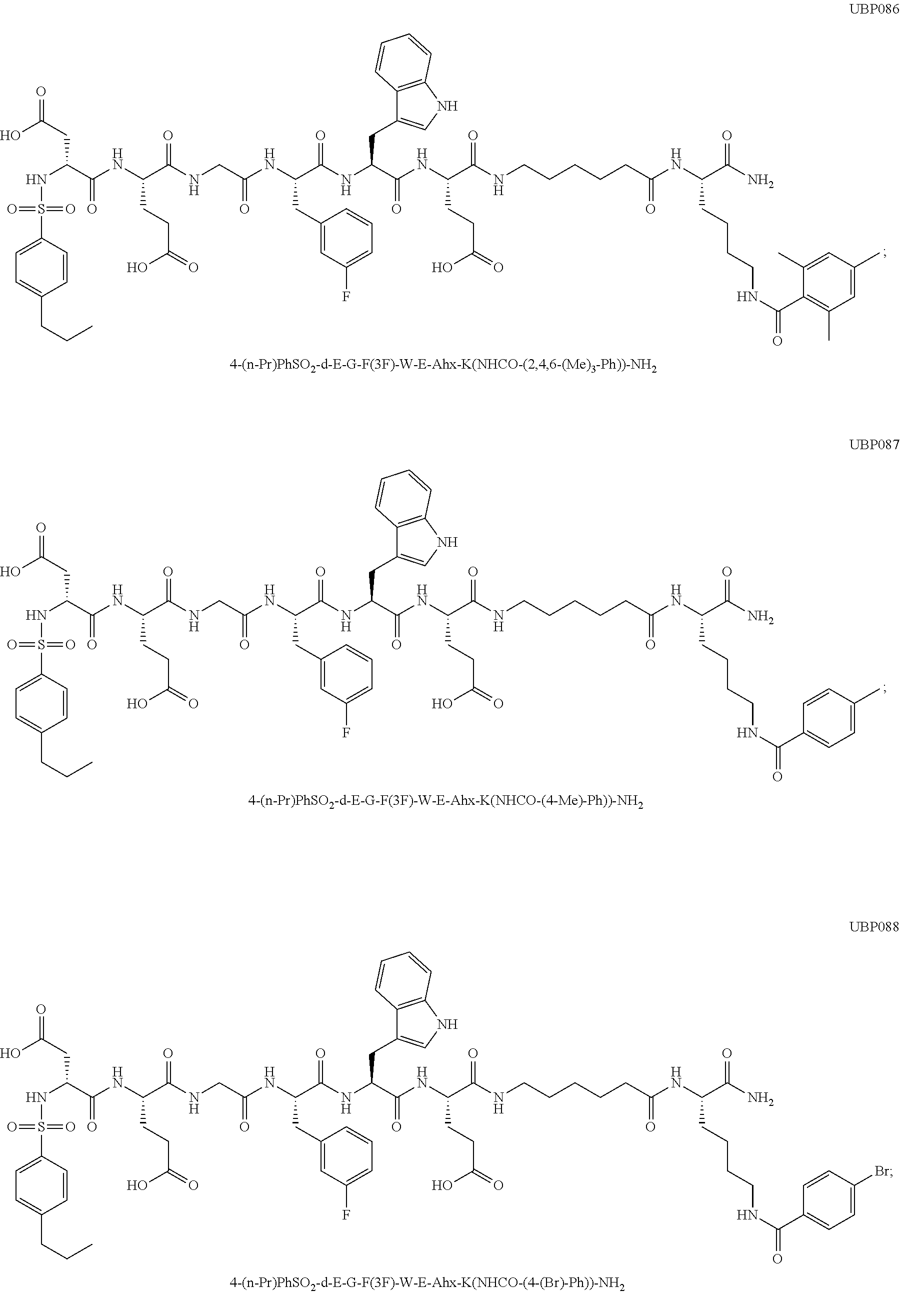

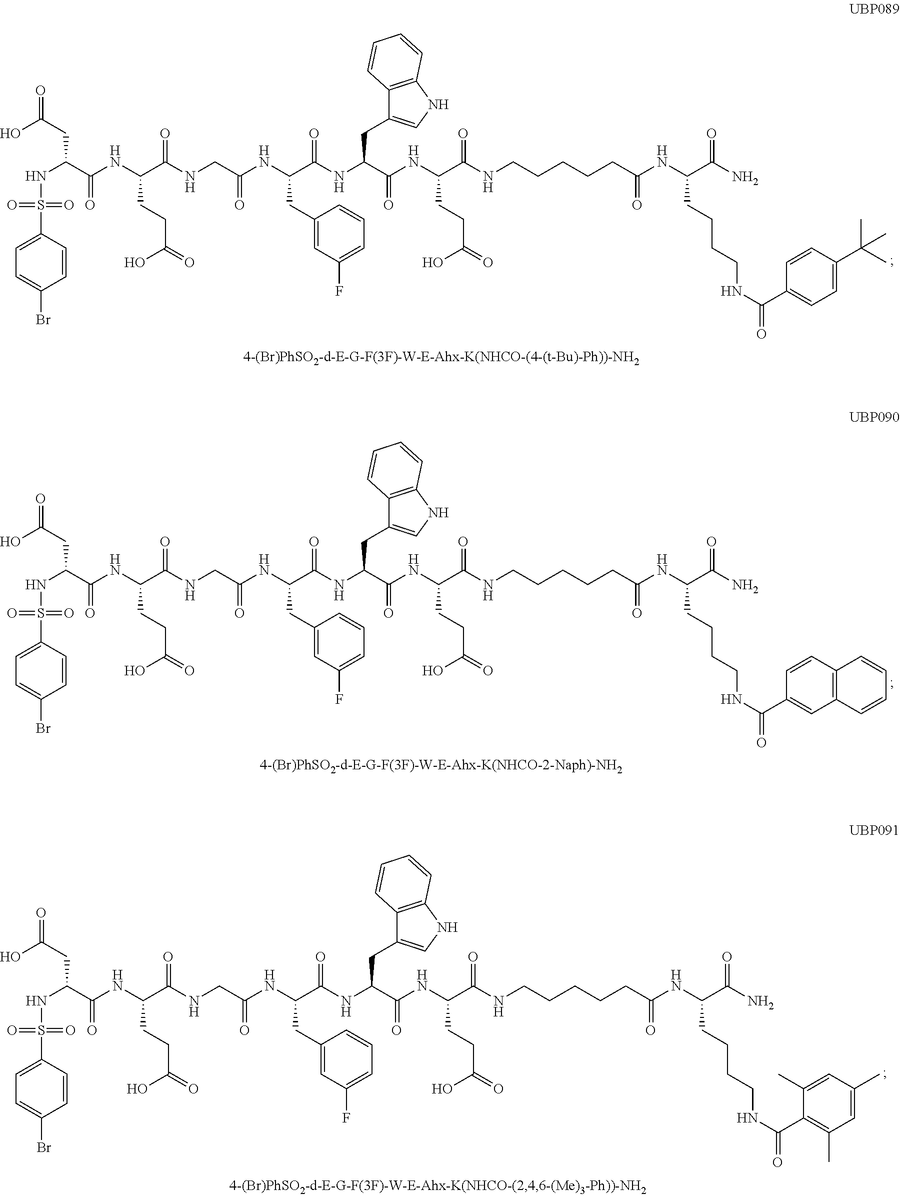

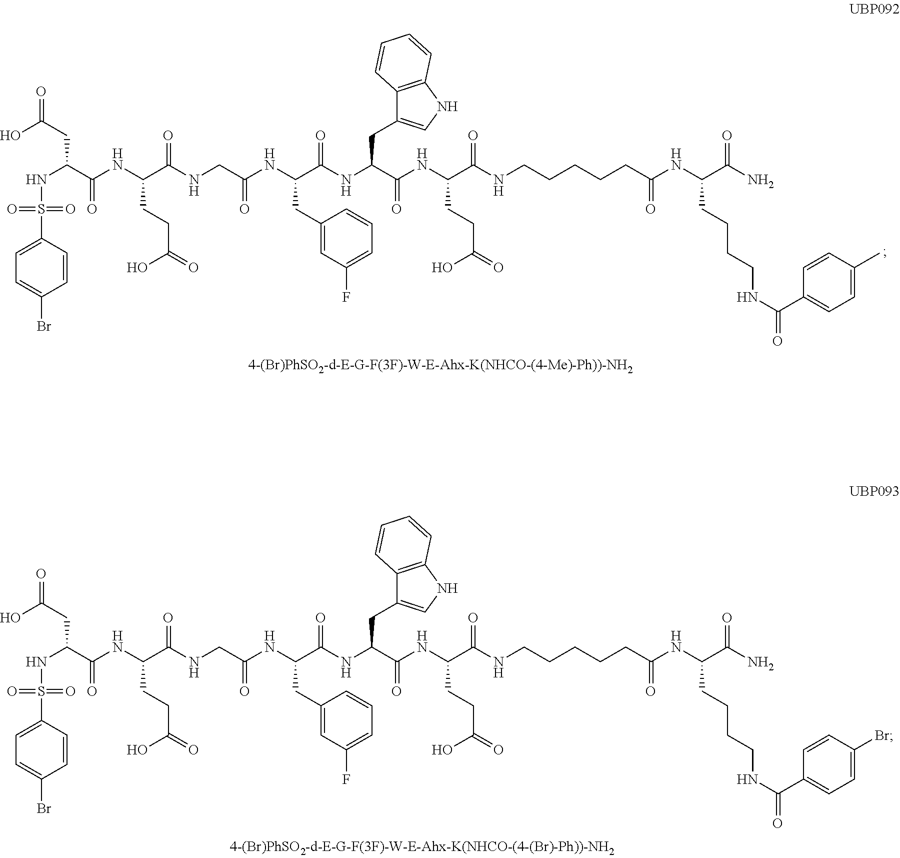 [SMILES: Cc1ccc(C(=O)NCCCC[C@H](NC(=O)CCCCCNC(=O)[C@H](CCC(=O)O)NC(=O)[C@H](Cc2c[nH]c3ccccc23)NC(=O)[C@H](Cc2cccc(F)c2)NC(=O)CNC(=O)[C@H](CCC(=O)O)NC(=O)[C@@H](CC(=O)O)NS(=O)(=O)c2ccc(Br)cc2)C(N)=O)cc1 ; NC(=O)[C@H](CCCCNC(=O)c1ccc(Br)cc1)NC(=O)CCCCCNC(=O)[C@H](CCC(=O)O)NC(=O)[C@H](Cc1c[nH]c2ccccc12)NC(=O)[C@H](Cc1cccc(F)c1)NC(=O)CNC(=O)[C@H](CCC(=O)O)NC(=O)[C@@H](CC(=O)O)NS(=O)(=O)c1ccc(Br)cc1]

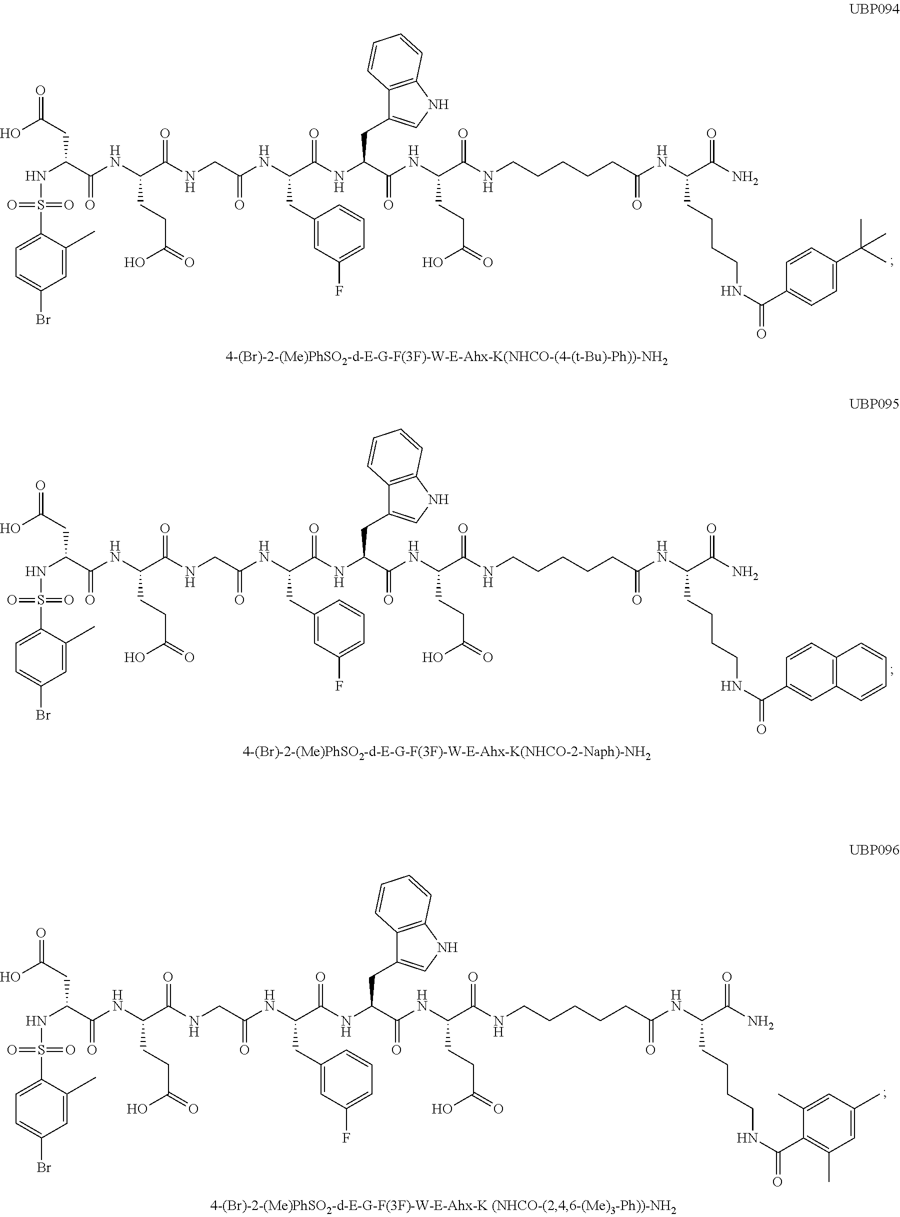

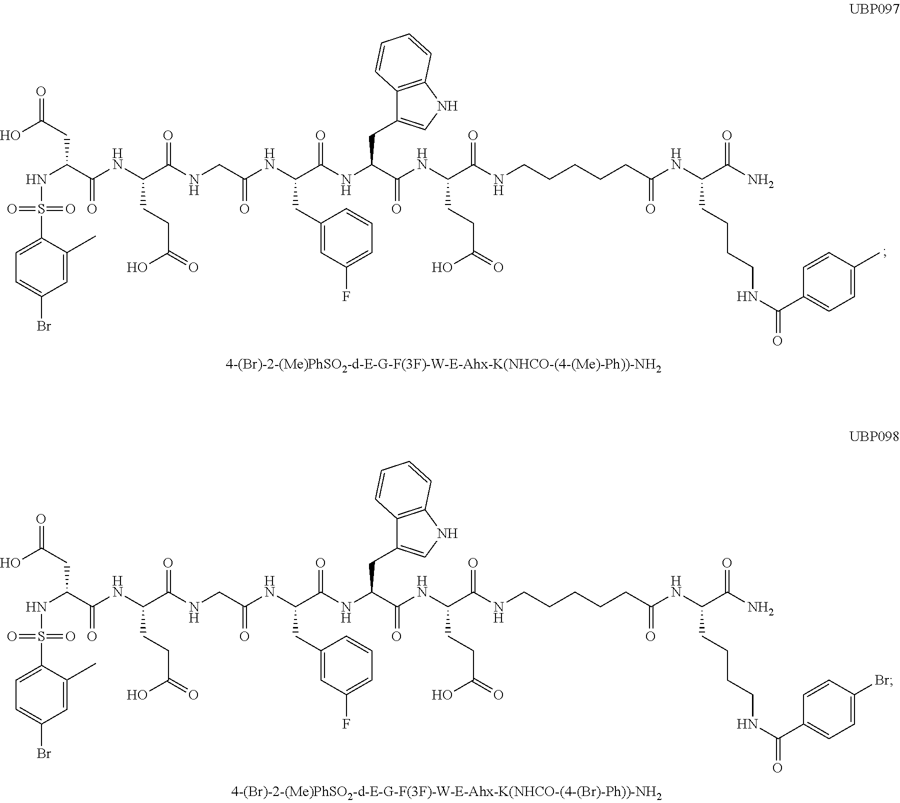 [SMILES: Cc1ccc(C(=O)NCCCC[C@H](NC(=O)CCCCCNC(=O)[C@H](CCC(=O)O)NC(=O)[C@H](Cc2c[nH]c3ccccc23)NC(=O)[C@H](Cc2cccc(F)c2)NC(=O)CNC(=O)[C@H](CCC(=O)O)NC(=O)[C@@H](CC(=O)O)NS(=O)(=O)c2ccc(Br)cc2C)C(N)=O)cc1 ; Cc1cc(Br)ccc1S(=O)(=O)N[C@H](CC(=O)O)C(=O)N[C@@H](CCC(=O)O)C(=O)NCC(=O)N[C@@H](Cc1cccc(F)c1)C(=O)N[C@@H](Cc1c[nH]c2ccccc12)C(=O)N[C@@H](CCC(=O)O)C(=O)NCCCCCC(=O)N[C@@H](CCCCNC(=O)c1ccc(Br)cc1)C(N)=O]

In one embodiment, any one or more of the residues included in the exemplary compounds described above may be an aza amino acid, wherein an “aza amino acid” is an L amino acid in which the α-carbon atom has been replaced by a nitrogen atom.

### Prodrugs

In one embodiment, the compound may be formulated for administration to a patient as a prodrug. The term “prodrug” means a precursor of a designated compound that, following administration to a subject yields the compound in vivo via a chemical or physiological process such as solvolysis or enzymatic cleavage, or under physiological conditions (e.g., a prodrug, on being brought to physiological pH is converted to a compound of Formula Ia, Ib, Ic, Id, Ie, If or Ig). Illustrative procedures for the selection and preparation of suitable prodrug derivatives are described, for example, in “Design of prodrugs”, H. Bundgaard et al. Prodrugs may be produced, for example, by derivatising free carboxylic acid groups of structures of Formula Ia, Ib, Ic, Id, Ie, If or Ig as amides or esters. In one embodiment, the prodrug is an alkyl, aryl, or heteroaryl ester of a compound of the invention. In another embodiment the prodrug may comprise or consist of a methyl, ethyl, propyl, butyl, pentyl, hexyl, or benzyl ester of a compound of the invention.

In a further embodiment, the prodrug may comprise a cycloalkyl ester, preferably a cyclopentyl ester of a compound of the present invention. In a further embodiment, the prodrug may comprise a —CO₂CH₂CH₂(heterocyclyl) ester, wherein heterocyclyl is preferably morpholino, of a compound of the present invention. In a further embodiment, the prodrug may comprise a —CO₂(CH₂CH₂O)₁₋₁₀—CH₂CH₃ (polyethylene glycol or PEG) ester of a compound of the present invention.

A prodrug may be a compound comprising an alcohol functionality, which when phosphorylated in vivo produces the active compound. For example, a compound comprising a serine residue may be a prodrug which, when subjected to physiological conditions is phosphorylated to form the corresponding phosphorylated serine residue, thereby producing the active compound.

### Example of Prodrug

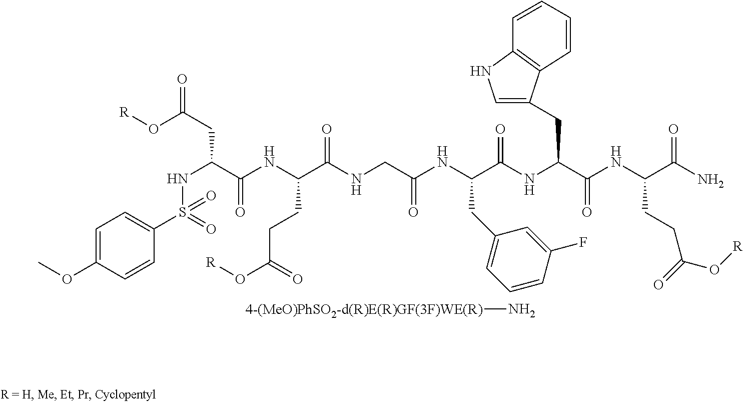

### Examples of PEG-Based Prodrugs

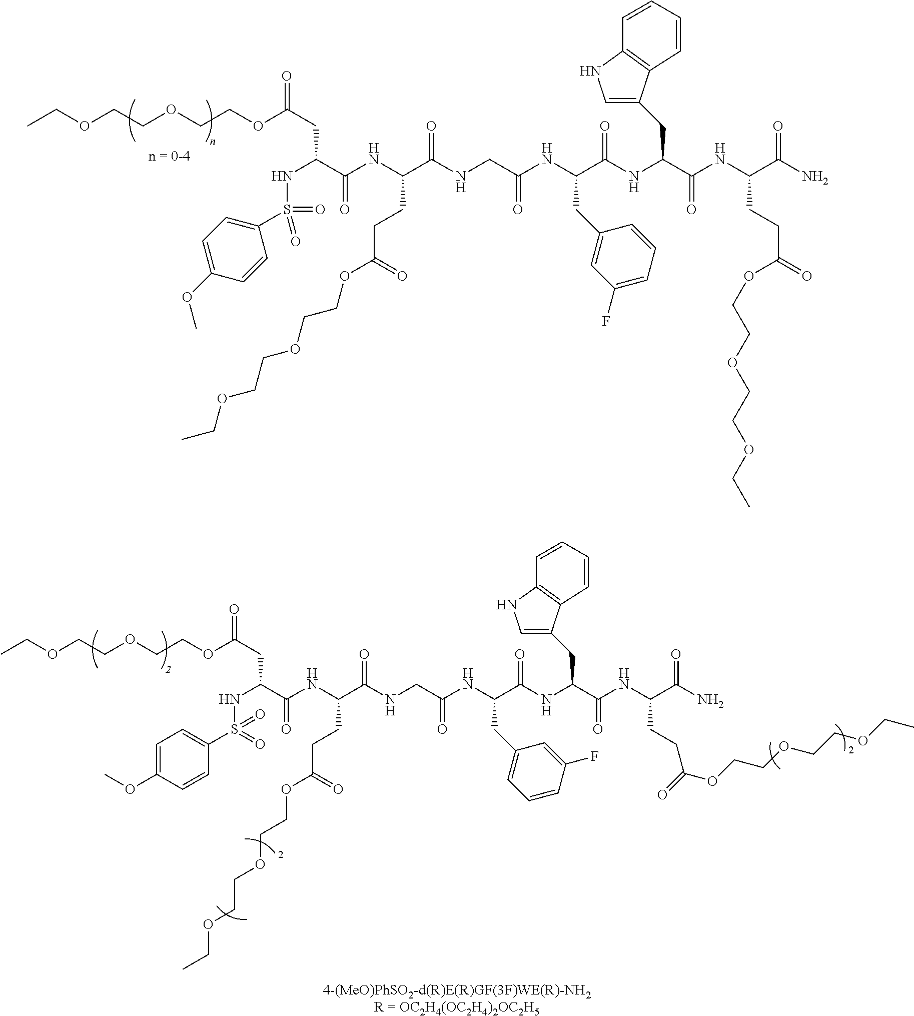

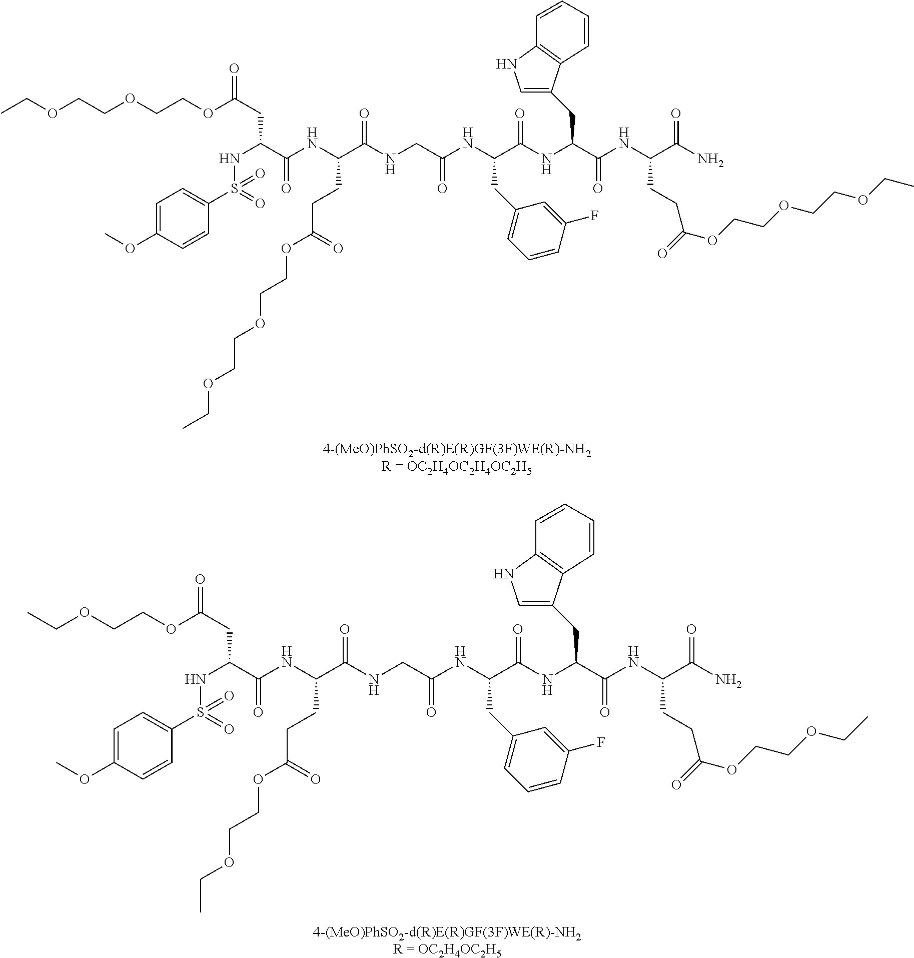

### Methods of Manufacture

Compounds of the invention were synthesised by the coupling of smaller fragments/subunits, usually amino acids.

Amino acids that were commercially available were purchased and used directly (following any appropriate protecting group modification). Unnatural amino acids were synthesised starting from the appropriate amino acid precursor.

### Carboxylic Acid Bioisostere Synthesis

Compounds of the present invention may comprise carboxylic acid bioisosteres. Such carboxylic acid bioisosteres may be synthesised by modification of the functionality of the side chain of an amino acid. Such functionality may be, for example, a carboxylic acid or amide. In this case, appropriate starting amino acids would include aspartic acid, glutamic acid, asparagine and glutamine. A representative scheme for the conversion of an amide functionality of the side chain of an amino acid into a tetrazole group is illustrated below (Tetrazole amino acids as competitive NMDA antagonists, Bioorganic & Medicinal Chemistry Letters, 1993):

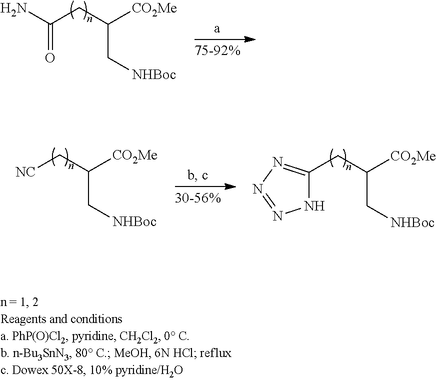

As will be appreciated by the skilled person, the scheme above is a representative procedure for the conversion of a natural amino acid into an unnatural amino acid. Using standard synthetic procedures, the person skilled in the art would be able to synthesise other unnatural amino acids in an analogous manner to that shown above.

Once synthesised, the smaller fragments/subunits (usually amino acids—natural/unnatural) are coupled together to form compounds of the present invention.

The smaller fragments/subunits are coupled together using a solid-phase peptide synthesis. Reagents and conditions for this technique are illustrated in FIG. 1.

Further experimental procedures are provided in the Examples section.

### Pharmaceutical Compositions

The compound, modified peptide or prodrug of the invention may be formulated into a pharmaceutical composition. The invention therefore includes a pharmaceutical composition comprising one or more of the compounds, modified peptides or produgs of the invention.

In one embodiment the pharmaceutical composition may additionally comprise a pharmaceutically-acceptable carrier, excipient, diluent or buffer. Suitable pharmaceutically acceptable carriers, excipients, diluents or buffers may include liquids such as water, saline, glycerol, ethanol or auxiliary substances such as wetting or emulsifying agents, pH buffering substances and the like. Excipients may enable the pharmaceutical compositions to be formulated into tablets, pills, capsules, liquids, gels, or syrups to aid intake by the subject. A thorough discussion of pharmaceutically acceptable carriers is available in Remington's Pharmaceutical Sciences.

The pharmaceutical composition may include a therapeutically effective amount of one or more of the compounds, modified peptides or produgs of the invention. A pharmaceutically effective amount is an amount able to treat the disease for which the composition is intended. The actual amount will depend on a number of factors including the size, weight, age, gender, and health of an individual, and the rate of blood clearance, and will be decided by a clinical practitioner. Generally a pharmaceutically effective amount will be between 1 g/kg body weight and 1 mg/kg body weight or less.

In one embodiment the pharmaceutical composition may include an additional pharmaceutically active agent such as a therapeutic component, in particular, a component useful for the treatment of hyperproliferative disorders such as cancer, inflammatory disorders involving the NFkB signalling pathway such as arthritis, osteoarthritis, rheumatoid arthritis, Crohn's Disease and Irritable Bowel Syndrome (IBS), infectious disorders or neurodegenerative disorders and may include chemotherapeutics, ERMs, SERMs, other E1, E3, E3 and deubiquitinating enzyme inhibitors, proteasome inhibitors, kinase inhibitors, HDAC inhibitors, PPAR inhibitors or specific biological targeted therapies e.g. Herceptin.

The invention also includes any medical device which may have the pharmaceutical composition of the invention inserted into it or coated onto it. Such devices include but are not limited to stents, pins, rods, meshes, beads, syringes, plasters, microchips, micro fluidic devices, and stitches.

### Methods of Treatment

In another aspect, the invention includes a compound, modified peptide, prodrug or pharmaceutical composition of the invention for use in medicine.

In one embodiment the invention provides a compound, modified peptide, prodrug or pharmaceutical composition of the invention for use in the treatment of a disease associated with aberrant protein degradation.

In one embodiment the invention provides a compound, modified peptide, prodrug or pharmaceutical composition of the invention for use in the treatment of a hyperproliferative disorder such as cancer, inflammatory disorders involving the NFkB signalling pathway such as arthritis, osteoarthritis, rheumatoid arthritis, Crohn's Disease and Irritable Bowel Syndrome (IBS), infectious disorders or neurodegenerative disorders.

In another embodiment the invention includes a method of treating a hyperproliferative disorder such as cancer, inflammatory disorders involving the NFkB signalling pathway such as arthritis, osteoarthritis, rheumatoid arthritis, Crohn's Disease and Irritable Bowel Syndrome (IBS), infectious disorders or neurodegenerative disorders comprising administering a pharmaceutically effective amount of a compound, modified peptide, prodrug or pharmaceutical composition of the invention to a patient in need of treatment.

In a particular embodiment, the invention includes a method of treating breast cancer or prostate cancer comprising administering a phatmaceutically effective amount of a compound, modified peptide, prodrug or pharmaceutical composition of the invention to a patient in need of treatment.

As used herein, the term “treatment” encompasses therapy, and can be prophylactic or therapeutic.

A pharmaceutically effective amount is an amount able to treat the disease for which the compound, modified peptide, prodrug or pharmaceutical composition has been administered. The actual amount will depend on a number of factors including the size, weight, age, gender, health of an individual, and the rate of blood clearance, and will be decided by a clinical practitioner. Generally a pharmaceutically effective amount will be between 1 g/kg body weight and 1 mg/kg body weight or less.

In another embodiment the invention includes the use of a compound, modified peptide, prodrug or pharmaceutical composition of the invention in the manufacture of a medicament for the treatment of a hyperproliferative disorder such as cancer, inflammatory disorders involving the NFkB signalling pathway such as arthritis, osteoarthritis, rheumatoid arthritis, Crohn's Disease and Irritable Bowel Syndrome (IBS), infectious disorders or neurodegenerative disorders.

In a particular embodiment, the invention includes the use of a compound, modified peptide, prodrug or pharmaceutical composition of the invention in the manufacture of a medicament for the treatment of breast cancer or prostate cancer.

The compound, modified peptide, prodrug or pharmaceutical composition of the invention may be used for the treatment of disease in any animal. The animal may be a mammal such as a camel, dog, cat, horse, cow, pig, sheep, camelid, mouse, rat, rabbit, hamster, guinea pig, pig, or sheep. In one embodiment, the mammal may be a human.

The compound, modified peptide, prodrug or pharmaceutical composition of the invention may be administered to a patient using any one or more of a number of modes of administration which will be known to a person skilled in the art. Such modes of administration may include parenteral injection (e.g. intravenously, subcutaneously, intraperitoneally, intramuscularly, or to the interstitial space of a tissue), or by rectal, oral, vaginal, topical, transdermal, intradermal, intrathecal, intranasal, ocular, aural, pulmonary or other mucosal administration. The precise mode of administration will depend on the disease or condition to be treated.

### Diagnostic Kits

In another aspect, the invention includes a diagnostic kit comprising a compound, modified peptide or prodrug of the invention. Within this aspect, the compound, modified peptide or prodrug may be labelled to allow its identification. Suitable labels may include, coloured labels, fluorescent labels, and radioactive labels. Detection may be performed by FACS, Western blot, immunoblot or any other technique known to be useful for the identification of labelled molecules.

Diagnostics kits may be used to identify patients having increased PTrCP expression. As discussed above, increased βTrCP expression can be associated with aberrant protein degradation mechanisms, which can lead to hyperproliferative disorders such as cancer through the increased degradation of pro-apoptotic factors. Increased βTrCP expression can also lead to inflammatory disorders involving the NFkB signalling pathway such as arthritis, osteoarthritis, rheumatoid arthritis, Crohn's Disease and Irritable Bowel Syndrome (IBS), infectious disorders and neurodegenerative disorders.

Diagnostics kits may also comprise instructions.

Various aspects and embodiments of the present invention will now be described in more detail by way of example. It will be appreciated that modification of detail may be made without departing from the scope of the invention.

### BRIEF DESCRIPTION OF FIGURES

FIG. 1a shows solid supported peptide synthesis.

Reagents and Conditions: a) Rink amide linker (3 equiv), oxyma (3 equiv), DIC (3 equiv), 0.1 M in DMF, 30 min; b) 20% piperidine in DMF (2×5 min); c) Amino acid (3 equiv), HBTU (3 equiv), DIPEA (6 equiv) 0.1 M in DMF, 40 min; d) TsCl (5 equiv), DMAP (0.1 equiv), DIPEA (10 equiv), 0.1 M in DMF, 40 min; e) TFA, 5% TIS, 5% DCM, 3 h.

FIG. 1b shows solid supported peptide synthesis for C-terminal modified peptides.

Reagents and Conditions: a) Rink amide linker (3 equiv), oxyma (3 equiv), DIC (3 equiv), 0.1 M in DMF, 30 min; b) 20% piperidine in DMF (2×5 min); c) Amino acid (3 equiv), HBTU (3 equiv), DIPEA (6 equiv) 0.1 M in DMF, 40 min; d) TsCl (5 equiv), DMAP (0.1 equiv), DIPEA (10 equiv), 0.1 M in DMF, 40 min; e) 2% Hydrazine in DMF (6×15 mins); f) BzCl (5 equiv), DMAP (0.1 equiv), DIPEA (10 equiv), 0.1 M in DMF, 40 min; g) TFA, 5% TIS, 5% DCM, 3 h.

FIG. 2 shows the abbreviations used to represent capping groups used in synthesis.

FIG. 3 shows the abbreviations used to represent acidic capping groups.

FIG. 4 shows the abbreviations used to represent non-natural amino acids.

FIG. 5 shows FP assay dose response curves. Peptides are numbered according to Table 11.

FIG. 6 shows Biotin pulldown assay results. Peptides are numbered according to Table 11.

FIG. 7 shows SPR assay results. A) Ts-DEGF(3Cl)W—(Me)E-NH₂; B) Ts-dEGF(3Cl)WE-NH₂; C) 4-(MeO)-PhSO₂-dEGF(3F)WE-NH₂; D) Ts-DEGF(3F)WE-NH₂; and E) Ts-dEGF(3F)W-(Me)E-NH₂. Peptides are numbered according to Table 11.

FIG. 8 shows ubiquitination assay results. Peptides are numbered according to Table 11.

FIG. 9 shows peptidomimetic selectivity vs other E3s. Peptides are numbered according to Table 11.

FIG. 10 shows blots of immunoprecipitated proteins from HeLa cells transfected with βTrCP and substrates and treated with cell-permeable βTrCP disruptor peptides. Peptides are numbered according to Table 11.

FIG. 11 shows the ELSDs, which shows the mass of the desired peptide. Peptides are numbered according to Table 11.

FIG. 12 shows the accumulation of PDCD4 following nucleofection with the peptide 4-(MeO)-PhSO₂-dEGF(3F)WE-NH₂ observed using an in cell Western assay, expressed as % activity.

FIG. 13 shows the accumulation of GFP-PDCD4 following nucleofection with the peptide 4-(MeO)-PhSO₂-dEGF(3F)WE-NH₂ observed using a fluoresecent reporter assay, expressed as % activity.

FIG. 14 shows the collation of the assay results for the cell permeable compounds.

FIG. 15 shows the accumulation of PDCD4 in MCF₇ cells as measured by in cell western assay following treatment with UBP036.

FIG. 16 shows the activity of round II compounds in relation to UBP036.

FIG. 17 shows accumulation of β-catenin in MCF7 cells following treatment with UBP036 measured by in cell western assay.

FIG. 18 shows GFP-PDCD4 accumulation in MCF7 cells following treatment with UBP036.

FIG. 19 shows PDCD4 accumulation in MCF7 cells following treatment with UBP036, UBP037 and UBP038 measured by traditional western blot.

FIG. 20 shows PDCD4 accumulation in LNCaP cells following treatment with UBP036, UBP037 and UBP038.

FIG. 21 shows cell viability of MCF7 cells following treatment with UBP036 measured on the xCELLigence platform.

FIG. 22 shows cell viability following compound treatment as measured by the xCELLigence platform (A) cell proliferation of UBP036, UBP037 and UBP038 at 20 uM; (B) dose response curve for UBP036, UBP037 and UBP038; (C) cell proliferation of UBP036 compared to the control compound.

FIG. 23 shows the inhibition of cancer cell growth compared to non-cancer cell growth following treatment with UBP036, UBP037 and UBP038.

FIG. 24 shows PDCD4 accumulation following nucleofection of UBP022 into MCF7 cells.

### EXAMPLES

### Experimental Procedures

The following experimental conditions were used throughout the examples unless other details are provided.

### General Conditions for the Solid-Phase Synthesis

All the coupling reactions were carried out at room temperature if no specifications are given. Solid-phase synthesis was performed manually using Isolute filtration reservoirs as the reaction vessel, fitted with polyethylene frits (Argonaut Technologies Inc). Amino acids are Fmoc protected at the N terminus, with suitable acid labile protecting groups on the side chains. For C-terminal modified peptides Fmoc-Lys(Dde)-OH was used to allow selective modification of the Lys side chain. Each coupling step of the synthesis was assessed for completion using either the Kaiser test for primary amines, or the chloranil test for secondary amines.

### Coupling the Linker to the Resin

Aminomethyl PS resin (loading 1.23 mmol/g, 0.30 g, 0.369 mmol) in a 6 mL reaction vessel was swollen for 5 minutes in DCM (3 mL), then washed with DCM (3×3 mL). To a solution of Rink amide linker (598 mg, 1.11 mmol) in DMF (3.69 mL) was added oxyma (157 mg, 1.11 mmol) and the solution shaken for 10 minutes. DIC (173 uL, 1.11 mmol) was added and the solution shaken for 2 minutes. The mixture was added to the resin and shaken for 30 minutes. The resin was filtered and washed with DMF (3×4 mL), DCM (3×4 mL) and MeOH (3×4 mL). Kaiser test negative. The resin was washed with Et₂O (3×4 mL) and dried under vacuum for storage.

### Coupling of Amino Acids/Spacer

Resin (˜0.049 mmol) in a 3 mL reaction vessel was swollen for 5 minutes in DCM (1.5 mL) and filtered. A solution of 20% piperidine in DMF (1.5 mL) was added, the vessel was shaken for 5 mins and the resin was filtered and washed with DMF (3×1.5 mL) and DCM (3×1.5 mL). Piperidine deprotection and washing cycle was repeated and the resin was dried under vacuum, Kaiser test positive. To a solution of the appropriate amino acid/spacer (0.15 mmol, 3 equiv) in DMF (0.49 mL) was added HBTU (0.15 mmol, 3 equiv) and the solution shaken for 2 minutes. DIPEA (0.30 mmol, 6 equiv) was added and the solution shaken for 1 minute. The mixture was added to the resin and shaken for 40 minutes. The resin was filtered and washed with DMF (3×1.5 mL), DCM (3×1.5 mL) and MeOH (3×1.5 mL). Kaiser test negative, otherwise treatment of activated amino acid repeated.

### Coupling to N-Alkylated Amino Acids

Resin (˜0.049 mmol) in a 3 mL reaction vessel was swollen for 5 minutes in DCM (1.5 mL) and filtered. A solution of 20% piperidine in DMF (1.5 mL) was added, the vessel was shaken for 5 mins and the resin was filtered and washed with DMF (3×1.5 mL) and DCM (3×1.5 mL). Piperidine deprotection and washing cycle was repeated and the resin was dried under vacuum, Choranil test positive. To a solution of the appropriate amino acid (0.15 mmol, 3 equiv) in DMF (0.49 mL) was added oxyma (0.15 mmol, 3 equiv) and the solution shaken for 10 minutes. DIC (0.15 mmol, 3 equiv) was added and the solution shaken for 2 minutes. The mixture was added to the resin and heated in a microwave at 60° C. for 20 minutes. The mixture was then shaken for an additional 20 minutres. The resin was filtered and washed with DMF (3×4 mL), DCM (3×4 mL) and MeOH (3×4 mL). Chloranil test negative, otherwise treatment of activated amino acid repeated.

### Example of the N-Terminus Capping

Resin (˜0.049 mmol) in a 3 mL reaction vessel was swollen for 5 minutes in DCM (1.5 mL) and filtered. A solution of 20% piperidine in DMF (1.5 mL) was added, the vessel was shaken for 5 mins and the resin was filtered and washed with DMF (3×1.5 mL) and DCM (3×1.5 mL). Piperidine addition and washing cycle was repeated and the resin was dried under vacuum, Kaiser test positive. To a solution of 4-toluenesulfonyl chloride (0.25 mmol, 5 equiv) in DCM:DMF (1:1, 0.49 mL) was added DMAP (0.005 mmol, 0.1 equiv) and the solution shaken for 2 minutes. DIPEA (0.50 mmol, 10 equiv) was added and the solution shaken for 1 minute. The mixture was added to the resin and shaken for 40 minutes. The resin was filtered and washed with DMF (3×1.5 mL), DCM (3×1.5 mL) and MeOH (3×1.5 mL). Kaiser test negative, otherwise treatment of with the capping group was repeated.

### Example of the C-Terminus Capping

After N-terminus capping, resin (—0.049 mmol) in a 3 mL reaction vessel was swollen for 5 minutes in DCM (1.5 mL) and filtered. A solution of 2% hydrazine monohydrate in DMF (1.5 mL) was added, the vessel was shaken for 15 mins and the resin was filtered and washed with DMF (3×1.5 mL) and DCM (3×1.5 mL). Hydrazine addition and washing cycle was repeated (×5) and the resin was dried under vacuum, Kaiser test positive. To a solution of benzoyl chloride (0.25 mmol, 5 equiv) in DCM:DMF (1:1, 0.49 mL) was added DMAP (0.005 mmol, 0.1 equiv) and the solution shaken for 2 minutes. DIPEA (0.50 mmol, 10 equiv) was added and the solution shaken for 1 minute. The mixture was added to the resin and shaken for 40 minutes. The resin was filtered and washed with DMF (3×1.5 mL), DCM (3×1.5 mL) and MeOH (3×1.5 mL). Kaiser test negative, otherwise treatment of with the capping group was repeated.

### Characterisation of Selected Examples:

| | | | RT |
|---|---|---|---|
| ID | Structure | MS | (mins) |
| UBP001 | DpSGIFE-NH₂ | 744.1^{a} | 4.272^{e} |
| UBP002 | Suc-EGFFE-NH₂ | 725.2^{a} | 5.107^{e} |
| UBP003 | Suc-EGF(2F)F(4NO₂)E-NH₂ | 788.2^{a} | 4.989^{e} |
| UBP004 | Suc-EGF(3F)F(4NO₂)E-NH₂ | 788.2^{a} | 5.073^{e} |
| UBP005 | Suc-EGF(4F)F(4NO₂)E-NH₂ | 788.2^{a} | 5.088^{e} |
| UBP006 | Suc-EGF(2F)Y(Me)E-NH₂ | 773.2^{a} | 5.023^{e} |
| UBP007 | Suc-EGYFE-NH₂ | 741.1^{a} | 4.500^{e} |
| UBP008 | Mal-EGF(3F)F(4NO₂)E-NH₂ | 786.1^{a} | 3.739^{e} |
| UBP009 | Suc-EGY1NalE-NH₂ | 791.2^{a} | 5.222^{e} |
| UBP010 | Suc-EGF(3F)1NalE-NH₂ | 793.0^{a} | 5.783^{e} |
| UBP011 | Suc-EGF(4NO₂)1NalE-NH₂ | 820.0^{a} | 5.775^{e} |
| UBP012 | Suc-QGF(3F)F(4NO₂)E-NH₂ | 787.0^{a} | 4.924^{e} |
| UBP013 | Suc-EGYF(4NO₂)E-NH₂ | 786.1^{a} | 4.422^{e} |
| UBP014 | Suc-EGF(3F)WE-NH₂ | 781.9^{a} | 4.597^{e} |
| UBP015 | Suc-EGF(3F)HE-NH₂ | 733.0^{a} | 3.257^{e} |
| UBP016 | Ac-dEGF(3F)1NalE-NH₂ | 850.0^{a} | 5.705^{e} |
| UBP017 | Ac-dEGF(3F)WE-NH₂ | 838.9^{a} | 4.976^{e} |
| UBP018 | Bz-dEGF(3F)WE-NH₂ | 901.0^{a} | 5.514^{e} |
| UBP019 | Et(CO)-dEGF(3F)WE-NH₂ | 853.0^{a} | 5.167^{e} |
| UBP020 | MeO(CO)-dEGF(3F)WE-NH₂ | 855.1^{a} | 5.138^{e} |
| UBP021 | Ts-dEGF(3F)WE-NH₂ | 951.2^{a} | 5.800^{e} |
| UBP022 | 4-(MeO)-PhSO₂-dEGF(3F)WE-NH₂ | 967.2^{a} | 5.690^{e} |
| UBP023 | EtO(CO)-dEGF(3F)WE-NH₂ | 896.2^{a} | 5.362^{e} |
| UBP024 | Ts-DEGF(3F)WE-NH₂ | 951.2^{a} | 5.673^{e} |
| UBP025 | Ts-dDGF(3F)WE-NH₂ | 937.2^{a} | 5.745^{e} |
| UBP026 | 4-(MeO)-PhSO₂-DEGF(3F)WE-NH₂ | 967.2^{a} | 5.534^{e} |
| UBP027 | Ts-dEGF(3F)WD-NH₂ | 937.2^{a} | 5.770^{e} |
| UBP028 | Ts-dDGF(3F)WD-NH₂ | 923.2^{a} | 5.729^{e} |
| UBP029 | 3,4-(MeO)₂-PhSO₂-dEGF(3F)WE-NH₂ | 997.2^{a} | 5.495^{e} |
| UBP030 | 4-(BuO)-PhSO₂-dEGF(3F)WE-NH₂ | 1009.4^{a} | 6.404^{e} |
| UBP031 | 2-NaphthylSO₂-dEGF(3F)WE-NH₂ | 987.2^{a} | 6.010^{e} |
| UBP032 | Ts-dEGF(3F)WE(Me)-NH₂ | 956.2^{a} | 5.891^{e} |
| UBP033 | Ts-dEGF(3Cl)WE(Me)-NH₂ | 981.2^{a} | 5.992^{e} |
| UBP034 | Ts-dEGF(3Cl)WE-NH₂ | 967.2^{a} | 6.086^{e} |
| UBP035 | 4-(MeO)PhSO₂-dEGF(3F)WE-Ahx-kkkkkkkkk-NH₂ | 2236.2^{b} | 3.547^{e} |
| UBP036 | 4-(MeO)PhSO₂-dEGF(3F)WE-Ahx-K(Ahx-Chol)-NH₂ | 1773.4^{d} | 7.970^{f} |
| UBP037 | 4-(MeO)PhSO₂-dEGF(3F)WE-Ahx-K(NHCOC₁₇H₃₅)-NH₂ | 1499.0^{c} | 10.398^{f} |
| UBP038 | 4-(MeO)PhSO₂-d EGF(3F)WE-Ahx-K(NHCOC₁₉H₃₉)-NH₂ | 1527.2^{c} | 11.247^{f} |
| UBP039 | 4-(t-Bu)-PhSO₂-dEGF(3F)WE-NH₂ | 1017.0^{c} | 9.502^{g} |
| UBP040 | 4-(i-Pr)-PhSO₂-dEGF(3F)WE-NH₂ | 1003.1^{a} | 9.688^{g} |
| UBP041 | 4-(Pr)-PhSO₂-dEGF(3F)WE-NH₂ | 1003.1^{c} | 9.471^{g} |
| UBP042 | 4-(Br)-PhSO₂-dEGF(3F)WE-NH₂ | 1039.2^{c} | 8.589^{g} |
| UBP043 | 4-(Br)-2-(CH₃)-PhSO₂-dEGF(3F)WE-NH₂ | 1053.0^{c} | 8.932^{g} |
| UBP044 | 2-Naph-SO₂-dEGF(3F)WE-NH₂ | 1011.2^{c} | 8.924^{g} |
| UBP045 | 4-(OCF₃)-PhSO₂-dEGF(3F)WE-NH₂ | 1045.0^{c} | 9.289^{g} |
| UBP046 | 4-(Br)-3-(CF₃)-PhSO₂-dEGF(3F)WE-NH₂ | 1107.0^{c} | 9.866^{g} |
| UBP047 | 4-(CF₃)-PhSO₂-dEGF(3F)WE-NH₂ | 1029.2^{c} | 9.319^{g} |
| UBP048 | 2,4-(Cl)₂-PhSO₂-dEGF(3F)WE-NH₂ | 1029.0^{c} | 9.437^{g} |
| UBP049 | 2,4-(Br)₂-PhSO₂-dEGF(3F)WE-NH₂ | 1117.0^{c} | 9.042^{g} |
| UBP050 | 3,5-(CH₃)₂-PhSO₂-dEGF(3F)WE-NH₂ | 989.0^{c} | 8.959^{g} |
| UBP051 | 4-(Br)-2-(OCF₃)-PhSO₂-dEGF(3F)WE-NH₂ | 1123.0^{c} | 9.499^{g} |
| UBP052 | 4-(I)-PhSO₂-dEGF(3F)WE-NH₂ | 1096.5^{c} | 11.312^{g} |
| UBP053 | 4-(Cl)-PhSO₂-dEGF(3F)WE-NH₂ | 995.2^{c} | 10.944^{g} |
| UBP054 | 4-(MeO)PhSO₂-dEGF(3F)WE-Ahx-K(NHCO-(4-(t-Bu)-Ph))-NH₂ | 1392.5^{c} | 10.163^{g} |
| UBP055 | 4-(MeO)PhSO₂-dEGF(3F)WE-Ahx-K(NHCO-2-Naph)-NH₂ | 1386.3^{c} | 9.834^{g} |
| UBP056 | 4-(MeO)PhSO₂-dEGF(3F)WE-Ahx-K (NHCO-(2,4,6-(Me)₃-Ph))-NH₂ | 1378.5^{c} | 9.313^{g} |
| UBP057 | 4-(MeO)PhSO₂-dEGF(3F)WE-Ahx-K (NHCO-(4-(Me)-Ph))-NH₂ | 1350.2^{c} | 9.462^{g} |
| UBP058 | 4-(MeO)PhSO₂-dEGF(3F)WE-Ahx-K (NHCO-(4-(Br)-Ph))-NH₂ | 1414.3^{c} | 9.497^{g} |
| UBP059 | 4-(MeO)PhSO₂-dEGF(3F)WE-Ahx-K (NHSO₂-(4-(Br)-Ph))-NH₂ | 1450.2^{c} | 9.903^{g} |
| UBP060 | 4-(MeO)PhSO₂-dEGF(3F)WE-Ahx-K (NHCO-(4-(Cl)-Ph))-NH₂ | 1370.3^{c} | 9.289^{g} |
| UBP061 | 4-(MeO)PhSO₂-dEGF(3F)WE-Ahx-K (NHCOPh)-NH₂ | 1335.6^{c} | 10.507^{g} |
| UBP062 | 4-(MeO)PhSO₂-dEGF(3F)WE-Ahx-K (NHCO-(3,5-(Cl)₂-Ph))-NH₂ | 1380.0^{a} | 10.035^{g} |
| UBP063 | 4-(MeO)PhSO₂-dEGF(3F)WE-Ahx-K (NHCO-(CH₂)₄CH₃)-NH₂ | 1330.6^{c} | 9.493^{g} |
| UBP064 | 4-(MeO)PhSO₂-dEGF(3F)WE-Ahx-K (NHCO-(4-(CF₃)-Ph))-NH₂ | 1380.3^{a} | 9.874^{g} |
| UBP065 | 4-(MeO)PhSO₂-dEGF(3F)WE-Ahx-K (NHCOO-Ph)-NH₂ | 1352.3^{c} | 9.575^{g} |
| UBP066 | 4-(MeO)PhSO₂-dEGF(3F)WE-Ahx-K (NHCO-(4-(OMe)-Ph))-NH₂ | 1366.2^{c} | 8.875^{g} |
| UBP067 | 4-(MeO)PhSO₂-dEGF(3F)WE-Ahx-K (NHCONH-Ph)-NH₂ | 1327.4^{a} | 9.389^{g} |
| UBP068 | 4-(MeO)PhSO₂-dEGF(3F)WE-Ahx-K (NHCO-CH₂CH(CH₃)₂)-NH₂ | 1316.0^{c} | 8.316^{g} |
| UBP069 | 4-(MeO)PhSO₂-dEGF(3F)WE-Ahx-K (NHOCO-1-Naph)-NH₂ | 1378.2^{a} | 10.118^{g} |
| UBP070 | 4-(MeO)PhSO₂-dEGF(3F)WE-Ahx-K (NHCO-(4-(Cl)-2,6(F)₂-Ph))-NH₂ | 1406.5^{c} | 10.815^{g} |
| UBP071 | 4-(MeO)PhSO₂-dEGF(3F)WE-Ahx-K (NHCO-(4-(Me₂N)-Ph))-NH₂ | 1379.3^{c} | 8.889^{g} |
| UBP072 | 4-(MeO)PhSO₂-dEGF(3F)WE-Ahx-K (NHSO₂-(4-(i-Pr)-Ph))-NH₂ | 1414.0^{c} | 10.271^{g} |
| UBP073 | 4-(MeO)PhSO₂-dEGF(3F)WE-Ahx-K (NHSO₂Ph)-NH₂ | 1348.2^{a} | 8.877^{g} |
| UBP074 | 4-(MeO)PhSO₂-dEGF(3F)WE-Ahx-K (NHSO₂-(4-(n-Pr)-Ph)-NH₂ | 1414.2^{c} | 10.269^{g} |
| UBP075 | 4-(t-Bu)PhSO₂-dEGF(3F)WE-Ahx-K(NHCO(4-(t-Bu)Ph))-NH₂ | 1394.3^{a} | 7.138^{e} |
| UBP076 | 4-(t-Bu)PhSO₂-dEGF(3F)WE-Ahx-K(NHCO(2-Naphth))-NH₂ | 1388.2^{a} | 6.922^{e} |
| UBP077 | 4-(t-Bu)PhSO₂-dEGF(3F)WE-Ahx-K(NHCO(2,4,6-(Me)₃-Ph))-NH₂ | 1380.3^{a} | 6.911^{e} |
| UBP078 | 4-(t-Bu)PhSO₂-dEGF(3F)WE-Ahx-K(NHCO(4-(Me)Ph))-NH₂ | 1352.3^{a} | 6.802^{e} |
| UBP079 | 4-(t-Bu)PhSO₂-dEGF(3F)WE-Ahx-K(NHCO(4-(Br)Ph))-NH₂ | 1418.2^{a} | 6.917^{e} |
| UBP080 | 4-(i-Pr)PhSO₂-dEGF(3F)WE-Ahx-K(NHCO(4-(t-Bu)Ph))-NH₂ | 1380.4^{a} | 7.037^{e} |
| UBP081 | 4-(i-Pr)PhSO₂-dEGF(3F)WE-Ahx-K(NHCO(2-Naphth))-NH₂ | 1374.2^{a} | 6.813^{e} |
| UBP082 | 4-(i-Pr)PhSO₂-dEGF(3F)WE-Ahx-K(NHCO(2,4,6-(Me)₃Ph))-NH₂ | 1366.3^{a} | 6.786^{e} |
| UBP083 | 4-(i-Pr)PhSO₂-dEGF(3F)WE-Ahx-K(NHCO((4-(Br)Ph))-NH₂ | 1402.2^{a} | 6.815^{e} |
| UBP084 | 4-(n-Pr)PhSO₂-dEGF(3F)WE-Ahx-K(NHCO(4-(tBu)Ph))-NH₂ | 1380.3^{a} | 7.175^{e} |
| UBP085 | 4-(n-Pr)PhSO₂-dEGF(3F)WE-Ahx-K(NHCO(2-Naphth))-NH₂ | 1374.3^{a} | 6.853^{e} |
| UBP086 | 4-(n-Pr)PhSO₂-dEGF(3F)WE-Ahx-K(NHCO((2,4,6-(Me)₃Ph))-NH₂ | 1366.3^{a} | 6.826^{e} |
| UBP087 | 4-(n-Pr)PhSO₂-dEGF(3F)WE-Ahx-K(NHCO(4-(Me)Ph))-NH₂ | 1338.3^{a} | 6.735^{e} |
| UBP088 | 4-(n-Pr)PhSO₂-dEGF(3F)WE-Ahx-K(NHCO(4-(Br)Ph))-NH₂ | 1404.2^{a} | 6.858^{e} |
| UBP089 | 4-(Br)PhSO₂-dEGF(3F)WE-Ahx-K(NHCO(4-(tBu)Ph))-NH₂ | 1418.2^{a} | 7.047^{e} |
| UBP090 | 4-(Br)PhSO₂-dEGF(3F)WE-Ahx-K(NHCO(2-NaPhth))-NH₂ | 1412.2^{a} | 6.848^{e} |
| UBP091 | 4-(Br)PhSO₂-dEGF(3F)WE-Ahx-K(NHCO(2,4,6-(Me)₃Ph))-NH₂ | 1404.2^{a} | 6.814^{e} |
| UBP092 | 4-(Br)PhSO₂-dEGF(3F)WE-Ahx-K(NHCO(4-(Me)Ph))-NH₂ | 1376.1^{a} | 6.732^{e} |
| UBP093 | 4-(Br)PhSO₂-dEGF(3F)WE-Ahx-K(NHCO(4-(Br)Ph))-NH₂ | 1442.1^{a} | 6.866^{e} |
| UBP094 | 4-(Br)-2-(Me)PhSO₂-dEGF(3F)WE-Ahx-K(NHCO(4-(tBu)Ph))-NH₂ | 1432.2^{a} | 7.036^{e} |
| UBP095 | 4-(Br)-2-(Me)PhSO₂-dEGF(3F)WE-Ahx-K(NHCO(2-Naphth))-NH₂ | 1426.3^{a} | 6.831^{e} |
| UBP096 | 4-(Br)-2-(Me)PhSO₂-dEGF(3F)WE-Ahx-K(NHCO(2,4,6-(Me)₃Ph))- | 1418.2^{a} | 6.805^{e} |
| | NH₂ | | |
| UBP097 | 4-(Br)-2-(Me)PhSO₂-dEGF(3F)WE-Ahx-K(NHCO(4-(Me)Ph))-NH₂ | 1390.2^{a} | 6.696^{e} |
| UBP098 | 4-(Br)-2-(Me)PhSO₂-dEGF(3F)WE-Ahx-K(NHCO(4-(Br)Ph))-NH₂ | 1452.0^{a} | 6.824^{e} |

^{a}Mass identified as [M − H]⁻;
^{b}Mass identified as [M + H]⁺;
^{c}Mass identified as [M + Na]⁺;
^{d}Mass identified as [M + K]⁺; HPLC analysis preformed using a Supleco Discovery C18 5 cm × 4.6 mm, 5 μm column, samples analysed by ELSD, 220 nM and 254 nM, conditions used where
^{e}5% to 95% MeOH (+ 0.1% formic acid) in H₂O (+ 0.1% formic acid) over 6 minutes, 3 minute hold, then 1 minute at 5% MeOH (+ 0.1% formic acid);
^{f}5% to 95% MeCN (+ 0.1% formic acid) in H₂O (+ 0.1% formic acid) over 10 minutes, 4 minute hold, then 1 minute at 5% MeCN (+ 0.1% formic acid);
^{g}5% to 95% MeOH (+0.1% formic acid) in H₂O (+ 0.1% formic acid) over 10 minutes, 4 minute hold, then 1 minute at 5% MeOH (+ 0.1% formic acid);

Cleavage from Resin

Resin (˜0.0.49 mmol) in a 3 mL reaction vessel was swollen for 5 minutes in DCM (2 mL) and filtered. A solution of TFA:TIS:DCM (90:5:5 0.49 mL) was added, and the vessel was shaken for 3 h. The resin was removed by filtration, and ice-cold Et₂O (10 mL) was added to the filtrate. The resultant solid was pelleted by centrifuge, and the solvent removed by decantation. Solid was dried under vacuum.

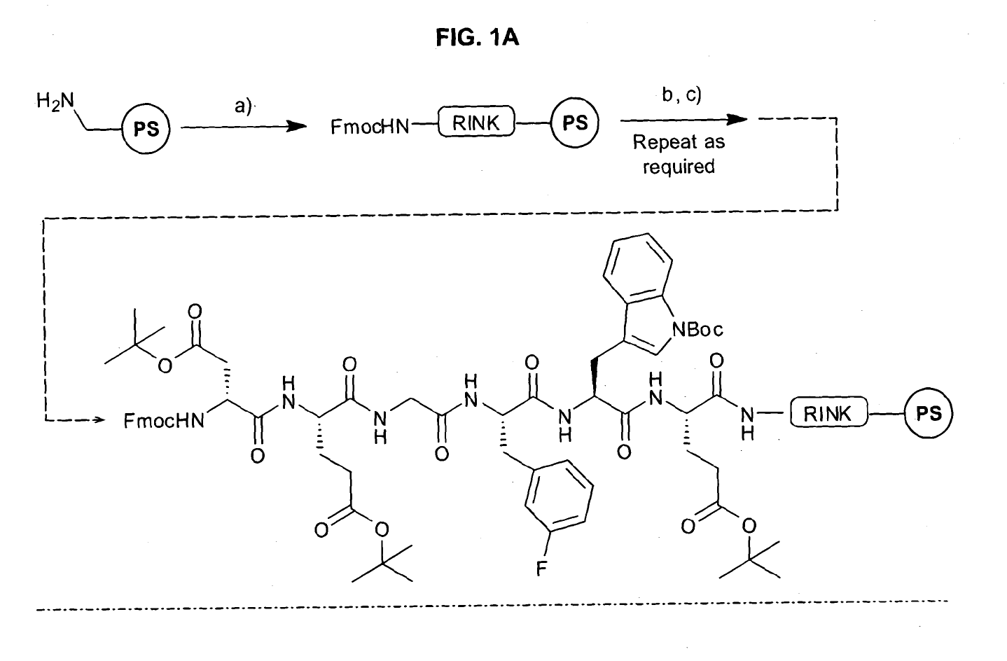

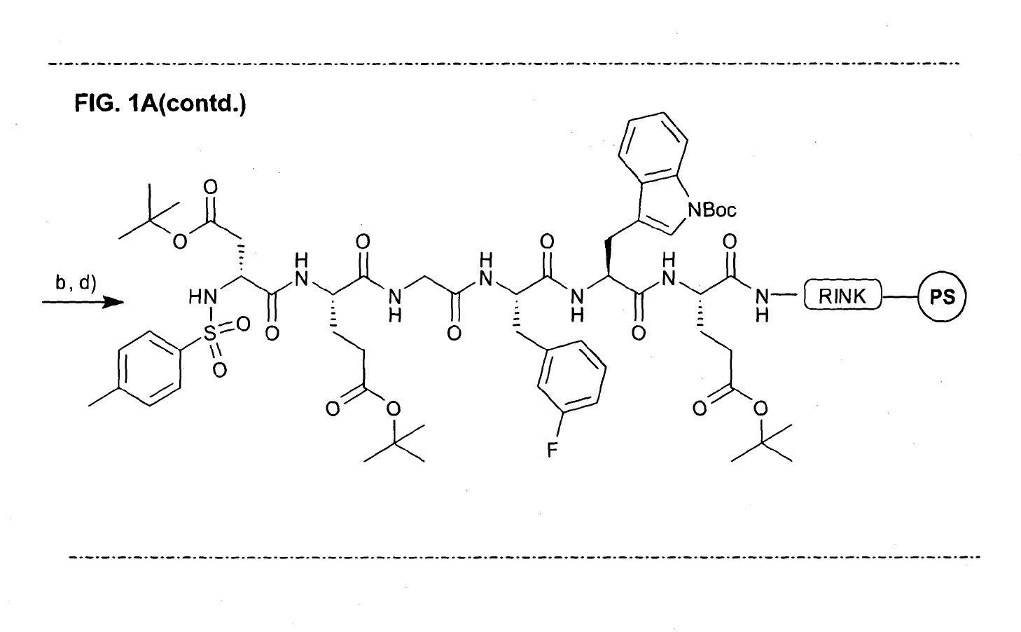

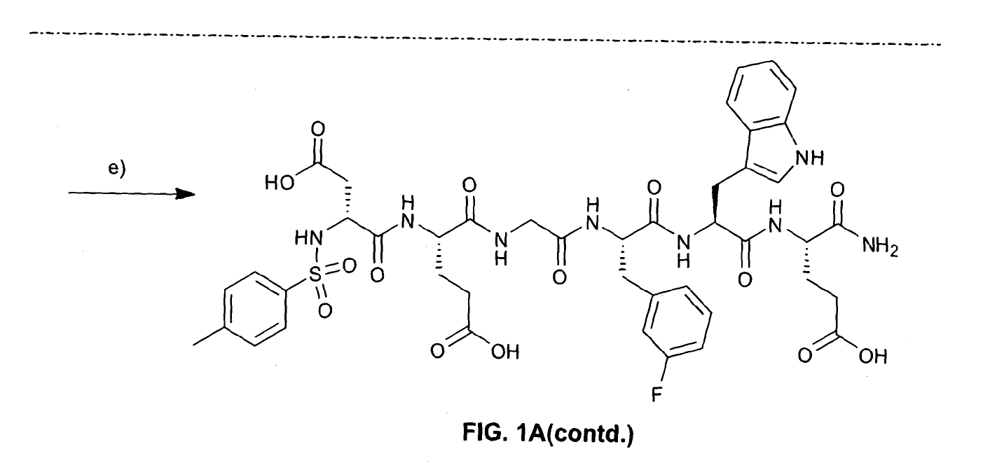

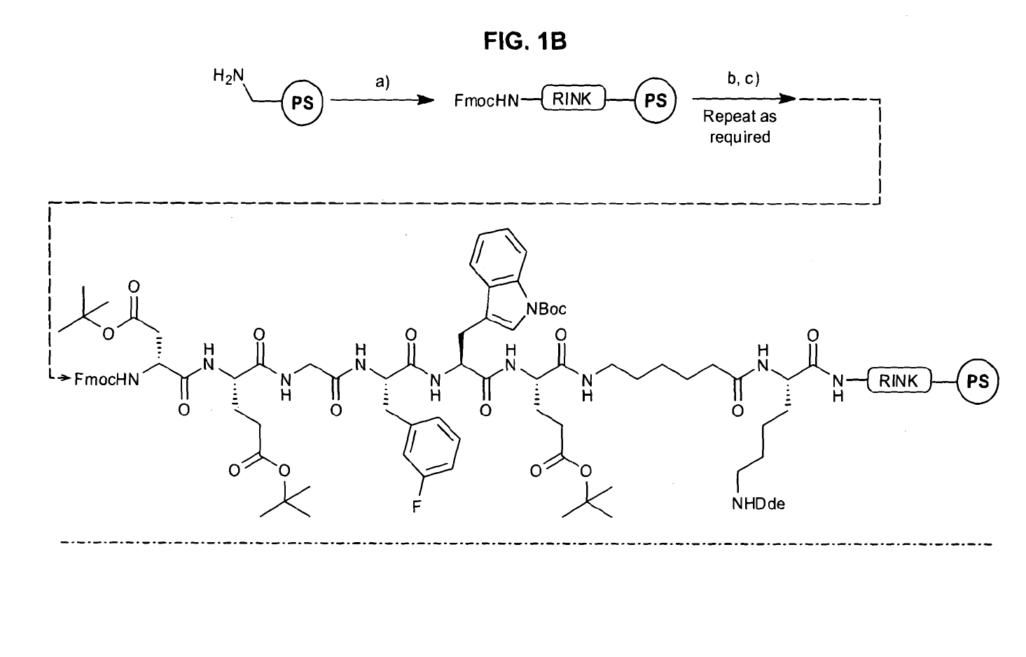

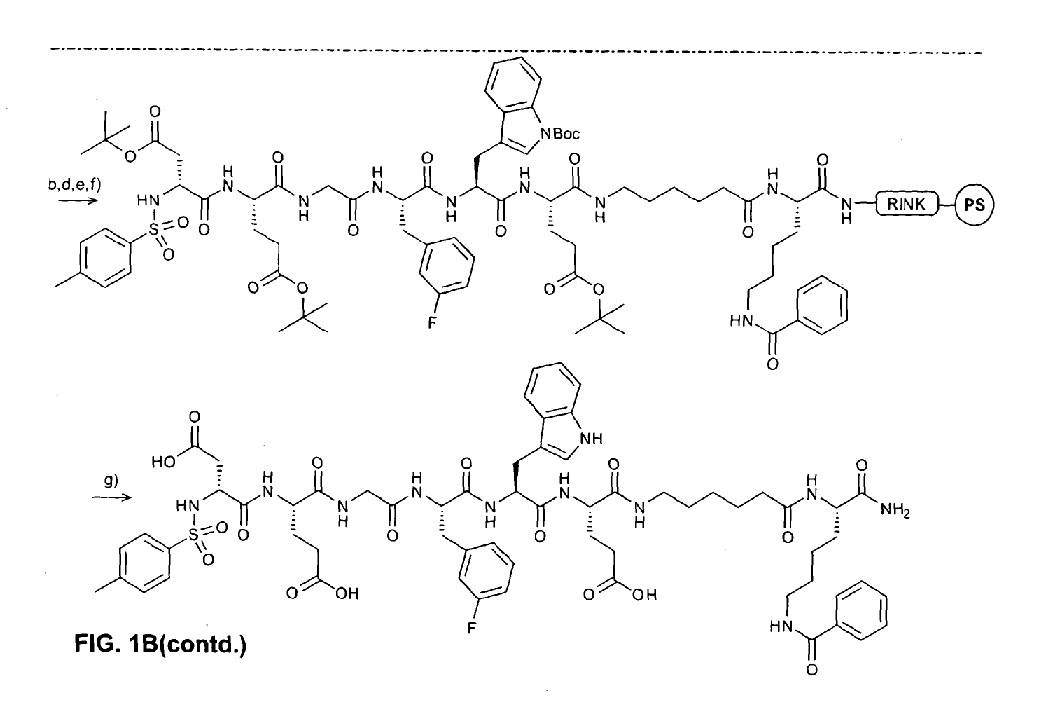

The experimental scheme for solid phase synthesis is shown in FIG. 1.

### Fluorescence Polarization Screening of βTrCP

### Assay Components

0.035 μM PTrCP (tag cleaved and complexed with Skp1)
10 nM fluorescein-RHDpSGLDpSMKD
50 mM Hepes pH 7.5

50 mM NaCl

1 mM DTT

0.1 mg/ml BSA (Bovine Serum Albumin)

50 M compound (in DMSO)

### Assay Protocol

Assay components (without compound) were premixed in a microcentrifuge tube and incubated for 1 hour to ensure equilibrium was achieved. Each compound was then added to one tube, mixed by vortexing, and then dispensed into 3 wells of a black 384-well plate and incubated for 30 minutes. Fluorescence polarization was then read (excitation 485 nM, emission 530 nM) using an Analyst-AD from Molecular Devices.

For dose-response curves to determine Ki, 10 different concentrations of compound were tested at equally spaced intervals. DMSO was added such that final concentration was 2%. Conditions are very tolerant to DMSO. Up to 10% DMSO has been tested previously, with no significant change to Kd values.

### Surface Plasmon Resonance (SPR)

Experiments were carried out using the Biacore T200 SPR detection system. This system exploits the phenomenon of surface plasmon resonance (SPR) to monitor interactions between molecules. The system involves the attachment of one interacting partner to a surface (an appropriate sensor chip) while the other interacting partner is passed over it in solution. The binding of molecules to the surface generates an SPR response (measured in response units (RU)) that is proportional to the mass and amount of the biomolecule (in this case βTrCP/Skp1) bound to the chip. The relative responses obtained are dependent on the concentration of the molecule binding. At RUₘₐₓᵢₘᵤₘ, the attached protein's binding sites are saturated. Binding events can be followed in real time and a range of interaction characteristics can be determined including kinetics, specificity of interactions and the concentration of specific molecules in a sample.

As βTrCP was His tagged, an NTA sensor chip was used. This sensor chip has a dextran surface matrix with immobilized nitrilotriacetic acid (NTA) which provides a means of capturing polyHis-tagged proteins through Nickel chelation. It was hoped that addition of Ni⁺ would orient the protein in a specific (and hopefully active) manner as it is covalently immobilised via amine coupling. Addition of EDC:NHS (N-ethyl-N′-(3-diethylaminopropyl)-carbodiimide:N-hydroxysuccinimide) converts carboxyl groups on the dextran sensor chip surface to succinamide esters which readily form covalent bonds with primary amines. Each chip contains four flow cells (each a separate surface) which means that compounds/peptides can be passed over different forms of βTrCP and a reference surface simultaneously. If binding to DTrCP is occurring, responses should be the same (accounting for differences in density of surface etc) on each surface.

Two different βTrCP protein complexes were immobilised on to an NTA biacore sensor chip. One included the GSTSkp1 fusion (HisβTrCP/GSTSkp1) while the other was immobilised after GST Removal by Thrombin (HisβTrCP/Skp1). To ensure that the GST moiety is completely removed from Skp1, βTrCP/GSTSkp1 was incubated with thrombin ((10 units/mg protein) for at least 16 hours at room temperature in 10 mMHEPES150 mMNaCl pH7.4+2 mM CaCl₂. Thrombin and GST were removed from βTrCP/Skp1 by buffer exchange through a 50 KDa MWCO vivaspin concentrator.

### Protein Immobilisation Procedure for NTA Chip

Ni⁺ (500 μM NiCl) loaded on to surface at a flow rate of 5 μl/min for 60 s. EDC/NHS (activates dextran carboxylates) loaded at a flow rate of 5 μl/min for 240 s. Protein ([protein]=100 nM to 1 μM) loaded at a flow rate of 101/min for 180 s. Strip solution (350 μM EDTA/1M NaCl) added at flow rate of 10 μl/min for 30 s (to chelate excess Ni+ and remove non-covalently bound protein e.g. protein oligomers). Quench solution (ethanolamine) at flow rate of 5 μl/min for 240 s (to deactivate surface molecules on the chip that have not crosslinked protein). The immobilisation buffer used was 10 mM HEPES pH 7.4, 150 mM NaCl,

### GST Capture Procedure (for Immobilisation via GSTSkp1)

EDC:NHS was injected at 5 μl/min for 4 min to activate surface for amine coupling (this converts carboxyl groups on the surface of the chip to succinamide esters that react with primary amines)

Anti-GST (60 μg/ml). was loaded at 10 μl/min for 4 min resulting in an increase in response units of 6730 (Biacore manual states it should result in ˜7000)

Ethanolamine was injected at 5 μl/min for 5 min to deactivate remaining unreacted esters at surface (quenching).

Injection of a low concentration of purified GST (from kit) was injected for 3 mins at 5 μl/min before running a regeneration cycle with glycine pH2.0 that disrupts the antibody-GST interaction. This step is recommended in the Biacore manual in order to “block” a minority of high affinity GST binding sites that may prevent regeneration and therefore reloading of fresh GST-protein of interest.

GSTSkp1/βTrCP (0.16 mg/ml) was then loaded at 10 μl/min for 4 min resulting in an increase of 1550 RU (2000RU is about the maximum to expect according to Biacore manual).

### SPR Assay Conditions

Small molecule/peptide samples to be assayed for binding to βTrCP surfaces, were provided as 10 mM stocks in 100% DMSO. All samples were tested in running buffer composed of 10 mM HEPES pH 7.4, 150 mM NaCl, 50 μM EDTA, 0.005% p20, and 1% DMSO. Serial dilutions were made using running buffer. Samples were tested over varying concentrations up to a maximum of 100 M. Two methods of measuring the SPR response were employed: single cycle kinetics which measures the response across different concentrations of sample within a single cycle (no regeneration of surface) and a method that measures the response at a given concentration in each cycle and includes a regeneration wash after each sample injection. The regeneration solution used was the same as running buffer, but included 500 mM NaCl. Data was fitted using Biacore T200 evaluation software. KD values calculated from binding curves from both surfaces (with or without the GST moiety) were averaged to produce apparent KD's for each sample tested.

### Biotin Pull Down Assay

### Assay Components

150 mM NaCl

10.01% NP40

1 mM DTT

0.3 μM βTrCP (1 μg)

0.3 μM biotinylated IκB peptide (KKERLLDDRHDpSGLDpSMKDEE)

100 μM compound (mabridge library: 50 μM)

### Protocol

βTrCP1 and biotinylated peptide were incubated in a volume of 25 μl at a final concentration of DMSO of 1% for 30 minutes to achieve equilibrium. Compounds were then added to a final concentration of 100 μM and allowed to incubate for an additional 30 minutes. 7.5 μl of streptavidin-agarose beads were then added to the reaction mix and allowed to incubate at room temperature for 30 minutes with gentle rocking. Beads were spun down and washed in buffer 3 times and then loaded onto a 10% SDS PAGE gel and visualized by GelCode blue staining.

### βTrCP Ubiquitination Assay and Selectivity Assays

### Assay Components

0.2 M E1 (Ube1)

2 μM E2 (UbCH5C)

0.25 μM E3 (Cull/Rbx1)

0.25 μM E3 (β-TrCP1/Skp1)

12 μM Ubiquitin

0.5 μM Peptide substrate (biotin)

10 mM MgCl₂

2 mM ATP

### Protocol

Master mixes were prepared in a 50 mM Herpes buffer at pH 7.5, in 75 mM NaCl and 1 mM DTTI without Mg or ATP and peptides were added to a final concentration of 100 μM. Reactions were incubated at room temperature for 30 minutes and then Mg/ATP was added to the mix. Reactions were further incubated for an additional 60 minutes and then stopped by adding SDS gel loading buffer and boiled for 5 minutes. Reactions were run on SDS-PAGE (10%) and transferred to nitrocellulose membranes and probed with HRP-Streptavidin.

FBW7 selectivity assays were performed in the same manner except using FBW7/Skp1 as E3 component and cyclin E as the substrate. Blots were probed using anti-cyclin E antibody.

### EXAMPLES

The following Examples illustrate the experiments performed by the inventors to arrive at the preset invention. It will be appreciated that modification of detail may be made without departing from the scope of the invention.

### Example 1

### Construction of Binding Peptides and Analysis Using Fluoresence Polarisation (FP) Assay

A consensus binding motif is known to be present in IkBa, Vpu and β-catenin, all of which bind βTrCP (J. Pons et al., Biochemistry, 2008, 47 (1), 14-29). The consensus motif has the sequence DpSGXXpS, wherein the two serine residues are phosphorylated.

A selection of compounds were prepared using the synthesis procedure described above, and shown in FIG. 1. Here, each residue of the consenus binding motif was replaced in turn as shown in Table 1.

Binding of the compounds to βTrCP was assessed using a fluorescence polarisation (FP) binding assay. The FP assay is an in vitro binding assay using a fluorescein-tagged IkB peptide at 10 nM to mimic substrate binding to βTrCP, and was performed as described above.

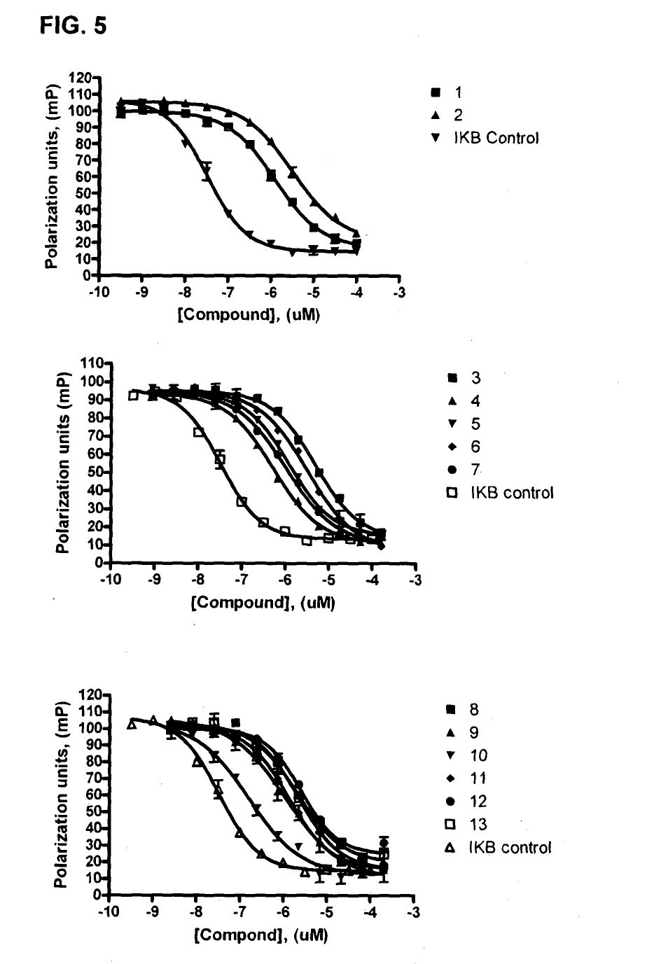

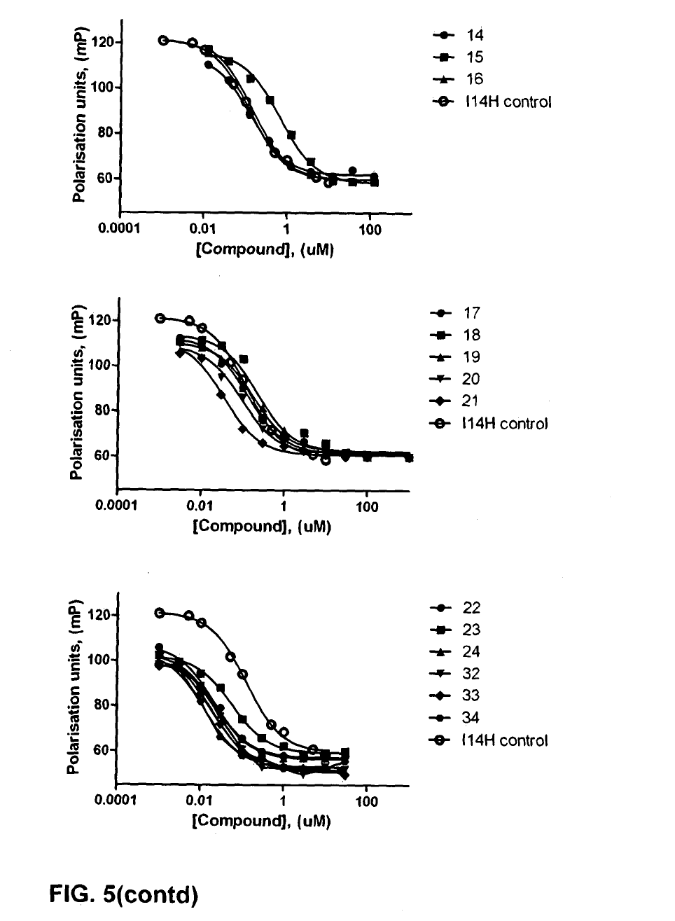

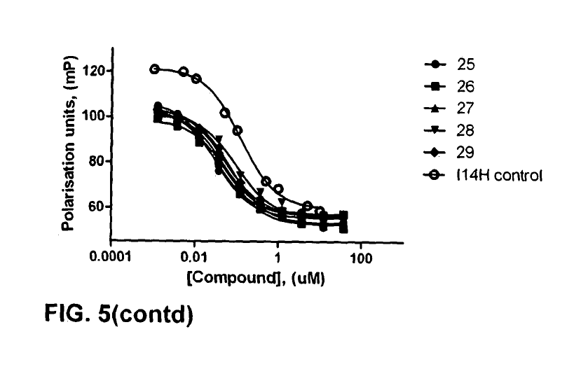

Dose response curves for a number of representaitve peptides are shown in FIG. 5.

TABLE 1
DpSGXXpS sequence modified to
determine alternate binding sequences

| | | FP Assay |
|---|---|---|
| Entry | Sequence | IC₅₀/μM |
| 1 | D-pS-GIHS-NH₂ | >100 |
| 2 | GD-pS-GIHS-NH₂ | >100 |
| 3 | AD-pS-GIHS-NH₂ | >100 |
| 4 | VD-pS-GIHS-NH₂ | >100 |
| 5 | DAGIHS-NH₂ | >100 |
| 6 | GDAGIHS-NH₂ | >100 |
| 7 | ADAGIHS-NH₂ | >100 |
| 8 | VDAGIHS-NH₂ | >100 |
| 9 | DDASGIHS-NH₂ | >100 |
| 10 | LDASGIHS-NH₂ | >100 |
| 11 | LD-pS-SGHIS-NH₂ | >100 |
| 12 | DAGIFE-NH₂ | >100 |
| 13 | EAGIFE-NH₂ | >100 |
| 14 | dEGIFE-NH₂ | >100 |
| 15 | dAGIFE-NH₂ | >100 |
| 16 | dAGIFR-NH₂ | >100 |
| 17 | dNGIFR-NH₂ | >100 |
| 18 | E-pS-GIFE-NH₂ | 34.5 |
| 19 | D-pS-GIFE-NH₂ | 24 |
| 20 | DAGNFE-NH₂ | >100 |
| 21 | DEGFFE-NH₂ | 43.6 |
| 22 | DAGFFE-NH₂ | >100 |
| 23 | dEGIFD-NH₂ | >100 |
| 24 | dAGIFD-NH₂ | >100 |
| 25 | DAGIFH-NH₂ | >100 |
| 26 | D-pS-GIFH-NH₂ | >100 |
| 27 | D-pS-GNFE-NH₂ | >100 |

The peptide DEGFFE-NH₂, having an IC₅₀ of 43.6 μM, was selected as a suitable non-phosphorylated candidate for further progression.

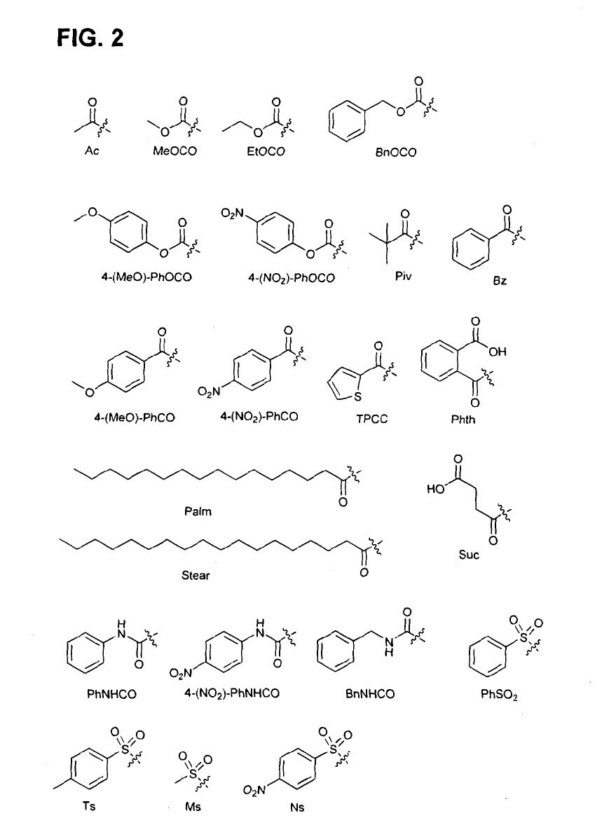

Starting from DEGFFE-NH₂, an array of compounds was prepared, as shown in Table 2, in which the N-terminal amino acid (D) was replaced with various capping groups. All of these compounds have an aminde at the C-terminus, as do those shown in Tables 5 and 6. FIG. 2 illustrates the capping groups used and their abbreviations.

TABLE 2
Optimisation of DEGFFE by replacing N-terminal Asp with capping group

| | P1 | P2 | P3 | P4 | P5 | P6 |
|---|---|---|---|---|---|---|
| 1 | D | F | G | I | F | E |
| 2 | D | E | G | Y | F | E |
| 3 | D | A | G | Y | F | E |
| 4 | Ac | E | G | I | F | E |
| 5 | MeOCO | E | G | I | F | E |
| 6 | EtOCO | E | G | I | F | E |
| 7 | BnOCO | E | G | I | F | E |
| 8 | 4-(MeO)—PhOCO | E | G | I | F | E |
| 9 | 4-(NO₂)—PhOCO | E | G | I | F | E |
| 10 | Piv | E | G | I | F | E |
| 11 | Bz | E | G | I | F | E |
| 12 | 4-(MeO)—PhCO | E | G | I | F | E |
| 13 | 4-(NO₂)—PhCO | E | G | I | F | E |
| 14 | Palm | E | G | I | F | E |
| 15 | Stear | E | G | I | F | E |
| 16 | TPCC | E | G | I | F | E |
| 17 | Phth | E | G | I | F | E |
| 18 | Suc | E | G | I | F | E |
| 19 | PhNHCO | E | G | I | F | E |
| 20 | BnNHCO | E | G | I | F | E |
| 21 | 4-(NO₂)—PhNHCO | E | G | I | F | E |
| 22 | Ms | E | G | 1 | F | E |
| 23 | PhSO₂ | E | G | I | F | E |
| 24 | Ts | E | G | I | F | E |
| 25 | Ns | E | G | I | F | E |
| 26 | D | E | G | F | F | E |
| 27 | Ac | E | G | F | F | E |
| 28 | MeOCO | E | G | F | F | E |
| 29 | EtOCO | E | G | F | F | E |
| 30 | BnOCO | E | G | F | F | E |
| 31 | 4-(MeO)—PhOCO | E | G | F | F | E |
| 32 | 4-(NO₂)—PhOCO | E | G | F | F | E |
| 33 | Piv | E | G | F | F | E |
| 34 | Bz | E | G | F | F | E |
| 35 | 4-(MeO)—PhCO | E | G | F | F | E |
| 36 | 4-(NO₂)—PhCO | E | G | F | F | E |
| 37 | Palm | E | G | F | F | E |
| 38 | Stear | E | G | F | F | E |
| 39 | TPCC | E | G | F | F | E |
| 40 | Phth | E | G | F | F | E |
| 41 | Suc | E | G | F | F | E |
| 42 | PhNHCO | E | G | F | F | E |
| 43 | BnNHCO | E | G | F | F | E |
| 44 | 4-(NO₂)—PhNHCO | E | G | F | F | E |
| 45 | Ms | E | G | F | F | E |
| 46 | PhSO₂ | E | G | F | F | E |
| 47 | Ts | E | G | F | F | E |
| 48 | Ns | E | G | F | F | E |

Four sequences were selected for re-synthesis and testing in the FP assay, which was performed as described above along with 4 negative controls. The results of the FP assay are shown in Table 3.

TABLE 3
FP assay results of selected capped sequences and negative controls

| | | FP Assay |
|---|---|---|
| Entry | Sequence | IC₅₀/μM |
| 1 | DEGIFE-NH₂ | >100 |
| 2 | Phth-EGIFE-NH₂ | >100 |
| 3 | Suc-AGIFE-NH₂ | >100 |
| 4 | Phth-AGIFE-NH₂ | >100 |
| 5 | Suc-EGIFE-NH₂ | >100 |
| 6 | Phth-EGFFE-NH₂ | 10.9 |
| 7 | Suc-EGFFE-NH₂ | 3.18 |
| 8 | Phth-AGFFE-NH₂ | >100 |
| 9 | Suc-AGFFE-NH₂ | >100 |

The FP assay identified Suc-EGFFE-NH₂ as a low μM inhibitor for further optimisation.

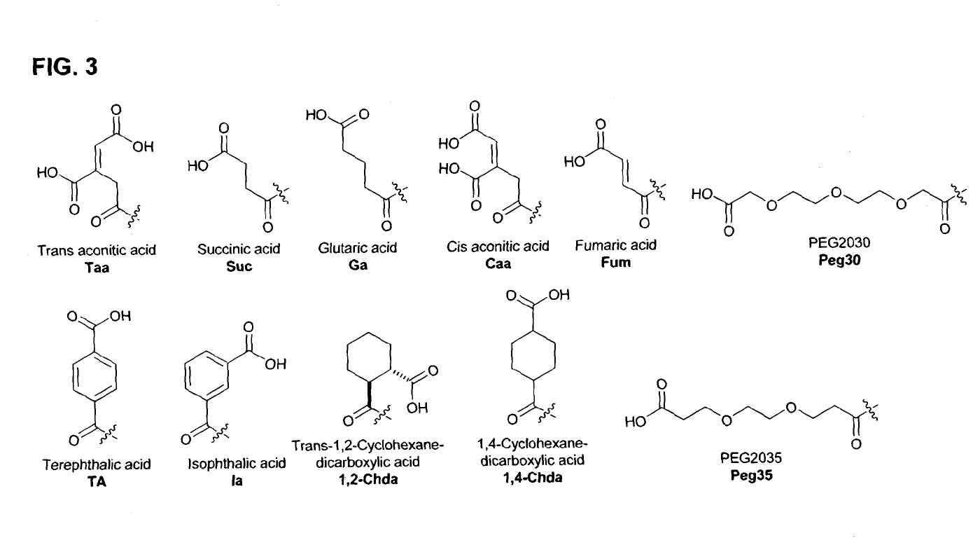

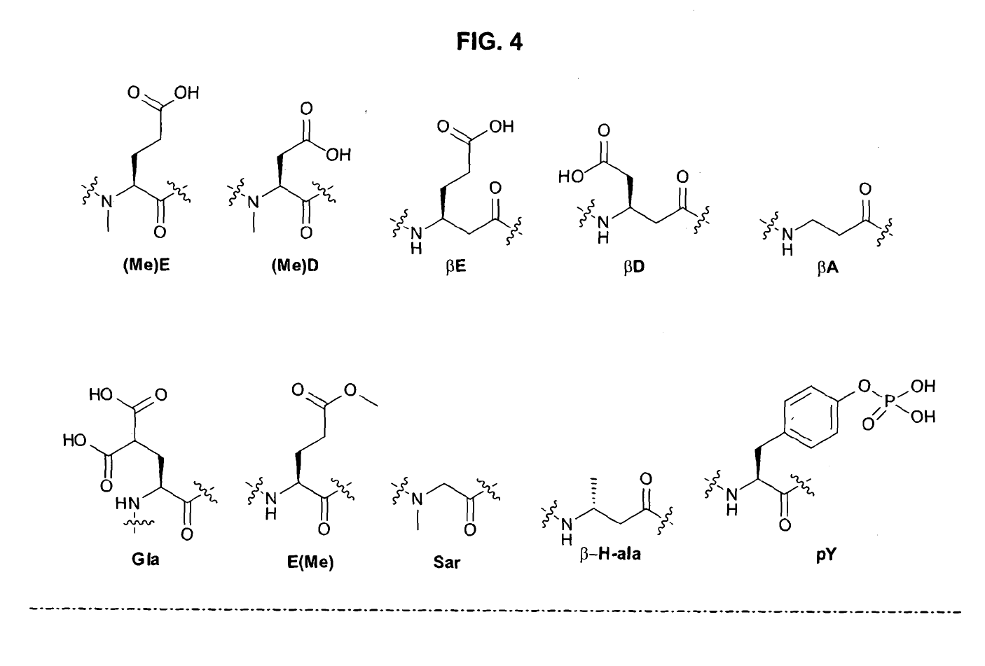

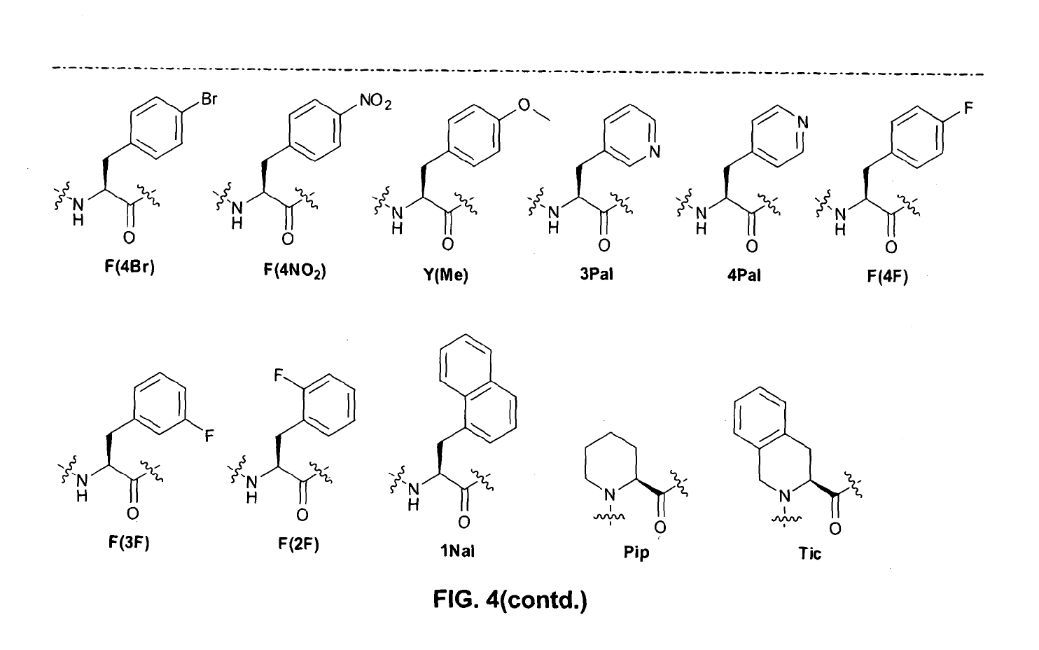

Further peptides were synthesised as described above by replacing residues in the Suc-EGFFE-NH₂ sequence with alternative acidic capping groups and non-natural amino acids. The sequences and abbreviations of the acidic capping groups and non-natural amino acids are shown in FIGS. 3 and 4, respectively, as well as in Table 4, and the generated peptide sequences are shown in Tables 5 and 6.

TABLE 4
Key to capping groups

| | |
|---|---|
| Succinic anhydride | Suc |
| PEG 2035 | Peg35 |
| PEG2030 | Peg30 |
| Trans aconitic acid | Taa |
| Cis aconitic acid | Caa |
| Fumaric acid | Fum |
| Terephthalic acid | TA |
| Isophthalic acid | Ia |
| 1,4 cyclohexanedicarboxylic acid | 1,4-Chda |
| trans-1,2 cyclohexanedicarboxylic acid | 1,2-Chda |
| Glutaric anhydride | Ga |

TABLE 5
Sequence of β-TrCP binding peptides (5-Mers) synthesised

| | P1 | P2 | P3 | P4 | P5 | P6 |
|---|---|---|---|---|---|---|
| 1 | Peg35 | E | G | F | F | E |
| 2 | Peg30 | E | G | F | F | E |
| 3 | Taa | E | G | F | F | E |
| 4 | Caa | E | G | F | F | E |
| 5 | Fum | E | G | F | F | E |
| 6 | TA | E | G | F | F | E |
| 7 | Ia | E | G | F | F | E |
| 8 | 1,4-Chda | E | G | F | F | E |
| 9 | 1,2-Chda | E | G | F | F | E |
| 10 | Ga | E | G | F | F | E |
| 11 | Suc | E | G | Y | F | E |
| 12 | Peg35 | E | G | Y | F | E |
| 13 | Peg30 | E | G | Y | F | E |
| 14 | Taa | E | G | Y | F | E |
| 15 | Caa | E | G | Y | F | E |
| 16 | Fum | E | G | Y | F | E |
| 17 | TA | E | G | Y | F | E |
| 18 | Ia | E | G | Y | F | E |
| 19 | 1,4-Chda | E | G | Y | F | E |
| 20 | 1,2-Chda | E | G | Y | F | E |
| 21 | Ga | E | G | Y | F | E |
| 22 | Suc | E | A | F | F | E |
| 23 | Peg35 | E | A | F | F | E |
| 24 | Peg30 | E | A | F | F | E |
| 25 | Taa | E | A | F | F | E |
| 26 | Caa | E | A | F | F | E |
| 27 | Fum | E | A | F | F | E |
| 28 | TA | E | A | F | F | E |
| 29 | Ia | E | A | F | F | E |
| 30 | 1,4-Chda | E | A | F | F | E |
| 31 | 1,2-Chda | E | A | F | F | E |
| 32 | Ga | E | A | F | F | E |
| 33 | Suc | E | A | F | F | E |
| 34 | Peg35 | E | A | F | F | E |
| 35 | Peg30 | E | A | F | F | E |
| 36 | Taa | E | A | F | F | E |
| 37 | Caa | E | A | F | F | E |
| 38 | Fum | E | A | F | F | E |
| 39 | TA | E | A | F | F | E |
| 40 | Ia | E | A | F | F | E |
| 41 | 1,4-Chda | E | A | F | F | E |
| 42 | 1,2-Chda | E | A | F | F | E |
| 43 | Ga | E | A | F | F | E |
| 44 | Suc | E | βA | F | F | E |
| 45 | Peg35 | E | βA | F | F | E |
| 46 | Peg30 | E | βA | F | F | E |
| 47 | Taa | E | βA | F | F | E |
| 48 | Caa | F | βA | F | F | E |
| 49 | Fum | E | βA | F | F | E |
| 50 | TA | E | βA | F | F | E |
| 51 | Ia | E | βA | F | F | E |
| 52 | 1,4-Chda | E | βA | F | F | E |
| 53 | 1,2-Chda | E | βA | F | F | E |
| 54 | Ga | E | βA | F | F | E |
| 55 | Suc | E | P | F | F | E |
| 56 | Peg35 | E | P | F | F | E |
| 57 | Peg30 | E | P | F | F | E |
| 58 | Taa | E | P | F | F | E |
| 59 | Caa | E | P | F | F | E |
| 60 | Fum | E | P | F | F | E |
| 61 | TA | E | P | F | F | E |
| 62 | Ia | E | P | F | F | E |
| 63 | 1,4-Chda | E | P | F | F | E |
| 64 | 1,2-Chda | E | P | F | F | E |
| 65 | Ga | E | P | F | F | E |
| 66 | Suc | E | p | F | F | E |
| 67 | Peg35 | E | p | F | F | E |
| 68 | Peg30 | E | p | F | F | E |
| 69 | Taa | E | p | F | F | E |
| 70 | Caa | E | p | F | F | E |
| 71 | Fum | E | p | F | F | E |
| 72 | TA | E | p | F | F | E |
| 73 | Ia | E | p | F | F | E |
| 74 | 1,4-Chda | E | p | F | F | E |
| 75 | 1,2-Chda | E | p | F | F | E |
| 76 | Ga | E | p | F | F | E |
| 77 | Suc | E | Sar | F | F | E |
| 78 | Peg35 | E | Sar | F | F | E |
| 79 | Peg30 | E | Sar | F | F | E |
| 80 | Taa | E | Sar | F | F | E |
| 81 | Caa | E | Sar | F | F | E |
| 82 | Fum | E | Sar | F | F | E |
| 83 | TA | E | Sar | F | F | E |
| 84 | Ia | E | Sar | F | F | E |
| 85 | 1,4-Chda | E | Sar | F | F | E |
| 86 | 1,2-Chda | E | Sar | F | F | E |
| 87 | Ga | E | Sar | F | F | E |
| 88 | Suc | E | β-H-ala | F | F | E |
| 89 | Peg35 | E | β-H-ala | F | F | E |
| 90 | Peg30 | E | β-H-ala | F | F | E |
| 91 | Taa | E | β-H-ala | F | F | E |
| 92 | Caa | E | β-H-ala | F | F | E |
| 93 | Fum | E | β-H-ala | F | F | E |
| 94 | TA | E | β-H-ala | F | F | E |
| 95 | Ia | E | β-H-ala | F | F | E |
| 96 | 1,4-Chda | E | β-H-ala | F | F | E |
| 97 | 1,2-Chda | E | β-H-ala | F | F | E |
| 98 | Ga | E | β-H-ala | F | F | E |
| 99 | Suc | E | G | F | F | E |
| 100 | Suc | Q | G | F | F | E |
| 101 | Suc | N | G | F | F | E |
| 102 | Suc | (Me)E | G | F | F | E |
| 103 | Suc | βE | G | F | F | E |
| 104 | Suc | (Me)D | G | F | F | E |
| 105 | Suc | βD | G | F | F | E |
| 106 | Suc | E(Me) | G | F | F | E |
| 107 | Suc | D | G | F | F | E |
| 108 | Suc | Gla | G | F | F | E |
| 109 | Suc | E | G | Y | F | E |
| 110 | Suc | Q | G | Y | F | E |
| 111 | Suc | N | G | Y | F | E |
| 112 | Suc | (Me)E | G | Y | F | E |
| 113 | Suc | βE | G | Y | F | E |
| 114 | Suc | (Me)D | G | Y | F | E |
| 115 | Suc | βD | G | Y | F | E |
| 116 | Suc | E(Me) | G | Y | F | E |
| 117 | Suc | D | G | Y | F | E |
| 118 | Suc | Gla | G | Y | F | E |
| 119 | Suc | E | G | W | F | E |
| 120 | Suc | Q | G | W | F | E |
| 121 | Suc | N | G | W | F | E |
| 122 | Suc | (Me)E | G | W | F | E |
| 123 | Suc | βE | G | W | F | E |
| 124 | Suc | (Me)D | G | W | F | E |
| 125 | Suc | βD | G | W | F | E |
| 126 | Suc | E(Me) | G | W | F | E |
| 127 | Suc | D | G | W | F | E |
| 128 | Suc | Gla | G | W | F | E |
| 129 | Suc | E | G | F(3Br) | F | E |
| 130 | Suc | Q | G | F(3Br) | F | E |
| 131 | Suc | N | G | F(3Br) | F | E |
| 132 | Suc | (Me)E | G | F(3Br) | F | E |
| 133 | Suc | βE | G | F(3Br) | F | E |
| 134 | Suc | (Me)D | G | F(3Br) | F | E |
| 135 | Suc | βD | G | F(3Br) | F | E |
| 136 | Suc | E(Me) | G | F(3Br) | F | E |
| 137 | Suc | D | G | F(3Br) | F | E |
| 138 | Suc | Gla | G | F(3Br) | F | E |
| 139 | Suc | E | G | F(4NO₂) | F | E |
| 140 | Suc | Q | G | F(4NO₂) | F | E |
| 141 | Suc | N | G | F(4NO₂) | F | E |
| 142 | Suc | (Me)E | G | F(4NO₂) | F | E |
| 143 | Suc | βE | G | F(4NO₂) | F | E |
| 144 | Suc | (Me)D | G | F(4NO₂) | F | E |
| 145 | Suc | βD | G | F(4NO₂) | F | E |
| 146 | Suc | E(Me) | G | F(4NO₂) | F | E |
| 147 | Suc | D | G | F(4NO₂) | F | E |
| 148 | Suc | Gla | G | F(4NO₂) | F | E |
| 149 | Suc | E | G | 3Pal | F | E |
| 150 | Suc | Q | G | 3Pal | F | E |
| 151 | Suc | N | G | 3Pal | F | E |
| 152 | Suc | (Me)E | G | 3Pal | F | E |
| 153 | Suc | βE | G | 3Pal | F | E |
| 154 | Suc | (Me)D | G | 3Pal | F | E |
| 155 | Suc | βD | G | 3Pal | F | E |
| 156 | Suc | E(Me) | G | 3Pal | F | E |
| 157 | Suc | D | G | 3Pal | F | E |
| 158 | Suc | Gla | G | 3Pal | F | E |
| 159 | Suc | E | G | Y(Me) | F | E |
| 160 | Suc | Q | G | Y(Me) | F | E |
| 161 | Suc | N | G | Y(Me) | F | E |
| 162 | Suc | (Me)E | G | Y(Me) | F | E |
| 163 | Suc | βE | G | Y(Me) | F | E |
| 164 | Suc | (Me)D | G | Y(Me) | F | E |
| 165 | Suc | βD | G | Y(Me) | F | E |
| 166 | Suc | E(Me) | G | Y(Me) | F | E |
| 167 | Suc | D | G | Y(Me) | F | E |
| 168 | Suc | Gla | G | Y(Me) | F | E |
| 169 | Suc | E | G | Pip | F | E |
| 170 | Suc | Q | G | Pip | F | E |
| 171 | Suc | N | G | Pip | F | E |
| 172 | Suc | (Me)E | G | Pip | F | E |
| 173 | Suc | βE | G | Pip | F | E |
| 174 | Suc | (Me)D | G | Pip | F | E |
| 175 | Suc | βD | G | Pip | F | E |
| 176 | Suc | E(Me) | G | Pip | F | E |
| 177 | Suc | D | G | Pip | F | E |
| 178 | Suc | Gla | G | Pip | F | E |
| 179 | Suc | E | G | Tic | F | E |
| 180 | Suc | Q | G | Tic | F | E |
| 181 | Suc | N | G | Tic | F | E |
| 182 | Suc | (Me)E | G | Tic | F | E |
| 183 | Suc | βE | G | Tic | F | E |
| 184 | Suc | (Me)D | G | Tic | F | E |
| 185 | Suc | βD | G | Tic | F | E |
| 186 | Suc | E(Me) | G | Tic | F | E |
| 187 | Suc | D | G | Tic | F | E |
| 188 | Suc | Gla | G | Tic | F | E |
| 189 | Suc | E | G | 1Nal | F | E |
| 190 | Suc | Q | G | 1Nal | F | E |
| 191 | Suc | N | G | 1Nal | F | E |
| 192 | Suc | (Me)E | G | 1Nal | F | E |
| 193 | Suc | βE | G | 1Nal | F | E |
| 194 | Suc | (Me)D | G | 1Nal | F | E |
| 195 | Suc | βD | G | 1Nal | F | E |
| 196 | Suc | E(Me) | G | 1Nal | F | E |
| 197 | Suc | D | G | 1Nal | F | E |
| 198 | Suc | Gla | G | 1Nal | F | E |
| 199 | Suc | E | G | F(2F) | F | E |
| 200 | Suc | Q | G | F(2F) | F | E |
| 201 | Suc | N | G | F(2F) | F | E |
| 202 | Suc | (Me)E | G | F(2F) | F | E |
| 203 | Suc | βE | G | F(2F) | F | E |
| 204 | Suc | (Me)D | G | F(2F) | F | E |
| 205 | Suc | βD | G | F(2F) | F | E |
| 206 | Suc | E(Me) | G | F(2F) | F | E |
| 207 | Suc | D | G | F(2F) | F | E |
| 208 | Suc | Gla | G | F(2F) | F | E |
| 209 | Suc | E | G | F(3F) | F | E |
| 210 | Suc | Q | G | F(3F) | F | E |
| 211 | Suc | N | G | F(3F) | F | E |
| 212 | Suc | (Me)E | G | F(3F) | F | E |
| 213 | Suc | βE | G | F(3F) | F | E |
| 214 | Suc | (Me)D | G | F(3F) | F | E |
| 215 | Suc | βD | G | F(3F) | F | E |
| 216 | Suc | E(Me) | G | F(3F) | F | E |
| 217 | Suc | D | G | F(3F) | F | E |
| 218 | Suc | Gla | G | F(3F) | F | E |
| 219 | Suc | E | G | F(4F) | F | E |
| 220 | Suc | Q | G | F(4F) | F | E |
| 221 | Suc | N | G | F(4F) | F | E |
| 222 | Suc | (Me)E | G | F(4F) | F | E |
| 223 | Suc | βE | G | F(4F) | F | E |
| 224 | Suc | (Me)D | G | F(4F) | F | E |
| 225 | Suc | βD | G | F(4F) | F | E |
| 226 | Suc | E(Me) | G | F(4F) | F | E |
| 227 | Suc | D | G | F(4F) | F | E |
| 228 | Suc | Gla | G | F(4F) | F | E |
| 229 | Suc | E | G | F | F | E |
| 230 | Suc | E | G | F | F | Q |
| 231 | Suc | E | G | F | F | N |
| 232 | Suc | E | G | F | F | (Me)E |
| 233 | Suc | E | G | F | F | βE |
| 234 | Suc | E | G | F | F | (Me)D |
| 235 | Suc | E | G | F | F | βD |
| 236 | Suc | E | G | F | F | E(Me) |
| 237 | Suc | E | G | F | F | D |
| 238 | Suc | E | G | F | F | Gla |
| 239 | Suc | E | G | F | Y | E |
| 240 | Suc | E | G | F | Y | Q |
| 241 | Suc | E | G | F | Y | N |
| 242 | Suc | E | G | F | Y | (Me)E |
| 243 | Suc | E | G | F | Y | βE |
| 244 | Suc | E | G | F | Y | (Me)D |
| 245 | Suc | E | G | F | Y | βD |
| 246 | Suc | E | G | F | Y | E(Me) |
| 247 | Suc | E | G | F | Y | D |
| 248 | Suc | E | G | F | Y | Gla |
| 249 | Suc | E | G | F | W | E |
| 250 | Suc | E | G | F | W | Q |
| 251 | Suc | E | G | F | W | N |
| 252 | Suc | E | G | F | W | (Me)E |
| 253 | Suc | E | G | F | W | βE |
| 254 | Suc | E | G | F | W | (Me)D |
| 255 | Suc | E | G | F | W | βD |
| 256 | Suc | E | G | F | W | E(Me) |
| 257 | Suc | E | G | F | W | D |
| 258 | Suc | E | G | F | W | Gla |
| 259 | Suc | E | G | F | F(3Br) | E |
| 260 | Suc | E | G | F | F(3Br) | Q |
| 261 | Suc | E | G | F | F(3Br) | N |
| 262 | Suc | E | G | F | F(3Br) | (Me)E |
| 263 | Suc | E | G | F | F(3Br) | βE |
| 264 | Suc | E | G | F | F(3Br) | (Me)D |
| 265 | Suc | E | G | F | F(3Br) | βD |
| 266 | Suc | E | G | F | F(3Br) | E(Me) |
| 267 | Suc | E | G | F | F(3Br) | D |
| 268 | Suc | E | G | F | F(3Br) | Gla |
| 269 | Suc | E | G | F | F(4NO₂) | E |
| 270 | Suc | E | G | F | F(4NO₂) | Q |
| 271 | Suc | E | G | F | F(4NO₂) | N |
| 272 | Suc | E | G | F | F(4NO₂) | (Me)E |
| 273 | Suc | E | G | F | F(4NO₂) | βE |
| 274 | Suc | E | G | F | F(4NO₂) | (Me)D |
| 275 | Suc | E | G | F | F(4NO₂) | βD |
| 276 | Suc | E | G | F | F(4NO₂) | E(Me) |
| 277 | Suc | E | G | F | F(4NO₂) | D |
| 278 | Suc | E | G | F | F(4NO₂) | Gla |
| 279 | Suc | E | G | F | 3Pal | E |
| 280 | Suc | E | G | F | 3Pal | Q |
| 281 | Suc | E | G | F | 3Pal | N |
| 282 | Suc | E | G | F | 3Pal | (Me)E |
| 283 | Suc | E | G | F | 3Pal | βE |
| 284 | Suc | E | G | F | 3Pal | (Me)D |
| 285 | Suc | E | G | F | 3Pal | βD |
| 286 | Suc | E | G | F | 3Pal | E(Me) |
| 287 | Suc | F | G | F | 3Pal | D |
| 288 | Suc | E | G | F | 3Pal | Gla |
| 289 | Suc | E | G | F | Y(Me) | E |
| 290 | Suc | E | G | F | Y(Me) | Q |
| 291 | Suc | E | G | F | Y(Me) | N |
| 292 | Suc | E | G | F | Y(Me) | (Me)E |
| 293 | Suc | E | G | F | Y(Me) | βE |
| 294 | Suc | E | G | F | Y(Me) | (Me)D |
| 295 | Suc | E | G | F | Y(Me) | βD |
| 296 | Suc | E | G | F | Y(Me) | E(Me) |
| 297 | Suc | E | G | F | Y(Me) | D |
| 298 | Suc | E | G | F | Y(Me) | Gla |
| 299 | Suc | E | G | F | Pip | E |
| 300 | Suc | E | G | F | Pip | Q |
| 301 | Suc | E | G | F | Pip | N |
| 302 | Suc | E | G | F | Pip | (Me)E |
| 303 | Suc | E | G | F | Pip | βE |
| 304 | Suc | E | G | F | Pip | (Me)D |
| 305 | Suc | E | G | F | Pip | βD |
| 306 | Suc | E | G | F | Pip | E(Me) |
| 307 | Suc | E | G | F | Pip | D |
| 308 | Suc | E | G | F | Pip | Gla |
| 309 | Suc | E | G | F | Tic | E |
| 310 | Suc | E | G | F | Tic | Q |
| 311 | Suc | E | G | F | Tic | N |
| 312 | Suc | E | G | F | Tic | (Me)E |
| 313 | Suc | E | G | F | Tic | βE |
| 314 | Suc | E | G | F | Tic | (Me)D |
| 315 | Suc | E | G | F | Tic | βD |
| 316 | Suc | E | G | F | Tic | E(Me) |
| 317 | Suc | E | G | F | Tic | D |
| 318 | Suc | E | G | F | Tic | Gla |
| 319 | Suc | E | G | F | 1Nal | E |
| 320 | Suc | E | G | F | 1Nal | Q |
| 321 | Suc | E | G | F | 1Nal | N |
| 322 | Suc | E | G | F | 1Nal | (Me)E |
| 323 | Suc | E | G | F | 1Nal | βE |
| 324 | Suc | E | G | F | 1Nal | (Me)D |
| 325 | Suc | E | G | F | 1Nal | βD |
| 326 | Suc | E | G | F | 1Nal | E(Me) |
| 327 | Suc | E | G | F | 1Nal | D |
| 328 | Suc | E | G | F | 1Nal | Gla |
| 329 | Suc | E | G | F | F(2F) | E |
| 330 | Suc | E | G | F | F(2F) | Q |
| 331 | Suc | E | G | F | F(2F) | N |
| 332 | Suc | E | G | F | F(2F) | (Me)E |
| 333 | Suc | E | G | F | F(2F) | βE |
| 334 | Suc | E | G | F | F(2F) | (Me)D |
| 335 | Suc | E | G | F | F(2F) | βD |
| 336 | Suc | E | G | F | F(2F) | E(Me) |
| 337 | Suc | E | G | F | F(2F) | D |
| 338 | Suc | E | G | F | F(2F) | Gla |
| 339 | Suc | E | G | F | F(3F) | E |
| 340 | Suc | E | G | F | F(3F) | Q |
| 341 | Suc | E | G | F | F(3F) | N |
| 342 | Suc | E | G | F | F(3F) | (Me)E |
| 343 | Suc | E | G | F | F(3F) | βE |
| 344 | Suc | E | G | F | F(3F) | (Me)D |
| 345 | Suc | E | G | F | F(3F) | βD |
| 346 | Suc | E | G | F | F(3F) | E(Me) |
| 347 | Suc | E | G | F | F(3F) | D |
| 348 | Suc | E | G | F | F(3F) | Gla |
| 349 | Suc | E | G | F | F(4F) | E |
| 350 | Suc | E | G | F | F(4F) | Q |
| 351 | Suc | E | G | F | F(4F) | N |
| 352 | Suc | E | G | F | F(4F) | (Me)E |
| 353 | Suc | E | G | F | F(4F) | βE |
| 354 | Suc | E | G | F | F(4F) | (Me)D |
| 355 | Suc | E | G | F | F(4F) | βD |
| 356 | Suc | E | G | F | F(4F) | E(Me) |
| 357 | Suc | E | G | F | F(4F) | D |
| 358 | Suc | E | G | F | F(4F) | Gla |
| 359 | Suc | E | G | F | F | E |
| 360 | Suc | E | G | Y | F | E |
| 361 | Suc | E | G | W | F | E |
| 362 | Suc | E | G | F(3Br) | F | E |
| 363 | Suc | E | G | F(4NO₂) | F | E |
| 364 | Suc | E | G | 3Pal | F | E |
| 365 | Suc | E | G | Y(Me) | F | E |
| 366 | Suc | E | G | Pip | F | E |
| 367 | Suc | E | G | Tic | F | E |
| 368 | Suc | E | G | 1Nal | F | E |
| 369 | Suc | E | G | F(2F) | F | E |
| 370 | Suc | E | G | F(3F) | F | E |
| 371 | Suc | E | G | F(4F) | F | E |
| 372 | Suc | E | G | F | Y | E |
| 373 | Suc | E | G | Y | Y | E |
| 374 | Suc | E | G | W | Y | E |
| 375 | Suc | E | G | F(3Br) | Y | E |
| 376 | Suc | E | G | F(4NO₂) | Y | E |
| 377 | Suc | E | G | 3Pal | Y | E |
| 378 | Suc | E | G | Y(Me) | Y | E |
| 379 | Suc | E | G | Pip | Y | E |
| 380 | Suc | E | G | Tic | Y | E |
| 381 | Suc | E | G | 1Nal | Y | E |
| 382 | Suc | E | G | F(2F) | Y | E |
| 383 | Suc | E | G | F(3F) | Y | E |
| 384 | Suc | E | G | F(4F) | Y | E |
| 385 | Suc | E | G | F | W | E |
| 386 | Suc | E | G | Y | W | E |
| 387 | Suc | E | G | W | W | E |
| 388 | Suc | E | G | F(3Br) | W | E |
| 389 | Suc | E | G | F(4NO₂) | W | E |
| 390 | Suc | E | G | 3Pal | W | E |
| 391 | Suc | E | G | Y(Me) | W | E |
| 392 | Suc | E | G | Pip | W | E |
| 393 | Suc | E | G | Tic | W | E |
| 394 | Suc | E | G | 1Nal | W | E |
| 395 | Suc | E | G | F(2F) | W | E |
| 396 | Suc | E | G | F(3F) | W | E |
| 397 | Suc | E | G | F(4F) | W | E |
| 398 | Suc | E | G | F | F(3Br) | E |
| 399 | Suc | E | G | Y | F(3Br) | E |
| 400 | Suc | E | G | W | F(3Br) | E |
| 401 | Suc | E | G | F(3Br) | F(3Br) | E |
| 402 | Suc | E | G | F(4NO₂) | F(3Br) | E |
| 403 | Suc | E | G | 3Pal | F(3Br) | E |
| 404 | Suc | E | G | Y(Me) | F(3Br) | E |
| 405 | Suc | E | G | Pip | F(3Br) | E |
| 406 | Suc | E | G | Tic | F(3Br) | E |
| 407 | Suc | E | G | 1Nal | F(3Br) | E |
| 408 | Suc | E | G | F(2F) | F(3Br) | E |
| 409 | Suc | E | G | F(3F) | F(3Br) | E |
| 410 | Suc | E | G | F(4F) | F(3Br) | E |
| 411 | Suc | E | G | F | F(4NO₂) | E |
| 412 | Suc | E | G | Y | F(4NO₂) | E |
| 413 | Suc | E | G | W | F(4NO₂) | E |
| 414 | Suc | E | G | F(3Br) | F(4NO₂) | E |
| 415 | Suc | E | G | F(4NO₂) | F(4NO₂) | E |
| 416 | Suc | E | G | 3Pal | F(4NO₂) | E |
| 417 | Suc | E | G | Y(Me) | F(4NO₂) | E |
| 418 | Suc | E | G | Pip | F(4NO₂) | E |
| 419 | Suc | E | G | Tic | F(4NO₂) | E |
| 420 | Suc | E | G | 1Nal | F(4NO₂) | E |
| 421 | Suc | E | G | F(2F) | F(4NO₂) | E |
| 422 | Suc | E | G | F(3F) | F(4NO₂) | E |
| 423 | Suc | E | G | F(4F) | F(4NO₂) | E |
| 424 | Suc | E | G | F | 3Pal | E |
| 425 | Suc | E | G | Y | 3Pal | E |
| 426 | Suc | E | G | W | 3Pal | E |
| 427 | Suc | E | G | F(3Br) | 3Pal | E |
| 428 | Suc | E | G | F(4NO₂) | 3Pal | E |
| 429 | Suc | E | G | 3Pal | 3Pal | E |
| 430 | Suc | E | G | Y(Me) | 3Pal | E |
| 431 | Suc | E | G | Pip | 3Pal | E |
| 432 | Suc | E | G | Tic | 3Pal | E |
| 433 | Suc | E | G | 1Nal | 3Pal | E |
| 434 | Suc | E | G | F(2F) | 3Pal | E |
| 435 | Suc | E | G | F(3F) | 3Pal | E |
| 436 | Suc | E | G | F(4F) | 3Pal | E |
| 437 | Suc | E | G | F | Y(Me) | E |
| 438 | Suc | E | G | Y | Y(Me) | E |
| 439 | Suc | E | G | W | Y(Me) | E |
| 440 | Suc | E | G | F(3Br) | Y(Me) | E |
| 441 | Suc | E | G | F(4NO₂) | Y(Me) | E |
| 442 | Suc | E | G | 3Pal | Y(Me) | E |
| 443 | Suc | E | G | Y(Me) | Y(Me) | E |
| 444 | Suc | E | G | Pip | Y(Me) | E |
| 445 | Suc | E | G | Tic | Y(Me) | E |
| 446 | Suc | E | G | 1Nal | Y(Me) | E |
| 447 | Suc | E | G | F(2F) | Y(Me) | E |
| 448 | Suc | E | G | F(3F) | Y(Me) | E |
| 449 | Suc | E | G | F(4F) | Y(Me) | E |
| 450 | Suc | E | G | F | Pip | E |
| 451 | Suc | E | G | Y | Pip | E |
| 452 | Suc | E | G | W | Pip | E |
| 453 | Suc | E | G | F(3Br) | Pip | E |
| 454 | Suc | E | G | F(4NO₂) | Pip | E |
| 455 | Suc | E | G | 3Pal | Pip | E |
| 456 | Suc | E | G | Y(Me) | Pip | E |
| 457 | Suc | E | G | Pip | Pip | E |
| 458 | Suc | E | G | Tic | Pip | E |
| 459 | Suc | E | G | 1Nal | Pip | E |
| 460 | Suc | E | G | F(2F) | Pip | E |
| 461 | Suc | E | G | F(3F) | Pip | E |
| 462 | Suc | E | G | F(4F) | Pip | E |
| 463 | Suc | E | G | F | Tic | E |
| 464 | Suc | E | G | Y | Tic | E |
| 465 | Suc | E | G | W | Tic | E |
| 466 | Suc | E | G | F(3Br) | Tic | E |
| 467 | Suc | E | G | F(4NO₂) | Tic | E |
| 468 | Suc | E | G | 3Pal | Tic | E |
| 469 | Suc | E | G | Y(Me) | Tic | E |
| 470 | Suc | E | G | Pip | Tic | E |
| 471 | Suc | E | G | Tic | Tic | E |
| 472 | Suc | E | G | 1Nal | Tic | E |
| 473 | Suc | E | G | F(2F) | Tic | E |
| 474 | Suc | E | G | F(3F) | Tic | E |
| 475 | Suc | E | G | F(4F) | Tic | E |
| 476 | Suc | E | G | F | 1Nal | E |
| 477 | Suc | E | G | Y | 1Nal | E |
| 478 | Suc | E | G | W | 1Nal | E |
| 479 | Suc | E | G | F(3Br) | 1Nal | E |
| 480 | Suc | E | G | F(4NO₂) | 1Nal | E |
| 481 | Suc | E | G | 3Pal | 1Nal | E |
| 482 | Suc | E | G | Y(Me) | 1Nal | E |
| 483 | Suc | E | G | Pip | 1Nal | E |
| 484 | Suc | E | G | Tic | 1Nal | E |
| 485 | Suc | E | G | 1Nal | 1Nal | E |
| 486 | Suc | E | G | F(2F) | 1Nal | E |
| 487 | Suc | E | G | F(3F) | 1Nal | E |
| 488 | Suc | E | G | F(4F) | 1Nal | E |
| 489 | Suc | E | G | F | F(2F) | E |
| 490 | Suc | E | G | Y | F(2F) | E |
| 491 | Suc | E | G | W | F(2F) | E |
| 492 | Suc | E | G | F(3Br) | F(2F) | E |
| 493 | Suc | E | G | F(4NO₂) | F(2F) | E |
| 494 | Suc | E | G | 3Pal | F(2F) | E |
| 495 | Suc | E | G | Y(Me) | F(2F) | E |
| 496 | Suc | E | G | Pip | F(2F) | E |
| 497 | Suc | E | G | Tic | F(2F) | E |
| 498 | Suc | E | G | 1Nal | F(2F) | E |
| 499 | Suc | E | G | F(2F) | F(2F) | E |
| 500 | Suc | E | G | F(3F) | F(2F) | E |
| 501 | Suc | E | G | F(4F) | F(2F) | E |
| 502 | Suc | E | G | F | F(3F) | E |
| 503 | Suc | E | G | Y | F(3F) | E |
| 504 | Suc | E | G | W | F(3F) | E |
| 505 | Suc | E | G | F(3Br) | F(3F) | E |
| 506 | Suc | E | G | F(4NO₂) | F(3F) | E |
| 507 | Suc | E | G | 3Pal | F(3F) | E |
| 508 | Suc | E | G | Y(Me) | F(3F) | E |
| 509 | Suc | E | G | Pip | F(3F) | E |
| 510 | Suc | E | G | Tic | F(3F) | E |
| 511 | Suc | E | G | 1Nal | F(3F) | E |
| 512 | Suc | E | G | F(2F) | F(3F) | E |
| 513 | Suc | E | G | F(3F) | F(3F) | E |
| 514 | Suc | E | G | F(4F) | F(3F) | E |
| 515 | Suc | E | G | F | F(4F) | E |
| 516 | Suc | E | G | Y | F(4F) | E |
| 517 | Suc | E | G | W | F(4F) | E |
| 518 | Suc | E | G | F(3Br) | F(4F) | E |
| 519 | Suc | E | G | F(4NO₂) | F(4F) | E |
| 520 | Suc | E | G | 3Pal | F(4F) | E |
| 521 | Suc | E | G | Y(Me) | F(4F) | E |
| 522 | Suc | E | G | Pip | F(4F) | E |
| 523 | Suc | E | G | Tic | F(4F) | E |
| 524 | Suc | E | G | 1Nal | F(4F) | E |
| 525 | Suc | E | G | F(2F) | F(4F) | E |
| 526 | Suc | E | G | F(3F) | F(4F) | E |
| 527 | Suc | E | G | F(4F) | F(4F) | E |
| 528 | Suc | E | G | F | F | E |
| 529 | Suc | Q | G | F | F | E |
| 530 | Suc | N | G | F | F | E |
| 531 | Suc | (Me)E | G | F | F | E |
| 532 | Suc | βE | G | F | F | E |
| 533 | Suc | (Me)D | G | F | F | E |
| 534 | Suc | βD | G | F | F | E |
| 535 | Suc | E(Me) | G | F | F | E |
| 536 | Suc | D | G | F | F | E |
| 537 | Suc | Gla | G | F | F | E |
| 538 | Suc | Q | G | F | F | Q |
| 539 | Suc | N | G | F | F | Q |
| 540 | Suc | (Me)E | G | F | F | Q |
| 541 | Suc | βE | G | F | F | Q |
| 542 | Suc | (Me)D | G | F | F | Q |
| 543 | Suc | βD | G | F | F | Q |
| 544 | Suc | E(Me) | G | F | F | Q |
| 545 | Suc | D | G | F | F | Q |
| 546 | Suc | Gla | G | F | F | Q |
| 547 | Suc | E | G | F | F | Q |
| 548 | Suc | Q | G | F | F | N |
| 549 | Suc | N | G | F | F | N |
| 550 | Suc | (Me)E | G | F | F | N |
| 551 | Suc | βE | G | F | F | N |
| 552 | Suc | (Me)D | G | F | F | N |
| 553 | Suc | βD | G | F | F | N |
| 554 | Suc | E(Me) | G | F | F | N |
| 555 | Suc | D | G | F | F | N |
| 556 | Suc | Gla | G | F | F | N |
| 557 | Suc | E | G | F | F | N |
| 558 | Suc | Q | G | F | F | (Me)E |
| 559 | Suc | N | G | F | F | (Me)E |
| 560 | Suc | (Me)E | G | F | F | (Me)E |
| 561 | Suc | βE | G | F | F | (Me)E |
| 562 | Suc | (Me)D | G | F | F | (Me)E |
| 563 | Suc | βD | G | F | F | (Me)E |
| 564 | Suc | E(Me) | G | F | F | (Me)E |
| 565 | Suc | D | G | F | F | (Me)E |
| 566 | Suc | Gla | G | F | F | (Me)E |
| 567 | Suc | E | G | F | F | (Me)E |
| 568 | Suc | Q | G | F | F | βE |
| 569 | Suc | N | G | F | F | βE |
| 570 | Suc | (Me)E | G | F | F | βE |
| 571 | Suc | βE | G | F | F | βE |
| 572 | Suc | (Me)D | G | F | F | βE |
| 573 | Suc | βD | G | F | F | βE |
| 574 | Suc | E(Me) | G | F | F | βE |
| 575 | Suc | D | G | F | F | βE |
| 576 | Suc | Gla | G | F | F | βE |
| 577 | Suc | E | G | F | F | βE |
| 578 | Suc | Q | G | F | F | (Me)D |
| 579 | Suc | N | G | F | F | (Me)D |
| 580 | Suc | (Me)E | G | F | F | (Me)D |
| 581 | Suc | βE | G | F | F | (Me)D |
| 582 | Suc | (Me)D | G | F | F | (Me)D |
| 583 | Suc | βD | G | F | F | (Me)D |
| 584 | Suc | E(Me) | G | F | F | (Me)D |
| 585 | Suc | D | G | F | F | (Me)D |
| 586 | Suc | Gla | G | F | F | (Me)D |
| 587 | Suc | E | G | F | F | (Me)D |
| 588 | Suc | Q | G | F | F | βD |
| 589 | Suc | N | G | F | F | βD |
| 590 | Suc | (Me)E | G | F | F | βD |
| 591 | Suc | βE | G | F | F | βD |
| 592 | Suc | (Me)D | G | F | F | βD |
| 593 | Suc | βD | G | F | F | βD |
| 594 | Suc | E(Me) | G | F | F | βD |
| 595 | Suc | D | G | F | F | βD |
| 596 | Suc | Gla | G | F | F | βD |
| 597 | Suc | E | G | F | F | βD |
| 598 | Suc | Q | G | F | F | E(Me) |
| 599 | Suc | N | G | F | F | E(Me) |
| 600 | Suc | (Me)E | G | F | F | E(Me) |
| 601 | Suc | βE | G | F | F | E(Me) |
| 602 | Suc | (Me)D | G | F | F | E(Me) |
| 603 | Suc | βD | G | F | F | E(Me) |
| 604 | Suc | E(Me) | G | F | F | E(Me) |
| 605 | Suc | D | G | F | F | E(Me) |
| 606 | Suc | Gla | G | F | F | E(Me) |
| 607 | Suc | E | G | F | F | E(Me) |
| 608 | Suc | Q | G | F | F | D |
| 609 | Suc | N | G | F | F | D |
| 610 | Suc | (Me)E | G | F | F | D |
| 611 | Suc | βE | G | F | F | D |
| 612 | Suc | (Me)D | G | F | F | D |
| 613 | Suc | βD | G | F | F | D |
| 614 | Suc | E(Me) | G | F | F | D |
| 615 | Suc | D | G | F | F | D |
| 616 | Suc | Gla | G | F | F | D |
| 617 | Suc | E | G | F | F | D |
| 618 | Suc | Q | G | F | F | Gla |
| 619 | Suc | N | G | F | F | Gla |
| 620 | Suc | (Me)E | G | F | F | Gla |
| 621 | Suc | βE | G | F | F | Gla |
| 622 | Suc | (Me)D | G | F | F | Gla |
| 623 | Suc | βD | G | F | F | Gla |
| 624 | Suc | E(Me) | G | F | F | Gla |
| 625 | Suc | D | G | F | F | Gla |
| 626 | Suc | Gla | G | F | F | Gla |
| 627 | Suc | E | G | F | F | Gla |
| 628 | Suc | E | G | F | F | E |
| 629 | Suc | E | G | F | F | F |
| 630 | Suc | E | G | F | F | Y |
| 631 | Suc | E | G | F | F | W |
| 632 | Suc | E | G | F | F | F(3Br) |
| 633 | Suc | E | G | F | F | F(4NO₂) |
| 634 | Suc | E | G | F | F | 3Pal |
| 635 | Suc | E | G | F | F | Y(Me) |
| 636 | Suc | E | G | F | F | Pip |
| 637 | Suc | E | G | F | F | Tic |
| 638 | Suc | E | G | F | F | 1Nal |
| 639 | Suc | E | G | F | F | F(2F) |
| 640 | Suc | E | G | F | F | F(3F) |
| 641 | Suc | E | G | F | F | F(4F) |
| 642 | Suc | E | G | Y | F | E |
| 643 | Suc | E | G | Y | F | F |
| 644 | Suc | E | G | Y | F | Y |
| 645 | Suc | E | G | Y | F | W |
| 646 | Suc | E | G | Y | F | F(3Br) |
| 647 | Suc | E | G | Y | F | F(4NO₂) |
| 648 | Suc | E | G | Y | F | 3Pal |
| 649 | Suc | E | G | Y | F | Y(Me) |
| 650 | Suc | E | G | Y | F | Pip |
| 651 | Suc | E | G | Y | F | Tic |
| 652 | Suc | E | G | Y | F | 1Nal |
| 653 | Suc | E | G | Y | F | F(2F) |
| 654 | Suc | E | G | Y | F | F(3F) |
| 655 | Suc | E | G | Y | F | F(4F) |
| 656 | Suc | E | G | F | F | E |
| 657 | Suc | E | A | F | F | E |
| 658 | Suc | E | a | F | F | E |
| 659 | Suc | E | βA | F | F | E |
| 660 | Suc | E | P | F | F | E |
| 661 | Suc | E | p | F | F | E |
| 662 | Suc | E | Sar | F | F | E |
| 663 | Suc | E | β-H-ala | F | F | E |
| 664 | Suc | E | G | Y | F | E |
| 665 | Suc | E | A | Y | F | E |
| 666 | Suc | E | a | Y | F | E |
| 667 | Suc | E | βA | Y | F | E |
| 668 | Suc | E | P | Y | F | E |
| 669 | Suc | E | p | Y | F | E |
| 670 | Suc | E | Car | V | F | E |
| 671 | Suc | E | β-H-ala | Y | F | E |
| 672 | Suc | E | G | F | F | E |
| 673 | Suc | E | G | F | F | E(Me) |
| 674 | Suc | E | G | Y | F | E(Me) |
| 675 | Suc | E | G | W | F | E(Me) |
| 676 | Suc | E | G | 1Nal | F | E(Me) |
| 677 | Suc | E | G | Tic | F | E(Me) |
| 678 | Suc | E | G | Pip | F | E(Me) |
| 679 | Suc | E | G | F(3Br) | F | E(Me) |
| 680 | Suc | E | G | 3Pal | F | E(Me) |
| 681 | Suc | E | G | F | Y | E(Me) |
| 682 | Suc | E | G | Y | Y | E(Me) |
| 683 | Suc | E | G | W | Y | E(Me) |
| 684 | Suc | E | G | 1Nal | Y | E(Me) |
| 685 | Suc | E | G | Tic | Y | E(Me) |
| 686 | Suc | E | G | Pip | Y | E(Me) |
| 687 | Suc | E | G | F(3Br) | Y | E(Me) |
| 688 | Suc | E | G | 3Pal | Y | E(Me) |
| 689 | Suc | E | G | F | W | E(Me) |
| 690 | Suc | E | G | Y | W | E(Me) |
| 691 | Suc | E | G | W | W | E(Me) |
| 692 | Suc | E | G | 1Nal | W | E(Me) |
| 693 | Suc | E | G | Tic | W | E(Me) |
| 694 | Suc | E | G | Pip | W | E(Me) |
| 695 | Suc | E | G | F(3Br) | W | E(Me) |
| 696 | Suc | E | G | 3Pal | W | E(Me) |
| 697 | Suc | E | G | F | 1Nal | E(Me) |
| 698 | Suc | E | G | Y | 1Nal | E(Me) |
| 699 | Suc | E | G | W | 1Nal | E(Me) |
| 700 | Suc | E | G | 1Nal | 1Nal | E(Me) |
| 701 | Suc | E | G | Tic | 1Nal | E(Me) |
| 702 | Suc | E | G | Pip | 1Nal | E(Me) |
| 703 | Suc | E | G | F(3Br) | 1Nal | E(Me) |
| 704 | Suc | E | G | 3Pal | 1Nal | E(Me) |
| 705 | Suc | E | G | F | Tic | E(Me) |
| 706 | Suc | E | G | Y | Tic | E(Me) |
| 707 | Suc | E | G | W | Tic | E(Me) |
| 708 | Suc | E | G | 1Nal | Tic | E(Me) |
| 709 | Suc | E | G | Tic | Tic | E(Me) |
| 710 | Suc | E | G | Pip | Tic | E(Me) |
| 711 | Suc | E | G | F(3Br) | Tic | E(Me) |
| 712 | Suc | E | G | 3Pal | Tic | E(Me) |
| 713 | Suc | E | G | F | Pip | E(Me) |
| 714 | Suc | E | G | Y | Pip | E(Me) |
| 715 | Suc | E | G | W | Pip | E(Me) |
| 716 | Suc | E | G | 1Nal | Pip | E(Me) |
| 717 | Suc | E | G | Tic | Pip | E(Me) |
| 718 | Suc | E | G | Pip | Pip | E(Me) |
| 719 | Suc | E | G | F(3Br) | Pip | E(Me) |
| 720 | Suc | E | G | 3Pal | Pip | E(Me) |
| 721 | Suc | E | G | F | F(3Br) | E(Me) |
| 722 | Suc | E | G | Y | F(3Br) | E(Me) |
| 723 | Suc | E | G | W | F(3Br) | E(Me) |
| 724 | Suc | E | G | 1Nal | F(3Br) | E(Me) |
| 725 | Suc | E | G | Tic | F(3Br) | E(Me) |
| 726 | Suc | E | G | Pip | F(3Br) | E(Me) |
| 727 | Suc | E | G | F(3Br) | F(3Br) | E(Me) |
| 728 | Suc | E | G | 3Pal | F(3Br) | E(Me) |
| 729 | Suc | E | G | F | 3Pal | E(Me) |
| 730 | Suc | E | G | Y | 3Pal | E(Me) |
| 731 | Suc | E | G | W | 3Pal | E(Me) |
| 732 | Suc | E | G | 1Nal | 3Pal | E(Me) |
| 733 | Suc | E | G | Tic | 3Pal | E(Me) |
| 734 | Suc | E | G | Pip | 3Pal | E(Me) |
| 735 | Suc | E | G | F(3Br) | 3Pal | E(Me) |
| 736 | Suc | E | G | 3Pal | 3Pal | E(Me) |
| 737 | Suc | E | Ahx | F | F | E |
| 738 | Suc | Ahx | G | F | F | E |
| 739 | Suc | E | Ahx | F | F | E |
| 740 | Suc | E | G | Ahx | F | E |
| 741 | Suc | E | G | F | Ahx | E |

TABLE 6
Sequence of βTrCP binding peptides (4-mers) synthesised

| | P1 | P2 | P3 | P4 | P5 | P6 |
|---|---|---|---|---|---|---|
| 1 | Suc | E | G | F | F | |
| 2 | Suc | E | G | Y | F | |
| 3 | Suc | E | G | W | F | |
| 4 | Suc | E | G | F(3Br) | F | |
| 5 | Suc | E | G | F(4NO₂) | F | |
| 6 | Suc | E | G | 3Pal | F | |
| 7 | Suc | E | G | Y(Me) | F | |
| 8 | Suc | E | G | Pip | F | |
| 9 | Suc | E | G | Tic | F | |
| 10 | Suc | E | G | 1Nal | F | |
| 11 | Suc | E | G | F(2F) | F | |
| 12 | Suc | E | G | F(3F) | F | |
| 13 | Suc | E | G | F(4F) | F | |
| 14 | Suc | E | G | F | Y | |
| 15 | Suc | E | G | Y | Y | |
| 16 | Suc | E | G | W | Y | |
| 17 | Suc | E | G | F(3Br) | Y | |
| 18 | Suc | E | G | F(4NO₂) | Y | |
| 19 | Suc | E | G | 3Pal | Y | |
| 20 | Suc | E | G | Y(Me) | Y | |
| 21 | Suc | E | G | Pip | Y | |
| 22 | Suc | E | G | Tic | Y | |
| 23 | Suc | E | G | 1Nal | Y | |
| 24 | Suc | E | G | F(2F) | Y | |
| 25 | Suc | E | G | F(3F) | Y | |
| 26 | Suc | E | G | F(4F) | Y | |
| 27 | Suc | E | G | F | W | |
| 28 | Suc | E | G | Y | W | |
| 29 | Suc | E | G | W | W | |
| 30 | Suc | E | G | F(3Br) | W | |
| 31 | Suc | E | G | F(4NO₂) | W | |
| 32 | Suc | E | G | 3Pal | W | |
| 33 | Suc | E | G | Y(Me) | W | |
| 34 | Suc | E | G | Pip | W | |
| 35 | Suc | E | G | Tic | W | |
| 36 | Suc | E | G | 1Nal | W | |
| 37 | Suc | E | G | F(2F) | W | |
| 38 | Suc | E | G | F(3F) | W | |
| 39 | Suc | E | G | F(4F) | W | |
| 40 | Suc | E | G | F | F(3Br) | |
| 41 | Suc | E | G | Y | F(3Br) | |
| 42 | Suc | E | G | W | F(3Br) | |
| 43 | Suc | E | G | F(3Br) | F(3Br) | |
| 44 | Suc | E | G | F(4NO₂) | F(3Br) | |
| 45 | Suc | E | G | 3Pal | F(3Br) | |
| 46 | Suc | E | G | Y(Me) | F(3Br) | |
| 47 | Suc | E | G | Pip | F(3Br) | |
| 48 | Suc | E | G | Tic | F(3Br) | |
| 49 | Suc | E | G | 1Nal | F(3Br) | |
| 50 | Suc | E | G | F(2F) | F(3Br) | |
| 51 | Suc | E | G | F(3F) | F(3Br) | |
| 52 | Suc | E | G | F(4F) | F(3Br) | |
| 53 | Suc | E | G | F | F(4NO₂) | |
| 54 | Suc | E | G | Y | F(4NO₂) | |
| 55 | Suc | E | G | W | F(4NO₂) | |
| 56 | Suc | E | G | F(3Br) | F(4NO₂) | |
| 57 | Suc | E | G | F(4NO₂) | F(4NO₂) | |
| 58 | Suc | E | G | 3Pal | F(4NO₂) | |
| 59 | Suc | E | G | Y(Me) | F(4NO₂) | |
| 60 | Suc | E | G | Pip | F(4NO₂) | |
| 61 | Suc | E | G | Tic | F(4NO₂) | |
| 62 | Suc | E | G | 1Nal | F(4NO₂) | |
| 63 | Suc | E | G | F(2F) | F(4NO₂) | |
| 64 | Suc | E | G | F(3F) | F(4NO₂) | |
| 65 | Suc | E | G | F(4F) | F(4NO₂) | |
| 66 | Suc | E | G | F | 3Pal | |
| 67 | Suc | E | G | Y | 3Pal | |
| 68 | Suc | E | G | W | 3Pal | |
| 69 | Suc | E | G | F(3Br) | 3Pal | |
| 70 | Suc | E | G | F(4NO₂) | 3Pal | |
| 71 | Suc | E | G | 3Pal | 3Pal | |
| 72 | Suc | E | G | Y(Me) | 3Pal | |
| 73 | Suc | E | G | Pip | 3Pal | |
| 74 | Suc | E | G | Tic | 3Pal | |
| 75 | Suc | E | G | 1Nal | 3Pal | |
| 76 | Suc | E | G | F(2F) | 3Pal | |
| 77 | Suc | E | G | F(3F) | 3Pal | |
| 78 | Suc | E | G | F(4F) | 3Pal | |
| 79 | Suc | E | G | F | Y(Me) | |
| 80 | Suc | E | G | Y | Y(Me) | |
| 81 | Suc | E | G | W | Y(Me) | |
| 82 | Suc | E | G | F(3Br) | Y(Me) | |
| 83 | Suc | E | G | F(4NO₂) | Y(Me) | |
| 84 | Suc | E | G | 3Pal | Y(Me) | |
| 85 | Suc | E | G | Y(Me) | Y(Me) | |
| 86 | Suc | E | G | Pip | Y(Me) | |
| 87 | Suc | E | G | Tic | Y(Me) | |
| 88 | Suc | E | G | 1Nal | Y(Me) | |
| 89 | Suc | E | G | F(2F) | Y(Me) | |
| 90 | Suc | E | G | F(3F) | Y(Me) | |
| 91 | Suc | E | G | F(4F) | Y(Me) | |
| 92 | Suc | E | G | F | Pip | |
| 93 | Suc | E | G | Y | Pip | |
| 94 | Suc | E | G | W | Pip | |
| 95 | Suc | E | G | F(3Br) | Pip | |
| 96 | Suc | E | G | F(4NO₂) | Pip | |
| 97 | Suc | E | G | 3Pal | Pip | |
| 98 | Suc | E | G | Y(Me) | Pip | |
| 99 | Suc | E | G | Pip | Pip | |
| 100 | Suc | E | G | Tic | Pip | |
| 101 | Suc | E | G | 1Nal | Pip | |
| 102 | Suc | E | G | F(2F) | Pip | |
| 103 | Suc | E | G | F(3F) | Pip | |
| 104 | Suc | E | G | F(4F) | Pip | |
| 105 | Suc | E | G | F | Tic | |
| 106 | Suc | E | G | Y | Tic | |
| 107 | Suc | E | G | W | Tic | |
| 108 | Suc | E | G | F(3Br) | Tic | |
| 109 | Suc | E | G | F(4NO₂) | Tic | |
| 110 | Suc | E | G | 3Pal | Tic | |
| 111 | Suc | E | G | Y(Me) | Tic | |
| 112 | Suc | E | G | Pip | Tic | |
| 113 | Suc | E | G | Tic | Tic | |
| 114 | Suc | E | G | 1Nal | Tic | |
| 115 | Suc | E | G | F(2F) | Tic | |
| 116 | Suc | E | G | F(3F) | Tic | |
| 117 | Suc | E | G | F(4F) | Tic | |
| 118 | Suc | E | G | F | 1Nal | |
| 119 | Suc | E | G | Y | 1Nal | |
| 120 | Suc | E | G | W | 1Nal | |
| 121 | Suc | E | G | F(3Br) | 1Nal | |
| 122 | Suc | E | G | F(4NO₂) | 1Nal | |
| 123 | Suc | E | G | 3Pal | 1Nal | |
| 124 | Suc | E | G | Y(Me) | 1Nal | |
| 125 | Suc | E | G | Pip | 1Nal | |
| 126 | Suc | E | G | Tic | 1Nal | |
| 127 | Suc | E | G | 1Nal | 1Nal | |
| 128 | Suc | E | G | F(2F) | 1Nal | |
| 129 | Suc | E | G | F(3F) | 1Nal | |
| 130 | Suc | E | G | F(4F) | 1Nal | |
| 131 | Suc | E | G | F | F(2F) | |
| 132 | Suc | E | G | Y | F(2F) | |
| 133 | Suc | E | G | W | F(2F) | |
| 134 | Suc | E | G | F(3Br) | F(2F) | |
| 135 | Suc | E | G | F(4NO₂) | F(2F) | |
| 136 | Suc | E | G | 3Pal | F(2F) | |
| 137 | Suc | E | G | Y(Me) | F(2F) | |
| 138 | Suc | E | G | Pip | F(2F) | |
| 139 | Suc | E | G | Tic | F(2F) | |
| 140 | Suc | E | G | 1Nal | F(2F) | |
| 141 | Suc | E | G | F(2F) | F(2F) | |
| 142 | Suc | E | G | F(3F) | F(2F) | |
| 143 | Suc | E | G | F(4F) | F(2F) | |
| 144 | Suc | E | G | F | F(3F) | |
| 145 | Suc | E | G | Y | F(3F) | |
| 146 | Suc | E | G | W | F(3F) | |
| 147 | Suc | E | G | F(3Br) | F(3F) | |
| 148 | Suc | E | G | F(4NO₂) | F(3F) | |
| 149 | Suc | E | G | 3Pal | F(3F) | |
| 150 | Suc | E | G | Y(Me) | F(3F) | |
| 151 | Suc | E | G | Pip | F(3F) | |
| 152 | Suc | E | G | Tic | F(3F) | |
| 153 | Suc | E | G | 1Nal | F(3F) | |
| 154 | Suc | E | G | F(2F) | F(3F) | |
| 155 | Suc | E | G | F(3F) | F(3F) | |
| 156 | Suc | E | G | F(4F) | F(3F) | |
| 157 | Suc | E | G | F | F(4F) | |
| 158 | Suc | E | G | Y | F(4F) | |
| 159 | Suc | E | G | W | F(4F) | |
| 160 | Suc | E | G | F(3Br) | F(4F) | |
| 161 | Suc | E | G | F(4NO₂) | F(4F) | |
| 162 | Suc | E | G | 3Pal | F(4F) | |
| 163 | Suc | E | G | Y(Me) | F(4F) | |
| 164 | Suc | E | G | Pip | F(4F) | |
| 165 | Suc | E | G | Tic | F(4F) | |
| 166 | Suc | E | G | 1Nal | F(4F) | |
| 167 | Suc | E | G | F(2F) | F(4F) | |
| 168 | Suc | E | G | F(3F) | F(4F) | |
| 169 | Suc | E | G | F(4F) | F(4F) | |
| 170 | Suc | E(Me) | G | F | F | |
| 171 | Suc | E(Me) | G | Y | F | |
| 172 | Suc | E(Me) | G | W | F | |
| 173 | Suc | E(Me) | G | F(3Br) | F | |
| 174 | Suc | E(Me) | G | F(4NO₂) | F | |
| 175 | Suc | E(Me) | G | 3Pal | F | |
| 176 | Suc | E(Me) | G | Y(Me) | F | |
| 177 | Suc | E(Me) | G | Pip | F | |
| 178 | Suc | E(Me) | G | Tic | F | |
| 179 | Suc | E(Me) | G | 1Nal | F | |
| 180 | Suc | E(Me) | G | F(2F) | F | |
| 181 | Suc | E(Me) | G | F(3F) | F | |
| 182 | Suc | E(Me) | G | F(4F) | F | |
| 183 | Suc | E(Me) | G | F | Y | |
| 184 | Suc | E(Me) | G | Y | Y | |
| 185 | Suc | E(Me) | G | W | Y | |
| 186 | Suc | E(Me) | G | F(3Br) | Y | |
| 187 | Suc | E(Me) | G | F(4NO₂) | Y | |
| 188 | Suc | E(Me) | G | 3Pal | Y | |
| 189 | Suc | E(Me) | G | Y(Me) | Y | |
| 190 | Suc | E(Me) | G | Pip | Y | |
| 191 | Suc | E(Me) | G | Tic | Y | |
| 192 | Suc | E(Me) | G | 1Nal | Y | |
| 193 | Suc | E(Me) | G | F(2F) | Y | |
| 194 | Suc | E(Me) | G | F(3F) | Y | |
| 195 | Suc | E(Me) | G | F(4F) | Y | |
| 196 | Suc | E(Me) | G | F | W | |
| 197 | Suc | E(Me) | G | Y | W | |
| 198 | Suc | E(Me) | G | W | W | |
| 199 | Suc | E(Me) | G | F(3Br) | W | |
| 200 | Suc | E(Me) | G | F(4NO₂) | W | |
| 201 | Suc | E(Me) | G | 3Pal | W | |
| 202 | Suc | E(Me) | G | Y(Me) | W | |
| 203 | Suc | E(Me) | G | Pip | W | |
| 204 | Suc | E(Me) | G | Tic | W | |
| 205 | Suc | E(Me) | G | 1Nal | W | |
| 206 | Suc | E(Me) | G | F(2F) | W | |
| 207 | Suc | E(Me) | G | F(3F) | W | |
| 208 | Suc | E(Me) | G | F(4F) | W | |
| 209 | Suc | E(Me) | G | F | F(3Br) | |
| 210 | Suc | E(Me) | G | Y | F(3Br) | |
| 211 | Suc | E(Me) | G | W | F(3Br) | |
| 212 | Suc | E(Me) | G | F(3Br) | F(3Br) | |
| 213 | Suc | E(Me) | G | F(4NO₂) | F(3Br) | |
| 214 | Suc | E(Me) | G | 3Pal | F(3Br) | |
| 215 | Suc | E(Me) | G | Y(Me) | F(3Br) | |
| 216 | Suc | E(Me) | G | Pip | F(3Br) | |
| 217 | Suc | E(Me) | G | Tic | F(3Br) | |
| 218 | Suc | E(Me) | G | 1Nal | F(3Br) | |
| 219 | Suc | E(Me) | G | F(2F) | F(3Br) | |
| 220 | Suc | E(Me) | G | F(3F) | F(3Br) | |
| 221 | Suc | E(Me) | G | F(4F) | F(3Br) | |
| 222 | Suc | E(Me) | G | F | F(4NO₂) | |
| 223 | Suc | E(Me) | G | Y | F(4NO₂) | |
| 224 | Suc | E(Me) | G | W | F(4NO₂) | |
| 225 | Suc | E(Me) | G | F(3Br) | F(4NO₂) | |
| 226 | Suc | E(Me) | G | F(4NO₂) | F(4NO₂) | |
| 227 | Suc | E(Me) | G | 3Pal | F(4NO₂) | |
| 228 | Suc | E(Me) | G | Y(Me) | F(4NO₂) | |
| 229 | Suc | E(Me) | G | Pip | F(4NO₂) | |
| 230 | Suc | E(Me) | G | Tic | F(4NO₂) | |
| 231 | Suc | E(Me) | G | 1Nal | F(4NO₂) | |
| 232 | Suc | E(Me) | G | F(2F) | F(4NO₂) | |
| 233 | Suc | E(Me) | G | F(3F) | F(4NO₂) | |
| 234 | Suc | E(Me) | G | F(4F) | F(4NO₂) | |
| 235 | Suc | E(Me) | G | F | 3Pal | |
| 236 | Suc | E(Me) | G | Y | 3Pal | |
| 237 | Suc | E(Me) | G | W | 3Pal | |
| 238 | Suc | E(Me) | G | F(3Br) | 3Pal | |
| 239 | Suc | E(Me) | G | F(4NO2) | 3Pal | |
| 240 | Suc | E(Me) | G | 3Pal | 3Pal | |
| 241 | Suc | E(Me) | G | Y(Me) | 3Pal | |
| 242 | Suc | E(Me) | G | Pip | 3Pal | |
| 243 | Suc | E(Me) | G | Tic | 3Pal | |
| 244 | Suc | E(Me) | G | 1Nal | 3Pal | |
| 245 | Suc | E(Me) | G | F(2F) | 3Pal | |
| 246 | Suc | E(Me) | G | F(3F) | 3Pal | |
| 247 | Suc | E(Me) | G | F(4F) | 3Pal | |
| 248 | Suc | E(Me) | G | F | Y(Me) | |
| 249 | Suc | E(Me) | G | V | Y(Me) | |
| 250 | Suc | E(Me) | G | W | Y(Me) | |
| 251 | Suc | E(Me) | G | F(3Br) | Y(Me) | |
| 252 | Suc | E(Me) | G | F(4NO₂) | Y(Me) | |
| 253 | Suc | E(Me) | G | 3Pal | Y(Me) | |
| 254 | Suc | E(Me) | G | Y(Me) | Y(Me) | |
| 255 | Suc | E(Me) | G | Pip | Y(Me) | |
| 256 | Suc | E(Me) | G | Tic | Y(Me) | |
| 257 | Suc | E(Me) | G | 1Nal | Y(Me) | |
| 258 | Suc | E(Me) | G | F(2F) | Y(Me) | |
| 259 | Suc | E(Me) | G | F(3F) | Y(Me) | |
| 260 | Suc | E(Me) | G | F(4F) | Y(Me) | |
| 261 | Suc | E(Me) | G | F | Pip | |
| 262 | Suc | E(Me) | G | Y | Pip | |
| 263 | Suc | E(Me) | G | W | Pip | |
| 264 | Suc | E(Me) | G | F(3Br) | Pip | |
| 265 | Suc | E(Me) | G | F(4NO₂) | Pip | |
| 266 | Suc | E(Me) | G | 3Pal | Pip | |
| 267 | Suc | E(Me) | G | Y(Me) | Pip | |
| 268 | Suc | E(Me) | G | Pip | Pip | |
| 269 | Suc | E(Me) | G | Tic | Pip | |
| 270 | Suc | E(Me) | G | 1Nal | Pip | |
| 271 | Suc | E(Me) | G | F(2F) | Pip | |
| 272 | Suc | E(Me) | G | F(3F) | Pip | |
| 273 | Suc | E(Me) | G | F(4F) | Pip | |
| 274 | Suc | E(Me) | G | F | Tic | |
| 275 | Suc | E(Me) | G | Y | Tic | |
| 276 | Suc | E(Me) | G | W | Tic | |
| 277 | Suc | E(Me) | G | F(3Br) | Tic | |
| 278 | Suc | E(Me) | G | F(4NO₂) | Tic | |
| 279 | Suc | E(Me) | G | 3Pal | Tic | |
| 280 | Suc | E(Me) | G | Y(Me) | Tic | |
| 281 | Suc | E(Me) | G | Pip | Tic | |
| 282 | Suc | E(Me) | G | Tic | Tic | |
| 283 | Suc | E(Me) | G | 1Nal | Tic | |
| 284 | Suc | E(Me) | G | F(2F) | Tic | |
| 285 | Suc | E(Me) | G | F(3F) | Tic | |
| 286 | Suc | E(Me) | G | F(4F) | Tic | |
| 287 | Suc | E(Me) | G | F | 1Nal | |
| 288 | Suc | E(Me) | G | Y | 1Nal | |
| 289 | Suc | E(Me) | G | W | 1Nal | |
| 290 | Suc | E(Me) | G | F(3Br) | 1Nal | |
| 291 | Suc | E(Me) | G | F(4NO₂) | 1Nal | |
| 292 | Suc | E(Me) | G | 3Pal | 1Nal | |
| 293 | Suc | E(Me) | G | Y(Me) | 1Nal | |
| 294 | Suc | E(Me) | G | Pip | 1Nal | |
| 295 | Suc | E(Me) | G | Tic | 1Nal | |
| 296 | Suc | E(Me) | G | 1Nal | 1Nal | |
| 297 | Suc | E(Me) | G | F(2F) | 1Nal | |
| 298 | Suc | E(Me) | G | F(3F) | 1Nal | |
| 299 | Suc | E(Me) | G | F(4F) | 1Nal | |
| 300 | Suc | E(Me) | G | F | F(2F) | |
| 301 | Suc | E(Me) | G | Y | F(2F) | |
| 302 | Suc | E(Me) | G | W | F(2F) | |
| 303 | Suc | E(Me) | G | F(3Br) | F(2F) | |
| 304 | Suc | E(Me) | G | F(4NO₂) | F(2F) | |
| 305 | Suc | E(Me) | G | 3Pal | F(2F) | |
| 306 | Suc | E(Me) | G | Y(Me) | F(2F) | |
| 307 | Suc | E(Me) | G | Pip | F(2F) | |
| 308 | Suc | E(Me) | G | Tic | F(2F) | |
| 309 | Suc | E(Me) | G | 1Nal | F(2F) | |
| 310 | Suc | E(Me) | G | F(2F) | F(2F) | |
| 311 | Suc | E(Me) | G | F(3F) | F(2F) | |
| 312 | Suc | E(Me) | G | F(4F) | F(2F) | |
| 313 | Suc | E(Me) | G | F | F(3F) | |
| 314 | Suc | E(Me) | G | Y | F(3F) | |
| 315 | Suc | E(Me) | G | W | F(3F) | |
| 316 | Suc | E(Me) | G | F(3Br) | F(3F) | |
| 317 | Suc | E(Me) | G | F(4NO₂) | F(3F) | |
| 318 | Suc | E(Me) | G | 3Pal | F(3F) | |
| 319 | Suc | E(Me) | G | Y(Me) | F(3F) | |
| 320 | Suc | E(Me) | G | Pip | F(3F) | |
| 321 | Suc | E(Me) | G | Tic | F(3F) | |
| 322 | Suc | E(Me) | G | 1Nal | F(3F) | |
| 323 | Suc | E(Me) | G | F(2F) | F(3F) | |
| 324 | Suc | E(Me) | G | F(3F) | F(3F) | |
| 325 | Suc | E(Me) | G | F(4F) | F(3F) | |
| 326 | Suc | E(Me) | G | F | F(4F) | |
| 327 | Suc | E(Me) | G | Y | F(4F) | |
| 328 | Suc | E(Me) | G | W | F(4F) | |
| 329 | Suc | E(Me) | G | F(3Br) | F(4F) | |
| 330 | Suc | E(Me) | G | F(4NO₂) | F(4F) | |
| 331 | Suc | E(Me) | G | 3Pal | F(4F) | |
| 332 | Suc | E(Me) | G | Y(Me) | F(4F) | |
| 333 | Suc | E(Me) | G | Pip | F(4F) | |
| 334 | Suc | E(Me) | G | Tic | F(4F) | |
| 335 | Suc | E(Me) | G | 1Nal | F(4F) | |
| 336 | Suc | E(Me) | G | F(2F) | F(4F) | |
| 337 | Suc | E(Me) | G | F(3F) | F(4F) | |
| 338 | Suc | E(Me) | G | F(4F) | F(4F) | |
| 339 | Suc | E | G | F | F | |
| 340 | Suc | Q | G | F | F | |
| 341 | Suc | N | G | F | F | |
| 342 | Suc | (MeE | G | F | F | |
| 343 | Suc | βE | G | F | F | |
| 344 | Suc | (MeD | G | F | F | |
| 345 | Suc | βD | G | F | F | |
| 346 | Suc | E(Me | G | F | F | |
| 347 | Suc | D | G | F | F | |
| 348 | Suc | Gla | G | F | F | |
| 349 | Suc | E | G | Y | F | |
| 350 | Suc | Q | G | Y | F | |
| 351 | Suc | N | G | Y | F | |
| 352 | Suc | (Me)E | G | Y | F | |
| 353 | Suc | βE | G | Y | F | |
| 354 | Suc | (Me)D | G | Y | F | |
| 355 | Suc | βD | G | Y | F | |
| 356 | Suc | E(Me) | G | Y | F | |
| 357 | Suc | D | G | Y | F | |
| 358 | Suc | Gla | G | Y | F | |
| 359 | Suc | E | G | W | F | |
| 360 | Suc | Q | G | W | F | |
| 361 | Suc | N | G | W | F | |
| 362 | Suc | (Me)E | G | W | F | |
| 363 | Suc | βE | G | W | F | |
| 364 | Suc | (Me)D | G | W | F | |
| 365 | Suc | βD | G | W | F | |
| 366 | Suc | E(Me) | G | W | F | |
| 367 | Suc | D | G | W | F | |
| 368 | Suc | Gla | G | W | F | |
| 369 | Suc | E | G | F(3Br) | F | |
| 370 | Suc | Q | G | F(3Br) | F | |
| 371 | Suc | N | G | F(3Br) | F | |
| 372 | Suc | (Me)E | G | F(3Br) | F | |
| 373 | Suc | βE | G | F(3Br) | F | |
| 374 | Suc | (Me)D | G | F(3Br) | F | |
| 375 | Suc | βD | G | F(3Br) | F | |
| 376 | Suc | E(Me) | G | F(3Br) | F | |
| 377 | Suc | D | G | F(3Br) | F | |
| 378 | Suc | Gla | G | F(3Br) | F | |
| 379 | Suc | E | G | F(4NO₂) | F | |
| 380 | Suc | Q | G | F(4NO₂) | F | |
| 381 | Suc | N | G | F(4NO₂) | F | |
| 382 | Suc | (Me)E | G | F(4NO₂) | F | |
| 383 | Suc | βE | G | F(4NO₂) | F | |
| 384 | Suc | (Me)D | G | F(4NO₂) | F | |
| 385 | Suc | βD | G | F(4NO₂) | F | |
| 386 | Suc | E(Me) | G | F(4NO₂) | F | |
| 387 | Suc | D | G | F(4NO₂) | F | |
| 388 | Suc | Gla | G | F(4NO₂) | F | |
| 389 | Suc | E | G | 3Pal | F | |
| 390 | Suc | Q | G | 3Pal | F | |
| 391 | Suc | N | G | 3Pal | F | |
| 392 | Suc | (Me)E | G | 3Pal | F | |
| 393 | Suc | βE | G | 3Pal | F | |
| 394 | Suc | (Me)D | G | 3Pal | F | |
| 395 | Suc | βD | 0 | 3Pal | F | |
| 396 | Suc | E(Me) | G | 3Pal | F | |
| 397 | Suc | D | G | 3Pal | F | |
| 398 | Suc | Gla | G | 3Pal | F | |
| 399 | Suc | E | G | Y(Me) | F | |
| 400 | Suc | Q | G | Y(Me) | F | |
| 401 | Suc | N | G | Y(Me) | F | |
| 402 | Suc | (Me)E | G | Y(Me) | F | |
| 403 | Suc | βE | G | Y(Me) | F | |
| 404 | Suc | (Me)D | G | Y(Me) | F | |
| 405 | Suc | βD | G | Y(Me) | F | |
| 406 | Suc | E(Me) | G | Y(Me) | F | |
| 407 | Suc | D | G | Y(Me) | F | |
| 408 | Suc | Gla | G | Y(Me) | F | |
| 409 | Suc | E | G | Pip | F | |
| 410 | Suc | Q | G | Pip | F | |
| 411 | Suc | N | G | Pip | F | |
| 412 | Suc | (Me)E | G | Pip | F | |
| 413 | Suc | βE | G | Pip | F | |
| 414 | Suc | (Me)D | G | Pip | F | |
| 415 | Suc | βD | G | Pip | F | |
| 416 | Suc | E(Me) | G | Pip | F | |
| 417 | Suc | D | G | Pip | F | |
| 418 | Suc | Gla | G | Pip | F | |
| 419 | Suc | E | G | Tic | F | |
| 420 | Suc | Q | G | Tic | F | |
| 421 | Suc | N | G | Tic | F | |
| 422 | Suc | (Me)E | G | Tic | F | |
| 423 | Suc | βE | G | Tic | F | |
| 424 | Suc | (Me)D | G | Tic | F | |
| 425 | Suc | βD | G | Tic | F | |
| 426 | Suc | E(Me) | G | Tic | F | |
| 427 | Suc | D | G | Tic | F | |
| 428 | Suc | Gla | G | Tic | F | |
| 429 | Suc | E | G | 1Nal | F | |
| 430 | Suc | Q | G | 1Nal | F | |
| 431 | Suc | N | G | 1Nal | F | |
| 432 | Suc | (Me)E | G | 1Nal | F | |
| 433 | Suc | βE | G | 1Nal | F | |
| 434 | Suc | (Me)D | G | 1Nal | F | |
| 435 | Suc | βD | G | 1Nal | F | |
| 436 | Suc | E(Me) | G | 1Nal | F | |
| 437 | Suc | D | G | 1Nal | F | |
| 438 | Suc | Gla | G | 1Nal | F | |
| 439 | Suc | E | G | F(2F) | F | |
| 440 | Suc | Q | G | F(2F) | F | |
| 441 | Suc | N | G | F(2F) | F | |
| 442 | Suc | (Me)E | G | F(2F) | F | |
| 443 | Suc | βE | G | F(2F) | F | |
| 444 | Suc | (Me)D | G | F(2F) | F | |
| 445 | Suc | βD | G | F(2F) | F | |
| 446 | Suc | E(Me) | G | F(2F) | F | |
| 447 | Suc | D | G | F(2F) | F | |
| 448 | Suc | Gla | G | F(2F) | F | |
| 449 | Suc | E | G | F(3F) | F | |
| 450 | Suc | Q | G | F(3F) | F | |
| 451 | Suc | N | G | F(3F) | F | |
| 452 | Suc | (Me)E | G | F(3F) | F | |
| 453 | Suc | βE | G | F(3F) | F | |
| 454 | Suc | (Me)D | G | F(3F) | F | |
| 455 | Suc | βD | G | F(3F) | F | |
| 456 | Suc | E(Me) | G | F(3F) | F | |
| 457 | Suc | D | G | F(3F) | F | |
| 458 | Suc | Gla | G | F(3F) | F | |
| 459 | Suc | E | G | F(4F) | F | |
| 460 | Suc | Q | G | F(4F) | F | |
| 461 | Suc | N | G | F(4F) | F | |
| 462 | Suc | (Me)E | G | F(4F) | F | |
| 463 | Suc | βE | G | F(4F) | F | |
| 464 | Suc | (Me)D | G | F(4F) | F | |
| 465 | Suc | βD | G | F(4F) | F | |
| 466 | Suc | E(Me) | G | F(4F) | F | |
| 467 | Suc | D | G | F(4F) | F | |
| 468 | Suc | Gla | G | F(4F) | F | |
| 469 | Suc | E | G | F | F | |
| 470 | Suc | Q | G | F | F | |
| 471 | Suc | N | G | F | F | |
| 472 | Suc | (Me)E | G | F | F | |
| 473 | Suc | βE | G | F | F | |
| 474 | Suc | (Me)D | G | F | F | |
| 475 | Suc | βD | G | F | F | |
| 476 | Suc | E(Me) | G | F | F | |
| 477 | Suc | D | G | F | F | |
| 478 | Suc | Gla | G | F | F | |
| 479 | Suc | E | G | F | Y | |
| 480 | Suc | Q | G | F | Y | |
| 481 | Suc | N | G | F | Y | |
| 482 | Suc | (Me)E | G | F | Y | |
| 483 | Suc | βE | G | F | Y | |
| 484 | Suc | (Me)D | G | F | Y | |
| 485 | Suc | βD | G | F | Y | |
| 486 | Suc | E(Me) | G | F | Y | |
| 487 | Suc | D | G | F | Y | |
| 488 | Suc | Gla | G | F | Y | |
| 489 | Suc | E | G | F | W | |
| 490 | Suc | Q | G | F | W | |
| 491 | Suc | N | G | F | W | |
| 492 | Suc | (Me)E | G | F | W | |
| 493 | Suc | βE | G | F | W | |
| 494 | Suc | (Me)D | G | F | W | |
| 495 | Suc | βD | G | F | W | |
| 496 | Suc | E(Me) | G | F | W | |
| 497 | Suc | D | G | F | W | |
| 498 | Suc | Gla | G | F | W | |
| 499 | Suc | E | G | F | F(3Br) | |
| 500 | Suc | Q | G | F | F(3Br) | |
| 501 | Suc | N | G | F | F(3Br) | |
| 502 | Suc | (Me)E | G | F | F(3Br) | |
| 503 | Suc | βE | G | F | F(3Br) | |
| 504 | Suc | (Me)D | G | F | F(3Br) | |
| 505 | Suc | βD | G | F | F(3Br) | |
| 506 | Suc | E(Me) | G | F | F(3Br) | |
| 507 | Suc | D | G | F | F(3Br) | |
| 508 | Suc | Gla | G | F | F(3Br) | |
| 509 | Suc | E | G | F | F(4NO₂) | |
| 510 | Suc | Q | G | F | F(4NO₂) | |
| 511 | Suc | N | G | F | F(4NO₂) | |
| 512 | Suc | (Me)E | G | F | F(4NO₂) | |
| 513 | Suc | βE | G | F | F(4NO₂) | |
| 514 | Suc | (Me)D | G | F | F(4NO₂) | |
| 515 | Suc | βD | G | F | F(4NO₂) | |
| 516 | Suc | E(Me) | G | F | F(4NO₂) | |
| 517 | Suc | D | G | F | F(4NO₂) | |
| 518 | Suc | Gla | G | F | F(4NO₂) | |
| 519 | Suc | E | G | F | 3Pal | |
| 520 | Suc | Q | G | F | 3Pal | |
| 521 | Suc | N | G | F | 3Pal | |
| 522 | Suc | (Me)E | G | F | 3Pal | |
| 523 | Suc | βE | G | F | 3Pal | |
| 524 | Suc | (Me)D | G | F | 3Pal | |
| 525 | Suc | βD | G | F | 3Pal | |
| 526 | Suc | E(Me) | G | F | 3Pal | |
| 527 | Suc | D | G | F | 3Pal | |
| 528 | Suc | Gla | G | F | 3Pal | |
| 529 | Suc | E | G | F | Y(Me) | |
| 530 | Suc | Q | G | F | Y(Me) | |
| 531 | Suc | N | G | F | Y(Me) | |
| 532 | Suc | (Me)E | G | F | Y(Me) | |
| 533 | Suc | βE | G | F | Y(Me) | |
| 534 | Suc | (Me)D | G | F | Y(Me) | |
| 535 | Suc | βD | G | F | Y(Me) | |
| 536 | Suc | E(Me) | G | F | Y(Me) | |
| 537 | Suc | D | G | F | Y(Me) | |
| 538 | Suc | Gla | G | F | Y(Me) | |
| 539 | Suc | E | G | F | Pip | |
| 540 | Suc | Q | G | F | Pip | |
| 541 | Suc | N | G | F | Pip | |
| 542 | Suc | (Me)E | G | F | Pip | |
| 543 | Suc | βE | G | F | Pip | |
| 544 | Suc | (Me)D | G | F | Pip | |
| 545 | Suc | βD | G | F | Pip | |
| 546 | Suc | E(Me) | G | F | Pip | |
| 547 | Suc | D | G | F | Pip | |
| 548 | Suc | Gla | G | F | Pip | |
| 549 | Suc | E | G | F | Tic | |
| 550 | Suc | Q | G | F | Tic | |
| 551 | Suc | N | G | F | Tic | |
| 552 | Suc | (Me)E | G | F | Tic | |
| 553 | Suc | βB | G | F | Tic | |
| 554 | Suc | (Me)D | G | F | Tic | |
| 555 | Suc | βD | G | F | Tic | |
| 556 | Suc | E(Me) | G | F | Tic | |
| 557 | Suc | D | G | F | Tic | |
| 558 | Suc | Gla | G | F | Tic | |
| 559 | Suc | E | G | F | 1Nal | |
| 560 | Suc | Q | G | F | 1Nal | |
| 561 | Suc | N | G | F | 1Nal | |
| 562 | Suc | (Me)E | G | F | 1Nal | |
| 563 | Suc | βE | G | F | 1Nal | |
| 564 | Suc | (Me)D | G | F | 1Nal | |
| 565 | Suc | βD | G | F | 1Nal | |
| 566 | Suc | E(Me) | G | F | 1Nal | |
| 567 | Suc | D | G | F | 1Nal | |
| 568 | Suc | Gla | G | F | 1Nal | |
| 569 | Suc | E | G | F | F(2F) | |
| 570 | Suc | Q | G | F | F(2F) | |
| 571 | Suc | N | G | F | F(2F) | |
| 572 | Suc | (Me)E | G | F | F(2F) | |
| 573 | Suc | βE | G | F | F(2F) | |
| 574 | Suc | (Me)D | G | F | F(2F) | |
| 575 | Suc | βD | G | F | F(2F) | |
| 576 | Suc | E(Me) | G | F | F(2F) | |
| 577 | Suc | D | G | F | F(2F) | |
| 578 | Suc | Gla | G | F | F(2F) | |
| 579 | Suc | E | G | F | F(3F) | |
| 580 | Suc | Q | G | F | F(3F) | |
| 581 | Suc | N | G | F | F(3F) | |
| 582 | Suc | (Me)E | G | F | F(3F) | |
| 583 | Suc | βE | G | F | F(3F) | |
| 584 | Suc | (Me)D | G | F | F(3F) | |
| 585 | Suc | βD | G | F | F(3F) | |
| 586 | Suc | E(Me) | G | F | F(3F) | |
| 587 | Suc | D | G | F | F(3F) | |
| 588 | Suc | Gla | G | F | F(3F) | |
| 589 | Suc | E | G | F | F(4F) | |
| 590 | Suc | Q | G | F | F(4F) | |
| 591 | Suc | N | G | F | F(4F) | |
| 592 | Suc | (Me)E | G | F | F(4F) | |
| 593 | Suc | βE | G | F | F(4F) | |
| 594 | Suc | (Me)D | G | F | F(4F) | |
| 595 | Suc | βD | G | F | F(4F) | |
| 596 | Suc | E(Me) | G | F | F(4F) | |
| 597 | Suc | D | G | F | F(4F) | |
| 598 | Suc | Gla | G | F | F(4F) | |
| 599 | Suc | E | G | F | F | |
| 600 | Peg35 | E | G | F | F | |
| 601 | Peg30 | E | G | F | F | |
| 602 | Taa | E | G | F | F | |
| 603 | Caa | E | G | F | F | |
| 604 | Fum | E | G | F | F | |
| 605 | TA | E | G | F | F | |
| 606 | Ia | E | G | F | F | |
| 607 | 1,4-Chda | E | G | F | F | |
| 608 | 1,2-Chda | E | G | F | F | |
| 609 | Ga | E | G | F | F | |
| 610 | Suc | E | G | Y | F | |
| 611 | Peg35 | E | G | Y | F | |
| 612 | Peg30 | E | G | Y | F | |
| 613 | Taa | E | G | Y | F | |
| 614 | Caa | E | G | Y | F | |
| 615 | Fum | E | G | Y | F | |
| 616 | TA | E | G | Y | F | |
| 617 | Ia | E | G | Y | F | |
| 618 | 1,4-Chda | E | G | Y | F | |
| 619 | 1,2-Chda | E | G | Y | F | |
| 620 | Ga | E | G | Y | F | |
| 621 | Suc | E | A | F | F | |
| 622 | Peg35 | E | A | F | F | |
| 623 | Peg30 | E | A | F | F | |
| 624 | Taa | E | A | F | F | |
| 625 | Caa | E | A | F | F | |
| 626 | Fum | E | A | F | F | |
| 627 | TA | E | A | F | F | |
| 628 | Ia | E | A | F | F | |
| 629 | 1,4-Chda | E | A | F | F | |
| 630 | 1,2-Chda | E | A | F | F | |
| 631 | Ga | E | A | F | F | |
| 632 | Suc | E | a | F | F | |
| 633 | Peg35 | E | a | F | F | |
| 634 | Peg30 | E | a | F | F | |
| 635 | Taa | E | a | F | F | |
| 636 | Caa | E | a | F | F | |
| 637 | Fum | E | a | F | F | |
| 638 | TA | E | a | F | F | |
| 639 | Ia | E | a | F | F | |
| 640 | 1,4-Chda | E | a | F | F | |
| 641 | 1,2-Chda | E | a | F | F | |
| 642 | Ga | E | a | F | F | |
| 643 | Suc | E | βA | F | F | |
| 644 | Peg35 | E | βA | F | F | |
| 645 | Peg30 | E | βA | F | F | |
| 646 | Taa | E | βA | F | F | |
| 647 | Caa | E | βA | F | F | |
| 648 | Fum | E | βA | F | F | |
| 649 | TA | E | βA | F | F | |
| 650 | Ia | E | βA | F | F | |
| 651 | 1,4-Chda | E | βA | F | F | |
| 652 | 1,2-Chda | E | βA | F | F | |
| 653 | Ga | E | βA | F | F | |
| 654 | Suc | E | P | F | F | |
| 655 | Peg35 | E | P | F | F | |
| 656 | Peg30 | E | P | F | F | |
| 657 | Taa | E | P | F | F | |
| 658 | Caa | E | P | F | F | |
| 659 | Fum | E | P | F | F | |
| 660 | TA | E | P | F | F | |
| 661 | Ia | E | P | F | F | |
| 662 | 1,4-Chda | E | P | F | F | |
| 663 | 1,2-Chda | E | P | F | F | |
| 664 | Ga | E | P | F | F | |
| 665 | Suc | E | p | F | F | |
| 666 | Peg35 | E | p | F | F | |
| 667 | Peg30 | E | p | F | F | |
| 668 | Taa | E | p | F | F | |
| 669 | Caa | E | p | F | F | |
| 670 | Fum | E | p | F | F | |
| 671 | TA | E | p | F | F | |
| 672 | Ia | E | p | F | F | |
| 673 | 1,4-Chda | E | p | F | F | |
| 674 | 1,2-Chda | E | p | F | F | |
| 675 | Ga | E | p | F | F | |
| 676 | Suc | E | Sar | F | F | |
| 677 | Peg35 | E | Sar | F | F | |
| 678 | Peg30 | E | Sar | F | F | |
| 679 | Taa | E | Sar | F | F | |
| 680 | Caa | E | Sar | F | F | |
| 681 | Fum | E | Sar | F | F | |
| 682 | TA | E | Sar | F | F | |
| 683 | Ia | E | Sar | F | F | |
| 684 | 1,4-Chda | E | Sar | F | F | |
| 685 | 1,2-Chda | E | Sar | F | F | |
| 686 | Ga | E | Sar | F | F | |
| 687 | Suc | E | β-H-ala | F | F | |
| 688 | Peg35 | E | β-H-ala | F | F | |
| 689 | Peg30 | E | β-H-ala | F | F | |
| 690 | Taa | E | β-H-ala | F | F | |
| 691 | Caa | E | β-H-ala | F | F | |
| 692 | Fum | E | β-H-ala | F | F | |
| 693 | TA | E | β-H-ala | F | F | |
| 694 | Ia | E | β-H-ala | F | F | |
| 695 | 1,4-Chda | E | β-H-ala | F | F | |
| 696 | 1,2-Chda | E | β-H-ala | F | F | |
| 697 | Ga | E | β-H-ala | F | F | |
| 698 | Suc | E | Ahx | F | F | |
| 699 | Peg35 | E | Ahx | F | F | |
| 700 | Peg30 | E | Ahx | F | F | |
| 701 | Taa | E | Ahx | F | F | |
| 702 | Caa | E | Ahx | F | F | |
| 703 | Fum | E | Ahx | F | F | |
| 704 | TA | E | Ahx | F | F | |
| 705 | Ia | E | Ahx | F | F | |
| 706 | 1,4-Chda | E | Ahx | F | F | |
| 707 | 1,2-Chda | E | Ahx | F | F | |
| 708 | Ga | E | Ahx | F | F | |
| 709 | Suc | E | F | F | | |
| 710 | Peg35 | E | F | F | | |
| 711 | Peg30 | E | F | F | | |
| 712 | Taa | E | F | F | | |
| 713 | Caa | E | F | F | | |
| 714 | Fum | E | F | F | | |
| 715 | TA | E | F | F | | |
| 716 | Ia | E | F | F | | |
| 717 | 1.4-Chda | E | E | F | | |
| 718 | 1,2-Chda | E | E | F | | |
| 719 | Ga | E | F | F | | |
| 720 | Suc | E | F | F | E | |
| 721 | Suc | E | Ahx | F | | |
| 722 | Suc | E | Ahx | F | E | |
| 723 | Suc | E | Ahx | F | Y | |
| 724 | Suc | E | Ahx | F | W | |
| 725 | Suc | E | Ahx | F | F(3Br) | |
| 726 | Suc | E | Ahx | F | F(4NO₂) | |
| 727 | Suc | E | Ahx | F | 3Pal | |
| 728 | Suc | E | Ahx | F | Y(Me) | |
| 729 | Suc | E | Ahx | F | Pip | |
| 730 | Suc | E | Ahx | F | Tic | |
| 731 | Suc | E | Ahx | F | 1Nal | |
| 732 | Suc | E | Ahx | F | F(2F) | |
| 733 | Suc | E | Ahx | F | F(3F) | |
| 734 | Suc | E | Ahx | F | F(4F) | |
| 735 | Suc | E | Ahx | F | F | |
| 736 | Suc | Q | Ahx | F | F | |
| 737 | Suc | N | Ahx | F | F | |
| 738 | Suc | (Me)E | Ahx | F | F | |
| 739 | Suc | βE | Ahx | F | F | |
| 740 | Suc | (Me)D | Ahx | F | F | |
| 741 | Suc | βD | Ahx | F | F | |
| 742 | Suc | E(Me) | Ahx | F | F | |
| 743 | Suc | D | Ahx | F | F | |
| 744 | Suc | Gla | Ahx | F | F | |
| 745 | Ga | E | G | F | Y(Me) | |
| 746 | Suc | E | Ahx | Y | F | |
| 747 | Suc | E | Ahx | W | F | |
| 748 | Suc | E | Ahx | F(3Br) | F | |
| 749 | Suc | E | Ahx | F(4NO₂) | F | |
| 750 | Suc | E | Ahx | 3Pal | F | |
| 751 | Suc | E | Ahx | Y(Me) | F | |
| 752 | Suc | E | Ahx | Pip | F | |
| 753 | Suc | E | Ahx | Tic | F | |
| 754 | Suc | E | Ahx | 1Nal | F | |
| 755 | Suc | E | Ahx | F(2F) | F | |
| 756 | Suc | E | Ahx | F(3F) | F | |
| 757 | Suc | E | Ahx | F(4F) | F | |
| 758 | Suc | E | Ahx | F | F | |
| 759 | Suc | Ahx | G | F | F | |
| 760 | Suc | E | Ahx | F | F | |
| 761 | Suc | E | G | Ahx | F | |
| 762 | Suc | E | G | F | Ahx | |
| 763 | Suc | E | F | F | E | |
| 764 | Peg35 | E | F | F | E | |
| 765 | Peg30 | E | F | F | E | |
| 766 | Taa | E | F | F | E | |
| 767 | Caa | E | F | F | E | |
| 768 | Fum | E | F | F | E | |
| 769 | TA | E | F | F | E | |
| 770 | Ia | E | F | F | E | |
| 771 | 1,4-Chda | E | F | F | E | |
| 772 | 1,2-Chda | E | F | F | E | |
| 773 | Ga | E | F | F | E | |
| 774 | Suc | E | Y | F | E | |
| 775 | Peg35 | E | Y | F | E | |
| 776 | Peg30 | E | Y | F | E | |
| 777 | Taa | E | Y | F | E | |
| 778 | Caa | E | Y | F | E | |
| 779 | Fum | E | Y | F | E | |
| 780 | TA | E | Y | F | E | |
| 781 | Ia | E | Y | F | E | |
| 782 | 1,4-Chda | E | Y | F | E | |
| 783 | 1,2-Chda | E | Y | F | E | |
| 784 | Ga | E | Y | F | E | |
| 785 | Peg35 | E | F | Y(Me) | E | |
| 786 | Peg30 | E | F | Y(Me) | E | |
| 787 | Taa | E | F | Y(Me) | E | |
| 788 | Caa | E | F | Y(Me) | E | |
| 789 | Fum | E | F | Y(Me) | E | |
| 790 | TA | E | F | Y(Me) | E | |
| 791 | Ia | E | F | Y(Me) | E | |
| 792 | 1,4-Chda | E | F | Y(Me) | E | |
| 793 | 1,2-Chda | E | F | Y(Me) | E | |
| 794 | Ga | E | F | Y(Me) | E | |

Ahx = aminohexanoic acid

Selected peptides from the arrays shown in Tables 5 and 6 were re-synthesised and analysed using the FP assay described above. The results of this assay are shown in Table 7.

TABLE 7
FP assay results of selected peptides

| | | FP Assay |
|---|---|---|
| Entry | Sequence | IC₅₀/μM |
| 1 | 1,4-Chda-EGFFE-NH₂ | >100 |
| 2 | EAFFE-NH₂ | 28 |
| 3 | Ga-EGFFE-NH₂ | 65 |
| 4 | Suc-EG-1Nal-F(4NO₂)-E-NH₂ | 68 |
| 5 | Suc-EG-1Nal-Y(Me)-E-NH₂ | 5.4 |
| 6 | Suc-EG-F(2F)-F(3F)-NH₂ | >100 |
| 7 | Suc-EG-F(2F)-F(4NO2)-E-NH₂ | 4.9 |
| 8 | Suc-EG-F(2F)-Y(4Me)-E-NH₂ | 3.2 |
| 9 | Suc-EG-F(3F)-3Pal-NH₂ | 70 |
| 10 | Suc-EG-F(3F)-F(3F)-NH₂ | 44 |
| 11 | Suc-EG-F(3F)-F(4NO2)-E-NH₂ | 0.52 |
| 12 | Suc-EG-F(3F)-F(4NO2)-NH₂ | >100 |
| 13 | Suc-EG-F(4Br)-F(3F)-NH₂ | 81 |
| 14 | Suc-EG-F(4Br)-F-NH₂ | 38 |
| 15 | Suc-EG-F(4Br)-F-NH₂ | 39 |
| 16 | Suc-EG-F(4F)-F(4NO₂)-E-NH₂ | 1.2 |
| 17 | Suc-EG-F(4NO₂)-1Nal-NH₂ | 14 |
| 18 | Suc-EG-F(4NO₂)-F(4NO2)-NH₂ | 78 |
| 19 | Suc-EGF-F(3F)-NH₂ | >100 |
| 20 | Suc-EGF-F(4F)-NH₂ | >100 |
| 21 | Suc-EGF-F(4NO₂)-NH₂ | >100 |
| 22 | Suc-EGFF-NH₂ | 46 |
| 23 | Suc-EGF-Y(Me)-E(OMe)-NH₂ | 71 |
| 24 | Suc-EGY-F(3F)-NH₂ | 91 |
| 25 | Suc-EGY-F(4F)-NH₂ | 71 |
| 26 | Suc-EGY-F(4NO₂)-NH₂ | 91 |
| 27 | Suc-EGYFE-NH₂ | 1 |
| 28 | Suc-EGYF-NH₂ | 31 |
| 29 | Suc-QGYF-NH₂ | 49 |
| 30 | Suc-βE-GYFE-NH₂ | 30 |

X-EGXXE-NH₂ was identified as a useful consensus binding motif and further peptides were designed and synthesised to increase potency. These peptides were tested in the FP assay described above and the results are shown in Table 8.

TABLE 8
Further modification of peptide sequence X-EGXXE-NH₂

| | | FP Assay |
|---|---|---|
| Entry | Sequence | IC₅₀/μM |
| 1 | (MeO)Suc-EG-F(3F)-1Nal-E-NH₂ | 1.6 |
| 2 | 2-NaphthylSO₂-dEG-F(3F)-WE-NH₂ | 0.044 |
| 3 | 3,4-(MeO)₂PhSO₂-dEG-F(3F)-WE-NH₂ | 0.042 |
| 4 | 4-(BuO)PhSO₂-dEG-F(3F)-WE-NH₂ | 0.056 |
| 5 | 4-(MeO)PhSO₂-dEG-F(3F)-WE-NH₂ | 0.017 |
| 6 | 4-(MeO)PhSO₂-DEG-F(3F)-WE-NH₂ | 0.027 |
| 7 | 4-(PhO)PhSO₂-dEG-F(3F)-WE-NH₂ | 0.063 |
| 8 | Ac-dEG-F(3F)-1Nal-E-NH₂ | 0.112 |
| 9 | Ac-dEG-F(3F)-WE-NH₂ | 0.102 |
| 10 | AG-F(3F)-F(4NO₂)-E-NH₂ | >100 |
| 11 | AGYFE-NH₂ | >100 |
| 12 | Bz-dEG-F(3F)-WE-NH₂ | 0.173 |
| 13 | EA-F(3F)-F(4NO₂)-E-NH₂ | >100 |
| 14 | EAFFE-NH₂ | >100 |
| 15 | EAYFE-NH₂ | >100 |
| 16 | EG-F(3F)-1Nal-E-NH₂ | >100 |
| 17 | EG-F(4NO₂)-1Nal-E-NH₂ | >100 |
| 18 | EGY-1Nal-E-NH₂ | >100 |
| 19 | EGY-F(4NO₂)-E-NH₂ | >100 |
| 20 | EtCO-dEG-F(3F)-WE-NH₂ | 0.145 |
| 21 | EtOCO-dEG-F(3F)-WE-NH₂ | 0.045 |
| 22 | Fum-EG-F(3F)-F(4NO₂)-E-NH₂ | 60.4 |
| 23 | Mal-EG-F(3F)-F(4NO₂)-E-NH₂ | 2.17 |
| 24 | MeOCO-dEG-F(3F)-WE-NH₂ | 0.076 |
| 25 | QG-F(3F)-F(4NO₂)-E-NH₂ | >100 |
| 26 | QGFFE-NH₂ | >100 |
| 27 | QGYFE-NH₂ | >100 |
| 28 | Suc-AG-F(3F)-F(4NO2)-E-NH₂ | 8.01 |
| 29 | Suc-AGYFE-NH₂ | 22.2 |
| 30 | Suc-EA-F(3F)-F(4NO₂)-E-NH₂ | 27.5 |
| 31 | Suc-EAFFE-NH₂ | 59.8 |
| 32 | Suc-EAYFE-NH₂ | 82.1 |
| 33 | Suc-EG-3Pal-1Nal-E-NH₂ | 3.63 |
| 34 | Suc-EG-F(3F)-1Nal-E-NH₂ | 0.157 |
| 35 | Suc-EG-F(3F)-1Nal-Q-NH₂ | 7.22 |
| 36 | Suc-EG-F(3F)-HE-NH₂ | 0.521 |
| 37 | Suc-EG-F(3F)-WE-NH₂ | 0.095 |
| 38 | Suc-EG-F(3F)-Y(Me)-E-NH₂ | 1.4 |
| 39 | Suc-EG-F(4NO₂)-1Nal-E-NH₂ | 1.31 |
| 40 | Suc-EGI-1Nal-E-NH₂ | 15.09 |
| 41 | Suc-EGY-1Nal-E-NH₂ | 1.15 |
| 42 | Suc-EGY-F(4NO₂)-E-NH₂ | 2.07 |
| 43 | Suc-QG-F(3F)-F(4NO₂)-E-NH₂ | 2.56 |
| 44 | Suc-QGFFE-NH₂ | 10 |
| 45 | Suc-QGYFE-NH₂ | 10.7 |
| 46 | Ts-dDG-F(3F)-WD-NH₂ | 0.081 |
| 47 | Ts-dDG-F(3F)-WE-NH₂ | 0.025 |
| 48 | Ts-dEGF(3Cl)WE(Me)-NH₂ | 0.017 |
| 49 | Ts-dEGF(3Cl)WE-NH₂ | 0.01 |
| 50 | Ts-dEG-F(3F)-WD-NH₂ | 0.034 |
| 51 | Ts-dEGF(3F)W-E(Me)-NH₂ | 0.022 |
| 52 | Ts-dEG-F(3F)-WE-NH₂ | 0.029 |
| 53 | Ts-DEG-F(3F)-WE-NH₂ | 0.018 |

### Example 2

### In Silico Biotin Capture Assay of Lead Test Compounds

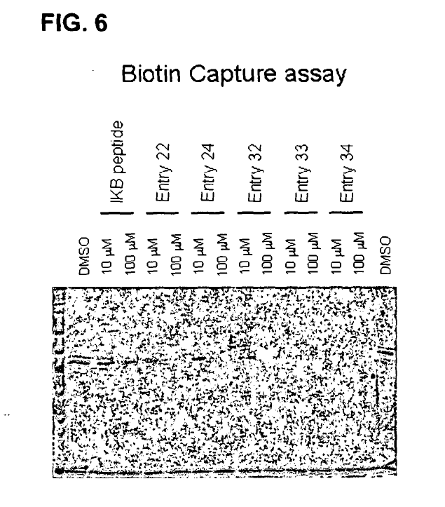

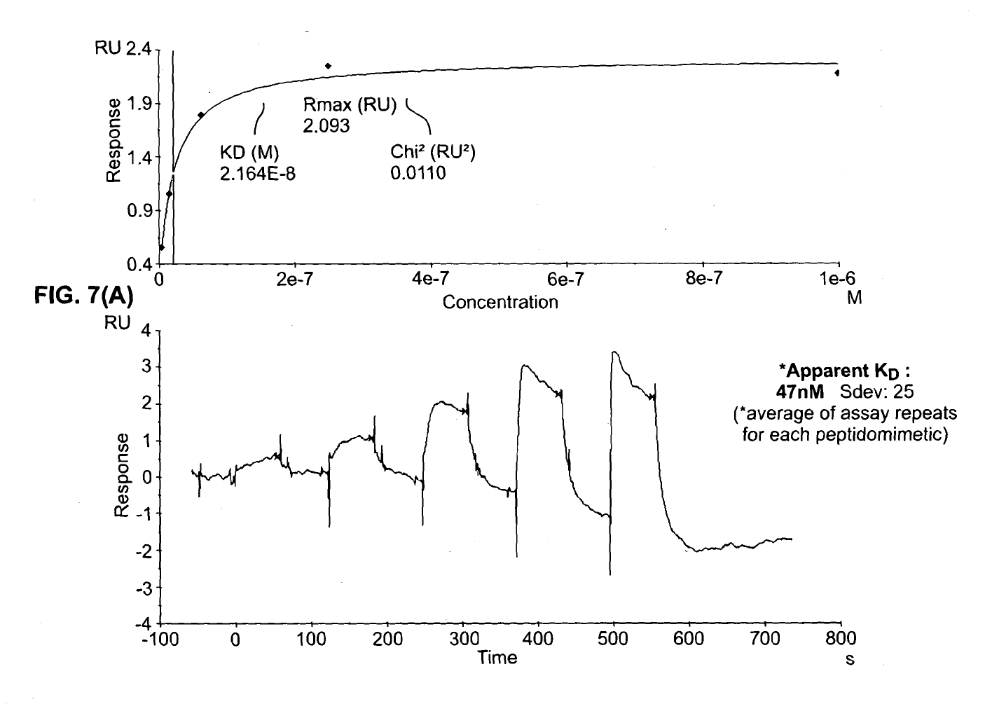

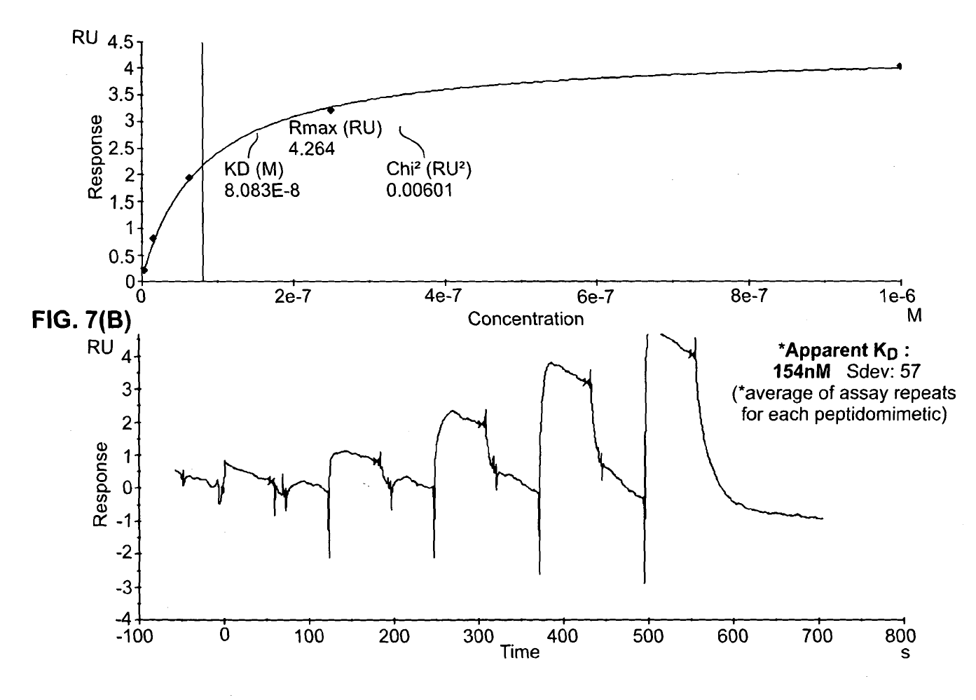

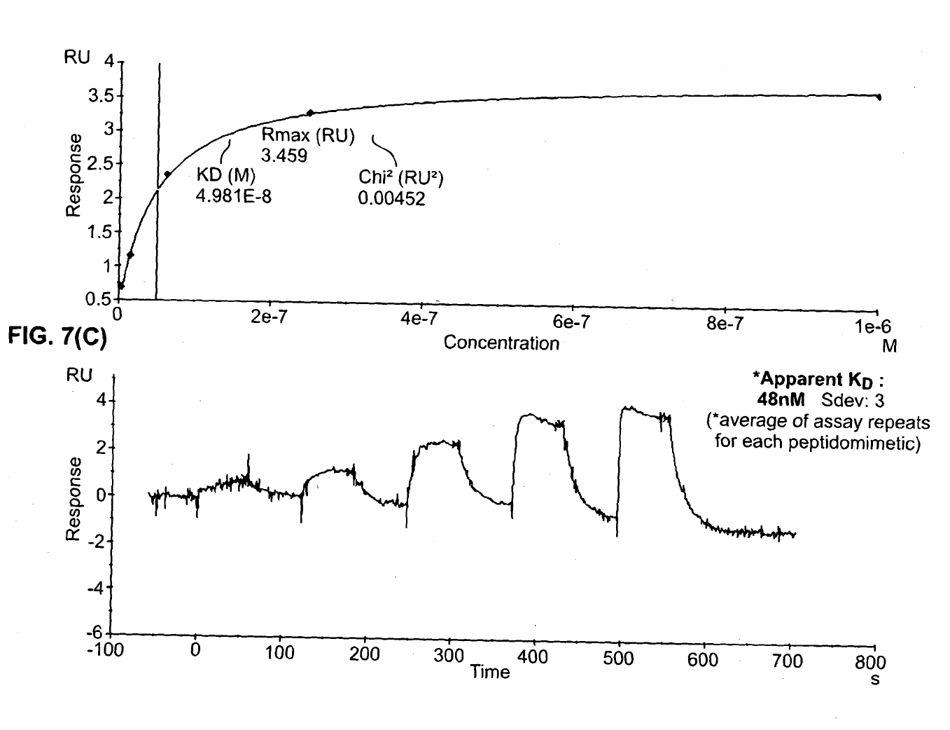

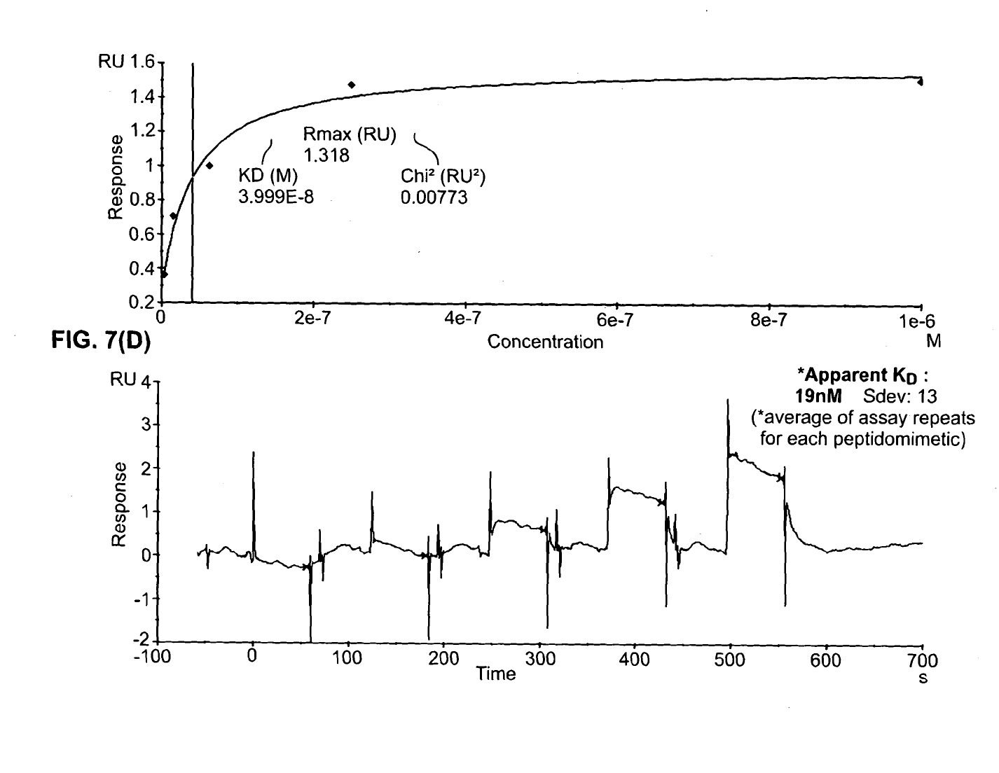

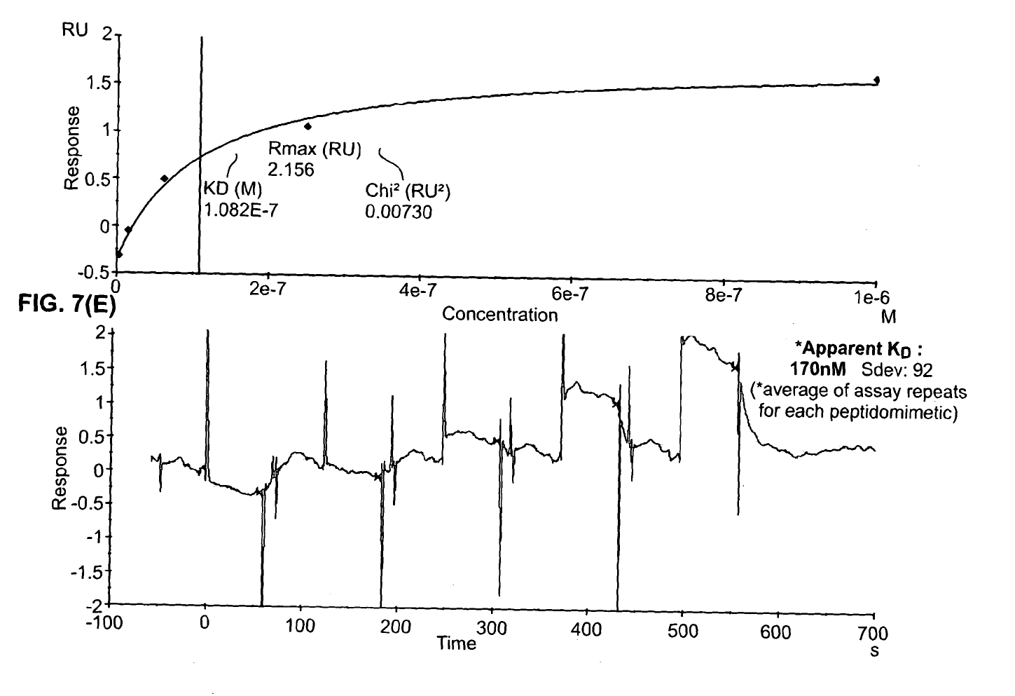

A non-fluorescent based assay was used to validate potential binding peptides. The phosphopeptide substrate KKERLLDDRHDpSGLDpSMKDEE was biotinylated by coupling a biotinamido-hexanoic acid succinimide ester to a lysine in the peptide. 0.5 μg of each protein was mixed with 50 picomoles of biotinylated peptide in a total volume of 50 μl. The buffer conditions were 50 mM Hepes 7.5 and 100 mM NaCl. Compounds were then added to a final concentration of 60 μM. The reaction was allowed to incubate for 1 hour at room temperature and then 5 μl of streptavidin-agarose beads was added and incubated for 1 hour. Beads were washed twice with buffer and run on an SDS-PAGE gel. Proteins were visualized by anti-His antibody. The results of this assay are shown in FIG. 6.

### Example 3

### Compound Hits from In-Vitro Assays Tested with SPR

Binding of SucEGF(4NO₂)1NalENH₂ to βTrCP1 was tested using the Surface Plasmon Resonance (SPR) method described above.

### Example 4

### Measurement of Inhibition of the E3 Ligase Cascades βTrCP(IκBα) and βTrCP(β-catenin)

Candidate compounds were tested in duplicate at both 10 and 100 μM against the E3 ligase cascades βTrCP(IκBα) and βTrCP(β-catenin). The E3 assays were carried out according to the component concentrations detailed in Table 9. In each case the E2 was HA,6His-UbCH3-(hu,FL), the E1 was 6H is-UBE1-(hu,FL) and the ubiquitin (Sigma U6253) was biotinylated at a 5:1 ratio. The E3 tetramer constructs and substrate pairings are shown in Table 10. Substrate phosphorylation was performed in the absence of compound; consequently any observed signal modulation should not reflect inhibition of the up-stream kinase reaction. All other steps (E1, E2 and substrate ubiquitination) were carried out in the presence of compound. The stopped reaction mix (10 μl) was added to an ECL plate loaded with anti c-Myc (1/500 Dilution, Millipore 05-724) and blocked with 5% BSA. Binding was allowed to proceed for 1 hour at RT before a wash step (3 40 μl PBST washes). Detection was achieved by binding SA-Ru TAG at 1 μg/ml (1 hour@RT, 3 40 μl PBST washes) before reading on an MSD Sector Imager 6000.

TABLE 9
Assay concentrations

| | βTrCP(IκBα) | | βTrCP(β-catenin) | |
|---|---|---|---|---|
| Ub | 2 | μM | 2 | μM |
| ATP | 10 | μM | 10 | μM |
| E2 | 250 | nM | 250 | nM |
| E1 | 2.5 | nM | 2.5 | nM |
| E3 Tetramer | 0.3 | μg/well | 0.3 | μg/well |
| Sub | 200 | nM | 200 | nM |

TABLE 10
E3 Ligase and substrate pairings

| E3 Tetramer | Substrate |
|---|---|
| GST-βTrCP1-(iso1,hu,53-end,E353D)/GST-Skp1- | cMyc, 6His-IκBα- |
| (hu,fl)/6His-Cul1-(hu,fl)/UT-Rbx1-(hu,fl) | (hu,FL) |
| GST-βTrCP1-(iso1,hu,53-end,E353D)/GST-Skp1- | cMyc,6His-β- |
| (hu,fl)/6His-Cul1-(hu,fl)/UT-Rbx1-(hu,fl) | catenin-(hu,FL) |

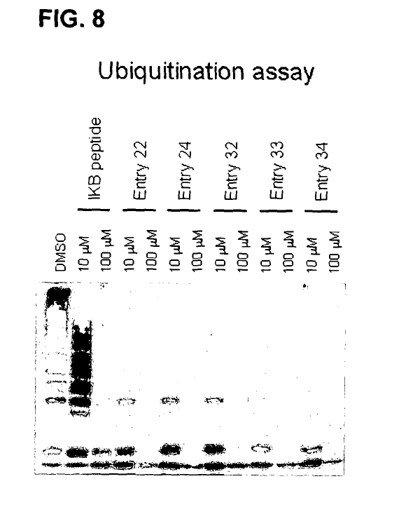

Assay results are shown in FIG. 8. Assays were performed as described above

### Example 5

### Selectivity Analysis of Top Compounds—FP Against Fbw7

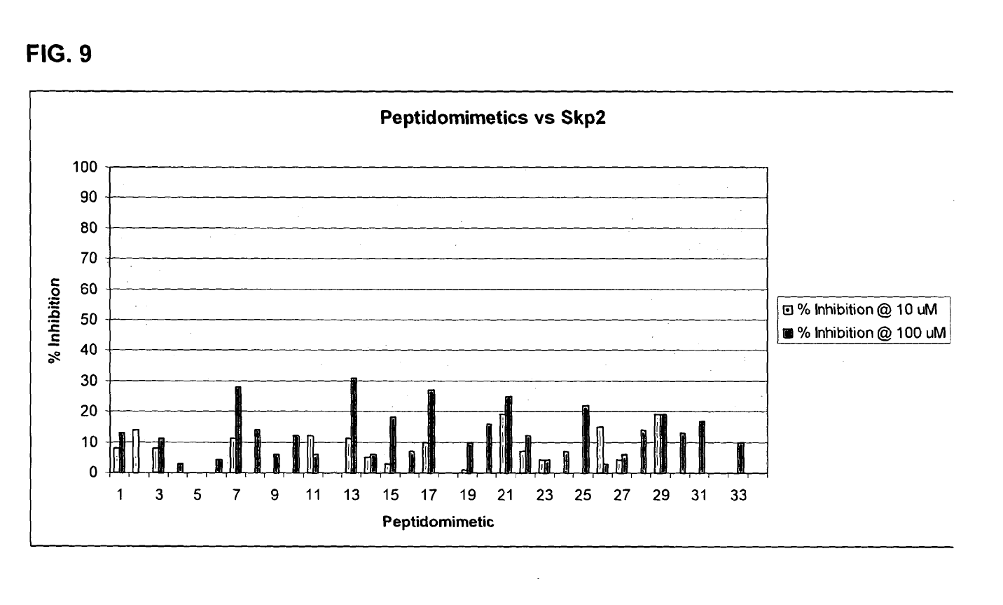

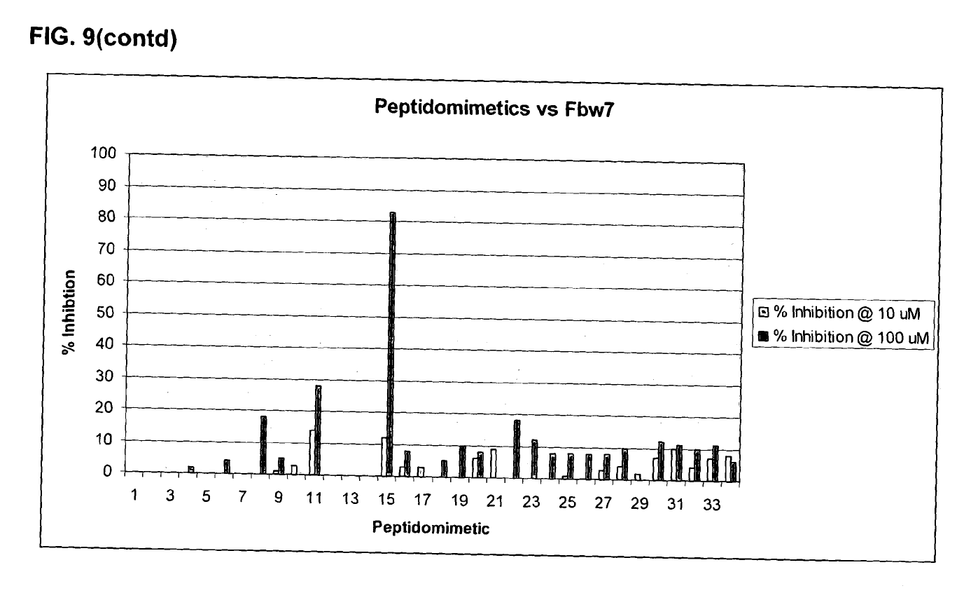

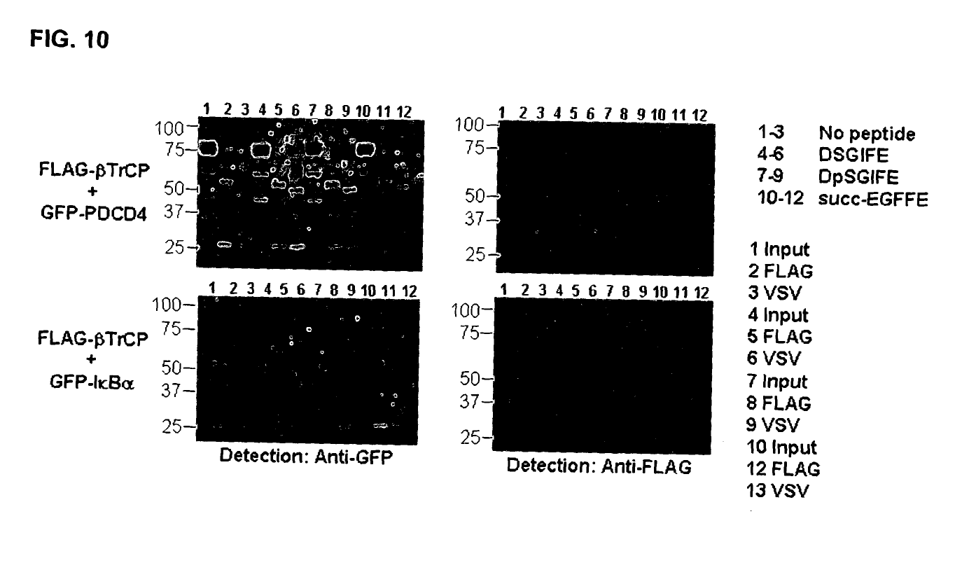

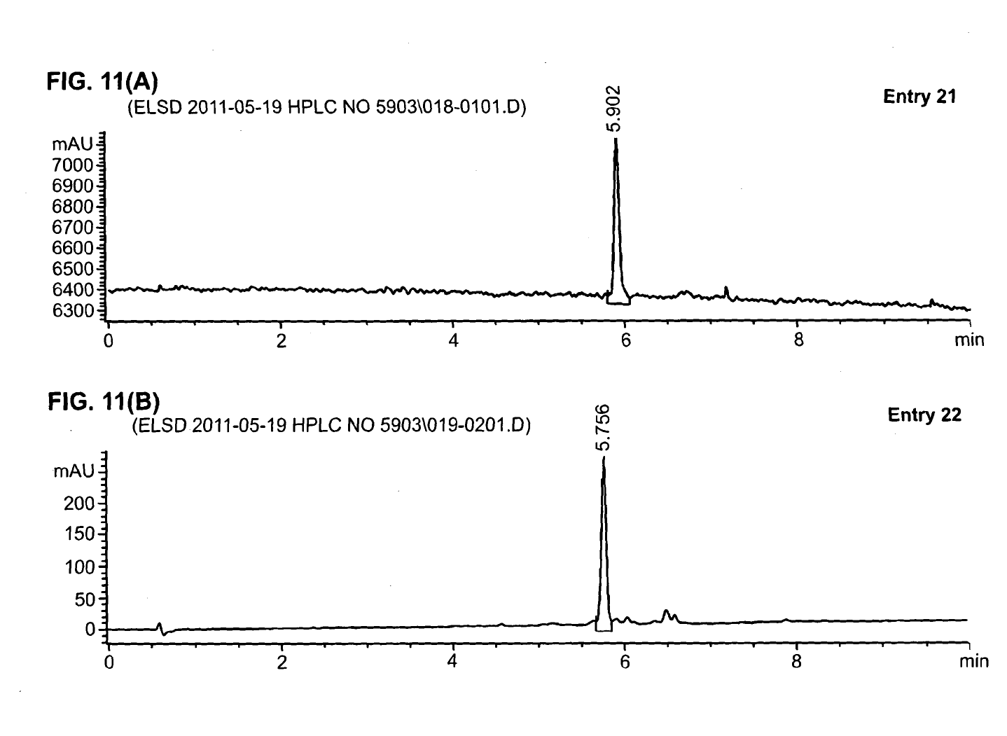

Candidate compounds were tested for inhibition of Fbw7 and Skp2 as described above. As shown in FIG. 9, none of them showed any activity above background at concentrations of 10 μM or 100 μM.

### Example 6

### Collation of Data Collected on Candidate Compounds

Table 11 (below) shows the collation of all data points collected for the most promising compounds.

TABLE 11
Summary of peptide data

| | | | | | | % Inhibitiom | | | |
|---|---|---|---|---|---|---|---|---|---|
| | | | | Biotin Pulldown | | Skp2 @ | | Fbw7 @ | |
| | | FP | SPR | Assay | Ub Assay | 10 μM | | 10 μM | |
| Entry | Sequence | IC₅₀/μM | Kd/μM | 10 μM/100 μM | 10 μM/100 μM | (100 μM) | | (100 μM) | |
| 1 | DpSGIFE-NH₂ | 1.24 ± 0.145 | 1.477 ± 0.286 | reduces/inhibits | reduces/inhibits | 8 | (13) | 0 | (0) |
| 2 | Suc-EGFFE-NH₂ | 3.18 ± 0.357 | 29.456 ± 4.613 | no effect/reduces | reduces/inhibits | 14 | (0) | 0 | (0) |
| 3 | Suc-EGF(2F)F(4NO₂)E-NH₂ | 4.91 ± 0.702 | 8.000 ± 0.260 | no effect/reduces | no effect/inhibits | 8 | (11) | 0 | (0) |
| 4 | Suc-EGF(3F)F(4NO₂)E-NH₂ | 0.522 ± 0.049 | 4.530 ± 0.681 | reduces/inhibits | inhibits/inhibits | 0 | (3) | 0 | (2) |
| 5 | Suc-EGF(4F)F(4NO2)E-NH₂ | 1.21 ± 0.093 | 0.507 ± 0.267 | no effect/inhibits | reduces/inhibits | 0 | (0) | 0 | (0) |
| 6 | Suc-EGF(2F)Y(Me)E-NH₂ | 3.28 ± 0.500 | 4.400 ± 0.820 | no effect/reduces | no effect/inhibits | 0 | (4) | 0 | (4) |
| 7 | Suc-EGYFE-NH₂ | 1.04 ± 0.101 | 0.537 ± 0.117 | no effect/inhibits | reduces/inhibits | 11 | (28) | 0 | (0) |
| 8 | Mal-EGF(3F)F(4NO₂)E-NH₂ | 2.17 ± 0.425 | 1.463 ± 0.035 | no effect/inhibits | no effect/inhibits | 0 | (14) | 0 | (18) |
| 9 | Suc-EGY1NalE-NH₂ | 1.15 ± 0.222 | 1.490 ± 0.625 | no effect/inhibits | reduces/inhibits | 0 | (6) | 1 | (5) |
| 10 | SucEGF(3F)1NalE-NH₂ | 0.157 ± 0.037 | 0.320 ± 0.240 | inhibits/inhibits | inhibits/inhibits | 0 | (12) | 3 | (0) |
| 11 | Suc-EGF(4NO2)1NalE-NH₂ | 1.31 ± 0.198 | 1.965 ± 0.635 | reduces/inhibits | reduces/inhibits | 12 | (6) | 14 | (28) |
| 12 | Suc-QGF(3F)F(4NO₂)E-NH₂ | 2.56 ± 0.374 | 5.491 ± 2.271 | no effect/reduces | no effect/inhibits | 0 | (0) | 0 | (0) |
| 13 | Suc-EGYF(4NO₂)E-NH₂ | 2.07 ± 0.346 | 17.713 ± 5.196 | no effect/inhibits | no effect/inhibits | 11 | (31) | 0 | (0) |
| 14 | Suc-EGF(3F)WE-NH₂ | 0.095 ± 0.009 | 0.455 ± 0.030 | reduces/inhibits | reduces/inhibits | 5 | (6) | 0 | (0) |
| 15 | Suc-EGF(3F)HE-NH₂ | 0.521 ± 0.059 | 0.400 ± 0.021 | reduces/inhibits | reduces/inhibits | 3 | (18) | 12 | (83) |
| 16 | Ac-dEGF(3F)1NalE-NH₂ | 0.112 ± 0.017 | 0.590 ± 0.282 | reduces/inhibits | reduces/inhibits | 0 | (7) | 3 | (8) |
| 17 | Ac-dEGF(3F)WE-NH₂ | 0.102 ± 0.017 | 0.250 ± 0.059 | reduces/inhibits | reduces/inhibits | 10 | (27) | 3 | (0) |
| 18 | Bz-dEGF(3F)WE-NH₂ | 0.173 ± 0.072 | 0.4240 ± 0.067 | reduces/inhibits | reduces/inhibits | 0 | (0) | 0 | (5) |
| 19 | Et(CO)-dEGF(3F)WE-NH₂ | 0.145 ± 0.026 | 0.150 ± 0.065 | reduces/inhibits | reduces/inhibits | 1 | (10) | 0 | (10) |
| 20 | MeO(CO)-dEGF(3F)WE-NH₂ | 0.076 ± 0.014 | 0.053 ± 0.015 | reduces/inhibits | inhibits/inhibits | 0 | (16) | 6 | (8) |
| 21 | Ts-dEGF(3F)WE-NH₂ | 0.029 ± 0.005 | 0.190 ± 0.042 | reduces/inhibits | inhibits/inhibits | 19 | (25) | 9 | (0) |
| 22 | 4-(MeO)—PhSO₂-dEGF(3F)WE-NH₂ | 0.017 ± 0.002 | 0.048 ± 0.003 | s. reduces/inhibits | inhibits/inhibits | 7 | (12) | 0 | (18) |
| 23 | EtO(CO)-dEGF(3F)WE-NH₂ | 0.045 ± 0.005 | 0.034 ± 0.017 | reduces/inhibits | inhibits/inhibits | 4 | (4) | 0 | (12) |
| 24 | Ts-DEGF(3F)WE-NH₂ | 0.018 ± 0.002 | 0.019 ± 0.013 | s. reduces/inhibits | inhibits/inhibits | 0 | (7) | 0 | (8) |
| 25 | Ts-dDGF(3F)WE-NH₂ | 0.025 ± 0.004 | 0.292 ± 0.106 | s. reduces/inhibits | inhibits/inhibits | 0 | (22) | 1 | (8) |
| 26 | 4-(MeO)—PhSO₂-DEGF(3F)WE-NH₂ | 0.027 ± 0.002 | 0.495 ± 0.163 | s. reduces/inhibits | inhibits/inhibits | 15 | (3) | 0 | (8) |
| 27 | Ts-dEGF(3F)WD-NH₂ | 0.034 ± 0.005 | 0.680 ± 0.152 | s. reduces/inhibits | inhibits/inhibits | 4 | (6) | 3 | (8) |

### Example 7

### Cell-Based Activity of Peptidomimetics

The activity of 4-(MeO)-PhSO₂-dEGF(3F)WE-NH₂ in a cell was investigated as an example of activity seen with this family of compounds.

### In-Cell Western Assay for PDCD4 Accumulation.

PDCD4 is a substrate of βTrCP and so an inhibitor of βTrCP should result in the stabilisation and accumulation of PDCD4 in cells. To measure this an in-cell western assay was used and the peptidomimetics were delivered to the cells by nucleofection.

### Nucleofection

MCF7 cells were grown in 10 cm dishes in 10 ml DMEM+10% FBS and 1% Pen/Strep at 37° C./5% CO₂. On the day of nucleofection, the dish was washed with 90% confluent MCF7 cells with 5 ml PBS, then 3 ml of Trypsin/EDTA was added and incubated at 37° C./5% CO₂ for approximately 5 mins until the cells detached from the plastic. 7 ml of normal growth media was added and the number of cells present was counted using a haemocytometer.

The cells were centrifuged cells at 90 g for 10 mins at RT and the supernatant was removed. 100 μl of Nucleofection Buffer V+Supplement was added (Lonza Biologics Cat no VCA-1003) per 8×10⁵ cells, and the cells were added to peptidomimetics dissolved in <3% DMSO.

The cell/peptidomimetic mix was added to cuvettes supplied for the nucleofector and the sample was nucleofected using the recommended MCF7 programme for high cell viability (i.e. E-014). 500 μl of TPA (12-O-tetradecanoylphorbol-13-acetate)-supplemented growth media (i.e. DMEM/10% FBS/1% Pen-Strep/10 nM TPA) was added to the cuvette and 100 μl of this solution was transferred to a well of a 96 well plate and incubated at 37° C./5% CO₂ for 8 hours.

### In Cell Western

The celled were fixed by removing the growth media, adding 3.7% Formaldehyde in PBS and incubating at RT for 20 mins. The plate was washed three times in PBS. And the cells were permeabilised by washing 5× for 5 mins each in PBS+0.1% Triton X-100.

The cells were blocked by adding 3% BSA in PBS-Tween to the cells and incubating at RT for 1.5 hours. An anti-PDCD4 antibody (Abcam cat no: ab80590) was added at a concentration of 1:1000 in 3% BSA in PBS-T (50 ul/well) and incubated at RT for 2.5 hours. The cells were washed 5× for 5 mins each with PBS-T before the secondary antibody (LiCor Biosciences Donkey Anti-Rabbit IRDye 800CW cat no 926-32213) was added at a concentration of 1:1000, along with the DNA stain DRAQ5 at a concentration of 1:10000, in 3% BSA in PBS-T (50 ul/well) and incubated at RT for 1 hour, protected from light.

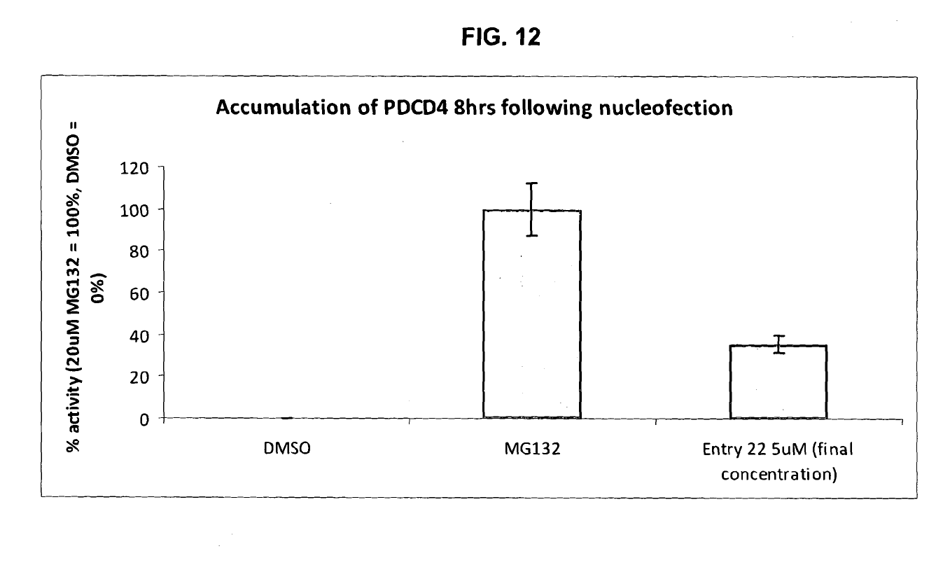

The cells were washed cells 5× for 5 mins each with PBS-T and all the liquid was removed from the wells before the plate was read on the LiCor Biosciences Odyssey at 700 nm and 800 nm. The reading was normalised in the 800 nm channel to that in the 700 nm channel. The data are shown in FIG. 12, where DMSO was used as a negative control and 20 μM MG132 was used as a positive control. The data is presented as percentage activity with DMSO=0% and 20 μM MG132=100% activity. Error bars represent standard deviation from 3 replicates.

### Fluorescence Reporter Assay for PDCD4 Accumulation

This assay uses two stable cell lines expressing PDCD4 fused to a GFP tag. One cell line (MCF7:GFP-PDCD4^{WT}) shows an increase in nuclear fluorescence when βTrCP is inhibited due to the accumulation of GFP-PDCD4 in the nucleus. The other cell line (MCF7:GFP-PDCD4^{S71A/S76A} or MCF7:GFP-PDCD4^{Mut}) does not show an increase in nuclear fluorescence when βTrCP is inhibited because of a mutation of two serine residues in the phosphodegron of PDCD4 that are required for βTrCP recognition. This allows false positives to be identified, where accumulation of PDCD4 is not due to stabilisation by inhibiting βTrCP.

### Nucleofection

The two stable cell lines MCF7:GFP-PDCD4^{WT} and MCF7:GFP-PDCD4^{S71A7S76A} were grown in 10 ml DMEM+10% FBS and 1% Pen/Strep supplemented with 2 mg/ml Geneticin at 37° C./5% CO₂. These cell lines were nucleofected as described above except for the additional supplementation of all media with 2 mg/ml Geneticin.

### Fluorescent Reporter Assay

The cells were fixed by removing the growth media and adding 3.7% Formaldehyde in PBS to the cells. The cells were then incubated at RT for 20 mins and the plates washed 3× in PBS.

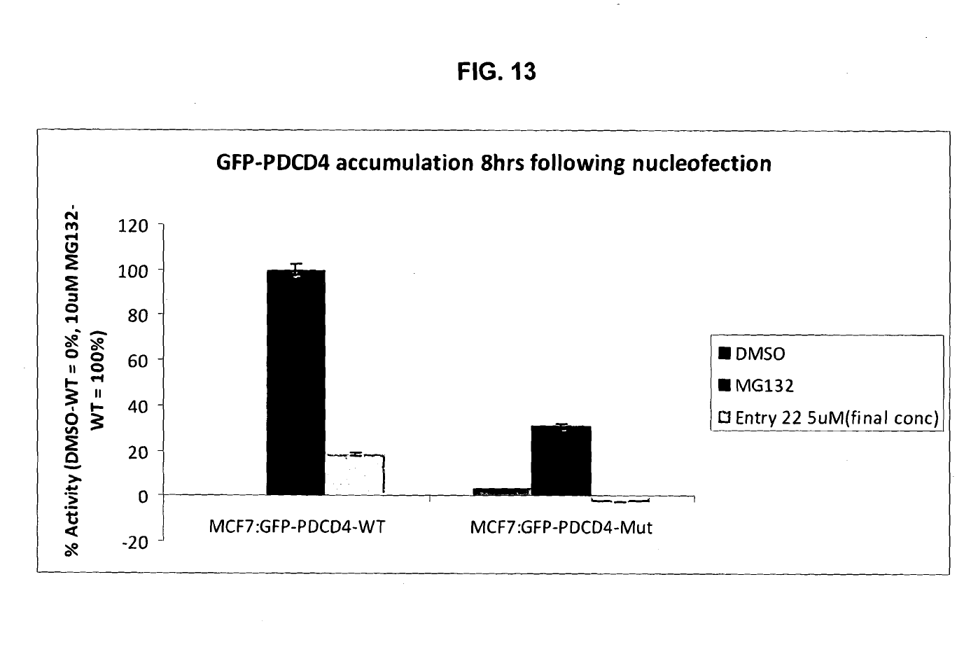

DRAQ5 DNA stain was added to the cells in PBS at a concentration of 1:10000 and incubated at RT for 1 hour protecting from light. The plate was washed 3× in PBS and read on the Perkin Elmer OPERA platform using the nuclei counting algorithm F in both the 488 nm channel (GFP) and 640 nm channel (DRAQ5). The number of GFP-positive nuclei was expressed as a percentage of total number of nuclei, and the data are shown in FIG. 13. DMSO was used as a negative control and 10 μM MG132 was used as a positive control. Error bars represent standard deviation from 6 replicates. The data are expressed as percentage activity with DMSO (WT)=0% and 10 μM MG132 (WT)=100% activity.

### Example 8

### Cell Based Activity of Cell Permeable Compounds

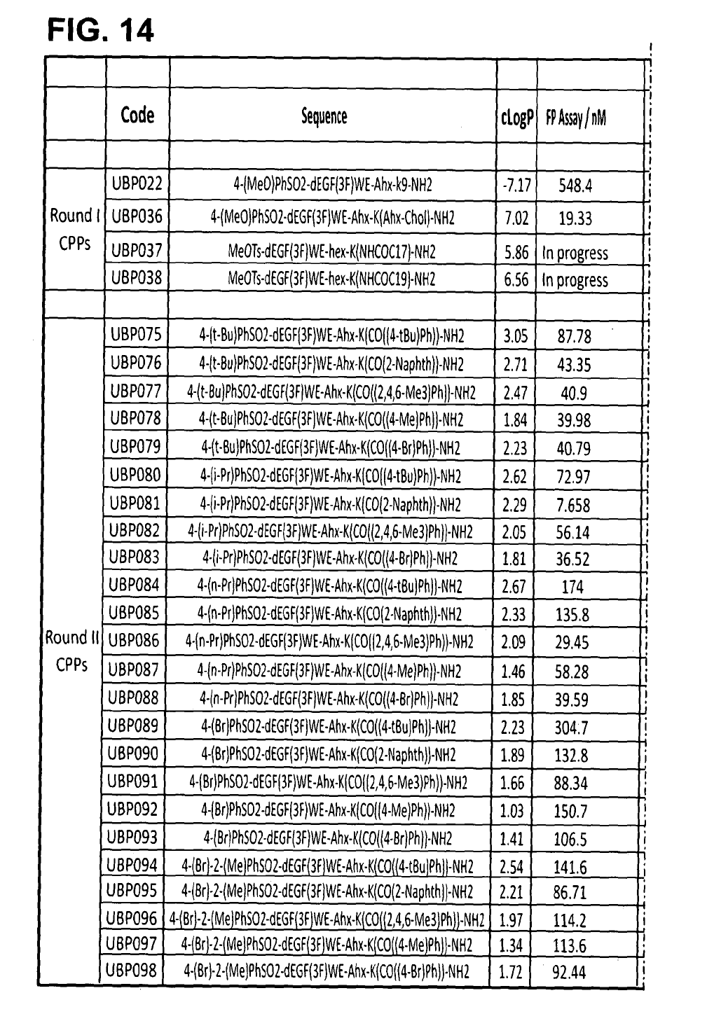

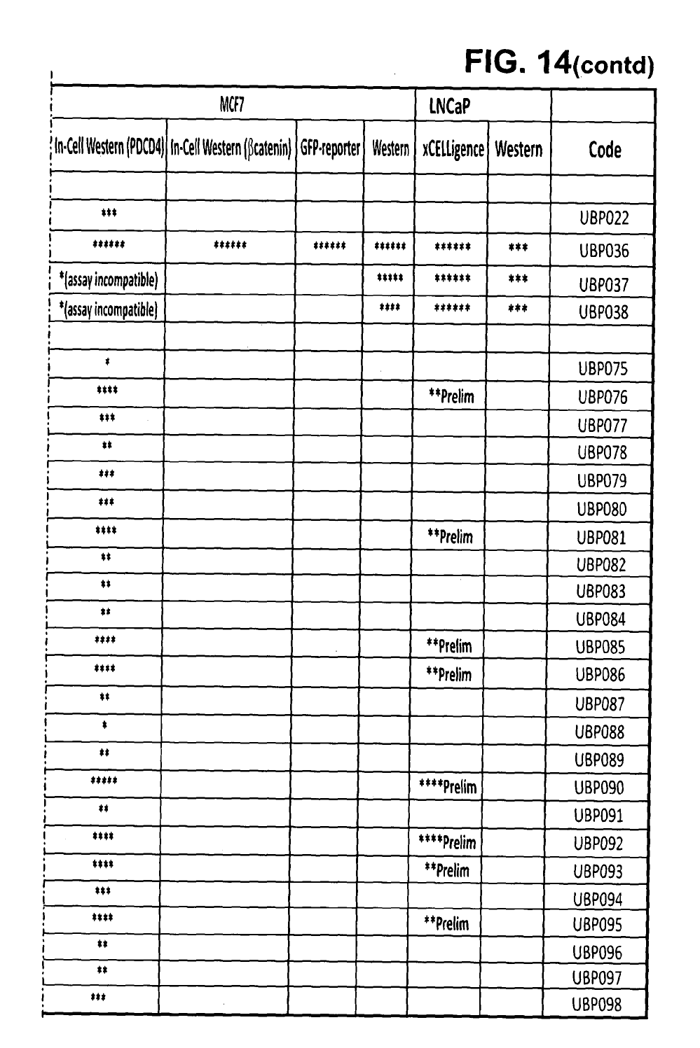

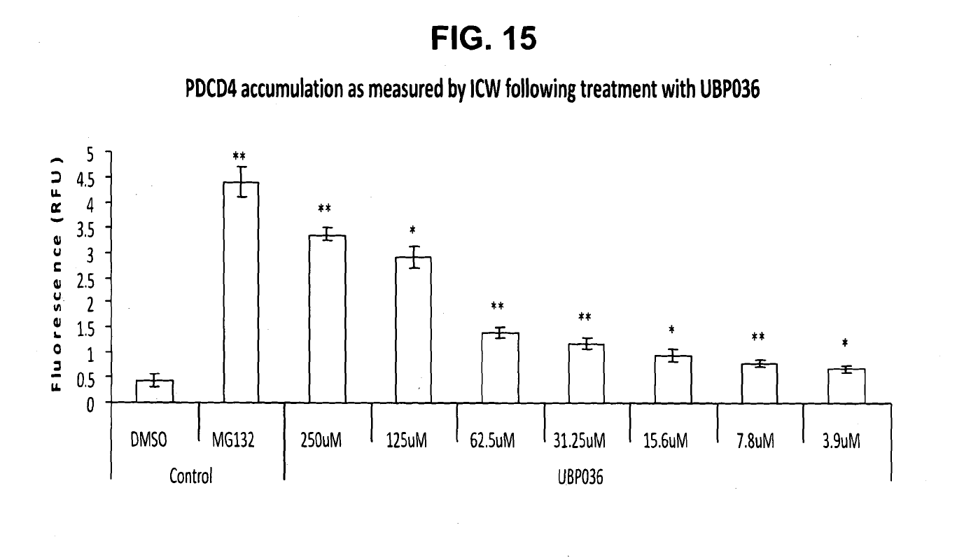

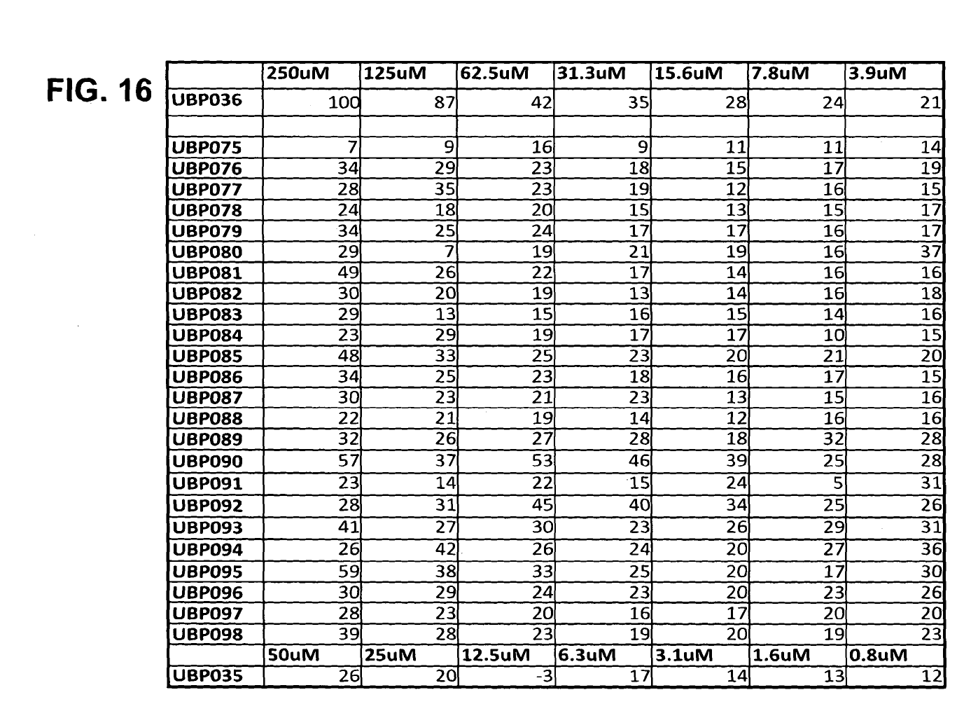

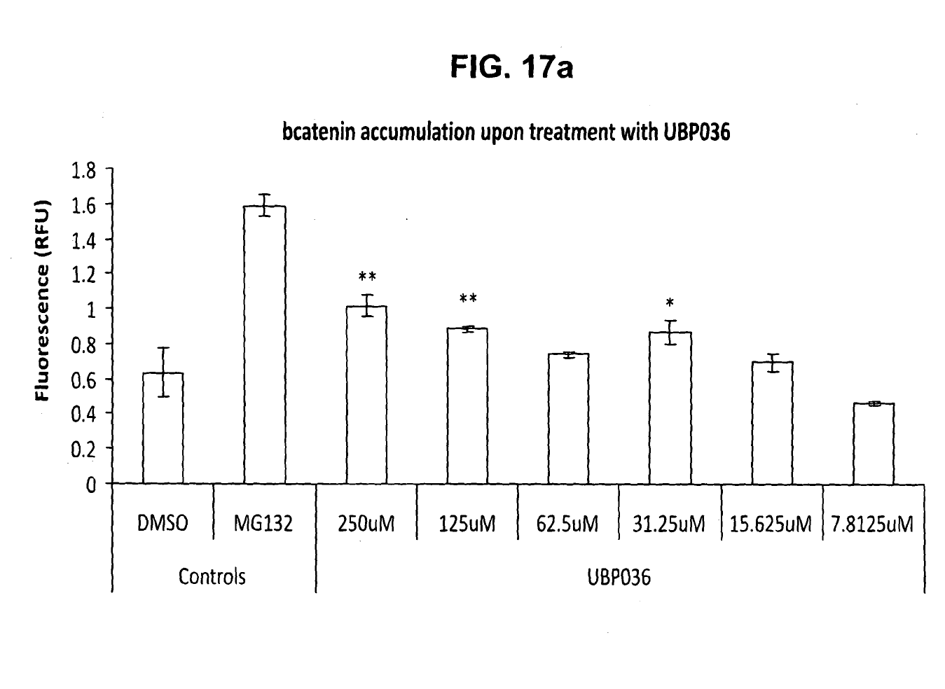

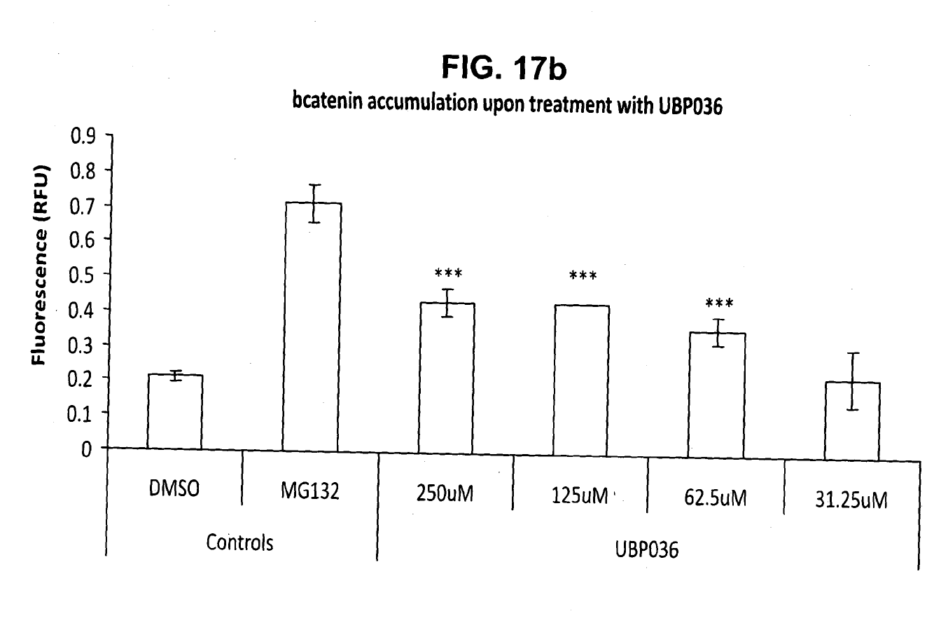

The compounds shown in FIG. 14 were synthesised to improve cell permeability. FIG. 15 shows the accumulation of PDCD4 as measured by the in cell western assay described above, following treatment of MCF7 cells with UBP036. FIG. 16 compares the round II compounds (shown in FIG. 14) with UBP036 in this assay. FIG. 17 shows the accumulation of β-catenin, a further substrate of βTrCP, as measured by the in cell western assay following treatment of MCF7 cells with UBP036. Inhibition of βTrCP, which is responsible for the degradation of both PDCD4 and β-catenin, causes a concomitant stabilisation and accumulation of levels of these proteins which can be measured by in cell western assay.

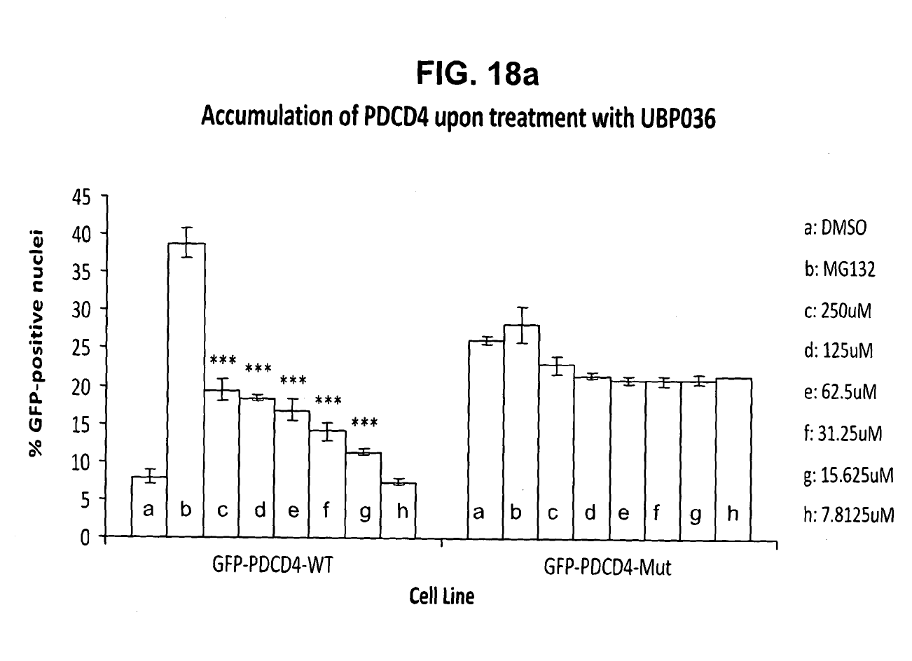

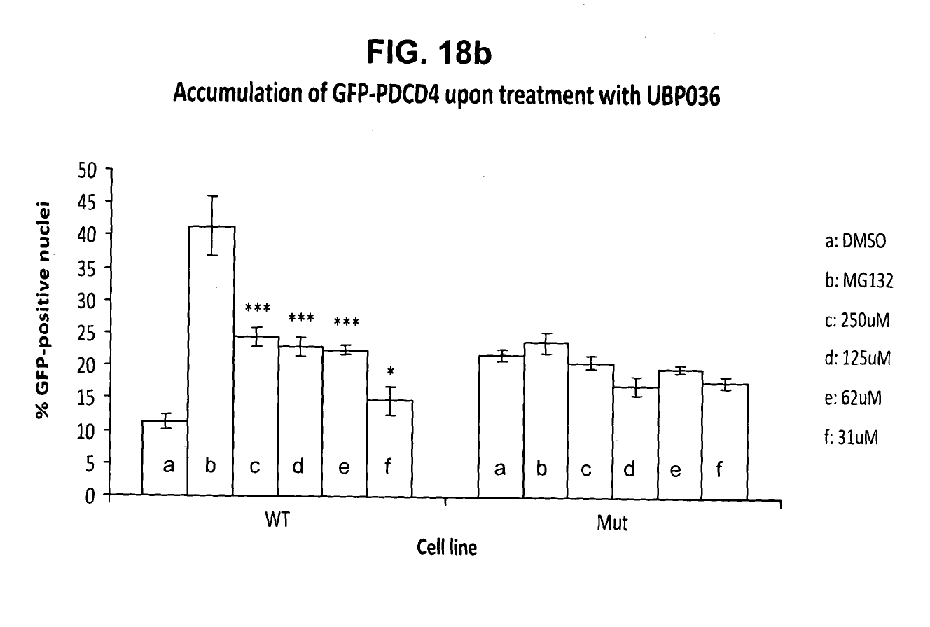

FIG. 18 shows the accumulation of PDCD4 as measured by the fluorescence reporter assay described in example 7. There is accumulation of GFP-PDCD4^{WT}, while GFP-PDCD4^{Mut}, which had been mutated such that the interaction between PDCD4 and βTrCP is abolished, shows no accumulation or stabilisation. This confirms that the stabilisation resulting from the compounds is dependent on the βTrCP/PDCD4 interaction and is not due to another mechanism such as a global increase in protein production.

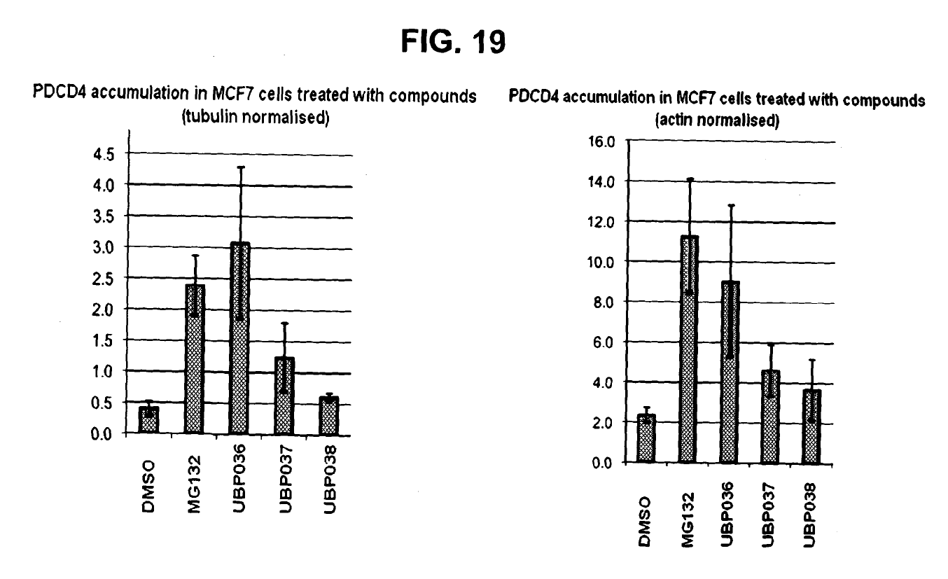

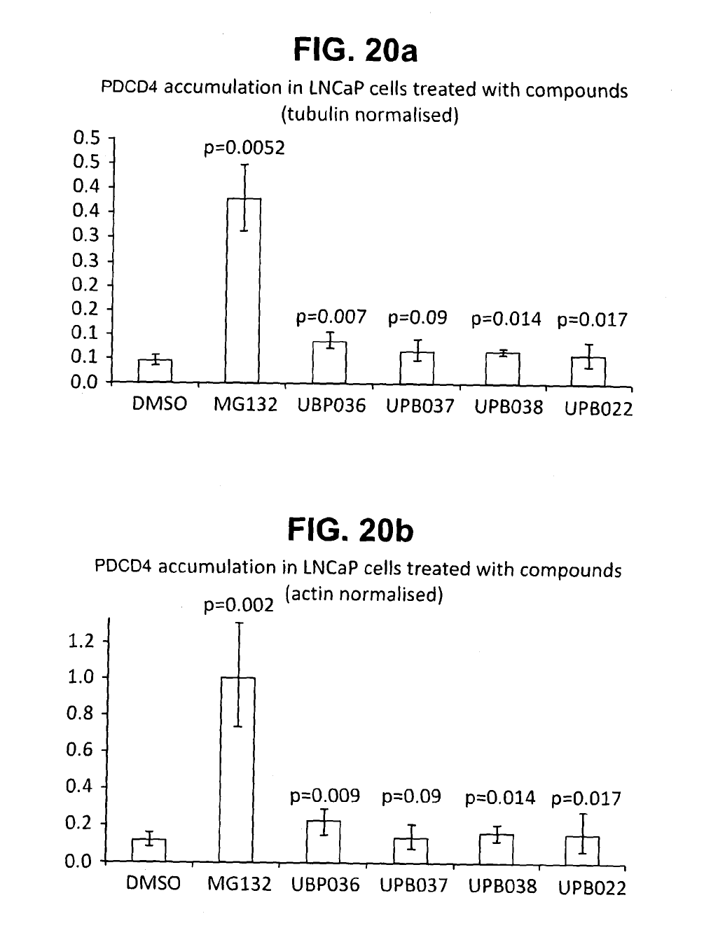

Testing of UBP037 and UBP038 by in cell western provided inconclusive results. The fluorescence readings were consistently under those of the DMSO negative control. This suggested that the compounds were interfering in some way with the fluorescence readout assay. When these compounds were tested by traditional western blot however, they exhibited cellular activity. The results for PDCD4 accumulation in MCF7 cells are shown in FIG. 19. The results for PDCD4 accumulation in LNCaP cells are shown in FIG. 20. The western blot and in cell western are based on the same scientific principles and differ only in the technology used. It was concluded that UBP037 and UBP038 are incompatible with the in cell western technology, but the western blot data shows that these compounds inhibit βTrCP.

### β-Catenin in Cell Western

Plating of the MCF7 cells onto 96 well plates—this step is carried out 1 day before the treatment of the cells to allow the cells to adhere well to the tissue culture plastic. MCF7 cells to be used for seeding should be less than 100% confluent in a 10 cm dish. Add 3 ml of RT trypsin/EDTA to the cells. Incubate at 37° C./5% CO₂ for a few minutes until the cells easily come away from the plastic by gentle swirling. Add 7 ml of media to the cells. Wash the bottom of the plate gently with the 10 ml to ensure all cells are captured and dispense into a 15 ml falcon. Add 100 μl of the cells to 100 μl of Trypan blue and add to haemocytometer to count the number of cells present. Make up approx 10 ml of cells in media at 2×10⁴ cells/1000 (well) with fresh media. Add this correct seeding density to a 10 cm dish. Dispense 100 μl of cells/well into a 96 well plate without using the outside wells. Incubate at 37° C./5% CO₂ overnight Treatment of MCF7 cells with compound of interest (COI) and controls—Put OptiMEM into 37° C. water bath to warm. Add 4 ul of 50 mM compound X for testing to eppendorf—this is tube 1. Add 2 μl DMSO to 6 tubes marked tube 2-7. Take 2 μl of 50 mM (COI) and add to 2 μl of DMSO in tube 2 and pipette up and down to mix. Take 2 μl from tube 2 and add to 21 DMSO in next tube and mix. Repeat until 2 μl in all 7 tubes and have 1:2 serial dilutions from tube 1 to 7 (final conc.: 250 μM to 2 μM). Add 3.5 μl DMSO in tube marked “DMSO” (final percentage 0.5% as for all compounds). Add 0.7 μl of 10 mM MG132 and 2.8 μl DMSO in tube marked “MG132” (final cone.: 10 μM). Add 0.7 μl of 10 mM MLN4924 and 2.8 μM DMSO in tube marked “MLN4924” (final conc. 10 uM). Add 400 μl of pre-warmed OptiMEM to each of tubes 1-7 with serial dilution of compound X. Add 700 ul of pre-warmed OptiMEM to tubes marked “DMSO”, “MG132” and “MLN4924”. Take plate with seeded MCF7 cells and remove the media from the first column of wells. Add 100 μl of “DMSO” to each of six wells in first column of wells. Remove media from the second column of wells and add 10 μl of “MG132” to each of six wells in second column of wells. Remove media from the third column of wells and add 100 μl of “MLN4924” to each of six wells in third column of wells. Remove media from the remaining columns of wells in small batches and add 100 μl of each dilution of compound X to three wells in fourth to tenth column of wells. Incubate at 37° C./5% CO₂ for 8 hrs. Add 1 ml of 37% formaldehyde to 9 ml PBS. Remove the media from the plate and add 100 μl of 3.7% formaldehyde to each well. Incubate at RT for 20 mins. Remove formaldehyde and add 100 μl of PBS to each well. Remove PBS and add another 100 μl PBS to each well. Store at 4° C. overnight.

Immunostaining of β-catenin using In Cell Western (ICW) protocol—Add 100 μl of PBS+0.1% triton to each well and incubate at RT with gentle mixing for 5 mins. Replace with fresh PBS+0.1% triton and repeat 4 times. Note—all washing steps must be carried out gently to avoid dislodging cells. Add 100 μl of 3% BSA in PBS-Tween to each well and incubate at RT with gentle mixing for 1 hour. Add 6.5 μl of β-catenin antibody to 6.5 ml of 3% BSA in PBS-Tween. Add 100 μl of primary antibody solution to all bar one of the DMSO-treated wells. This will act as a negative control. It is also possible to not add primary antibody to one well of each treated column of wells in order to have a no primary control for all positive controls and each concentration of COI. Incubate at RT with gentle mixing for 2.5 hours or overnight at 4° C. Add 100 μl of PBS-Tween to each well and incubate at RT with gentle mixing for 5 mins. Replace with fresh PBS-Tween and repeat 4 times. Spin down the vial of anti-rabbit IR800 (LI-COR Biosciences: cat no 926-32213 Donkey Anti-Rabbit IRDye 800CW) at top speed for a few seconds and add 6.5 μl of this to 6.5 ml of 3% BSA in PBS-Tween. Add 50 μl of this secondary antibody solution to one well as a control for DRAQ5. Add 0.65 μl of DRAQ5 to the secondary antibody solution and add 100 μl of this to all other wells. Incubate at RT with gentle mixing for 1 hr with the plates protected from light with tin foil. Add 100 μl of PBS-Tween to each well and incubate at RT with gentle mixing for 5 mins. Replace with fresh PBS-Tween and repeat 4 times

### Traditional Western Blot

Seed MCF7 or LNCaP cells in 12 well plates at 100,000 cells/well in complete medium (RPMI 1640, 10% fetal bovine serum, 100 units/mL penicillin, 100 ug/mL streptomycin, and 2 mmol/L glutamine, all from GIBCO) and incubate at 37° C./5% CO₂ overnight.
24 hrs later change media to complete media+compound of interest +10 nM TPA and incubate for 8 hrs
After incubation, wash cells three times in cold PBS and lyse with 3T3 lysis buffer containing protease inhibitors (Roche).
Protein quantification was determined by the Bradford assay (BIORAD).
Run 5 ug of protein samples on 4-12% NuPAGE Bis-Tris gels (Invitrogen) and transfer onto nitroceullulose membrane (Whatman).
Block membranes in 50% PBS, 50% Odyssey block (LI-COR) for 1 hour.
Incubate blots with primary antibodies diluted in 49% PBST, 49% Odyssey block and 0.5% 10% Tween-20 (Sigma) overnight at the following concentrations:
   Anti-PDCD4 (Abcam) at 1/10000
   Anti-Tubulin (Abcam) at 1/20000
   Anti-Actin (Abcam) at 1/5000.
Wash blots 3 times for 5 min in PBST.
Incubate with secondary antibodies on blots for 1 hour, protected from light.
   Antibodies were diluted in the same solution as primary antibodies
   1/20000 goat anti-mouse alexa 680 (LI-COR),
   1/10000 donkey anti-rabbit alexa 800 (LI-COR).
Wash blots 3 times for 5 min in PBST.
Observe protein bands using L¹-COR Odyssey reader and quantitate strength of bands with Odyssey software.

### Example 9

### Cell Based Activity of Cell Permeable Compounds on the xCELLigence Platform

The effects of the compounds shown in FIG. 14 were tested on cancer cell lines MCF7 (breast cancer) and LNCaP (prostate cancer) using the xCELLigence platform (http://www.roche-applied-science.com/sis/xcelligence/ezhome.html). This method of measuring cell viability via measuring cell index is known in the art.

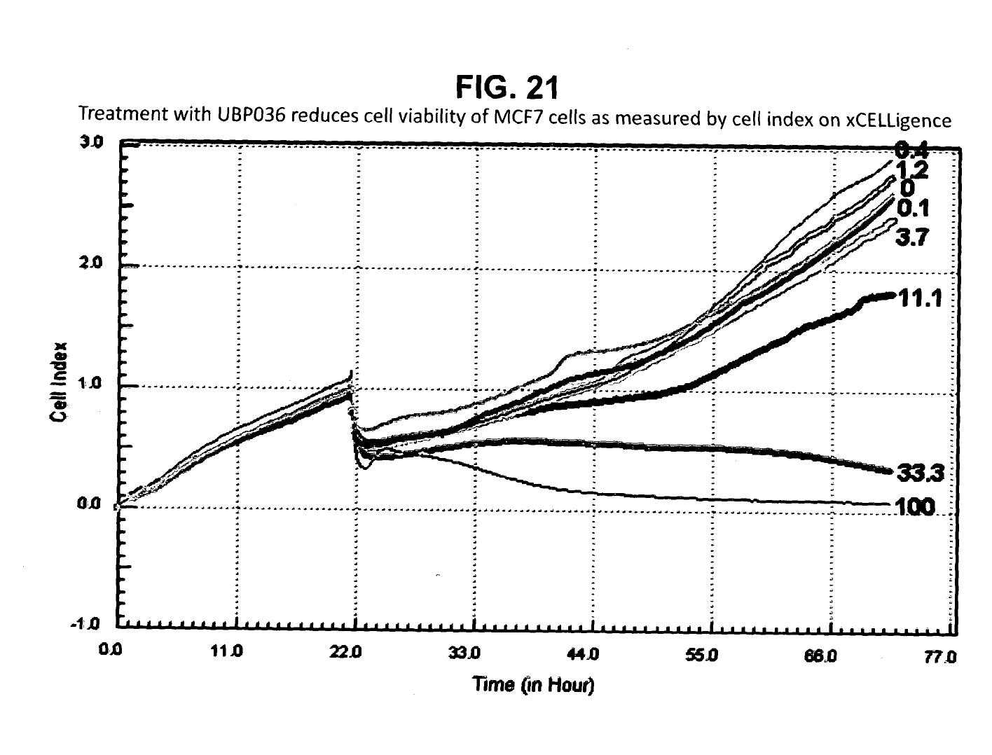

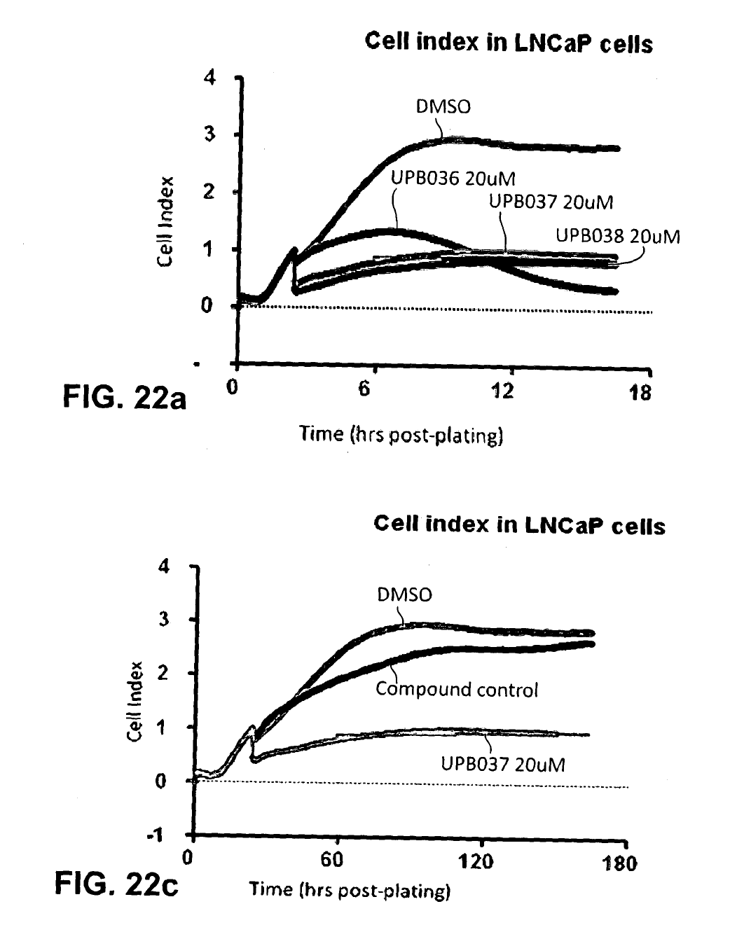

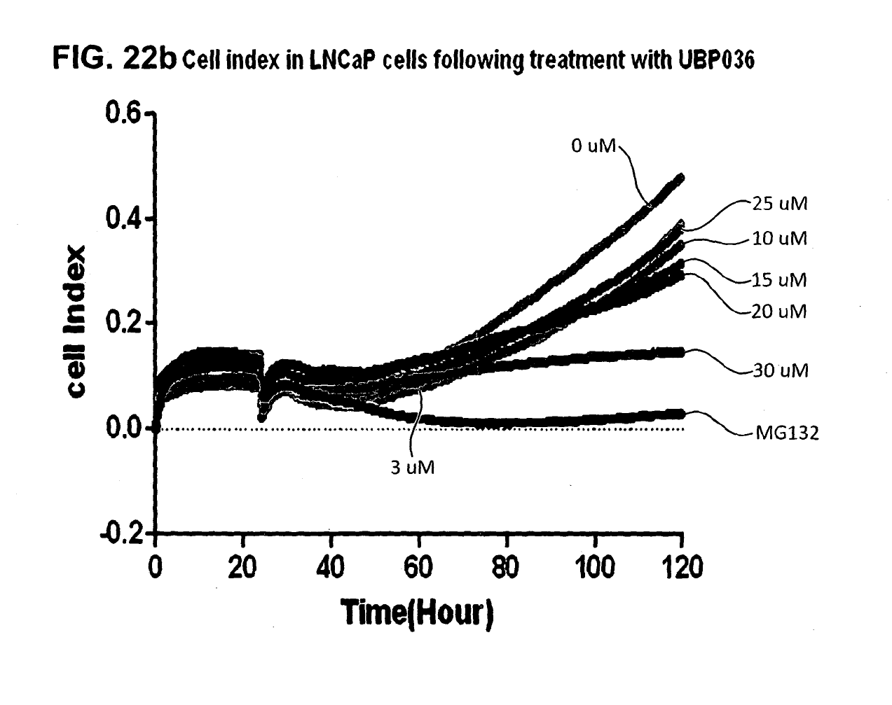

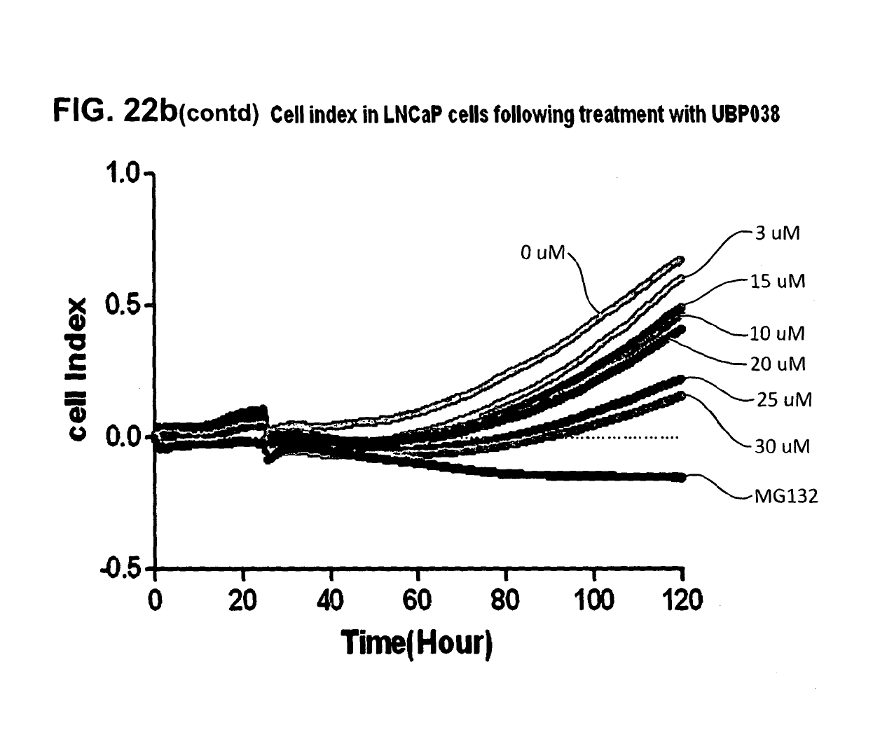

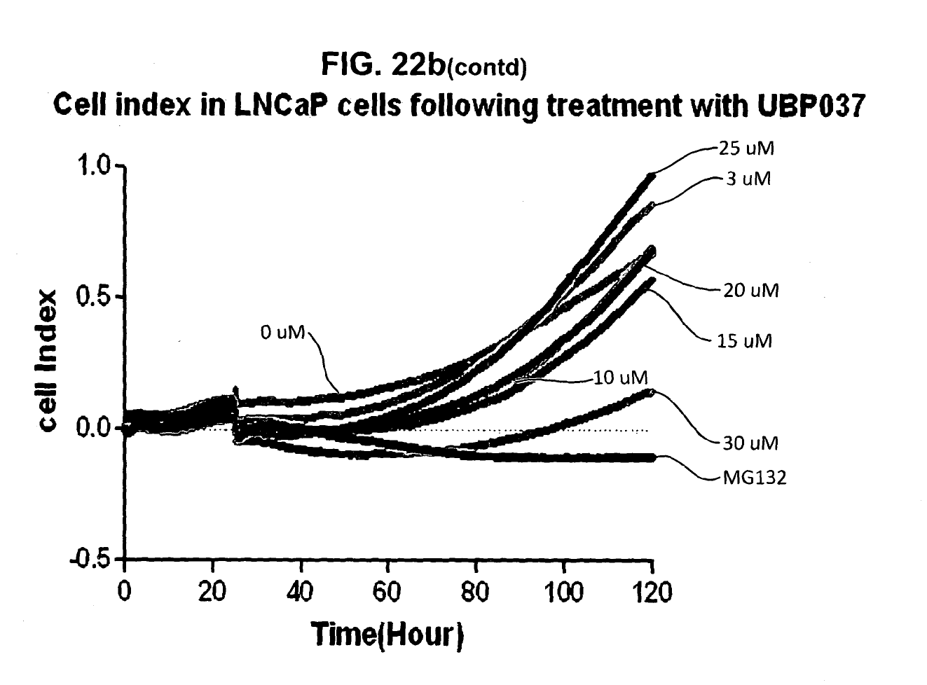

Different doses of the compounds were tested on MCF7 cells to see if there would be an effect on their cell viability. FIG. 21 shows that increasing compound concentration is concomitant with decreased cell viability. These experiments were repeated with LNCaP cells as shown in FIG. 22 (B).

In order to prove that this activity was specific to the active compounds of the invention, a control compound was developed that is identical to the active compound except for the amino acid sequence that confers the specificity of the active compound. The control compound was compared to its partner active compound (UBP037). FIG. 22 (C) shows that the loss in cell viability (LNCaP cells) seen with the active species is not seen with the control compound.

xCELLigence Method

Before starting the experiment add 50 μL of medium (RPMI 1640, 10% fetal bovine serum, 100 units/mL penicillin, 100 ug/mL streptomycin, and 2 mmol/L glutamine, all from GIBCO) to each well of 96 well E-plate (Roche) and place the plate in the xCELLigence platform to measure the background.
LNCaP/PNT-1 cells are seeded (LNCaPs 80000 cells/well; PNT1 3000 cells/well) in the plate in complete medium and equilibrated for 60 mins at RT before returning to the xCELLigence platform and incubating at 37° C./5% CO₂.
24 hrs after seeding, remove 100 μL of medium was removed from each well and cells were treated with compounds or DMSO diluted in 100 μL of OptiMEM (GIBCO).
Replace the plate in the xCELLigence platform
Cells growth can then be monitored in real-time by the cell index profile on the xCELLigence readout screen
Cell growth can be expressed as normalized cell index and doubling time/slope calculated using the RTCA software.

### Example 10

### Activity in Cancer Cells Lines Compared to Non-Cancer Cell Lines

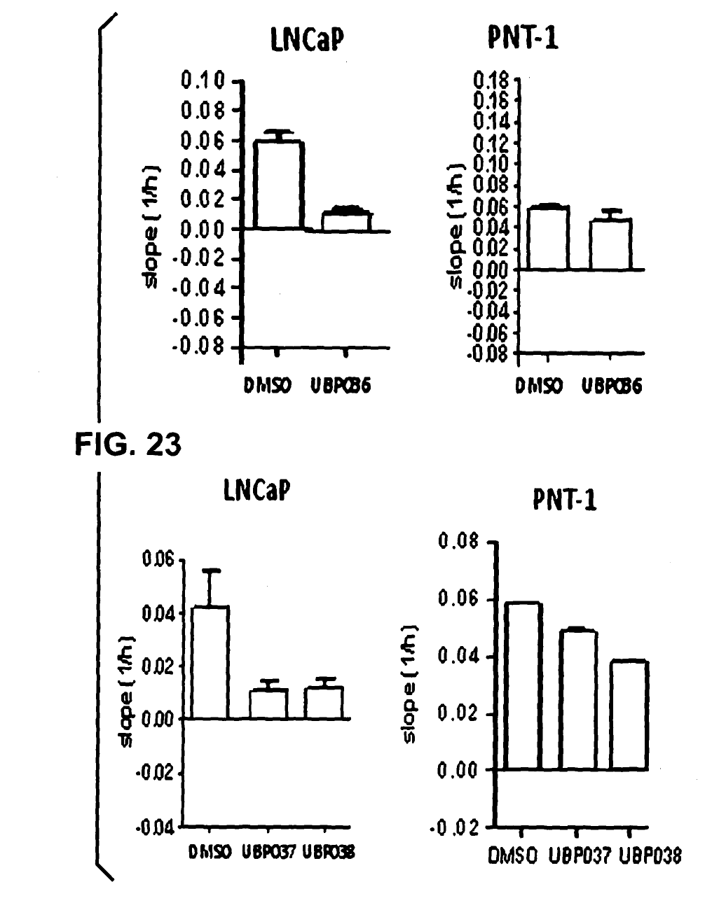

The susceptibility of cancer cell lines versus non-cancer cell lines following treatment with the compounds was also investigated. FIG. 23 shows that UBP036 and UBP037 and UPB038 inhibit the growth of the cancer cell line LNCaP, while having far less effect on the non-malignant PNT-1 cell line.

### Analysis

### 1. Demonstration of Cell Based Activity by Active Compounds in Comparison to an Inactive Compound of a Similar Physicochemical Nature

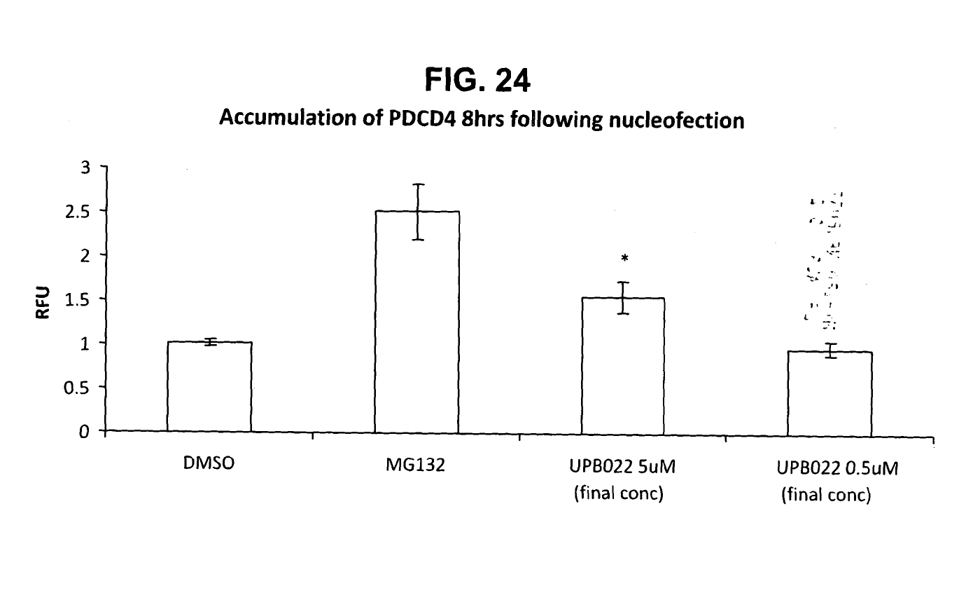

This has been demonstrated by the xCELLigence assay that compared UBP037 with the control compound (Ts-EdFEGW-Ahx-K(Stearic)-NH2, a scrambled version of a compound of the present invention (see FIG. 22 (C)). UBP037 severely restricts the proliferation of the LNCaP cells, while the control compound shows levels of cell proliferation similar to that seen with DMSO. Therefore, the activity seen in UBP037 is entirely due to the active peptide moiety and not the delivery vehicle (stearic acid). In addition, UBP036, UBP022, UBP090 all display cell based activity. The delivery vehicle in all these examples is very different (cholesterol, poly-lysine, LogP manipulation) with the only common feature being the active species. If cell based activity is due to an off-target effect, it is highly unlikely that all three vehicles would hit the same non-specific target. Finally the potency of these cell active species is exactly that seen with the “naked” active peptide upon nucleofection into the cells (see FIG. 24) and thus any off-target effect on the same range of biomarkers seen by three separate CPP delivery moieties would be highly unlikely.

### 2. Demonstration of Target-Specific Activity in a Cell Based Assay Format

This is illustrated for compound UBP036 when examined in the OFP reporter (FIG. 18). When PDCD4 is mutated to eliminate the PDCD4-βTrCP interaction, there is no accumulation of PDCD4 in response to treatment with UBP036. This implies that any increase in PDCD4 seen in the wild type construct is entirely dependent on the interaction between βTrCP and PDCD4.

In addition, the use of two βTrCP substrates; PDCD4 and β-catenin, in the in cell western assay would also suggest that any effect seen is due to βTrCP inhibition.

Again the active species of all the compounds in FIG. 14 is identical, the only difference between them being the cell delivery vehicle. As discussed, there appears to be no activity associated with the cell-delivery vehicle, and therefore the PDCD4 biomarker activity and cellular proliferation activity seen for all the compounds is βTrCP-dependent.

### 3. Demonstration of Activity in Several Cancer Cell Lines

In the development of the assays for a βTrCP inhibitor a number of cell-line—biomarker combinations were surveyed to identify the most sensitive assay for βTrCP inhibitors. The breast cancer cell-line MCF7 proved extremely responsive to βTrCP inhibition and this could be measured by the rapid and robust accumulation of the βTrCP substrates PDCD4 and β-catenin.

As can been seen from the data presented here all compounds exhibit this activity (to various degrees) in MCF7 cells.

The next phase of development involved addressing the potential therapeutic benefit of βTrCP inhibition in a cell line that could be replicated in an animal model. Here LNCaPs were chosen given previous evidence that βTrCP inhibition inhibited cell proliferation both in vitro and in vivo (PLoS One. 2010 Feb. 5; 5(2):e9060.)

Again it can be seen from the data that all compounds tested exhibit a reduction in proliferation (to various degrees) in LNCaP cells.

In addition to the cellular activity seen in each of these cell lines, it is also becoming apparent that the biomarker activity assays in MCF7 cells appear to predict the therapeutic activity seen in LNCaP cells.

There is also cell viability inhibition in MCF7 cells upon treatment with these compounds (see FIG. 22) revealing the possibility of further therapeutic uses for JTrCP inhibitors in a breast cancer model (and a potential mechanism of action in PDCD4 accumulation).

### Medical Applications of βTrCP Inhibitors

There are a number of potential indications for βTrCP inhibitors including many forms of cancer.

Two key indications exemplified are prostate cancer and breast cancer.

### Breast Cancer:

There is clinical evidence that βTrCP2 is over expressed in a number of cancers including breast cancer [J Biol. Chem. 2002, 277, 36624-30]. This study also demonstrated that the cell lines used to model breast cancer such as MCF7 cells also display this same overexpression when compared to non-cancer cell lines such as MCF10A. This implies that work done on βTrCP inhibition in these cancer cell lines could indeed reflect potential outcomes in a clinical setting.

There have been numerous in vive studies to demonstrate the importance of PTrCP in mammary development. In βTrCP1−/− mice there is a hypoplastic phenotype observed where cell proliferation is reduced by 50% in the mammary gland with other organs unaffected. Furthermore, when there is exogenous high expression of βTrCP1 introduced in the mammary epithelia, approx 40% of mice develop carcinomas. [Mol Cell Biol. 2004, 24, 8184-94.]. The value of this study is two-fold. It demonstrates that despite the widespread expression of βTrCP, a systemic reduction in βTrCP levels (via the genetic ablation of βTrCP I) has a preferential effect on the mammary gland. Also it reveals that overexpression of βTrCP1 can in itself result in an increased cancer risk in this tissue. This suggests inhibition of βTrCP may be of value in both breast cancers that do not display βTrCP overexpression (as inhibition of βTrCP in healthy animals appears to preferentially target the mammary gland for reduced cell proliferation) and those that do (due to the potential causative effect of βTrCP mis-regulation).

In addition to the value of inhibiting βTrCP alone to affect favourable outcomes in breast cancer, there is also work to suggest that combining βTrCP inhibition with some of the current therapies for breast cancer could improve the outcome of these therapies. Inhibition of βTrCP by an RNAi approach suppressed growth and survival of human breast cancer cells [Cancer Res. 2005 Mar. 1; 65(5):1904-8]. It is worth noting that these experiments were carried out on both ER-positive and ER-negative breast cancer cell lines with βTrCP inhibition having a similar impact on both. In addition, inhibition of βTrCP augmented the anti-proliferative effects of anticancer drugs such as doxorubicin, tamoxifen, and paclitaxel on human breast cancer cells. These data suggest that βTrCP inhibition could be effective as a front line adjuvant therapy or in combination with an existing breast cancer treatment regime.

We have shown that the βTrCP inhibitors described here inhibit binding of βTrCP to IκBα (a well-known βTrCP substrate) in in vitro binding assays and stabilise several JTrCP substrates in cell based assays. In addition this inhibitor can reduce the cell viability of a breast cancer cell line in a similar fashion to βTrCP RNAi.

These data and the studies described above show that βTrCP is a validated, novel target in breast cancer, and that its inhibition is tractable and of clinical significance.

### Prostate Cancer:

Here the main evidence for the role of βTrCP is from the work of Yinon Ben-Neriah and Eli Pikarsky [PLoS One. 2010 Feb. 5; 5(2):e9060.] Their key finding here was not only that inhibition of βTrCP resulted in the loss of cell viability of LNCaP cells in vitro—but also that when this inhibition is transferred from cells to animals through the use of LNCaP xenografts—it results in a loss of growth of prostate tumours and in combination with androgen ablation—the lack of tumour growth entirely.

### Example 11

### Effect of Capping Groups on Peptide Activity

A number of different C-terminal and N-terminal capping groups were added to peptide d-E-G-F(3F)-W-E-NH₂ in order to demonstrate how the inhibitory activity of d-E-G-F(3F)-W-E-NH₂ is affected by particular capping groups, which act to increase cell penetration (as illustrated by AcLogP, relative to UBP022). Kᵢ and ΔcLogP values for the capped compounds are shown in Table 12.

TABLE 12
Kᵢ and ΔcLogP for N- and C-terminal capped peptide d-E-G-F(3F)-W-E-NH₂

| | | Average | | Ki/ |
|---|---|---|---|---|
| Code | Sequence | Ki/nM | ΔcLogP | ΔcLogP |
| UBP022 | 4-(MeO)PhSO₂-dEGF(3F)WE-NH₂ | 5.775 | | |
| UBP054 | 4-(MeO)PhSO₂-dEGF(3F)WE-Ahx-K(NHCO-(4-(t-Bu)-Ph))-NH₂ | 1.938 | 3.84 | 0.505 |
| UBP055 | 4-(MeO)PhSO₂-dEGF(3F)WE-Ahx-K(NHCO-2-Naph)-NH₂ | 1.851 | 3.51 | 0.527 |
| UBP056 | 4-(MeO)PhSO₂-dEGF(3F)WE-Ahx-K(NHCO-(2,4,6-(Me)₃-Ph))-NH₂ | 3.387 | 3.27 | 1.036 |
| UBP057 | 4-(MeO)PhSO₂-dEGF(3F)WE-Ahx-K(NHCO-(4-(Me)-Ph))-NH₂ | 2.884 | 2.64 | 1.092 |
| UBP058 | 4-(MeO)PhSO₂-dEGF(3F)WE-Ahx-K(NHCO-(4-(Br)-Ph))-NH₂ | 3.3455 | 3.03 | 1.104 |
| UBP059 | 4-(MeO)PhSO₂-dEGF(3F)WE-Ahx-K(NHSO₂-(4-(Br)-Ph))-NH₂ | 2.960 | 2.40 | 1.2333 |
| UBP060 | 4-(MeO)PhSO₂-dEGF(3F)WE-Ahx-K(NHOO-(4-(Cl)-Ph))-NH₂ | 3.641 | 2.94 | 1.238 |
| UBP061 | 4-(MeO)PhSO₂ dEGF(3F)WE-Ahx-K(NHOOPh)-NH₂ | 3.105 | 2.33 | 1.333 |
| UBP062 | 4-(MeO)PhSO₂-dEGF(3F)WE-Ahx-K(NHOO-(3,5-(Cl)₂-Ph))-NH₂ | 6.229 | 3.55 | 1.755 |
| UBP063 | 4-(MeO)PhSO₂-dEGF(3F)WE-Ahx-K(NHOO-(CH₂)₄CH₃)-NH₂ | 5.374 | 2.71 | 1.983 |
| UBP064 | 4-(MeO)PhSO₂-dEGF(3F)WE-Ahx-K(NHOO-(4-(CF₃)-Ph))-NH₂ | 6.391 | 3.09 | 2.068 |
| UBP065 | 4-(MeO)PhSO₂-dEGF(3F)WE-Ahx-K(NHCOO-Ph)-NH₂ | 5.939 | 2.55 | 2.329 |
| UBP066 | 4-(MeO)PhSO₂-dEGF(3F)WE-Ahx-K(NHCO-(4-(OMe)-Ph))-NH₂ | 5.1745 | 2.22 | 2.331 |
| UBP067 | 4-(MeO)PhSO₂-dEGF(3F)WE-Ahx-K(NHCONH-Ph)-NH₂ | 5.608 | 2.22 | 2.526 |
| UBP068 | 4-(MeO)PhSO₂ dEGF(3F)WE-Ahx-K(NHCO-CH₂CH₂CH(CH₃)₂)-NH₂ | 5.378 | 2.12 | 2.537 |
| UBP069 | 4-(MeO)PhSO₂-dEGF(3F)WE-Ahx-K(NHCOO-1-Naph)-NH₂ | 11.608 | 3.73 | 3.112 |
| UBP070 | 4-(MeO)PhSO₂ dEGF(3F)WE-Ahx-K(NHCO-(4-(Cl)-2,6(Fl₂-Ph))-NH₂ | 11.455 | 3.06 | 3.743 |
| UBP071 | 4-(MeO)PhSO₂ dEGF(3F)WE-Ahx-K(NHCO-(4-(Me₂N)-Ph))-NH₂ | 9.506 | 2.33 | 4.080 |
| UBP072 | 4-(MeO)PhSO₂ -dEGF(3F)WE-Ahx-K(NHSO₂-(4-(i-Pr)-Ph))-NH₂ | 12.941 | 2.80 | 4.622 |
| UBP073 | 4-(MeO)PhSO₂-dEGF(3F)WE-Ahx-K(NHSO₂Ph)-NH₂ | 13.005 | 1.70 | 7.65 |
| UBP074 | 4-(MeO)PhSO₂-dEGF(3F)WE-Ahx-K(NHSO₂-(4-(n-Pr)-Ph)-NH₂ | 28.585 | 2.84 | 10.065 |
| UBP040 | 4-i-Pr-PhSO₂-dEGF(3F)WE-NH₂ | 1.603 | 1.20 | 1.336 |
| UBP041 | 4-Pr-PhSO₂-dEGF(3F)WE-NH₂ | 1.768 | 1.24 | 1.426 |
| UBP042 | 4-Br-PhSO₂-dEGF(3F)WE-NH₂ | 2.519 | 0.8 | 3.14 |
| UBP043 | 4-Br-2-CH₃-PhSO₂-dEGF(3F)WE-NH₂ | 3.961 | 1.12 | 3.537 |
| UBP044 | 2-Naph-SO₂-dEGF(3F)WE-NH₂ | 5.814 | 1.29 | 4.507 |
| UBP045 | 4-OCF₃-PhSO₂-dEGF(3F)WE-NH₂ | 4.774 | 0.94 | 5.079 |
| UBP046 | 4-Br-3-CF₃-PhSO₂-dEGF(3F)WE-NH₂ | 14.217 | 1.57 | 9.055 |
| UBP047 | 4-CF₃-PhSO₂-dEGF(3F)WE-NH₂ | 9.450 | 0.94 | 10.862 |
| UBP048 | 2,4-(Cl)₂-PhSO₂-dEGF(3F)WE-NH₂ | 27.025 | 1.33 | 20.320 |
| UBP049 | 2,4-(Br)₂-PhSO₂-dEGF(3F)WE-NH₂ | 45.255 | 1.50 | 30.17 |
| UBP050 | 3,5-(CH₃)₂-PhSO₂-dEGF(3F)WE-NH₂ | 24.645 | 0.74 | 33.30 |
| UBP051 | 4-Br-2-OCF₃-PhSO₂-dEGF(3F)WE-NH₂ | 109.55 | 1.63 | 67.209 |
| UBP052 | 4-I-PhSO₂-dEGF(3F)WE-NH₂ | 252.9 | 1.04 | 243.17 |
| UBP053 | 4-Cl-PhSO₂-dEGF(3F)WE-NH₂ | 330.5 | 0.72 | 459.0 |

As suggested by the data in table 12, modification of the C-terminal capping group has little effect upon activity. Modification of the N-terminal capping group has a more profound effect. The function of the capping groups is to aid cell penetration as demonstrated by the ΔcLogP values. cLogP values are calculated by means well known to the person skilled in the art.

### REFERENCES

Pons et al. (2008) Biochemistry 47, pg. 14-29
Tapia et al. (2008) J. Pept. Sci. 14, pg. 1309-1314
Rautio et al. (2008) Nat. Rev. Drug Discov. 7, pg 255-270. using anti-His and anti-βTrCP antibodies
Remington's Pharmaceutical Sciences
Stocks et al. (2007) On Medicinal Chemistry
Werle et al. (1997) British Journal of Cancer
Bungaard et al. Design of Prodrugs
Ornstein et al. (1993) Bioorg. Med. Chem. Lett
Lakshmann et al (2008) Expert Opinion in Therapeutic Targets 12(7):855-870.
Frescas and Pagano (2008) Nature Reviews Cancer Jun; 8(6):438-49
Nalepa, Rolfe and Harper (2006). Nature Reviews Drug Discovery 5:596-613
Crosetto, Bienko and Dikic (2006) Molecular Cancer Research 4(12): 899-904

SEQUENCES

(consensus sequence)
SEQ ID NO: 1
XXGFXX
(preferred peptide)
SEQ ID NO: 2
dEGF(3F)WE
(preferred peptide)
SEQ ID NO: 3
DEGF(3F)WE
(preferred peptide)
SEQ ID NO: 4
DEGF(3F)WD
(preferred peptide)
SEQ ID NO: 5
dDGF(3F)WD
(preferred peptide)
SEQ ID NO: 6
EGF(3F)WE
(preferred peptide)
SEQ ID NO: 7
dEGF(3F)1NalE
(preferred peptide)
SEQ ID NO: 8
EGF(3F)1NalE
(phosphopeptide substrate)
SEQ ID NO: 9
KKERLLDDRHDpSGLDpSMKDEE
(optimisation starting sequence)
SEQ ID NO: 10
LDpSGIHS
(Vpu phosphodegeneron)
SEQ ID NO: 11
DpSGIHS
(binding peptide)
SEQ ID NO: 12
DpSGIFE
(binding peptide)
SEQ ID NO: 13
DEGIFE
(binding peptide)
SEQ ID NO: 14
ERAEDpSGNEpSEGEIS
(binding peptide)
SEQ ID NO: 15
ERAEDAGNEpSEGEIS
(binding peptide)
SEQ ID NO: 16
ERAEDpSGNEpSEGEHS
(binding peptide)
SEQ ID NO: 17
ERADDpSGNEpSEGEIS
(binding peptide)
SEQ ID NO: 18
dEGIFE
(binding peptide)
SEQ ID NO: 19
dEGIFD
(binding peptide)
SEQ ID NO: 20
dNGIFR
(binding peptide)
SEQ ID NO: 21
DEGFFE
(binding peptide)
SEQ ID NO: 22
dNGFFR
(binding peptide)
SEQ ID NO: 23
EGIFE
(binding peptide)
SEQ ID NO: 24
EGFFE
(binding peptide)
SEQ ID NO: 25
DEGYFE
(binding peptide)
SEQ ID NO: 26
EpSGIFE
(binding peptide)
SEQ ID NO: 27
DpSGIFH
(binding peptide)
SEQ ID NO: 28
DpSGNFE
(binding peptide)
SEQ ID NO: 29
DDpSSGIHS
(binding peptide)
SEQ ID NO: 30
LDpSSGIHS
(binding peptide)
SEQ ID NO: 31
GDpSGIHS
(binding peptide)
SEQ ID NO: 32
ADpSGIHS
(binding peptide)
SEQ ID NO: 33
VDpSGIHS
(control peptide)
SEQ ID NO: 34
DAGIFE
(control peptide)
SEQ ID NO: 35
AGIFE
(control peptide)
SEQ ID NO: 36
AGFFE
(control peptide)
SEQ ID NO: 37
dAGIFR
(control peptide)
SEQ ID NO: 38
dAGIFD
(control peptide)
SEQ ID NO: 39
dAGIFE
(control peptide)
SEQ ID NO: 40
DAGFFE
(control peptide)
SEQ ID NO: 41
DAGYFE
(control peptide)
SEQ ID NO: 42
EAGIFE
(control peptide)
SEQ ID NO: 43
DAGIFH
(control peptide)
SEQ ID NO: 44
DAGNFE
(control peptide)
SEQ ID NO: 45
DAGIHS
(control peptide)
SEQ ID NO: 46
DDASGIHS
(control peptide)
SEQ ID NO: 47
LDASGIHS
(control peptide)
SEQ ID NO: 48
GDAGIHS
(control peptide)
SEQ ID NO: 49
ADAGIHS
(control peptide)
SEQ ID NO: 50
VDAGIHS
(binding peptide)
SEQ ID NO: 51
EGF(3F)HE-NH2
(binding peptide)
SEQ ID NO: 52
dEGF(3F)1Nal-E-NH2
(binding peptide)
SEQ ID NO: 53
EGI1NalE-NH2
(binding peptide)
SEQ ID NO: 54
EGF(3F)1NalQ-NH2
(binding peptide)
SEQ ID NO: 55
EGF(3F)Y(4Me)E-NH2
(binding peptide)
SEQ ID NO: 56
dEGF(3F)WD-NH2

1-107. (canceled)

108. A modified peptide comprising a sequence of amino acids
X¹-E/D/pS-G-X⁴-X⁵-E/D/pS-NHR^{N2} Formula Ic
wherein,
each of the amino acids are selected from L-amino acids, D-amino acids, aza-amino acids and substituted amino acids; and
wherein,
X¹ is a group A¹-B—Z¹—;
X⁴ is a group —N(R^{c})—Y³(-L²-A³)-Z⁴—;
X⁵ is a group —N(R^{d})—Y⁴(-L³-A⁴)-Z⁵—;
wherein,
B is C₁-C₁₀ alkyl, C₂-C₁₀ alkenyl or C₂-C₁₀ alkynyl;
wherein, B may be substituted with one or more R^{E}, wherein R^{E} is selected from the group consisting of C₁-C₄ alkyl, —NH₂, —NH(R^{N2}) and —N(R^{N2})₂;
R^{c} and R^{d} are each independently selected from the group consisting of —H, C₁-C₁₀ alkyl, aryl and heteroaryl;
L² and L³ are each independently C₀-C₅ alkyl, C₂-C₅ alkenyl or C₂-C₅ alkynyl;
wherein,
L² may be substituted with one or more R^{L2}, wherein R^{L2} is C₁-C₄ alkyl or C₂-C₄ alkenyl;
L³ may be substituted with one or more R^{L3}, wherien R^{L3} is C₁-C₄ alkyl;
Y³ and Y⁴ are each independently CH or N;
Z¹ is a bond, C═O, C═S, CH₂, S═O, S(O)₂, C═N(C₁-C₄ alkyl) or C═NH;
Z⁴ and Z⁵ are independently selected from the group consisting of C═O, C═S, CH₂, S═O, S(O)₂, C═N(C₁-C₄ alkyl) and C═NH;
A¹ is carboxylic acid (—CO₂H) or a bioisostere thereof;
wherein, A¹ may be substituted with one or more R^{A1}, wherein R^{A1} is selected from the group consisting of —H, C₁-C₄ alkyl, C₂-C₄ alkenyl and aryl;
A³ and A⁴ are each independently aryl or heteroaryl;
wherein,
A³ may be substituted with one or more R^{A3}, wherein, R^{A3} is selected from the group consisting of —H, —F, —Cl, —Br, —I, —OH, —O(C₁-C₁₀ alkyl), —CN, —NO₂, —CF₃, —OCF₃, —CO₂H, —C₁-C₁₀ alkyl, —NH₂, —NH(C₁-C₂ alkyl) and —N(C₁-C₂ alkyl)₂;
A⁴ may be substituted with one or more R^{A4}, wherein R^{A4} is selected from the group consisting of —H, —F, —Cl, —Br, —I, —OH, —O(C₁-C₁₀ alkyl), —CN, —NO₂, —CF₃, —OCF₃, —CO₂H, —C₁-C₁₀ alkyl, —NH₂, —NH(C₁-C₂ alkyl) and —N(C₁-C₂ alkyl)₂;
wherein;
R^{N2} is selected from the group consisting of R^{N1}, —(CH₂)₀₋₁₀—(Z⁷)₀₋₁-A^{a}, —(CH₂O)O₀₋₁₀—CH₂—(Z⁷)₀₋₁-A^{a}, —(CH₂CH₂O)₁₋₁₀—CH₂CH₃, —(CH₂CH₂O)₁₋₁₀—(CH₂)₁₋₃—(Z⁷)₀₋₁-A^{a};
wherein,
Z⁷ is (C═O);
A^{a} is —OH, —NH₂, —C(O)NH₂, a cholesteryl derivative, a chain of one or more non-naturally occurring amino acids, or a chain of one or more naturally occurring amino acids or a chain of a mixture of one or more naturally occurring amino acids and one or more non-naturally occurring amino acids; wherein the one or more non-naturally occurring or naturally occurring amino acids are independently selected from the group consisting of L-amino acids, D-amino acids and aza-amino acids;
R^{N1} is selected from the group consisting of —H, —C₁-C₁₀ alkyl and aryl.

109. The modified peptide according to claim 108, wherein said modified peptide is of Formula Ig:
Capping group-X¹-E/D/pS-G-X⁴—X⁵-E/D/pS-NHRN² Formula Ig

110. The modified peptide according to claim 108, wherein X¹ is selected from the group consisting of aspartyl, glutamyl, succinyl, maleyl and fumaryl; preferably, wherein X¹ is selected from the group consisting of aspartyl, glutamyl and succinyl; preferably, wherein X¹ is aspartyl.

111. The modified peptide according to claim 108, wherein said modified peptide is of Formula Iv
d-E-G-F(3F)-W-E-NHR^{N2}

112. The modified peptide according to claim 108, wherein the modified peptide comprises an amino/amine group in which a hydrogen atom of said amino/amine group has been replaced by a capping group.

113. The modified peptide according to claim 108, wherein the capping group is selected from the group consisting of 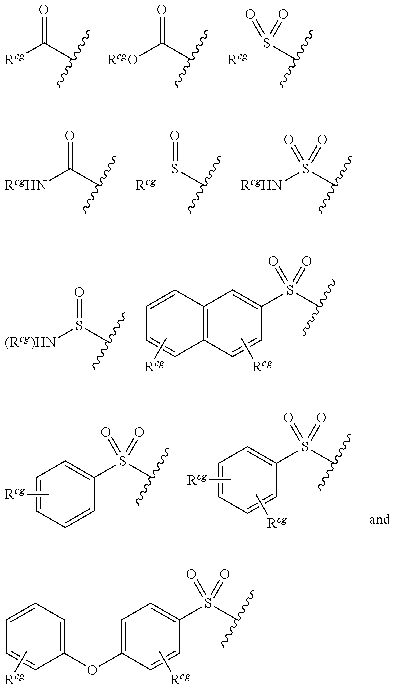
wherein,
R^{cg} is selected from the group consisting of —H, —F, —Cl, —Br, —I, —OH, —O(C₁-C₁₀ alkyl), —CN, —NO₂, —CF₃, —OCF₃, —CO₂H, —NH₂, —NH(C₁-C₂ alkyl), —N(C₁-C₂ alkyl)₂, —C₁-C₁₀ alkyl, aryl and heteroaryl.

114. The modified peptide according to claim 108, wherein the capping group is selected from the group consisting of

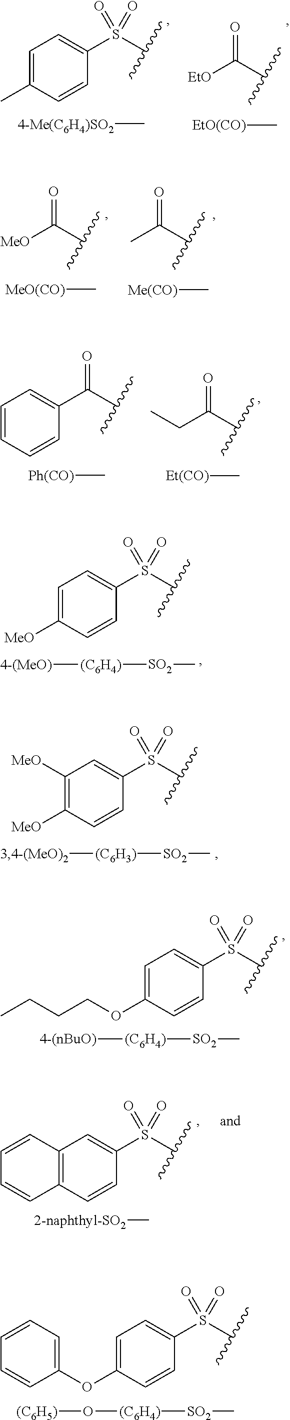

115. The modified peptide according to claim 108, wherein the capping group is selected from List 1 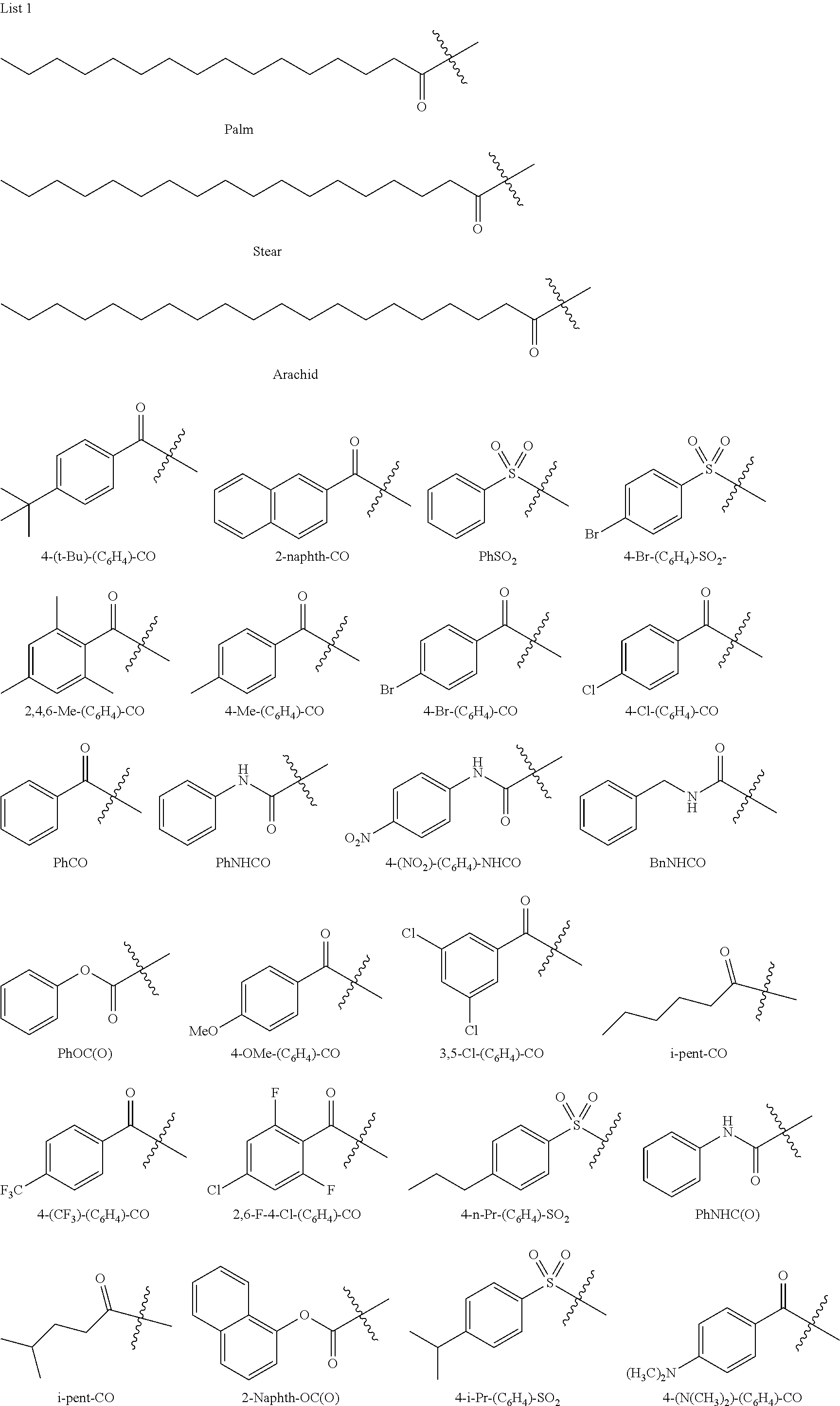 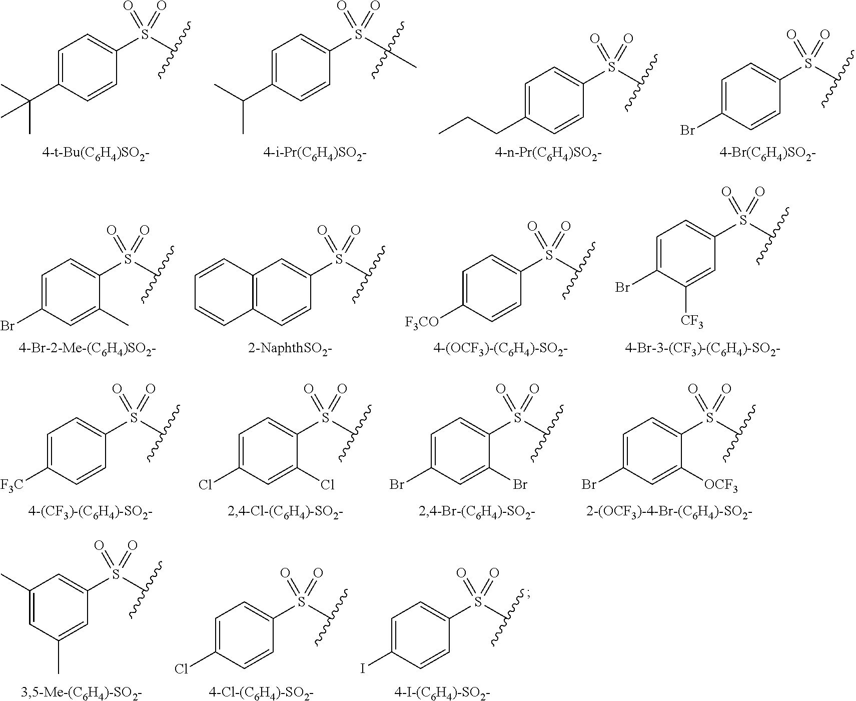
or,
wherein the amino/amine group to be capped is a substituent of group B; or,
wherein the amino/amine to be capped is a substituent of A^{a}; or
wherein the amino/amine to be capped is a substituent of group B, and the capping group is selected from List 2: 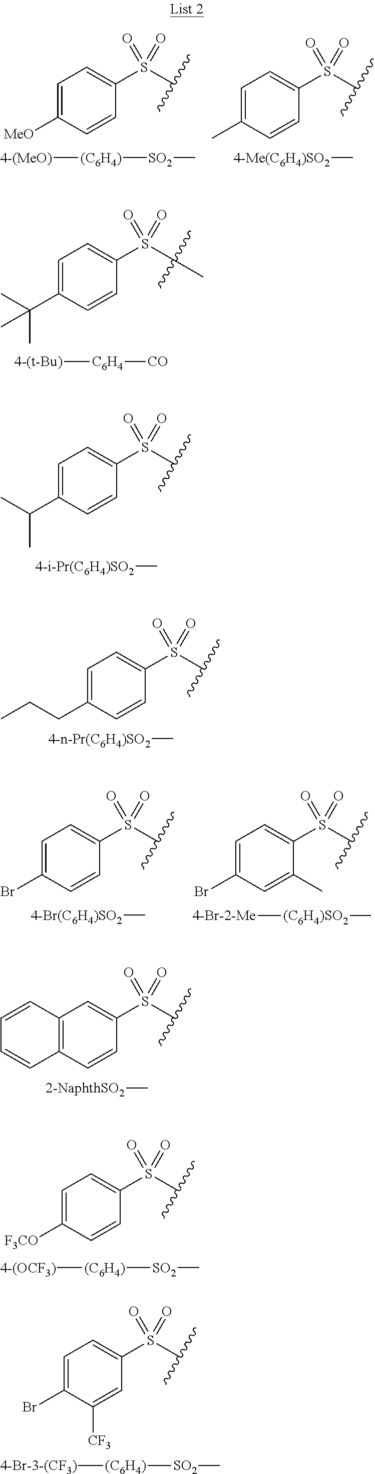 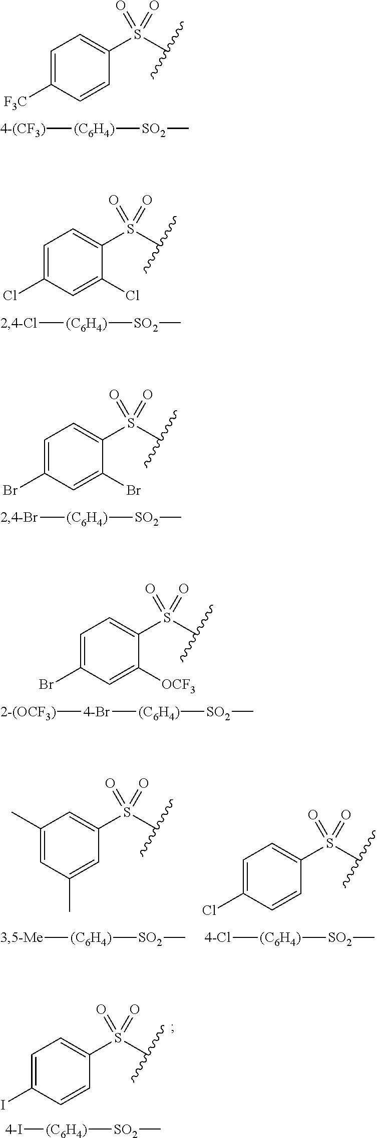
or,
wherein the amino/amine to be capped is a substituent of A^{a}, and the capping group is selected from List 3;

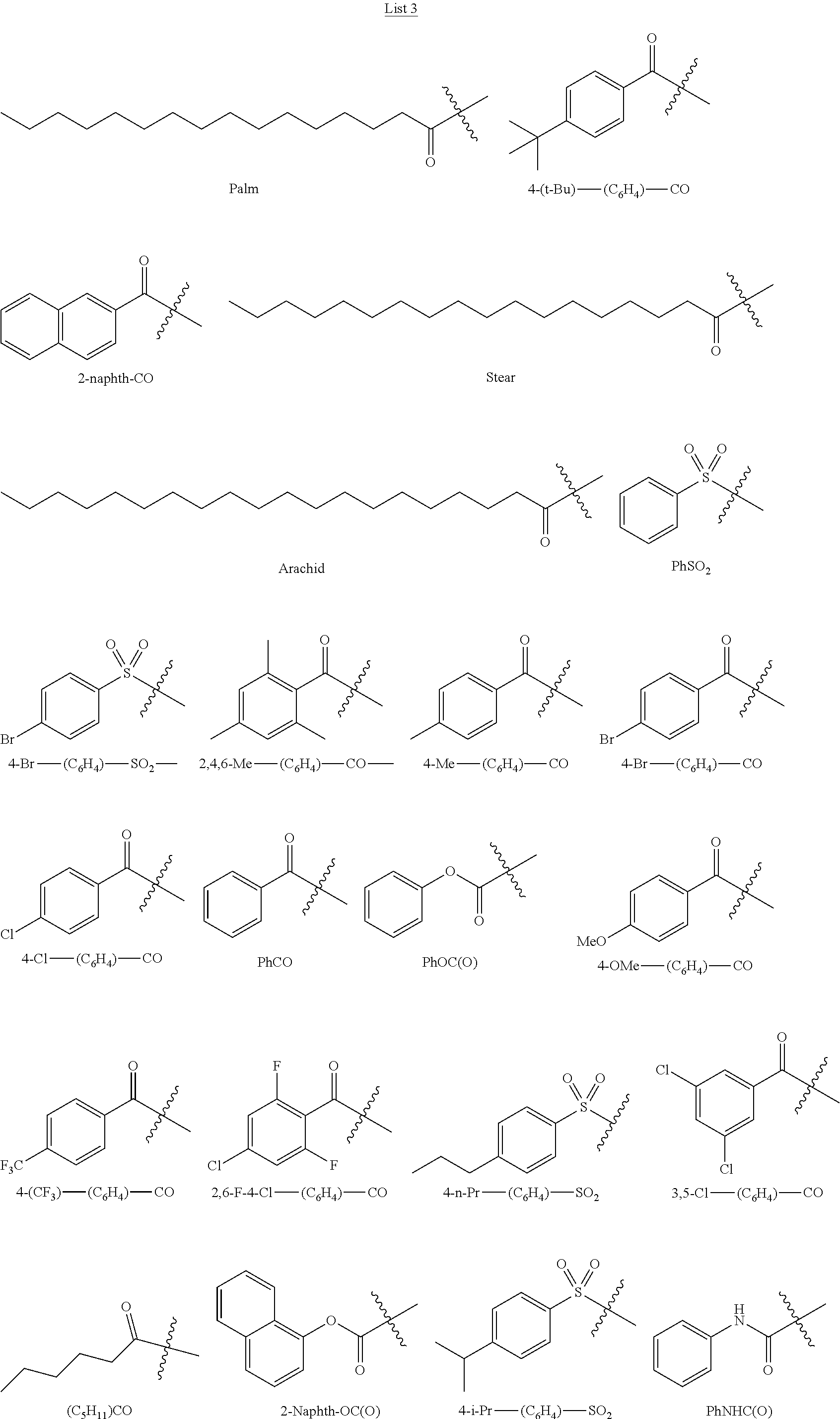

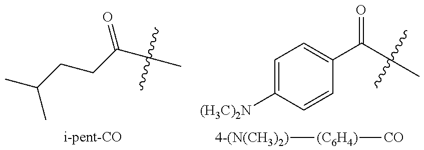

116. The modified peptide according claim 108, wherein X⁴ is phenylalanine substituted with one or more substituents selected from the group consisting of —H, —F, —Cl, —Br, —I, —OH, —O(C₁-C₁₀ alkyl), C₁-C₁₀ alkyl and —NO₂; preferably,
wherein X⁴ is phenylalanine substituted with one or more substituents selected from the group consisting of —H, —F, —OH and —NO₂; preferably,
wherein X⁴ is phenylalanine substituted with —F at one or more of positions 2, 3 and 4; preferably,
wherein X⁴ is F(2F), F(3F), F(4F), F(2Cl), F(3Cl) or F(4Cl).

117. The modified peptide according to claim 108, wherein the modified peptide is cyclised.

118. A modified peptide consisting of the sequence according to claim 108.

119. The modified peptide according to claim 118 having a sequence selected from the group consisting of:
4MeOPhSO2-d-E-G-F(3F)-W-E-NH2;
Ts-D-E-G-F(3F)-W-E-NH2;
Ts-d-E-G-F(3F)-W-E-NH2;
Ts-d-D-G-F(3F)-W-E-NH₂;
4(MeO)PhSO2-D-E-G-F(3F)-W-E-NH₂;
Ts-D-E-G-F(3F)-W-D-NH₂;
3,4-(MeO)₂-PhSO₂-d-E-G-F(3F)-W-E NH₂;
2-NaphthylSO₂-d-E-G-F(3F)-W-E-NH₂;
EtOCO-d-E-G-F(3F)-W-E-NH2;
4-(BuO)-PhSO2-d-E-G-F(3F)-W-E-NH₂;
4-(PhO)-PhSO2-d-E-G-F(3F)-W-E-NH₂;
MeOCO-d-E-G-F(3F)-W-E-NH2;
Ts-d-D-G-F(3F)-W-D-NH₂;
Ac-d-E-G-F(3F)-W-E-NH2;
Suc-E-G-F(3F)-W-E-NH₂;
Ac-d-E-G-F(3F)-1Nal-E-NH₂;
EtCO-d-E-G-F(3F)-W-E-NH₂; and
Suc-E-G-F(3F)-1Nal-E-NH₂.

120. A prodrug comprising a methyl, ethyl, propyl, butyl, pentyl, hexyl, benzyl, aryl or heteroaryl ester of the modified peptide of claim 108.

121. A prodrug comprising a —CO₂(CH₂CH₂O)₁₋₁₀CH₂CH₃ ester of the modified peptide of claim 108.

122. A pharmaceutical composition comprising the modified peptide of claim 108.

123. A method of treating a disease associated with aberrant protein degradation comprising administering the modified peptide of claim 108 in a pharmaceutically effective amount.

124. A method of treating a disease associated with aberrant protein degradation comprising administering the prodrug of claim 120 in a pharmaceutically effective amount.

125. A method of treating a disease associated with aberrant protein degradation comprising administering the prodrug of claim 121 in a pharmaceutically effective amount.

126. A method of treating a disease associated with aberrant protein degradation comprising administering the pharmaceutical composition of claim 122 in a pharmaceutically effective amount.

127. A diagnostic kit comprising the modified peptide of claim 108.

128. A diagnostic kit comprising the prodrug of claim 120.

129. A diagnostic kit comprising the prodrug of claim 121.
